(19) United States
(12) Patent Application Publication
(10) Pub. No.: US 2018/0258429 A1
(43) Pub. Date: Sep. 13, 2018

(54) SARNA COMPOSITIONS AND METHODS OF USE

(71) Applicant: MiNA THERAPEUTICS LIMITED, London (GB)

(72) Inventors: Pål Sætrom, Trondheim (NO); Endre Bakken Stovner, Trondheim (NO)

(21) Appl. No.: 15/978,670

(22) Filed: May 14, 2018

Related U.S. Application Data
(63) Continuation of application No. 15/305,910, filed on Oct. 21, 2016.
(60) Provisional application No. 61/982,429, filed on Apr. 22, 2014.

(51) Int. Cl. C12N 15/113

(52) U.S. Cl. CPC C12N 15/113; C12N 2310/14; C12N 2310/113

(57) ABSTRACT

The invention relates to a saRNAs useful in upregulating the expression of a target gene and therapeutic compositions comprising the saRNA. Methods of using the saRNA and the therapeutic compositions are also provided.

### CROSS-REFERENCE TO RELATED APPLICATIONS

This application is a continuation application of U.S. application Ser. No. 15/305,910 filed Oct. 21, 2016, entitled saRNA Compositions and Method of Use, which is a 35 U.S.C. § 371 U.S. National Stage Entry of International Application No. PCT/GB2015/051189 filed Apr. 22, 2015, entitled saRNA Compositions and Method of Use, which claims priority to U.S. Provisional Application No. 61/982,429 filed Apr. 22, 2014, entitled saRNA Compositions and Method of Use, the contents of each of which are incorporated herein by reference in its entirety.

### REFERENCE TO SEQUENCE LISTING

The instant application contains a Sequence Listing which has been submitted electronically in ASCII format and is hereby incorporated by reference in its entirety. Said ASCII copy, created on May 14, 2018 is named 2058-1014USCON_SL.txt and is 2,208,086 bytes in size.

### FIELD OF THE DISCLOSURE

The invention relates to oligonucleotide, specifically saRNA, compositions for modulating target gene expression and to the methods of using the compositions in diagnostic and therapeutic applications such as treating metabolic disorders, hyperproliferative diseases, and regulating the nervous system and/or immune system.

### BACKGROUND OF THE INVENTION

Recently it has been discovered that short RNAs can regulate transcription by destructing transcripts that are sense or antisense to a given mRNA and which are presumed to be non-coding transcripts. Destruction of the non-coding transcripts which are sense, or identical to, the given mRNA results in transcriptional repression of that mRNA, whereas destruction of the non-coding transcripts which are antisense to the given mRNA results in transcriptional activation and/or increased expression of the mRNA or the level of protein encoded by the mRNA. By targeting such non-coding transcripts, short RNAs can therefore be used to upregulate specific genes at either the nucleic acid or protein. Small duplex RNAs have also been discovered to increase gene expression by targeting ncRNAs that overlap gene promoters (Janowski et al., Nature Chemical Biology, vol. 3:166-173 (2007), the contents of which are incorporated herein by reference in their entirety).

Any short RNA which leads to up-regulation of the expression of a target gene by any mechanism is termed a short activating RNA or small activating RNA (saRNA). Known methods of up-regulating a target gene by use of saRNAs can involve the detection of an RNA transcript which is antisense to the target gene of interest and designing short RNA molecules which down-regulate the identified transcript. For instance, U.S. Pat. No. 8,288,354 to Wahlestedt, the contents of which are incorporated herein by reference in their entirety, discloses a method of modulating expression of a target gene comprising targeting a nucleic acid molecule to a naturally-occurring anti-sense transcript (NAT) of a sense strand of the targeted gene in a target cell, wherein the nucleic acid molecule targeting the NAT is complementary to the NAT. The NAT may be a coding RNA transcript or a non-coding RNA transcript lacking any extensive open reading frame. In another example, WO 2012/065143 to Krieg et al., the contents of which are incorporated herein by reference in their entirety, teaches a method of activating expression of a target gene comprising blocking the binding of a long non-coding RNA (Inc-RNA) to Polycomb repressive complex 2 (PRC2) protein by a single-stranded oligonucleotide, thereby preventing the Inc-RNA from suppressing the target gene.

There remains, however, a need for compositions and methods for the targeted modulation of genes via activation with saRNA which do not require the a priori identification of a NAT or rely on interactions at the polycomb complex for prophylactic diagnostic and/or therapeutic purposes.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a schematic illustrating the relationships among the nucleic acid moieties involved in the function of an saRNA of the invention.

FIG. 2A-2E show transfections of APOA1-saRNAs in HepG2 cells upregulate the expression of APOA1 gene.

FIG. 3A-3E show transfections of LDLR-saRNAs in AML12 cells upregulate the expression of LDLR gene.

### SUMMARY OF THE INVENTION

The present invention provides compositions, methods and kits for the design, preparation, manufacture, formulation and/or use of short activating RNA (saRNA) that modulates target gene expression and/or function for therapeutic purposes, including diagnosing and prognosis.

It is understood by those of skill in the art that the term saRNA may refer to a single saRNA or saRNA in the plural (saRNAs).

One aspect of the invention provides an saRNA that targets an antisense RNA transcript of a target gene. The antisense RNA transcript of the target gene is referred to thereafter as the target antisense RNA transcript. The target antisense RNA transcript is transcribed from the coding strand of the target gene.

Non-limited examples of the target gene according to the present invention include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), and coagulation factor VIII (F8).

Another aspect of the invention provides a pharmaceutical composition comprising a saRNA that targets an antisense RNA transcript of a target gene and at least one pharmaceutically acceptable excipient, wherein the expression of the target gene is up-regulated.

Another aspect of the invention provides a method of modulating the expression of a target gene comprising administering a saRNA that targets an antisense RNA transcript of the target gene. Non-limited examples of the target gene include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), and coagulation factor VIII (F8).

Another aspect of the invention provides treating or preventing a disease comprising administering a saRNA that targets an antisense RNA transcript of a target gene, wherein the target gene is associated with the disease. Non-limited examples of the target gene include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), and coagulation factor VIII (F8). Non-limited examples of diseases include a metabolic disorder, a hyperproliferative disorder, an immune disorder and/or a neurological disorder.

The details of various embodiments of the invention are set forth in the description below. Other features, objects, and advantages of the invention will be apparent from the description and the drawings, and from the claims.

### DETAILED DESCRIPTION

The present invention provides compositions, methods and kits for modulating target gene expression and/or function for therapeutic purposes. These compositions, methods and kits comprise at least one saRNA that upregulates the expression of a target gene.

I. Design and Synthesis of saRNA

One aspect of the present invention provides a method to design and synthesize saRNA.

The terms “small activating RNA”, “short activating RNA”, or “saRNA” in the context of the present invention means a single-stranded or double-stranded RNA that upregulates or has a positive effect on the expression of a specific gene. The saRNA may be single-stranded of 14 to 30 nucleotides. The saRNA may also be double-stranded, each strand comprising 14 to 30 nucleotides. The gene is called the target gene of the saRNA. As used herein, the target gene is a double-stranded DNA comprising a coding strand and a template strand. For example, an saRNA that upregulates the expression of the APOA1 gene is called an “APOA1-saRNA” and the APOA1 gene is the target gene of the APOA1-saRNA. A target gene may be any gene of interest. In some embodiments, a target gene has a promoter region on the template strand.

By “upregulation” or “activation” of a gene is meant an increase in the level of expression of a gene, or levels of the polypeptide(s) encoded by a gene or the activity thereof, or levels of the RNA transcript(s) transcribed from the template strand of a gene above that observed in the absence of the saRNA of the present invention. The saRNA of the present invention may have a direct upregulating effect on the expression of the target gene.

The saRNAs of the present invention may have an indirect upregulating effect on the RNA transcript(s) transcribed from the template strand of the target gene and/or the polypeptide(s) encoded by the target gene or mRNA. The RNA transcript transcribed from the target gene is referred to thereafter as the target transcript. The target transcript may be an mRNA of the target gene. The target transcript may exist in the mitochondria. The saRNAs of the present invention may have a downstream effect on a biological process or activity. In such embodiments, an saRNA targeting a first transcript may have an effect (either upregulating or downregulating) on a second, non-target transcript.

### Target Antisense RNA Transcript of a Target Gene

In one embodiment, the saRNAs of the present invention is designed to be complementary to a target antisense RNA transcript of a target gene, and it may exert its effect on the target gene expression and/or function by down-regulating the target antisense RNA transcript. The target antisense RNA transcript is transcribed from the coding strand of the target gene and may exist in the nucleus of a cell.

The term “complementary to” in the context means being able to hybridize with the target antisense RNA transcript under stringent conditions.

The term “antisense” when used to describe a target antisense RNA transcript in the context of the present invention means that the sequence is complementary to a sequence on the coding strand of a gene.

It is to be understood that thymidine of the DNA is replaced by uridine in RNA and that this difference does not alter the understanding of the terms “antisense” or “complementarity”.

The target antisense RNA transcript may be transcribed from a locus on the coding strand between up to 100, 80, 60, 40, 20 or 10 kb upstream of a location corresponding to the target gene's transcription start site (TSS) and up to 100, 80, 60, 40, 20 or 10 kb downstream of a location corresponding to the target gene's transcription stop site.

In one embodiment, the target antisense RNA transcript is transcribed from a locus on the coding strand located within +/−1 kb of the target gene's transcription start site.

In another embodiment, the target antisense RNA transcript is transcribed from a locus on the coding strand located within +/−500, +/−250 or +/−100 of the target gene's transcription start site.

In another embodiment, the target antisense RNA transcript is transcribed from a locus on the coding strand located +/−2000 nucleotides of the target gene's transcription start site.

In another embodiment, the locus on the coding strand is no more than 1000 nucleotides upstream or downstream from a location corresponding to the target gene's transcription start site.

In another embodiment, the locus on the coding strand is no more than 500 nucleotides upstream or downstream from a location corresponding to the target gene's transcription start site.

The term “transcription start site” (TSS) as used herein means a nucleotide on the template strand of a gene corresponding to or marking the location of the start of transcription. The TSS may be located within the promoter region on the template strand of the gene.

The term “transcription stop site” as used herein means a region, which can be one or more nucleotides, on the template strand of a gene, which has at least one feature such as, but not limited to, a region which encodes at least one stop codon of the target transcript, a region encoding a sequence preceding the 3′UTR of the target transcript, a region where the RNA polymerase releases the gene, a region encoding a splice site or an area before a splice site and a region on the template strand where transcription of the target transcript terminates.

The phrase “is transcribed from a particular locus” in the context of the target antisense RNA transcript of the invention means the transcription of the target antisense RNA transcript starts at the particular locus.

The target antisense RNA transcript is complementary to the coding strand of the genomic sequence of the target gene, and any reference herein to “genomic sequence” is shorthand for “coding strand of the genomic sequence”.

The “coding strand” of a gene has the same base sequence as the mRNA produced, except T is replayed by U in the mRNA. The “template strand” of a gene is therefore complementary and antiparallel to the mRNA produced.

Thus, the target antisense RNA transcript may comprise a sequence which is complementary to a genomic sequence located between 100, 80, 60, 40, 20 or 10 kb upstream of the target gene's transcription start site and 100, 80, 60, 40, 20 or 10 kb downstream of the target gene's transcription stop site.

In one embodiment, the target antisense RNA transcript comprises a sequence which is complementary to a genomic sequence located between 1 kb upstream of the target gene's transcription start site and 1 kb downstream of the target gene's transcription stop site.

In another embodiment, the target antisense RNA transcript comprises a sequence which is complementary to a genomic sequence located between 500, 250 or 100 nucleotides upstream of the target gene's transcription start site and ending 500, 250 or 100 nucleotides downstream of the target gene's transcription stop site.

The target antisense RNA transcript may comprise a sequence which is complementary to a genomic sequence which includes the coding region of the target gene. The target antisense RNA transcript may comprise a sequence which is complementary to a genomic sequence that aligns with the target gene's promoter region on the template strand. Genes may possess a plurality of promoter regions, in which case the target antisense RNA transcript may align with one, two or more of the promoter regions. An online database of annotated gene loci may be used to identify the promoter regions of genes. The terms ‘align’ and ‘alignment’ when used in the context of a pair of nucleotide sequences mean the pair of nucleotide sequences are complementary to each other or have sequence identity with each other.

The region of alignment between the target antisense RNA transcript and the promoter region of the target gene may be partial and may be as short as a single nucleotide in length, although it may be at least 15 or at least 20 nucleotides in length, or at least 25 nucleotides in length, or at least 30, 35, 40, 45 or 50 nucleotides in length, or at least 55, 60, 65, 70 or 75 nucleotides in length, or at least 100 nucleotides in length. Each of the following specific arrangements is intended to fall within the scope of the term “alignment”:

a) The target antisense RNA transcript and the target gene's promoter region are identical in length and they align (i.e. they align over their entire lengths).

b) The target antisense RNA transcript is shorter than the target gene's promoter region and aligns over its entire length with the target gene's promoter region (i.e. it aligns over its entire length to a sequence within the target gene's promoter region).

c) The target antisense RNA transcript is longer than the target gene's promoter region and the target gene's promoter region is aligned fully by it (i.e. the target gene's promoter region is aligns over its entire length to a sequence within the target antisense RNA transcript).

d) The target antisense RNA transcript and the target gene's promoter region are of the same or different lengths and the region of alignment is shorter than both the length of the target antisense RNA transcript and the length of the target gene's promoter region.

The above definition of “align” and “alignment” applies mutatis mutandis to the description of other overlapping, e.g., aligned sequences throughout the description. Clearly, if a target antisense RNA transcript is described as aligning with a region of the target gene other than the promoter region then the sequence of the target antisense RNA transcript aligns with a sequence within the noted region rather than within the promoter region of the target gene.

In one embodiment, the target antisense RNA transcript is at least 1 kb, or at least 2, 3, 4, 5, 6, 7, 8, 9 or 10, e.g., 20, 25, 30, 35 or 40 kb long.

In one embodiment, the target antisense RNA transcript comprises a sequence which is at least 75%, or at least 85%, or at least 90%, or at least 95% complementary along its full length to a sequence on the coding strand of the target gene.

The present invention provides saRNAs targeting the target antisense RNA transcript and may effectively and specifically down-regulate such target antisense RNA transcripts. This can be achieved by saRNA having a high degree of complementarity to a region within the target antisense RNA transcript. The saRNA will have no more than 5, or no more than 4 or 3, or no more than 2, or no more than 1, or no mismatches with the region within the target antisense RNA transcript to be targeted.

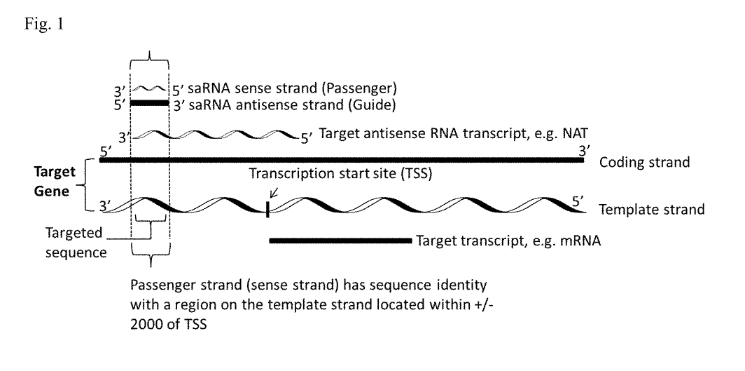

Referring to FIG. 1, as the target antisense RNA transcript has sequence identity with a region of the template strand of the target gene, the target antisense RNA transcript will be in part identical to a region within the template strand of the target gene allowing reference to be made either to the template strand of the gene or to a target antisense RNA transcript. The location at which the saRNA hybridizes or binds to the target antisense RNA transcript (and hence the same location on the template strand) is referred to as the “targeted sequence” or “target site”.

The antisense strand of the saRNA (whether single- or double-stranded) may be at least 80%, 90%, 95%, 98%, 99% or 100% identical with the reverse complement of the targeted sequence. Thus, the reverse complement of the antisense strand of the saRNA has a high degree of sequence identity with the targeted sequence. The targeted sequence may have the same length, i.e., the same number of nucleotides, as the saRNA and/or the reverse complement of the saRNA.

In some embodiments, the targeted sequence comprises at least 14 and less than 30 nucleotides.

In some embodiments, the targeted sequence has 19, 20, 21, 22, or 23 nucleotides.

In some embodiments, the location of the targeted sequence is situated within a promoter area of the template strand.

In some embodiments, the targeted sequence is located within a TSS (transcription start site) core of the template stand. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence.

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located upstream of the TSS in the TSS core. The targeted sequence may be less than 2000, less than 1000, less than 500, less than 250, or less than 100 nucleotides upstream of the TSS.

In some embodiments, the targeted sequence is located downstream of the TSS in the TSS core. The targeted sequence may be less than 2000, less than 1000, less than 500, less than 250, or less than 100 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence overlaps begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 4001, the targeted sequence is considered downstream of the TSS. When the 5′ most end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600^{th} nucleotide of the TSS core, the targeted sequence starts at position 1600 of the TSS core and is considered to be upstream of the TSS.

In one embodiment, the saRNA of the present invention may have two strands that form a duplex, one strand being a guide strand. The saRNA duplex is also called a double-stranded saRNA. A double-stranded saRNA or saRNA duplex, as used herein, is a saRNA that includes more than one, and preferably, two, strands in which interstrand hybridization can form a region of duplex structure. The two strands of a double-stranded saRNA are referred to as an antisense strand or a guide strand, and a sense strand or a passenger strand.

The antisense strand of an saRNA duplex, used interchangeably with antisense strand saRNA or antisense saRNA, has a high degree of complementarity to a region within the target antisense RNA transcript. The antisense strand may have no more than 5, or no more than 4 or 3, or no more than 2, or no more than 1, or no mismatches with the region within the target antisense RNA transcript or targeted sequence. Therefore, the antisense strand has a high degree of complementary to the targeted sequence on the template strand. The sense strand of the saRNA duplex, used interchangeably with sense strand saRNA or sense saRNA, has a high degree of sequence identity with the targeted sequence on the template strand. In some embodiments, the targeted sequence is located within the promoter area of the template strand. In some embodiments, the targeted sequence is located within the TSS core of the template stand.

The location of the antisense strand and/or sense strand of the saRNA duplex, relative to the targeted sequence is defined by making reference to the TSS core sequence. For example, when the targeted sequence is downstream of the TSS, the antisense saRNA and the sense saRNA start downstream of the TSS. In another example, when the targeted sequence starts at position 200 of the TSS core, the antisense saRNA and the sense saRNA start upstream of the TSS.

The relationships among the saRNAs, a target gene, a coding strand of the target gene, a template strand of the target gene, a target antisense RNA transcript, a target transcript, a targeted sequence/target site, and the TSS are shown in FIG. 1.

A “strand” in the context of the present invention means a contiguous sequence of nucleotides, including non-naturally occurring or modified nucleotides. Two or more strands may be, or each form a part of, separate molecules, or they may be connected covalently, e.g., by a linker such as a polyethyleneglycol linker. At least one strand of an saRNA may comprise a region that is complementary to a target antisense RNA. Such a strand is called an antisense or guide strand of the saRNA duplex. A second strand of an saRNA that comprises a region complementary to the antisense strand of the saRNA is called a sense or passenger strand.

An saRNA duplex may also be formed from a single molecule that is at least partly self-complementary forming a hairpin structure, including a duplex region. In such case, the term “strand” refers to one of the regions of the saRNA that is complementary to another internal region of the saRNA. The guide strand of the saRNA will have no more than 5, or no more than 4 or 3, or no more than 2, or no more than 1, or no mismatches with the sequence within the target antisense RNA transcript.

In some embodiments, the passenger strand of an saRNA may comprise at least one nucleotide that is not complementary to the corresponding nucleotide on the guide strand, called a mismatch with the guide strand. The mismatch with the guide strand may encourage preferential loading of the guide strand (Wu et al., PLoS ONE, vol. 6 (12):e28580 (2011), the contents of which are incorporated herein by reference in their entirety). In one embodiment, the at least one mismatch with the guide strand may be at 3′ end of the passenger strand. In one embodiment, the 3′ end of the passenger strand may comprise 1-5 mismatches with the guide strand. In one embodiment, the 3′ end of the passenger strand may comprise 2-3 mismatches with the guide strand. In one embodiment, the 3′ end of the passenger strand may comprise 6-10 mismatches with the guide strand.

A saRNA duplex may have siRNA-like complementarity to a region of a target antisense RNA transcript; that is, 100% complementarity between nucleotides 2-6 from the 5′ end of the guide strand in the saRNA duplex and a region of the target antisense RNA transcript. Other nucleotides of the saRNA may, in addition, have at least 80%, 90%, 95%, 98%, 99% or 100% complementarity to a region of the target antisense RNA transcript. For example, nucleotides 7 (counted from the 5′ end) until the 3′ end of the saRNA may have least 80%, 90%, 95%, 98%, 99% or 100% complementarity to a region of the target antisense RNA transcript.

The terms “small interfering RNA” or “siRNA” in the context mean a double-stranded RNA typically 20-25 nucleotides long involved in the RNA interference (RNAi) pathway and interfering with or inhibiting the expression of a specific gene. The gene is the target gene of the siRNA. For example, siRNA that interferes the expression of APOA1 gene is called “APOA1-siRNA” and the APOA1 gene is the target gene. An siRNA is usually about 21 nucleotides long, with 3′ overhangs (e.g., 2 nucleotides) at each end of the two strands.

An siRNA inhibits target gene expression by binding to and promoting the cleavage of one or more RNA transcripts of the target gene at specific sequences. Typically in RNAi the RNA transcripts are mRNA, so cleavage of mRNA results in the down-regulation of gene expression. In the present invention, not willing to be bound with any theory, one of the possible mechanisms is that saRNA of the present invention may modulate the target gene expression by cleavage of the target antisense RNA transcript.

A double-stranded saRNA may include one or more single-stranded nucleotide overhangs. The term “overhang” or “tail” in the context of double-stranded saRNA and siRNA refers to at least one unpaired nucleotide that protrudes from the duplex structure of saRNA or siRNA. For example, when a 3′-end of one strand of a saRNA extends beyond the 5′-end of the other strand, or vice versa, there is a nucleotide overhang. A saRNA may comprise an overhang of at least one nucleotide; alternatively the overhang may comprise at least two nucleotides, at least three nucleotides, at least four nucleotides, at least five nucleotides or more. A nucleotide overhang may comprise of consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a saRNA. Where two oligonucleotides are designed to form, upon hybridization, one or more single-stranded overhangs, and such overhangs shall not be regarded as mismatches with regard to the determination of complementarity. For example, a saRNA comprising one oligonucleotide 19 nucleotides in length and another oligonucleotide 21 nucleotides in length, wherein the longer oligonucleotide comprises a sequence of 19 nucleotides that is fully complementary to the shorter oligonucleotide, can yet be referred to as “fully complementary” for the purposes described herein.

In one embodiment, the antisense strand of a double-stranded saRNA has a 1-10 nucleotide overhang at the 3′ end and/or the 5′ end. In one embodiment, the antisense strand of a double-stranded saRNA has 1-4 nucleotide overhang at its 3′ end, or 1-2 nucleotide overhang at its 3′ end. In one embodiment, the sense strand of a double-stranded saRNA has a 1-10 nucleotide overhang at the 3′ end and/or the 5′ end. In one embodiment, the sense strand of a double-stranded saRNA has 1-4 nucleotide overhang at its 3′ end, or 1-2 nucleotide overhang at its 3′ end. In one embodiment, both the sense strand and the antisense strand of a double-stranded saRNA have 3′ overhangs. The 3′ overhangs may comprise one or more uracils, e.g., the sequences UU or UUU. In one embodiment, one or more of the nucleotides in the overhang is replaced with a nucleoside thiophosphate, wherein the internucleoside linkage is thiophosphate. In one embodiment, the overhang comprises one or more deoxyribonucleoside, e.g., the sequence dTdT or dTdTdT. In one embodiments, the overhang comprises the sequence dT*dT, wherein * is a thiophosphate internucleoside linkage.

The skilled person will appreciate that it is convenient to define the saRNA of the present invention by reference to the target antisense RNA transcript or the targeted sequence, regardless of the mechanism by which the saRNA modulates the target gene expression. However, the saRNA of the present invention may alternatively be defined by reference to the target gene. The target antisense RNA transcript is complementary to a genomic region on the coding strand of the target gene, and the saRNA of the present invention is in turn complementary to a region of the target antisense RNA transcript, so the saRNA of the present invention may be defined as having sequence identity to a region on the coding strand of the target gene. All of the features discussed herein with respect to the definition of the saRNA of the present invention by reference to the target antisense RNA transcript apply mutatis mutandis to the definition of the saRNA of the present invention by reference to the target gene so any discussion of complementarity to the target antisense RNA transcript should be understood to include identity to the genomic sequence of the target gene. Thus, the saRNA of the present invention may have a high percent identity, e.g. at least 80%, 90%, 95%, 98% or 99%, or 100% identity, to a genomic sequence on the target gene. The genomic sequence may be up to 2000, 1000, 500, 250, or 100 nucleotides upstream or downstream of the target gene's transcription start site. It may align with the target gene's promoter region. Thus, the saRNA may have sequence identity to a sequence that aligns with the promoter region of the target gene.

In one embodiment, the existence of the target antisense RNA transcript does not need to be determined to design the saRNA of the present invention. In another word, the design of the saRNA does not require the identification of the target antisense RNA transcript. For example, the nucleotide sequence of the TSS core, i.e., the sequence in the region 2000 nucleotides upstream of the target gene's transcription start site to 2000 nucleotides downstream of the target gene's transcription start may be obtained by the genomic sequence of the coding strand of the target gene, by sequencing or by searching in a database. Targeted sequence within the TSS core starting at any position from position 1 to position 4001 of the TSS core on the template strand can be selected and can then be used to design saRNA sequences. As discussed above, the saRNA has a high degree of sequence identity with the reverse complement of the targeted sequence.

The saRNA sequence's off-target hit number in the whole genome, 0 mismatch (0 mm) hit number, and 1 mismatch (1 mm) hit number are then determined. The term “off-target hit number” refers to the number of other sites in the whole genome that are identical to the saRNA's targeted sequence on the template strand of the target gene. The term “0 mm hit number” refers to the number of known protein coding transcript other than the target transcript of the saRNA, the complement of which the saRNA may hybridize with or bind to with 0 mismatch. In another word, “0 mm hit number” counts the number of known protein coding transcript, other than the target transcript of the saRNA that comprises a region completely identical with the saRNA sequence. The term “1 mm hit number” refers to the number of known protein coding transcript other than the target transcript of the saRNA, the complement of which the saRNA may hybridize with or bind to with 1 mismatch. In another word, “1 mm hit number” counts the number of known protein coding transcript, other than the target transcript of the saRNA that comprises a region identical with the saRNA sequence with only 1 mismatch. In one embodiment, only saRNA sequences that have no off-target hit, no 0 mm hit and no 1 mm hit are selected. For those saRNA sequences disclosed in the present application, each has no off-target hit, no 0 mm hit and no 1 mm hit.

The method disclosed in US 2013/0164846 filed Jun. 23, 2011 (saRNA algorithm), the contents of which are incorporated herein by reference in their entirety, may also be used to design saRNA. The design of saRNA is also disclosed in U.S. Pat. No. 8,324,181 and U.S. Pat. No. 7,709,566 to Corey et al., US Pat. Pub. No. 2010/0210707 to Li et al., and Voutila et al., Mol Ther Nucleic Acids, vol. 1, e35 (2012), the contents of each of which are incorporated herein by reference in their entirety.

“Determination of existence” means either searching databases of ESTs and/or antisense RNA transcripts around the locus of the target gene to identify a suitable target antisense RNA transcript, or using RT PCR or any other known technique to confirm the physical presence of a target antisense RNA transcript in a cell.

In some embodiments, the saRNA of the present invention may be single or, double-stranded. Double-stranded molecules comprise a first strand and a second strand. If double-stranded, each strand of the duplex may be at least 14, or at least 18, e.g. 19, 20, 21 or 22 nucleotides in length. The duplex may be hybridized over a length of at least 12, or at least 15, or at least 17, or at least 19 nucleotides. Each strand may be exactly 19 nucleotides in length. Preferably, the length of the saRNA is less than 30 nucleotides since oligonucleotide duplex exceeding this length may have an increased risk of inducing the interferon response. In one embodiment, the length of the saRNA is 19 to 25 nucleotides. The strands forming the saRNA duplex may be of equal or unequal lengths.

In one embodiment, the saRNAs of the present invention comprise a sequence of at least 14 nucleotides and less than 30 nucleotides which has at least 80%, 90%, 95%, 98%, 99% or 100% complementarity to the targeted sequence. In one embodiment, the sequence which has at least 80%, 90%, 95%, 98%, 99% or 100% complementarity to the targeted sequence is at least 15, 16, 17, 18 or 19 nucleotides in length, or 18-22 or 19 to 21, or exactly 19.

The saRNA of the present invention may include a short 3′ or 5′ sequence which is not complementary to the target antisense RNA transcript. In one embodiment, such a sequence is at 3′ end of the strand. The sequence may be 1-5 nucleotides in length, or 2 or 3. The sequence may comprises uracil, so it may be a 3′ stretch of 2 or 3 uracils. The sequence may comprise one or more deoxyribonucleoside, such as dT. In one embodiment, one or more of the nucleotides in the sequence is replaced with a nucleoside thiophosphate, wherein the internucleoside linkage is thiophosphate. As a non-limiting example, the sequence comprises the sequence dT*dT, wherein * is a thiophosphate internucleoside linkage. This non-complementary sequence may be referred to as “tail”. If a 3′ tail is present, the strand may be longer, e.g., 19 nucleotides plus a 3′ tail, which may be UU or UUU. Such a 3′ tail shall not be regarded as mismatches with regard to determine complementarity between the saRNA and the target antisense RNA transcript.

Thus, the saRNA of the present invention may consist of (i) a sequence having at least 80% complementarity to a region of the target antisense RNA transcript; and (ii) a 3′ tail of 1-5 nucleotides, which may comprise or consist of uracil residues. The saRNA will thus typically have complementarity to a region of the target antisense RNA transcript over its whole length, except for the 3′ tail, if present. Any of the saRNA sequences disclosed in the present application may optionally include such a 3′ tail. Thus, any of the saRNA sequences disclosed in the saRNA Tables and Sequence Listing may optionally include such a 3′ tail. The saRNA of the present invention may further comprise Dicer or Drosha substrate sequences.

The saRNA of the present invention may contain a flanking sequence. The flanking sequence may be inserted in the 3′ end or 5′ end of the saRNA of the present invention. In one embodiment, the flanking sequence is the sequence of a miRNA, rendering the saRNA to have miRNA configuration and may be processed with Drosha and Dicer. In a non-limiting example, the saRNA of the present invention has two strands and is cloned into a microRNA precursor, e.g., miR-30 backbone flanking sequence.

The saRNA of the present invention may comprise a restriction enzyme substrate or recognition sequence. The restriction enzyme recognition sequence may be at the 3′ end or 5′ end of the saRNA of the present invention. Non-limiting examples of restriction enzymes include NotI and AscI.

In one embodiment, the saRNA of the present invention consists of two strands stably base-paired together. In some embodiments, the passenger strand may comprise at least one nucleotide that is not complementary to the corresponding nucleotide on the guide strand, called a mismatch with the guide strand. In one embodiment, the at least one mismatch with the guide strand may be at 3′ end of the passenger strand. In one embodiment, the 3′ end of the passenger strand may comprise 1-5 mismatches with the guide strand. In one embodiment, the 3′ end of the passenger strand may comprise 2-3 mismatches with the guide strand. In one embodiment, the 3′ end of the passenger strand may comprise 6-10 mismatches with the guide strand.

In some embodiments, the double-stranded saRNA may comprise a number of unpaired nucleotides at the 3′ end of each strand forming 3′ overhangs. The number of unpaired nucleotides forming the 3′ overhang of each strand may be in the range of 1 to 5 nucleotides, or 1 to 3 nucleotides, or 2 nucleotides. The 3′ overhang may be formed on the 3′ tail mentioned above, so the 3′ tail may be the 3′ overhang of a double-stranded saRNA.

Thus, the saRNA of the present invention may be single-stranded and consists of (i) a sequence having at least 80% complementarity to a region of the target antisense RNA transcript; and (ii) a 3′ tail of 1-5 nucleotides, which may comprise uracil residues. The saRNA of the present invention may have complementarity to a region of the target antisense RNA transcript over its whole length, except for the 3′ tail, if present. As mentioned above, instead of “complementary to the target antisense RNA transcript” the saRNA of the present invention may also be defined as having “identity” to the coding strand of the target gene. The saRNA of the present invention may be double-stranded and consists of a first strand comprising (i) a first sequence having at least 80% complementarity to a region of the target antisense RNA transcript and (ii) a 3′ overhang of 1-5 nucleotides; and a second strand comprising (i) a second sequence that forms a duplex with the first sequence and (ii) a 3′ overhang of 1-5 nucleotides.

As described herein, the genomic sequence of the target gene may be used to design saRNA of the target gene. The sequence of a target antisense RNA transcript may be determined from the sequence of the target gene for designing saRNA of the target gene. However, the existence of such a target antisense RNA transcript does not need to be determined.

One aspect of the present invention provides a saRNA that modulates the expression of a target gene. Also provided is a saRNA that modulates the level of a target transcript. In some embodiments, the target transcript is a coding transcript, e.g., mRNA. Another aspect of the present invention provides a saRNA that modulates the level of a protein encoded by the coding target transcript.

Non-limited examples of target genes include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), and coagulation factor VIII (F8). Sequences of these non-limited examples of target genes of saRNA of the present invention are provided in Table 1.

TABLE 1
Sequences of Example Target Genes

| | | mRNA transcribed | | |
|---|---|---|---|---|
| Target | Location on | from the target gene | Protein encoded | Nature |
| Gene | chromosome | (target transcript) | by the target gene | of gene |
| APOA1 human | 11q23-q24 | SEQ ID NO. 1 | SEQ ID NO. 7 | Coding |
| | | (NM_000039) | (NP_000030.1) | |
| APOA1 mouse | 9 25.36 cM | SEQ ID NO. 2 | SEQ ID NO. 8 | Coding |
| | | (NM_009692) | (NP_033822.2) | |
| APOA1 rat | 8q23-q24 | SEQ ID NO. 3 | SEQ ID NO. 9 | Coding |
| | | (NM_012738) | (NP_036870.1) | |
| LDLR human | 19p13.2 | SEQ ID NO. 4 | SEQ ID NO. 10 | Coding |
| | | (NM_000527) | (NP_000518.1) | |
| LDLR mouse | 9 7.87 cM | SEQ ID NO. 5 | SEQ ID NO. 11 | Coding |
| | | (NM_010700) | (NP_034830.2) | |
| LDLR rat | 8q13 | SEQ ID NO. 6 | SEQ ID NO. 12 | Coding |
| | | (NM_175762) | (NP_786938.1) | |
| DMD human | Xp21.2 | SEQ ID NO. 13 | SEQ ID NO. 49 | Coding |
| | | (NM_000109) | (NP_000100.2) | |
| | | SEQ ID NO. 14 | | |
| | | (NM_004006) | | |
| | | SEQ ID NO. 15 | | |
| | | (NM_004009) | | |
| | | SEQ ID NO. 16 | | |
| | | (NM_004012) | | |
| | | SEQ ID NO. 17 | | |
| | | (NM_004014) | | |
| | | SEQ ID NO. 18 | | |
| | | (NM_004019) | | |
| | | SEQ ID NO. 19 | | |
| | | (NM_004021) | | |
| PAX5 human | 9p13 | SEQ ID NO. 20 | SEQ ID NO. 50 | Coding |
| | | (NM_016734) | (NP_057953.1) | |
| SCN1A human | 2q24.3 | SEQ ID NO. 21 | SEQ ID NO. 51 | Coding |
| | | (NM_001202435) | (NP_001189364.1) | |
| | | SEQ ID NO. 22 | | |
| | | (NM_006920) | | |
| IDUA human | 4p16.3 | SEQ ID NO. 23 | SEQ ID NO. 52 | Coding |
| | | (NM_000203) | (NP_000194.2) | |
| FNDC5 human | 1p35.1 | SEQ ID NO. 24 | SEQ ID NO. 53 | Coding |
| | | (NM_001171941) | (NP_001165412.1) | |
| | | SEQ ID NO. 25 | | |
| | | (NM_153756) | | |
| FOXA2 human | 20p11 | SEQ ID NO. 26 | SEQ ID NO. 54 | Coding |
| | | (NM_021784) | (NP_068556.2) | |
| | | SEQ ID NO. 27 | | |
| | | (NM_153675) | | |
| FOXP3 human | Xp11.23 | SEQ ID NO. 28 | SEQ ID NO. 55 | Coding |
| | | (NM_014009) | (NP_054728.2) | |
| HNF4A human | 20q13.12 | SEQ ID NO. 29 | SEQ ID NO. 56 | Coding |
| | | (NM_000457) | (NP_000448.3) | |
| | | SEQ ID NO. 30 | | |
| | | (NM_175914) | | |
| IFNG human | 12q14 | SEQ ID NO. 31 | SEQ ID NO. 57 | Coding |
| | | (NM_000619) | (NP_000610.2) | |
| IL10 human | 1q31-q32 | SEQ ID NO. 32 | SEQ ID NO. 58 | Coding |
| | | (NM_000572) | (NP_000563.1) | |
| IL2 human | 4q26-q27 | SEQ ID NO. 33 | SEQ ID NO. 59 | Coding |
| | | (NM_000586) | (NP_000577.2) | |
| IL19 human | 1q32.2 | SEQ ID NO. 34 | SEQ ID NO. 60 | Coding |
| | | (NM_013371) | (NP_037503.2) | |
| LMX1A human | 1q24.1 | SEQ ID NO. 35 | SEQ ID NO. 61 | Coding |
| | | (NM_001174069) | (NP_001167540.1) | |
| | | SEQ ID NO. 36 | | |
| | | (NM_177398) | | |
| METRNL human | 17q25.3 | SEQ ID NO. 37 | SEQ ID NO. 62 | Coding |
| | | (NM_001004431) | (NP_001004431.1) | |
| NR4A2 human | 2q22-q23 | SEQ ID NO. 38 | SEQ ID NO. 63 | Coding |
| | | (NM_006186) | (NP_006177.1) | |
| SIRT1 human | 10q21.3 | SEQ ID NO. 39 | SEQ ID NO. 64 | Coding |
| | | (NM_001142498) | (NP_001135970.1) | |
| TH human | 11p15.5 | SEQ ID NO. 40 | SEQ ID NO. 65 | Coding |
| | | (NM_000360) | (NP_000351.2) | |
| EPO human | 7q22 | SEQ ID NO. 41 | SEQ ID NO. 66 | Coding |
| | | (NM_000799) | (NP_000790.2) | |
| CDKN1B human | 12p13.1-p12 | SEQ ID NO. 42 | SEQ ID NO. 67 | Coding |
| | | (NM_004064) | (NP_004055.1) | |
| MDM2 human | 12q14.3-q15 | SEQ ID NO. 43 | SEQ ID NO. 68 | Coding |
| | | (NM_001145337) | (NP_001138809.1) | |
| | | SEQ ID NO. 44 | | |
| | | (NM_002392) | | |
| C19orf80 human | 19p13.2 | SEQ ID NO. 45 | SEQ ID NO. 69 | Coding |
| | | (NM_018687) | (NP_061157.3) | |
| F7 human | 13q34 | SEQ ID NO. 46 | SEQ ID NO. 70 | Coding |
| | | (NM_019616) | (NP_062562.1) | |
| F8 human | Xq28 | SEQ ID NO. 47 | SEQ ID NO. 71 | Coding |
| | | (NM_000132) | (NP_000123.1) | |
| | | SEQ ID NO. 48 | | |
| | | (NM_019863) | | |

The saRNA of the present invention may be produced by any suitable method, for example synthetically or by expression in cells using standard molecular biology techniques which are well-known to a person of ordinary skill in the art. For example, the saRNA of the present invention may be chemically synthesized or recombinantly produced using methods known in the art.

### Bifunction Oligonucleotides

Bifunction or dual-functional oligonucleotides, e.g., saRNA may be designed to upregulate the expression of a first gene and down-regulate the expression of at least one second gene. One strand of the dual-functional oligonucleotide activates the expression of the first gene and the other strand inhibits the expression of the second gene. Each strand might further comprise a Dicer substrate sequence.

Chemical Modifications of saRNA

Herein, in saRNA, the terms “modification” or, as appropriate, “modified” refer to structural and/or chemical modifications with respect to A, G, U or C ribonucleotides. Nucleotides in the saRNAs of the present invention may comprise non-standard nucleotides, such as non-naturally occurring nucleotides or chemically synthesized nucleotides or deoxynucleotides. The saRNA of the present invention may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. In a non-limiting example, the 2′-OH of U is substituted with 2′-OMe.

In one embodiment, the saRNAs of the present invention may comprise at least one modification described herein.

In another embodiment, the saRNA is an saRNA duplex and the sense strand and antisense sequence may independently comprise at least one modification. As a non-limiting example, the sense sequence may comprises a modification and the antisense strand may be unmodified. As another non-limiting example, the antisense sequence may comprises a modification and the sense strand may be unmodified. As yet another non-limiting example, the sense sequence may comprises more than one modification and the antisense strand may comprise one modification. As a non-limiting example, the antisense sequence may comprise more than one modification and the sense strand may comprise one modification.

The saRNA of the present invention can include a combination of modifications to the sugar, the nucleobase, and/or the internucleoside linkage. These combinations can include any one or more modifications described herein or in International Application Publication WO2013/052523 filed Oct. 3, 2012, in particular Formulas (Ia)-(Ia-5), (Ib)-(If), (IIa)-(IIp), (IIb-1), (IIb-2), (IIc-1)-(IIc-2), (IIn-1), (IIn-2), (IVa)-(IVl), and (IXa)-(IXr)), the contents of which are incorporated herein by reference in their entirety.

The saRNA of the present invention may or may not be uniformly modified along the entire length of the molecule. For example, one or more or all types of nucleotide (e.g., purine or pyrimidine, or any one or more or all of A, G, U, C) may or may not be uniformly modified in the saRNA of the invention. In some embodiments, all nucleotides X in a saRNA of the invention are modified, wherein X may be any one of nucleotides A, G, U, C, or any one of the combinations A+G, A+U, A+C, G+U, G+C, U+C, A+G+U, A+G+C, G+U+C or A+G+C.

Different sugar modifications, nucleotide modifications, and/or internucleoside linkages (e.g., backbone structures) may exist at various positions in a saRNA. One of ordinary skill in the art will appreciate that the nucleotide analogs or other modification(s) may be located at any position(s) of a saRNA such that the function of saRNA is not substantially decreased. The saRNA of the present invention may contain from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the saRNA of the present invention may be modified to be a spherical nucleic acid (SNA) or a circular nucleic acid. The terminals of the saRNA of the present invention may be linked by chemical reagents or enzymes, producing spherical saRNA that has no free ends. Spherical saRNA is expected to be more stable than its linear counterpart and to be resistant to digestion with RNase R exonuclease. Spherical saRNA may further comprise other structural and/or chemical modifications with respect to A, G, U or C ribonucleotides.

In some embodiments, the saRNA of the present invention may comprise inverted deoxy abasic modifications on the passenger strand. The at least one inverted deoxy abasic modification may be on 5′ end, or 3′ end, or both ends of the passenger strand. The inverted deoxy basic modification may encourage preferential loading of the guide strand.

The saRNA of the present invention may be modified with any modifications of an oligonucleotide or polynucleotide disclosed in pages 136 to 247 of PCT Publication WO2013/151666 published Oct. 10, 2013, the contents of which are incorporated herein by reference in their entirety.

saRNA Conjugates and Combinations

Conjugation may result in increased stability and/or half-life and may be particularly useful in targeting the saRNA of the present invention to specific sites in the cell, tissue or organism. The saRNA of the present invention can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. Suitable conjugates for nucleic acid molecules are disclosed in International Publication WO 2013/090648 filed Dec. 14, 2012, the contents of which are incorporated herein by reference in their entirety.

According to the present invention, saRNA of the present invention may be administered with, or further include one or more of RNAi agents, small interfering RNAs (siRNAs), small hairpin RNAs (shRNAs), long non-coding RNAs (lncRNAs), enhancer RNAs, enhancer-derived RNAs or enhancer-driven RNAs (eRNAs), microRNAs (miRNAs), miRNA binding sites, antisense RNAs, ribozymes, catalytic DNA, tRNA, RNAs that induce triple helix formation, aptamers or vectors, and the like to achieve different functions. The one or more RNAi agents, small interfering RNAs (siRNAs), small hairpin RNAs (shRNAs), long non-coding RNAs (lncRNA), microRNAs (miRNAs), miRNA binding sites, antisense RNAs, ribozymes, catalytic DNA, tRNA, RNAs that induce triple helix formation, aptamers or vectors may comprise at least one modification or substitution.

In some embodiments, the modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In one embodiment, the high molecular weight, non-immunogenic compound is polyalkylene glycol, or polyethylene glycol (PEG).

In one embodiment, saRNA of the present invention may be attached to a transgene so it can be co-expressed from an RNA polymerase II promoter. In a non-limiting example, saRNA of the present invention is attached to green fluorescent protein gene (GFP).

In one embodiment, saRNA of the present invention may be attached to a DNA or RNA aptamer, thereby producing saRNA-aptamer conjugate. Aptamers are oligonucleotides or peptides with high selectivity, affinity and stability. They assume specific and stable three-dimensional shapes, thereby providing highly specific, tight binding to target molecules. An aptamer may be a nucleic acid species that has been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Nucleic acid aptamers have specific binding affinity to molecules through interactions other than classic Watson-Crick base pairing. Nucleic acid aptamers, like peptides generated by phage display or monoclonal antibodies (mAbs), are capable of specifically binding to selected targets and, through binding, block their targets' ability to function. In some cases, aptamers may also be peptide aptamers. For any specific molecular target, nucleic acid aptamers can be identified from combinatorial libraries of nucleic acids, e.g. by SELEX. Peptide aptamers may be identified using a yeast two hybrid system. A skilled person is therefore able to design suitable aptamers for delivering the saRNAs or cells of the present invention to target cells such as liver cells. DNA aptamers, RNA aptamers and peptide aptamers are contemplated. Administration of saRNA of the present invention to the liver using liver-specific aptamers is preferred.

As used herein, a typical nucleic acid aptamer is approximately 10-15 kDa in size (20-45 nucleotides), binds its target with at least nanomolar affinity, and discriminates against closely related targets. Nucleic acid aptamers may be ribonucleic acid, deoxyribonucleic acid, or mixed ribonucleic acid and deoxyribonucleic acid. Aptamers may be single-stranded ribonucleic acid, deoxyribonucleic acid or mixed ribonucleic acid and deoxyribonucleic acid. Aptamers may comprise at least one chemical modification.

A suitable nucleotide length for an aptamer ranges from about 15 to about 100 nucleotides (nt), and in various other embodiments, 15-30 nt, 20-25 nt, 30-100 nt, 30-60 nt, 25-70 nt, 25-60 nt, 40-60 nt, 25-40 nt, 30-40 nt, any of 22, 23, 24, 25, 26, 27, 28, 29, 30, 31, 32, 33, 34, 35, 36, 37, 38, 39 or 40 nt or 40-70 nt in length. However, the sequence can be designed with sufficient flexibility such that it can accommodate interactions of aptamers with two targets at the distances described herein. Aptamers may be further modified to provide protection from nuclease and other enzymatic activities. The aptamer sequence can be modified by any suitable methods known in the art.

The saRNA-aptamer conjugate may be formed using any known method for linking two moieties, such as direct chemical bond formation, linkage via a linker such as streptavidin and so on.

In one embodiment, saRNA of the present invention may be attached to an antibody. Methods of generating antibodies against a target cell surface receptor are well known. The saRNAs of the invention may be attached to such antibodies with known methods, for example using RNA carrier proteins. The resulting complex may then be administered to a subject and taken up by the target cells via receptor-mediated endocytosis.

In one embodiment, saRNA of the present invention may be conjugated with lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-5-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937), the content of each of which is herein incorporated by reference in its entirety.

In one embodiment, the saRNA of the present invention is conjugated with a ligand. In one non-limiting example, the ligand may be any ligand disclosed in US 20130184328 to Manoharan et al., the contents of which are incorporated herein by reference in their entirety. The conjugate has a formula of Ligand-[linker]ₒₚₜᵢₒₙₐₗ-[tether]ₒₚₜᵢₒₙₐₗ-oligonucleotide agent. The oligonucleotide agent may comprise a subunit having formulae (I) disclosed by US 20130184328 to Manoharan et al., the contents of which are incorporated herein by reference in their entirety. In another non-limiting example, the ligand may be any ligand disclosed in US 20130317081 to Akinc et al., the contents of which are incorporated herein by reference in their entirety, such as a lipid, a protein, a hormone, or a carbohydrate ligand of Formula II-XXVI. The ligand may be coupled with the saRNA with a bivalent or trivalent branched linker in Formula XXXI-XXXV disclosed in Akinc.

Representative U.S. patents that teach the preparation of such nucleic acid/lipid conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the content of each of which is herein incorporated by reference in its entirety.

The saRNA of the present invention may be provided in combination with other active ingredients known to have an effect in the particular method being considered. The other active ingredients may be administered simultaneously, separately, or sequentially with the saRNA of the present invention. In one embodiment, saRNA of the present invention is administered with saRNA modulating a different target gene. Non-limiting examples include saRNA that modulates albumin, insulin or HNF4A genes. Modulating any gene may be achieved using a single saRNA or a combination of two or more different saRNAs. Non-limiting examples of saRNA that can be administered with saRNA of the present invention include saRNA modulating albumin or HNF4A disclosed in International Publication WO 2012/175958 filed Jun. 20, 2012, saRNA modulating insulin disclosed in International Publications WO 2012/046084 and WO 2012/046085 both filed Oct. 10, 2011, saRNA modulating human progesterone receptor, human major vault protein (hMVP), E-cadherin gene, p53 gene, or PTEN gene disclosed in U.S. Pat. No. 7,709,456 filed Nov. 13, 2006 and US Pat. Publication US 2010/0273863 filed Apr. 23, 2010, saRNAs targeting p21 gene disclosed in International Publication WO 2006/113246 filed Apr. 11, 2006, any nucleic acid disclosed in WO2012/065143 filed Nov. 12, 2011 that upregulates the expression of genes in Table 8 of WO 2012/065143 or increases the expression of a tumor suppressor, any oligonucleotide that activates target genes in Table 4 of WO2013/173635 filed May 16, 2013, any oligonucleotide that activates target genes in Table 4 of WO2013/173637 filed May 16, 2013, any oligonucleotide complementary to a sequence selected from the sequences in SEQ ID NOs: 1-1212 of WO2013/173652 filed May 16, 2013, any oligonucleotide modulating APOA1 and ABCA1 gene expressions disclosed in WO2013173647 filed May 16, 2013, any oligonucleotide modulating SMN family gene expressions disclosed in WO2013173638 filed May 16, 2013, any oligonucleotide modulating PTEN gene expression disclosed in WO2013173605 filed May 16, 2013, any oligonucleotide modulating MECP2 gene expression disclosed in WO2013173608 filed May 16, 2013, any oligonucleotide modulating ATP2A2 gene expression disclosed in WO2013173598 filed May 16, 2013, any oligonucleotide modulating UTRN gene expression disclosed in WO2013173645 filed May 16, 2013, any nucleic acid molecule that modulates the expression of CD97, TS-α, C/EBP delta, CDC23, PINK1, HIF1α, Gnbp3g, Adrenomedullin AM1 receptor, 3-oxoacid CoA transferase, Cathepsin W or BACE1 disclosed in U.S. Pat. No. 8,288,354 filed Dec. 28, 2006, antagoNAT with formula (I) disclosed in US 2013/0245099 filed Nov. 17, 2011, any antagoNAT that upregulates the expression of hemoglobin (HBF/HBG) polynucleotides disclosed in U.S. Pat. No. 8,318,690 filed Apr. 30, 2010, any antisense oligonucleotide that increases the expression of apolipoprotein (ApoA1) polynucleotide disclosed in U.S. Pat. No. 8,153,696 filed Oct. 2, 2009 (CURNA), the contents of each of which are incorporated herein by reference in their entirety.

In on embodiment, the saRNA is conjugated with a carbohydrate ligand, such as any carbohydrate ligand disclosed in U.S. Pat. Nos. 8,106,022 and 8,828,956 to Manoharan et al. (Alnylam Pharmaceuticals), the contents of which are incorporated herein by reference in their entirety. For example, the carbohydrate ligand may be monosaccharide, disaccharide, trisaccharide, tetrasaccharide, oligosaccharide, or polysaccharide. These carbohydrate-conjugated RNA agents may target the parenchymal cells of the liver. In one embodiment, the saRNA is conjugated with more than one carbohydrate ligand, preferably two or three. In one embodiment, the saRNA is conjugated with one or more galactose moiety. In another embodiment, the saRNA is conjugated at least one (e.g., two or three or more) lactose molecules (lactose is a glucose coupled to a galactose). In another embodiment, the saRNA is conjugated with at least one (e.g., two or three or more) N-Acetyl-Galactosamine (GalNAc), N-Ac-Glucosamine (GluNAc), or mannose (e.g., mannose-6-phosphate). In one embodiment, the saRNA is conjugated with at least one mannose ligand, and the conjugated saRNA targets macrophages.

In one embodiment, saRNA of the present invention is administered with a small interfering RNA or siRNA that inhibits the expression of a gene.

In one embodiment, saRNA of the present invention is administered with one or more drugs for therapeutic purposes.

### II. Composition of the Invention

One aspect of the present invention provides pharmaceutical compositions comprising a small activating RNA (saRNA) that upregulates a target gene, and at least one pharmaceutically acceptable carrier.

### Formulation, Delivery, Administration, and Dosing

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, compositions are administered to humans, human patients or subjects. For the purposes of the present disclosure, the phrase “active ingredient” generally refers to saRNA to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one saRNA. As a non-limiting example, the formulations may contain 1, 2, 3, 4 or 5 saRNAs with different sequences. In one embodiment, the formulation contains at least three saRNAs with different sequences. In one embodiment, the formulation contains at least five saRNAs with different sequences.

The saRNA of the present invention can be formulated using one or more excipients to: (1) increase stability; (2) increase cell transfection; (3) permit the sustained or delayed release (e.g., from a depot formulation of the saRNA); (4) alter the biodistribution (e.g., target the saRNA to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with saRNA (e.g., for transplantation into a subject), hyaluronidase, nanoparticle mimics and combinations thereof. Accordingly, the formulations of the invention can include one or more excipients, each in an amount that together increases the stability of the saRNA and/or increases cell transfection by the saRNA. Further, the saRNA of the present invention may be formulated using self-assembled nucleic acid nanoparticles. Pharmaceutically acceptable carriers, excipients, and delivery agents for nucleic acids that may be used in the formulation with the saRNA of the present invention are disclosed in International Publication WO 2013/090648 filed Dec. 14, 2012, the contents of which are incorporated herein by reference in their entirety.

In one embodiment, the saRNA of the present invention comprises two single RNA strands that are 21 nucleotides in length each that are annealed to form a double-stranded saRNA as the active ingredient. The composition further comprises a salt buffer composed of 50 mM Tris-HCl, pH 8.0, 100 mM NaCl and 5 mM EDTA.

In another embodiment, the saRNA of the present invention may be delivered with dendrimers. Dendrimers are highly branched macromolecules. In one embodiment, the saRNA of the present invention is complexed with structurally flexible poly(amidoamine) (PAMAM) dendrimers for targeted in vivo delivery. The complex is called saRNA-dendrimers. Dendrimers have a high degree of molecular uniformity, narrow molecular weight distribution, specific size and shape characteristics, and a highly-functionalized terminal surface. The manufacturing process is a series of repetitive steps starting with a central initiator core. Each subsequent growth step represents a new generation of polymers with a larger molecular diameter and molecular weight, and more reactive surface sites than the preceding generation.

PAMAM dendrimers are efficient nucleotide delivery systems that bear primary amine groups on their surface and also a tertiary amine group inside of the structure. The primary amine group participates in nucleotide binding and promotes their cellular uptake, while the buried tertiary amino groups act as a proton sponge in endosomes and enhance the release of nucleic acid into the cytoplasm. These dendrimers protect the saRNA carried by them from ribonuclease degradation and achieves substantial release of saRNA over an extended period of time via endocytosis for efficient gene targeting. The in vivo efficacy of these nanoparticles have previously been evaluated where biodistribution studies show that the dendrimers preferentially accumulate in peripheral blood mononuclear cells and live with no discernible toxicity (see Zhou et al., Molecular Ther. 2011 Vol. 19, 2228-2238, the contents of which are incorporated herein by reference in their entirety). PAMAM dendrimers may comprise a triethanolamine (TEA) core, a diaminobutane (DAB) core, a cystamine core, a diaminohexane (HEX) core, a diamonododecane (DODE) core, or an ethylenediamine (EDA) core. In one embodiment, PAMAM dendrimers comprise a TEA core or a DAB core.

### Lipidoids

The synthesis of lipidoids has been extensively described and formulations containing these compounds are particularly suited for delivery of oligonucleotides or nucleic acids (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

While these lipidoids have been used to effectively deliver double-stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010; all of which is incorporated herein in their entirety), the present disclosure contemplates their formulation and use in delivering saRNA. Complexes, micelles, liposomes or particles can be prepared containing these lipidoids and therefore, can result in an effective delivery of the saRNA following the injection of a lipidoid formulation via localized and/or systemic routes of administration. Lipidoid complexes of saRNA can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, oligonucleotide to lipid ratio, and biophysical parameters such as, but not limited to, particle size (Akinc et al., Mol Ther. 2009 17:872-879; the contents of which are herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010); the contents of which are herein incorporated by reference in its entirety), C12-200 (including derivatives and variants), and MD1, can be tested for in vivo activity.

The lipidoid referred to herein as “98N12-5” is disclosed by Akinc et al., Mol Ther. 2009 17:872-879 and the contents of which is incorporated by reference in its entirety.

The lipidoid referred to herein as “C12-200” is disclosed by Love et al., Proc Natl Acad Sci USA. 2010 107:1864-1869 and Liu and Huang, Molecular Therapy. 2010 669-670; the contents of both of which are herein incorporated by reference in their entirety. The lipidoid formulations can include particles comprising either 3 or 4 or more components in addition to the saRNA. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a saRNA formulated with a lipidoid for systemic intravenous administration can target the liver. For example, a final optimized intravenous formulation using saRNA and comprising a lipid molar composition of 42% 98N12-5, 48% cholesterol, and 10% PEG-lipid with a final weight ratio of about 7.5 to 1 total lipid to saRNA and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879; the contents of which are herein incorporated by reference in its entirety). In another example, an intravenous formulation using a C12-200 (see published international application WO2010129709, the contents of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to nucleic acid and a mean particle size of 80 nm may be effective to deliver saRNA (see, Love et al., Proc Natl Acad Sci USA. 2010 107:1864-1869, the contents of which are herein incorporated by reference in its entirety).

In another embodiment, an MD1 lipidoid-containing formulation may be used to effectively deliver saRNA to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879, the contents of which are herein incorporated by reference in its entirety), use of a lipidoid-formulated saRNA to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8^{th} International Judah Folkman Conference, Cambridge, Mass. Oct. 8-9, 2010; the contents of each of which is herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of saRNA for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011 29:1005-1010; the contents of which are herein incorporated by reference in its entirety). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and saRNA.

### Liposomes, Lipoplexes, and Lipid Nanoparticles

The saRNA of the invention can be formulated using one or more liposomes, lipoplexes, or lipid nanoparticles. In one embodiment, pharmaceutical compositions of saRNA include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; the contents of which are herein incorporated by reference in its entirety) and liposomes which may deliver small molecule drugs such as, but not limited to, DOXIL® from Janssen Biotech, Inc. (Horsham, Pa.).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; the contents of each of which are incorporated herein in their entireties). The original manufacture method by Wheeler et al. was a detergent dialysis method, which was later improved by Jeffs et al. and is referred to as the spontaneous vesicle formation method. The liposome formulations may be composed of 3 to 4 lipid components in addition to the saRNA. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In another example, the nucleic acid-lipid particle may comprise a cationic lipid comprising from about 50 mol % to about 85 mol % of the total lipid present in the particle; a non-cationic lipid comprising from about 13 mol % to about 49.5 mol % of the total lipid present in the particle; and a conjugated lipid that inhibits aggregation of particles comprising from about 0.5 mol % to about 2 mol % of the total lipid present in the particle as described in WO2009127060 to Maclachlan et al, the contents of which are incorporated herein by reference in their entirety. In another example, the nucleic acid-lipid particle may be any nucleic acid-lipid particle disclosed in US2006008910 to Maclachlan et al., the contents of which are incorporated herein by reference in their entirety. As a non-limiting example, the nucleic acid-lipid particle may comprise a cationic lipid of Formula I, a non-cationic lipid, and a conjugated lipid that inhibits aggregation of particles.

In one embodiment, the saRNA of the present invention may be formulated in a lipid vesicle which may have crosslinks between functionalized lipid bilayers.

In one embodiment, the liposome may contain a sugar-modified lipid disclosed in U.S. Pat. No. 5,595,756 to Bally et al., the contents of which are incorporated herein by reference in their entirety. The lipid may be a ganglioside and cerebroside in an amount of about 10 mol percent.

In one embodiment, the saRNA of the present invention may be formulated in a liposome comprising a cationic lipid. The liposome may have a molar ratio of nitrogen atoms in the cationic lipid to the phosphates in the saRNA (N:P ratio) of between 1:1 and 20:1 as described in International Publication No. WO2013006825, the contents of which are herein incorporated by reference in its entirety. In another embodiment, the liposome may have a N:P ratio of greater than 20:1 or less than 1:1.

In one embodiment, the saRNA of the present invention may be formulated in a lipid-polycation complex. The formation of the lipid-polycation complex may be accomplished by methods known in the art and/or as described in U.S. Pub. No. 20120178702, the contents of which are herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326; herein incorporated by reference in its entirety. In another embodiment, the saRNA may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; the contents of which are herein incorporated by reference in its entirety), the liposome formulation was composed of 57.1% cationic lipid, 7.1% dipalmitoylphosphatidylcholine, 34.3% cholesterol, and 1.4% PEG-c-DMA.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the saRNA of the present invention may be formulated in a lipid nanoparticle such as the lipid nanoparticles described in International Publication No. WO2012170930, the contents of which are herein incorporated by reference in its entirety.

In one embodiment, the cationic lipid which may be used in formulations of the present invention may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; the contents of each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; the contents of each of which are herein incorporated by reference in their entirety. In yet another embodiment, the cationic lipid may be a multivalent cationic lipid such as the cationic lipid disclosed in U.S. Pat. No. 7,223,887 to Gaucheron et al., the contents of which are incorporated herein by reference in their entirety. The cationic lipid may have a positively-charged head group including two quaternary amine groups and a hydrophobic portion including four hydrocarbon chains as described in U.S. Pat. No. 7,223,887 to Gaucheron et al., the contents of which are incorporated herein by reference in their entirety. In yet another embodiment, the cationic lipid may be biodegradable as the biodegradable lipids disclosed in US20130195920 to Maier et al., the contents of which are incorporated herein by reference in their entirety. The cationic lipid may have one or more biodegradable groups located in a lipidic moiety of the cationic lipid as described in formula I-IV in US 20130195920 to Maier et al., the contents of which are incorporated herein by reference in their entirety.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-1 6, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl] pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1 S,2S)-2-{[(1R,2R)-2-pentylcyclopropyl]methyl}cyclopropyl]nonadecan-10-amine, N,N-dimethyl-1-[(1 S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1 S,2R)-2-octylcyclopropyl]heptyl} dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1 S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, the contents of which are herein incorporated by reference in their entirety.

In one embodiment, the nanoparticles described herein may comprise at least one cationic polymer described herein and/or known in the art.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; the contents of each of which is herein incorporated by reference in their entirety.

In one embodiment, the LNP formulations of the saRNA may contain PEG-c-DOMG at 3% lipid molar ratio. In another embodiment, the LNP formulations of the saRNA may contain PEG-c-DOMG at 1.5% lipid molar ratio.

In one embodiment, the pharmaceutical compositions of the saRNA may include at least one of the PEGylated lipids described in International Publication No. 2012099755, the contents of which is herein incorporated by reference in its entirety.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety). As another non-limiting example, the saRNA described herein may be formulated in a nanoparticle to be delivered by a parenteral route as described in U.S. Pub. No. 20120207845; the contents of which is herein incorporated by reference in its entirety. The cationic lipid may also be the cationic lipids disclosed in US20130156845 to Manoharan et al. and US 20130129785 to Manoharan et al., WO 2012047656 to Wasan et al., WO 2010144740 to Chen et al., WO 2013086322 to Ansell et al., or WO 2012016184 to Manoharan et al., the contents of each of which are incorporated herein by reference in their entirety.

In one embodiment, the saRNA of the present invention may be formulated with a plurality of cationic lipids, such as a first and a second cationic lipid as described in US20130017223 to Hope et al., the contents of which are incorporated herein by reference in their entirety. The first cationic lipid can be selected on the basis of a first property and the second cationic lipid can be selected on the basis of a second property, where the properties may be determined as outlined in US20130017223, the contents of which are herein incorporated by reference in its entirety. In one embodiment, the first and second properties are complementary.

In another embodiment, the saRNA may be formulated with a lipid particle comprising one or more cationic lipids and one or more second lipids, and one or more nucleic acids, wherein the lipid particle comprises a solid core, as described in US Patent Publication No. US20120276209 to Cullis et al., the contents of which are incorporated herein by reference in their entirety.

In one embodiment, the saRNA of the present invention may be complexed with a cationic amphiphile in an oil-in-water (o/w) emulsion such as described in EP2298358 to Satishchandran et al., the contents of which are incorporated herein by reference in their entirety. The cationic amphiphile may be a cationic lipid, modified or unmodified spermine, bupivacaine, or benzalkonium chloride and the oil may be a vegetable or an animal oil. As a non-limiting example, at least 10% of the nucleic acid-cationic amphiphile complex is in the oil phase of the oil-in-water emulsion (see e.g., the complex described in European Publication No. EP2298358 to Satishchandran et al., the contents of which are herein incorporated by reference in its entirety).

In one embodiment, the saRNA of the present invention may be formulated with a composition comprising a mixture of cationic compounds and neutral lipids. As a non-limiting example, the cationic compounds may be formula (I) disclosed in WO 1999010390 to Ansell et al., the contents of which are disclosed herein by reference in their entirety, and the neutral lipid may be selected from the group consisting of diacylphosphatidylcholine, diacylphosphatidylethanolamine, ceramide and sphingomyelin. In another non-limiting example, the lipid formulation may comprise a cationic lipid of formula A, a neutral lipid, a sterol and a PEG or PEG-modified lipid disclosed in US 20120101148 to Akinc et al., the contents of which are incorporated herein by reference in their entirety.

In one embodiment, the LNP formulation may be formulated by the methods described in International Publication Nos. WO2011127255 or WO2008103276, each of which are herein incorporated by reference in their entirety. As a non-limiting example, the saRNA of the present invention may be encapsulated in any of the lipid nanoparticle (LNP) formulations described in WO2011127255 and/or WO2008103276; the contents of each of which are herein incorporated by reference in their entirety.

In one embodiment, the LNP formulations described herein may comprise a polycationic composition. As a non-limiting example, the polycationic composition may be selected from formula 1-60 of US Patent Publication No. US20050222064; the contents of which is herein incorporated by reference in its entirety. In another embodiment, the LNP formulations comprising a polycationic composition may be used for the delivery of the saRNA described herein in vivo and/or in vitro.

In one embodiment, the LNP formulations described herein may additionally comprise a permeability enhancer molecule. Non-limiting permeability enhancer molecules are described in US Patent Publication No. US20050222064; the contents of which is herein incorporated by reference in its entirety.

In one embodiment, the pharmaceutical compositions may be formulated in liposomes such as, but not limited to, DiLa2 liposomes (Marina Biotech, Bothell, Wash.), SMARTICLES®/NOV340 (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); the contents of which is herein incorporated by reference in its entirety) and hyaluronan-coated liposomes (Quiet Therapeutics, Israel).

In some embodiments, the pharmaceutical compositions may be formulated with any amphoteric liposome disclosed in WO 2008/043575 to Panzner and U.S. Pat. No. 8,580,297 to Essler et al. (Marina Biotech), the contents of which are incorporated herein by reference in their entirety. The amphoteric liposome may comprise a mixture of lipids including a cationic amphiphile, an anionic amphiphile and optional one or more neutral amphiphiles. The amphoteric liposome may comprise amphoteric compounds based on amphiphilic molecules, the head groups of which being substituted with one or more amphoteric groups. In some embodiments, the pharmaceutical compositions may be formulated with an amphoteric lipid comprising one or more amphoteric groups having an isoelectric point between 4 and 9, as disclosed in US 20140227345 to Essler et al. (Marina Biotech), the contents of which are incorporated herein by reference in their entirety.

In some embodiments, the pharmaceutical composition may be formulated with liposomes comprising a sterol derivative as disclosed in U.S. Pat. No. 7,312,206 to Panzner et al. (Novosom), the contents of which are incorporated herein by reference in their entirety. In some embodiments, the pharmaceutical composition may be formulated with amphoteric liposomes comprising at least one amphipathic cationic lipid, at least one amphipathic anionic lipid, and at least one neutral lipid, or liposomes comprise at least one amphipathic lipid with both a positive and a negative charge, and at least one neutral lipid, wherein the liposomes are stable at pH 4.2 and pH 7.5, as disclosed in U.S. Pat. No. 7,780,983 to Panzner et al. (Novosom), the contents of which are incorporated herein by reference in their entirety. In some embodiments, the pharmaceutical composition may be formulated with liposomes comprising a serum-stable mixture of lipids taught in US 20110076322 to Panzner et al, the contents of which are incorporated herein by reference in their entirety, capable of encapsulating the saRNA of the present invention. The lipid mixture comprises phosphatidylcholine and phosphatidylethanolamine in a ratio in the range of about 0.5 to about 8. The lipid mixture may also include pH sensitive anionic and cationic amphiphiles, such that the mixture is amphoteric, being negatively charged or neutral at pH 7.4 and positively charged at pH 4. The drug/lipid ratio may be adjusted to target the liposomes to particular organs or other sites in the body. In some embodiments, liposomes loaded with the saRNA of the present invention as cargo, are prepared by the method disclosed in US 20120021042 to Panzner et al., the contents of which are incorporated herein by reference in their entirety. The method comprises steps of admixing an aqueous solution of a polyanionic active agent and an alcoholic solution of one or more amphiphiles and buffering said admixture to an acidic pH, wherein the one or more amphiphiles are susceptible of forming amphoteric liposomes at the acidic pH, thereby to form amphoteric liposomes in suspension encapsulating the active agent.

The nanoparticle formulations may be a carbohydrate nanoparticle comprising a carbohydrate carrier and a nucleic acid molecule (e.g., saRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which is herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the saRNA may be formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; the contents of each of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; the contents of each of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; the contents of which is herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; the contents of each of which are incorporated herein by reference in its entirety).

In one embodiment, the saRNA is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; the contents of which are herein incorporated by reference in its entirety).

In one embodiment, the saRNA of the present invention can be formulated for controlled release and/or targeted delivery. As used herein, “controlled release” refers to a pharmaceutical composition or compound release profile that conforms to a particular pattern of release to effect a therapeutic outcome. In one embodiment, the saRNA may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulated” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In another embodiment, the saRNA may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc., Deerfield, Ill.), PEG-based sealants, and COSEAL® (Baxter International, Inc., Deerfield, Ill.).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the saRNA formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the saRNA of the present invention may be formulated with a targeting lipid with a targeting moiety such as the targeting moieties disclosed in US20130202652 to Manoharan et al., the contents of which are incorporated herein by reference in their entirety. As a non-limiting example, the targeting moiety of formula I of US 20130202652 to Manoharan et al. may selected in order to favor the lipid being localized with a desired organ, tissue, cell, cell type or subtype, or organelle. Non-limiting targeting moieties that are contemplated in the present invention include transferrin, anisamide, an RGD peptide, prostate specific membrane antigen (PSMA), fucose, an antibody, or an aptamer.

In one embodiment, the saRNA of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which are herein incorporated by reference in its entirety.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the saRNA of the present invention (see International Pub No. 2010075072 and US Pub No. US20100216804, US20110217377 and US20120201859, the contents of each of which are herein incorporated by reference in their entirety).

In one embodiment, the therapeutic nanoparticles may be formulated to be target specific. As a non-limiting example, the therapeutic nanoparticles may include a corticosteroid (see International Pub. No. WO2011084518; the contents of which are herein incorporated by reference in its entirety). In one embodiment, the therapeutic nanoparticles may be formulated to be cancer specific. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, the contents of each of which are herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which is herein incorporated by reference in its entirety).

In one embodiment, the therapeutic nanoparticle may comprise a multiblock copolymer such as, but not limited to the multiblock copolymers described in U.S. Pat. Nos. 8,263,665 and 8,287,910; the contents of each of which are herein incorporated by reference in its entirety.

In one embodiment, the block copolymers described herein may be included in a polyion complex comprising a non-polymeric micelle and the block copolymer. (See e.g., U.S. Pub. No. 20120076836; the contents of which are herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; the contents of which are herein incorporated by reference in its entirety) and combinations thereof.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; the contents of which are herein incorporated by reference in its entirety).

In one embodiment, the therapeutic nanoparticle may be formulated in an aqueous solution which may be used to target cancer (see International Pub No. WO2011084513 and US Pub No. US20110294717, the contents of each of which is herein incorporated by reference in their entirety).

In one embodiment, the saRNA may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, the contents of each of which are herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, the contents of each of which are herein incorporated by reference in their entirety. In another embodiment, the synthetic nanocarrier formulations may be lyophilized by methods described in International Pub. No. WO2011072218 and U.S. Pat. No. 8,211,473; the contents of each of which are herein incorporated by reference in their entirety.

In one embodiment, the synthetic nanocarriers may contain reactive groups to release the saRNA described herein (see International Pub. No. WO20120952552 and US Pub No. US20120171229, the contents of each of which are herein incorporated by reference in their entirety).

In one embodiment, the synthetic nanocarriers may be formulated for targeted release. In one embodiment, the synthetic nanocarrier may be formulated to release the saRNA at a specified pH and/or after a desired time interval. As a non-limiting example, the synthetic nanoparticle may be formulated to release the saRNA after 24 hours and/or at a pH of 4.5 (see International Pub. Nos. WO2010138193 and WO2010138194 and US Pub Nos. US20110020388 and US20110027217, the contents of each of which is herein incorporated by reference in their entireties).

In one embodiment, the synthetic nanocarriers may be formulated for controlled and/or sustained release of the saRNA described herein. As a non-limiting example, the synthetic nanocarriers for sustained release may be formulated by methods known in the art, described herein and/or as described in International Pub No. WO2010138192 and US Pub No. 20100303850, the contents each of which is herein incorporated by reference in their entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; the contents of which are herein incorporated by reference in its entirety.

In one embodiment, the saRNA of the present invention may be formulated in a modular composition such as described in U.S. Pat. No. 8,575,123 to Manoharan et al., the contents of which are herein incorporated by reference in their entirety. As a non-limiting example, the modular composition may comprise a nucleic acid, e.g., the saRNA of the present invention, at least one endosomolytic component, and at least one targeting ligand. The modular composition may have a formula such as any formula described in U.S. Pat. No. 8,575,123 to Manoharan et al., the contents of which are herein incorporated by reference in their entirety.

In one embodiment, the saRNA of the present invention may be encapsulated in the lipid formulation to form a stable nucleic acid-lipid particle (SNALP) such as described in U.S. Pat. No. 8,546,554 to de Fougerolles et al., the contents of which are incorporated here by reference in their entirety. The lipid may be cationic or non-cationic. In one non-limiting example, the lipid to nucleic acid ratio (mass/mass ratio) (e.g., lipid to saRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or 5:1, 6:1, 7:1, 8:1, 9:1, 10:1, or 11:1. In another example, the SNALP includes 40% 2,2-Dilinoleyl-4-dimethylaminoethyl-[1,3]-dioxolane (Lipid A), 10% dioleoylphosphatidylcholine (DSPC), 40% cholesterol, 10% polyethyleneglycol (PEG)-C-DOMG (mole percent) with a particle size of 63.0±20 nm and a 0.027 nucleic acid/lipid ratio. In another embodiment, the saRNA of the present invention may be formulated with a nucleic acid-lipid particle comprising an endosomal membrane destabilizer as disclosed in U.S. Pat. No. 7,189,705 to Lam et al., the contents of which are incorporated herein by reference in their entirety. As a non-limiting example, the endosomal membrane destabilizer may be a Ca²⁺ ion.

In one embodiment, the saRNA of the present invention may be formulated with formulated lipid particles (FLiPs) disclosed in U.S. Pat. No. 8,148,344 to Akinc et al., the contents of which are herein incorporated by reference in their entirety. Akinc et al. teach that FLiPs may comprise at least one of a single or double-stranded oligonucleotide, where the oligonucleotide has been conjugated to a lipophile and at least one of an emulsion or liposome to which the conjugated oligonucleotide has been aggregated, admixed or associated. These particles have surprisingly been shown to effectively deliver oligonucleotides to heart, lung and muscle disclosed in U.S. Pat. No. 8,148,344 to Akinc et al., the contents of which are herein incorporated by reference in their entirety.

In one embodiment, the saRNA of the present invention may be delivered to a cell using a composition comprising an expression vector in a lipid formulation as described in U.S. Pat. No. 6,086,913 to Tam et al., the contents of which are incorporated herein by reference in their entirety. The composition disclosed by Tam is serum-stable and comprises an expression vector comprising first and second inverted repeated sequences from an adeno associated virus (AAV), a rep gene from AAV, and a nucleic acid fragment. The expression vector in Tam is complexed with lipids.

In one embodiment, the saRNA of the present invention may be formulated with a lipid formulation disclosed in US 20120270921 to de Fougerolles et al., the contents of which are incorporated herein by reference in their entirety. In one non-limiting example, the lipid formulation may include a cationic lipid having the formula A described in US 20120270921, the contents of which are herein incorporated by reference in its entirety. In another non-limiting example, the compositions of exemplary nucleic acid-lipid particles disclosed in Table A of US 20120270921, the contents of which are incorporated herein by reference in their entirety, may be used with the saRNA of the present invention.

In one embodiment, the saRNA of the present invention may be fully encapsulated in a lipid particle disclosed in US 20120276207 to Maurer et al., the contents of which are incorporated herein by reference in their entirety. The particles may comprise a lipid composition comprising preformed lipid vesicles, a charged therapeutic agent, and a destabilizing agent to form a mixture of preformed vesicles and therapeutic agent in a destabilizing solvent, wherein the destabilizing solvent is effective to destabilize the membrane of the preformed lipid vesicles without disrupting the vesicles.

In one embodiment, the saRNA of the present invention may be formulated with a conjugated lipid. In a non-limiting example, the conjugated lipid may have a formula such as described in US 20120264810 to Lin et al., the contents of which are incorporated herein by reference in their entirety. The conjugate lipid may form a lipid particle which further comprises a cationic lipid, a neutral lipid, and a lipid capable of reducing aggregation.

In one embodiment, the saRNA of the present invention may be formulated in a neutral liposomal formulation such as disclosed in US 20120244207 to Fitzgerald et al., the contents of which are incorporated herein by reference in their entirety. The phrase “neutral liposomal formulation” refers to a liposomal formulation with a near neutral or neutral surface charge at a physiological pH. Physiological pH can be, e.g., about 7.0 to about 7.5, or, e.g., about 7.5, or, e.g., 7.0, 7.1, 7.2, 7.3, 7.4, or 7.5, or, e.g., 7.3, or, e.g., 7.4. An example of a neutral liposomal formulation is an ionizable lipid nanoparticle (iLNP). A neutral liposomal formulation can include an ionizable cationic lipid, e.g., DLin-KC2-DMA.

In one embodiment, the saRNA of the present invention may be formulated with a charged lipid or an amino lipid. As used herein, the term “charged lipid” is meant to include those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.” In a non-limiting example, the amino lipid may be any amino lipid described in US20110256175 to Hope et al., the contents of which are incorporated herein by reference in their entirety. For example, the amino lipids may have the structure disclosed in Tables 3-7 of Hope, such as structure (II), DLin-K-C2-DMA, DLin-K2-DMA, DLin-K6-DMA, etc. The resulting pharmaceutical preparations may be lyophilized according to Hope. In another non-limiting example, the amino lipids may be any amino lipid described in US 20110117125 to Hope et al., the contents of which are incorporated herein by reference in their entirety, such as a lipid of structure (I), DLin-K-DMA, DLin-C-DAP, DLin-DAC, DLin-MA, DLin-S-DMA, etc. In another non-limiting example, the amino lipid may have the structure (I), (II), (III), or (IV), or 4-(R)-DUn-K-DMA (VI), 4-(S)-DUn-K-DMA (V) as described in WO2009132131 to Manoharan et al., the contents of which are incorporated herein by reference in their entirety. In another non-limiting example, the charged lipid used in any of the formulations described herein may be any charged lipid described in EP2509636 to Manoharan et al., the contents of which are incorporated herein by reference in their entirety.

In one embodiment, the saRNA of the present invention may be formulated with an association complex containing lipids, liposomes, or lipoplexes. In a non-limiting example, the association complex comprises one or more compounds each having a structure defined by formula (I), a PEG-lipid having a structure defined by formula (XV), a steroid and a nucleic acid disclosed in U.S. Pat. No. 8,034,376 to Manoharan et al., the contents of which are incorporated herein by reference in their entirety. The saRNA may be formulated with any association complex described in U.S. Pat. No. 8,034,376, the contents of which are herein incorporated by reference in its entirety.

In one embodiment, the saRNA of the present invention may be formulated with reverse head group lipids. As a non-limiting example, the saRNA may be formulated with a zwitterionic lipid comprising a headgroup wherein the positive charge is located near the acyl chain region and the negative charge is located at the distal end of the head group, such as a lipid having structure (A) or structure (I) described in WO2011056682 to Leung et al., the contents of which are incorporated herein by reference in their entirety.

In one embodiment, the saRNA of the present invention may be formulated in a lipid bilayer carrier. As a non-limiting example, the saRNA may be combined with a lipid-detergent mixture comprising a lipid mixture of an aggregation-preventing agent in an amount of about 5 mol % to about 20 mol %, a cationic lipid in an amount of about 0.5 mol % to about 50 mol %, and a fusogenic lipid and a detergent, to provide a nucleic acid-lipid-detergent mixture; and then dialyzing the nucleic acid-lipid-detergent mixture against a buffered salt solution to remove the detergent and to encapsulate the nucleic acid in a lipid bilayer carrier and provide a lipid bilayer-nucleic acid composition, wherein the buffered salt solution has an ionic strength sufficient to encapsulate of from about 40% to about 80% of the nucleic acid, described in WO1999018933 to Cullis et al., the contents of which are incorporated herein by reference in their entirety.

In one embodiment, the saRNA of the present invention may be formulated in a nucleic acid-lipid particle capable of selectively targeting the saRNA to a heart, liver, or tumor tissue site. For example, the nucleic acid-lipid particle may comprise (a) a nucleic acid; (b) 1.0 mole % to 45 mole % of a cationic lipid; (c) 0.0 mole % to 90 mole % of another lipid; (d) 1.0 mole % to 10 mole % of a bilayer stabilizing component; (e) 0.0 mole % to 60 mole % cholesterol; and (f) 0.0 mole % to 10 mole % of cationic polymer lipid as described in EP1328254 to Cullis et al., the contents of which are incorporated herein by reference in their entirety. Cullis teaches that varying the amount of each of the cationic lipid, bilayer stabilizing component, another lipid, cholesterol, and cationic polymer lipid can impart tissue selectivity for heart, liver, or tumor tissue site, thereby identifying a nucleic acid-lipid particle capable of selectively targeting a nucleic acid to the heart, liver, or tumor tissue site.

### Polymers, Biodegradable Nanoparticles, and Core-Shell Nanoparticles

The saRNA of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (PHASERX®, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers. RONDEL™ (RNAi/Oligonucleotide Nanoparticle Delivery) polymers (Arrowhead Research Corporation, Pasadena, Calif.) and pH responsive co-block polymers such as, but not limited to, PHASERX® (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering oligonucleotides in vivo into the cell cytoplasm (reviewed in de Fougerolles Hum Gene Ther. 2008 19:125-132; herein incorporated by reference in its entirety). Two polymer approaches that have yielded robust in vivo delivery of nucleic acids, in this case with small interfering RNA (siRNA), are dynamic polyconjugates and cyclodextrin-based nanoparticles. The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887; herein incorporated by reference in its entirety). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887; herein incorporated by reference in its entirety). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005 65: 8984-8982; herein incorporated by reference in its entirety) and siRNA formulated in these nanoparticles was well tolerated in non-human primates (Heidel et al., Proc Natl Acad Sci USA 2007 104:5715-21; herein incorporated by reference in its entirety). Both of these delivery strategies incorporate rational approaches using both targeted delivery and endosomal escape mechanisms.

The polymer formulation can permit the sustained or delayed release of saRNA (e.g., following intramuscular or subcutaneous injection). The altered release profile for the saRNA can result in, for example, translation of an encoded protein over an extended period of time. Biodegradable polymers have been previously used to protect nucleic acids from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; de Fougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070; each of which is herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example saRNA may be formulated in PLGA microspheres by preparing the PLGA microspheres with tunable release rates (e.g., days and weeks) and encapsulating the saRNA in the PLGA microspheres while maintaining the integrity of the saRNA during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; each of which is herein incorporated by reference in its entirety).

The saRNA of the invention may be formulated with or in a polymeric compound. The polymer may include at least one polymer such as, but not limited to, polyethenes, polyethylene glycol (PEG), poly(1-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the saRNA of the invention may be formulated with the polymeric compound of PEG grafted with PLL as described in U.S. Pat. No. 6,177,274; herein incorporated by reference in its entirety. The formulation may be used for transfecting cells in vitro or for in vivo delivery of the saRNA. In another example, the saRNA may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the saRNA of the invention may be formulated with a PLGA-PEG block copolymer (see US Pub. No. US20120004293 and U.S. Pat. No. 8,236,330, herein incorporated by reference in their entireties) or PLGA-PEG-PLGA block copolymers (See U.S. Pat. No. 6,004,573, herein incorporated by reference in its entirety). As a non-limiting example, the saRNA of the invention may be formulated with a diblock copolymer of PEG and PLA or PEG and PLGA (see U.S. Pat. No. 8,246,968, herein incorporated by reference in its entirety).

A polyamine derivative may be used to deliver nucleic acids or to treat and/or prevent a disease or to be included in an implantable or injectable device (U.S. Pub. No. 20100260817 herein incorporated by reference in its entirety). As a non-limiting example, a pharmaceutical composition may include the saRNA and the polyamine derivative described in U.S. Pub. No. 20100260817 (the contents of which are incorporated herein by reference in its entirety. As a non-limiting example the saRNA of the present invention may be delivered using a polyaminde polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

In one embodiment, the saRNA of the present invention may be formulated with at least one polymer and/or derivatives thereof described in International Publication Nos. WO2011115862, WO2012082574 and WO2012068187 and U.S. Pub. No. 20120283427, the contents of each of which are herein incorporated by reference in their entireties. In another embodiment, the saRNA of the present invention may be formulated with a polymer of formula Z as described in WO2011115862, herein incorporated by reference in its entirety. In yet another embodiment, the saRNA may be formulated with a polymer of formula Z, Z′ or Z″ as described in International Pub. Nos. WO2012082574 or WO2012068187 and U.S. Pub. No. 2012028342, the contents of each of which are herein incorporated by reference in their entireties. The polymers formulated with the saRNA of the present invention may be synthesized by the methods described in International Pub. Nos. WO2012082574 or WO2012068187, the contents of each of which are herein incorporated by reference in their entireties.

The saRNA of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

Formulations of saRNA of the invention may include at least one amine-containing polymer such as, but not limited to polylysine, polyethylene imine, poly(amidoamine) dendrimers or combinations thereof.

For example, the saRNA of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The saRNA of the invention may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The saRNA of the invention may be formulated with at least one crosslinkable polyester. Crosslinkable polyesters include those known in the art and described in US Pub. No. 20120269761, herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the saRNA described herein may be conjugated with another compound. Non-limiting examples of conjugates are described in U.S. Pat. Nos. 7,964,578 and 7,833,992, each of which are herein incorporated by reference in their entireties. In another embodiment, saRNA of the present invention may be conjugated with conjugates of formula 1-122 as described in U.S. Pat. Nos. 7,964,578 and 7,833,992, each of which are herein incorporated by reference in their entireties. The saRNA described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the saRNA described herein may be conjugated and/or encapsulated in gold-nanoparticles. (International Pub. No. WO201216269 and U.S. Pub. No. 20120302940; each of which is herein incorporated by reference in its entirety).

As described in U.S. Pub. No. 20100004313, herein incorporated by reference in its entirety, a gene delivery composition may include a nucleotide sequence and a poloxamer. For example, the saRNA of the present invention may be used in a gene delivery composition with the poloxamer described in U.S. Pub. No. 20100004313.

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety.

The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The saRNA of the invention may be formulated in a polyplex of one or more polymers (U.S. Pub. No. 20120237565 and 20120270927; each of which is herein incorporated by reference in its entirety). In one embodiment, the polyplex comprises two or more cationic polymers. The cationic polymer may comprise a poly(ethylene imine) (PEI) such as linear PEI.

The saRNA of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so to delivery of the saRNA may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; herein incorporated by reference in its entirety).

Biodegradable calcium phosphate nanoparticles in combination with lipids and/or polymers may be used to deliver saRNA in vivo. In one embodiment, a lipid coated calcium phosphate nanoparticle, which may also contain a targeting ligand such as anisamide, may be used to deliver the saRNA of the present invention. For example, to effectively deliver siRNA in a mouse metastatic lung model a lipid coated calcium phosphate nanoparticle was used (Li et al., J Contr Rel. 2010 142: 416-421; Li et al., J Contr Rel. 2012 158:108-114; Yang et al., Mol Ther. 2012 20:609-615; herein incorporated by reference in its entirety). This delivery system combines both a targeted nanoparticle and a component to enhance the endosomal escape, calcium phosphate, in order to improve delivery of the siRNA.

In one embodiment, calcium phosphate with a PEG-polyanion block copolymer may be used to delivery saRNA (Kazikawa et al., J Contr Rel. 2004 97:345-356; Kazikawa et al., J Contr Rel. 2006 111:368-370; herein incorporated by reference in its entirety).

In one embodiment, a PEG-charge-conversional polymer (Pitella et al., Biomaterials. 2011 32:3106-3114) may be used to form a nanoparticle to deliver the saRNA of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver saRNA to mouse hepatocytes after they covalently attach cholesterol to the nanoparticle.

In one embodiment, a hollow lipid core comprising a middle PLGA layer and an outer neutral lipid layer containing PEG may be used to delivery of the saRNA of the present invention. As a non-limiting example, in mice bearing a luciferase-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031; herein incorporated by reference in its entirety).

In one embodiment, the lipid nanoparticles may comprise a core of the saRNA disclosed herein and a polymer shell. The polymer shell may be any of the polymers described herein and are known in the art. In an additional embodiment, the polymer shell may be used to protect the modified nucleic acids in the core.

Core-shell nanoparticles for use with the saRNA of the present invention may be formed by the methods described in U.S. Pat. No. 8,313,777 herein incorporated by reference in its entirety.

In one embodiment, the core-shell nanoparticles may comprise a core of the saRNA disclosed herein and a polymer shell. The polymer shell may be any of the polymers described herein and are known in the art. In an additional embodiment, the polymer shell may be used to protect the saRNA in the core. As a non-limiting example, the core-shell nanoparticle may be used to treat an eye disease or disorder (See e.g. US Publication No. 20120321719, herein incorporated by reference in its entirety).

In one embodiment, the polymer used with the formulations described herein may be a modified polymer (such as, but not limited to, a modified polyacetal) as described in International Publication No. WO2011120053, herein incorporated by reference in its entirety.

### Delivery

The present disclosure encompasses the delivery of saRNA for any of therapeutic, prophylactic, pharmaceutical, diagnostic or imaging by any appropriate route taking into consideration likely advances in the sciences of drug delivery. Delivery may be naked or formulated.

The saRNA of the present invention may be delivered to a cell naked. As used herein in, “naked” refers to delivering saRNA free from agents which promote transfection. For example, the saRNA delivered to the cell may contain no modifications. The naked saRNA may be delivered to the cell using routes of administration known in the art and described herein.

The saRNA of the present invention may be formulated, using the methods described herein. The formulations may contain saRNA which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated saRNA may be delivered to the cell using routes of administration known in the art and described herein.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like. The saRNA of the present invention may also be cloned into a retroviral replicating vector (RRV) and transduced to cells.

### Administration

The saRNA of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Routes of administration disclosed in International Publication WO 2013/090648 filed Dec. 14, 2012, the contents of which are incorporated herein by reference in their entirety, may be used to administer the saRNA of the present invention.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous). Liquid dosage forms, injectable preparations, pulmonary forms, and solid dosage forms described in International Publication WO 2013/090648 filed Dec. 14, 2012, the contents of which are incorporated herein by reference in their entirety may be used as dosage forms for the saRNA of the present invention.

### III. Methods of Use

One aspect of the present invention provides methods of using saRNA of the present invention and pharmaceutical compositions comprising the saRNA and at least one pharmaceutically acceptable carrier. The saRNA of the present invention modulates the expression of its target gene. In one embodiment is provided a method of regulating the expression of a target gene in vitro and/or in vivo comprising administering the saRNA of the present invention. In one embodiment, the expression of the target gene is increased by at least 5, 10, 20, 30, 40%, or at least 45, 50, 55, 60, 65, 70, 75%, or at least 80% in the presence of the saRNA of the present invention compared to the expression of the target gene in the absence of the saRNA of the present invention. In a further embodiment, the expression of the target gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present invention compared to the expression of the target gene in the absence of the saRNA of the present invention.

The target gene may be any gene of the human genome.

Non-limited examples include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), and coagulation factor VIII (F8).

### APOA1 Gene

As a non-limited example, provided is a method of modulating the expression of APOA1 gene comprising administering saRNA of the present invention, wherein the saRNA targets an antisense RNA transcript of APOA1 gene. These saRNAs are called APOA1-saRNA. In one embodiment, the expression of APOA1 gene is increased by at least 20, 30, 40%, or at least 45, 50, 55, 60, 65, 70, 75%, or at least 80% in the presence of the APOA1-saRNA of the present invention compared to the expression of APOA1 gene in the absence of the APOA1-saRNA of the present invention. In a further embodiment, the expression of APOA1 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the APOA1-saRNA of the present invention compared to the expression of APOA1 gene in the absence of the APOA1-saRNA of the present invention. The modulation of the expression of APOA1 gene may be reflected or determined by the change of APOA1 mRNA levels.

APOA1-saRNAs may be single-stranded and comprise 14-30 nucleotides. The sequence of a single-stranded APOA1-saRNA may have at least 60%, 70%, 80% or 90% identity with a sequence selected from the sequences of the antisense strands in Table 2-2. In one embodiment, the single-stranded saRNA comprises a sequence selected from the sequences of the antisense strands in Table 2-2. In one embodiment, the single-stranded APOA1-saRNA may have a 3′ tail. The sequence of a single-stranded APOA1-saRNA with a 3′ tail may have at least 60%, 70%, 80% or 90% identity with a sequence selected from the sequences of the antisense strands in Table 3. In one embodiment, the single-stranded saRNA comprises a sequence selected from the sequences of the antisense strands in Table 3.

APOA1-saRNAs may be double-stranded. The two strands form a duplex and each strand comprises 14-30 nucleotides. The first strand of a double-stranded APOA1-saRNA may have at least 60%, 70%, 80% or 90% identity with a sequence selected from the sequences of the antisense strands in Table 2-2. In one embodiment, the first strand of the double-stranded saRNA comprises a sequence selected from the sequences of the antisense strands in Table 2-2. The second strand of a double-stranded APOA1-saRNA may have at least 60%, 70%, 80% or 90% identity with a sequence selected from the sequences of the sense strands in Table 2-2. In one embodiment, the second strand of the double-stranded saRNA comprises a sequence selected from the sequences of the sense strands in Table 2-2. In one embodiment, the double-stranded APOA1-saRNA may have a 3′ overhang on each strand. The first strand of a double-stranded APOA1-saRNA may have at least 60%, 70%, 80% or 90% identity with a sequence selected from the sequences of the antisense strands in Table 3. In one embodiment, the first strand of the double-stranded saRNA comprises a sequence selected from the sequences of the antisense strands in Table 3. The second strand of a double-stranded APOA1-saRNA may have at least 60%, 70%, 80% or 90% identity with a sequence selected from the sequences of the sense strands in Table 3. In one embodiment, the second strand of the double-stranded saRNA comprises a sequence selected from the sequences of the sense strands in Table 3.

APOA1-saRNAs may be modified or unmodified.

The APOA1 gene encodes a protein called apolipoprotein AI (APOA-I or APOA1). ApoA-I is a component of high-density lipoprotein (HDL). HDL is a molecule that transports cholesterol and certain fats called phospholipids through the bloodstream from the body's tissues to the liver. Once in the liver, cholesterol and phospholipids are redistributed to other tissues or removed from the body. ApoA-I protein attaches to cell membranes and promotes the movement of cholesterol and phospholipids from inside the cell to the outer surface. Once outside the cell, these substances combine with apoA-I protein to form HDL. ApoA-I protein also triggers a reaction called cholesterol esterification that converts cholesterol to a form that can be fully integrated into HDL and transported through the bloodstream. HDL is often referred to as “good cholesterol” because high levels of this substance reduce the chances of developing heart and blood vessel (cardiovascular) disease. The process of removing excess cholesterol from cells is extremely important for balancing cholesterol levels and maintaining cardiovascular health. Mutations in APOA1 gene cause various diseases such as familial HDL deficiency and familial visceral amyloidosis (Santos et al., J Lipid Res, vol. 49 (2):349 (2008); Rowczenio et al., Am J Pathol, vol. 179 (4): 1978 (2011), the contents of each of which are incorporated herein by reference in their entirety). Familial HDL deficiency is characterized by low levels of HDL in the blood, which increases the risk for cardiovascular disease. Familial visceral amyloidosis is characterized by an abnormal accumulation of proteins (amyloidosis) in internal organs (viscera), in particular liver, kidneys, and heart. People with visceral amyloidosis might develop an enlarged liver, chronic kidney disease, or heart diseases such as cardiomyopathy.

Table 2-1 describes the target gene, the targeted sequence, the genomic location of the targeted sequence and the relative location of APOA1-saRNAs with no 3′ overhang. In Table 2-1, the targeted sequence is defined as a region on the template strand of the target gene which is identical to a region on the target antisense RNA transcript to which the antisense strand of an saRNA hybridizes. In Table 2-1, the relative location is the distance from the 5′ end of the targeted sequence to the TSS. A negative number represents a location upstream of the TSS and a positive number represents a location downstream of the TSS.

TABLE 2-1
Sequences of APOA1-saRNAs (with no 3′ overhang)

| | | | Genomic Location of | Relative |
|---|---|---|---|---|
| saRNA ID | Target Gene | Targeted sequence | the Targeted sequence | Location |
| APOA1-Pr-1 | APOA1 | ccagcattccagggagact | chr11: 116708231 plus | 107 |
| | human | (SEQ ID NO. 72) | strand | |
| APOA1-Pr-2 | APOA1 | ggctgagatctgagccgaa | chr11: 116707412 plus | 926 |
| | human | (SEQ ID NO. 73) | strand | |
| APOA1-Pr-4 | APOA1 | cccagctcatcagatatta | chr11: 116707610 plus | 728 |
| | human | (SEQ ID NO. 74) | strand | |
| APOA1-mm10-Pr-1 | APOA1 | gcgagatatccacggtatt | chr11: 116707639 plus | 699 |
| | mouse | (SEQ ID NO. 75) | strand | |
| APOA1-mm10-Pr-2 | APOA1 | gtgtatgtcttcttcaaga | chr11: 116708035 plus | 303 |
| | mouse | (SEQ ID NO. 76) | strand | |
| APOA1-mm10-Pr-3 | APOA1 | gaggcattgtcatggaaag | chr11: 116707568 plus | 770 |
| | mouse | (SEQ ID NO. 77) | strand | |
| APOA1-mm10-Pr-4 | APOA1 | gggatttgtctttccagat | chr11: 116708227 plus | 111 |
| | mouse | (SEQ ID NO. 78) | strand | |
| APOA1-rn5-Pr-1 | APOA1 rat | cagctttcatcctgaagga | chr11: 116707587 plus | 751 |
| | | (SEQ ID NO. 79) | strand | |
| APOA1-rn5-Pr-2 | APOA1 rat | cgttgcattccaagaggtt | chr11: 116708255 plus | 83 |
| | | (SEQ ID NO. 80) | strand | |
| APOA1-rn5-Pr-3 | APOA1 rat | ggaacttagagttcaagga | chr11: 116707948 plus | 390 |
| | | (SEQ ID NO. 81) | strand | |
| APOA1-rn5-Pr-4 | APOA1 rat | gggacagatcccagatatc | chr11: 116707712 plus | 626 |
| | | (SEQ ID NO. 82) | strand | |
| APOA1.NM_000039- | APOA1 | gggcataatctcagaatta | chr11: 116709049 plus | −711 |
| Pr-1 or APOA1- | human | (SEQ ID NO. 83) | strand | |
| Pr-3 | | | | |
| APOA1.NM_000039- | APOA1 | agggcataatctcagaatt | chr11: 116709048 plus | −710 |
| Pr-3 | human | (SEQ ID NO. 84) | strand | |
| APOA1.NM_000039- | APOA1 | ggcataatctcagaattac | chr11: 116709050 plus | −712 |
| Pr-6 | human | (SEQ ID NO. 85) | strand | |
| APOA1.NM_000039- | APOA1 | cccagctcatcagatatta | chr11: 116707610 plus | 728 |
| Pr-16 | human | (SEQ ID NO. 86) | strand | |
| APOA1.NM_000039- | APOA1 | cttaagagttcaaggatca | chr11: 116708450 plus | −112 |
| Pr-21 | human | (SEQ ID NO. 87) | strand | |
| APOA1.NM_000039- | APOA1 | agatattgtccgcctccaa | chr11: 116708972 plus | −634 |
| Pr-23 | human | (SEQ ID NO. 88) | strand | |
| APOA1.NM_000039- | APOA1 | agatcgtgggcaagttata | chr11: 116709259 plus | −921 |
| Pr-25 | human | (SEQ ID NO. 89) | strand | |
| APOA1.NM_000039- | APOA1 | ggctgagatctgagccgaa | chr11: 116707912 plus | 426 |
| Pr-37 | human | (SEQ ID NO. 90) | strand | |

TABLE 2-2
Sequences of APOA1-saRNAs (with no 3′ overhang)

| saRNA ID | Sense (passenger) | Antisense (guide) |
|---|---|---|
| APOA1-Pr-1 | CCAGCAUUCCAGGGAGACU | AGUCUCCCUGGAAUGCUGG |
| | (SEQ ID NO. 91) | (SEQ ID NO. 92) |
| APOA1-Pr-2 | GGCUGAGAUCUGAGCCGAA | UUCGGCUCAGAUCUCAGCC |
| | (SEQ ID NO. 93) | (SEQ ID NO. 94) |
| APOA1-Pr-4 | CCCAGCUCAUCAGAUAUUA | UAAUAUCUGAUGAGCUGGG |
| | (SEQ ID NO. 95) | (SEQ ID NO. 96) |
| APOA1-mm10-Pr-1 | GCGAGAUAUCCACGGUAUU | AAUACCGUGGAUAUCUCGC |
| | (SEQ ID NO. 97) | (SEQ ID NO. 98) |
| APOA1-mm10-Pr-2 | GUGUAUGUCUUCUUCAAGA | UCUUGAAGAAGACAUACAC |
| | (SEQ ID NO. 99) | (SEQ ID NO. 100) |
| APOA1-mm10-Pr-3 | GAGGCAUUGUCAUGGAAAG | CUUUCCAUGACAAUGCCUC |
| | (SEQ ID NO. 101) | (SEQ ID NO. 102) |
| APOA1-mm10-Pr-4 | GGGAUUUGUCUUUCCAGAU | AUCUGGAAAGACAAAUCCC |
| | (SEQ ID NO. 103) | (SEQ ID NO. 104) |
| APOA1-rn5-Pr-1 | CAGCUUUCAUCCUGAAGGA | UCCUUCAGGAUGAAAGCUG |
| | (SEQ ID NO. 105) | (SEQ ID NO. 106) |
| APOA1-rn5-Pr-2 | CGUUGCAUUCCAAGAGGUU | AACCUCUUGGAAUGCAACG |
| | (SEQ ID NO. 107) | (SEQ ID NO. 108) |
| APOA1-rn5-Pr-3 | GGAACUUAGAGUUCAAGGA | UCCUUGAACUCUAAGUUCC |
| | (SEQ ID NO. 109) | (SEQ ID NO. 110) |
| APOA1-rn5-Pr-4 | GGGACAGAUCCCAGAUAUC | GAUAUCUGGGAUCUGUCCC |
| | (SEQ ID NO. 111) | (SEQ ID NO. 112) |
| APOA1.NM_000039-Pr-1 | GGGCAUAAUCUCAGAAUUA | UAAUUCUGAGAUUAUGCCC |
| or APOA1-Pr-3 | (SEQ ID NO. 113) | (SEQ ID NO. 114) |
| APOA1.NM_000039-Pr-3 | AGGGCAUAAUCUCAGAAUU | AAUUCUGAGAUUAUGCCCU |
| | (SEQ ID NO. 115) | (SEQ ID NO. 116) |
| APOA1.NM_000039-Pr-6 | GGCAUAAUCUCAGAAUUAC | GUAAUUCUGAGAUUAUGCC |
| | (SEQ ID NO. 117) | (SEQ ID NO. 118) |
| APOA1.NM_000039-Pr- | CCCAGCUCAUCAGAUAUUA | UAAUAUCUGAUGAGCUGGG |
| 16 | (SEQ ID NO. 119) | (SEQ ID NO. 120) |
| APOA1.NM_000039-Pr- | CUUAAGAGUUCAAGGAUCA | UGAUCCUUGAACUCUUAAG |
| 21 | (SEQ ID NO. 121) | (SEQ ID NO. 122) |
| APOA1.NM_000039-Pr- | AGAUAUUGUCCGCCUCCAA | UUGGAGGCGGACAAUAUCU |
| 23 | (SEQ ID NO. 123) | (SEQ ID NO. 124) |
| APOA1.NM_000039-Pr- | AGAUCGUGGGCAAGUUAUA | UAUAACUUGCCCACGAUCU |
| 25 | (SEQ ID NO. 125) | (SEQ ID NO. 126) |
| APOA1.NM_000039-Pr- | GGCUGAGAUCUGAGCCGAA | UUCGGCUCAGAUCUCAGCC |
| 37 | (SEQ ID NO. 127) | (SEQ ID NO. 128) |

TABLE 3
Sequences of APOA1-saRNAs (with 3′ overhang)

| saRNA ID | Sense (passenger) | Antisense (guide) |
|---|---|---|
| APOA1-Pr-1′ | CCAGCAUUCCAGGGAGACUUU | AGUCUCCCUGGAAUGCUGGUU |
| | (SEQ ID NO. 129) | (SEQ ID NO. 130) |
| APOA1-Pr-2′ | GGCUGAGAUCUGAGCCGAAUU | UUCGGCUCAGAUCUCAGCCUU |
| | (SEQ ID NO. 131) | (SEQ ID NO. 132) |
| APOA1-Pr-4′ | CCCAGCUCAUCAGAUAUUAUU | UAAUAUCUGAUGAGCUGGGUU |
| | (SEQ ID NO. 133) | (SEQ ID NO. 134) |
| APOA1-mm10-Pr-1′ | GCGAGAUAUCCACGGUAUUUU | AAUACCGUGGAUAUCUCGCUU |
| | (SEQ ID NO. 135) | (SEQ ID NO. 136) |
| APOA1-mm10-Pr-2′ | GUGUAUGUCUUCUUCAAGAUU | UCUUGAAGAAGACAUACACUU |
| | (SEQ ID NO. 137) | (SEQ ID NO. 138) |
| APOA1-mm10-Pr-3′ | GAGGCAUUGUCAUGGAAAGUU | CUUUCCAUGACAAUGCCUCUU |
| | (SEQ ID NO. 139) | (SEQ ID NO. 140) |
| APOA1-mm10-Pr-4′ | GGGAUUUGUCUUUCCAGAUUU | AUCUGGAAAGACAAAUCCCUU |
| | (SEQ ID NO. 141) | (SEQ ID NO. 142) |
| APOA1-rn5-Pr-1′ | CAGCUUUCAUCCUGAAGGAUU | UCCUUCAGGAUGAAAGCUGUU |
| | (SEQ ID NO. 143) | (SEQ ID NO. 144) |
| APOA1-rn5-Pr-2′ | CGUUGCAUUCCAAGAGGUUUU | AACCUCUUGGAAUGCAACGUU |
| | (SEQ ID NO. 145) | (SEQ ID NO. 146) |
| APOA1-rn5-Pr-3′ | GGAACUUAGAGUUCAAGGAUU | UCCUUGAACUCUAAGUUCCUU |
| | (SEQ ID NO. 147) | (SEQ ID NO. 148) |
| APOA1-rn5-Pr-4′ | GGGACAGAUCCCAGAUAUCUU | GAUAUCUGGGAUCUGUCCCUU |
| | (SEQ ID NO. 149) | (SEQ ID NO. 150) |
| APOA1.NM_000039- | GGGCAUAAUCUCAGAAUUAUU | UAAUUCUGAGAUUAUGCCCUU |
| Pr-1′ or APOA1-Pr- | (SEQ ID NO. 151) | (SEQ ID NO. 152) |
| 3′ | | |
| APOA1.NM_000039- | AGGGCAUAAUCUCAGAAUUUU | AAUUCUGAGAUUAUGCCCUUU |
| Pr-3′ | (SEQ ID NO. 153) | (SEQ ID NO. 154) |
| APOA1.NM_000039- | GGCAUAAUCUCAGAAUUACUU | GUAAUUCUGAGAUUAUGCCUU |
| Pr-6′ | (SEQ ID NO. 155) | (SEQ ID NO. 156) |
| APOA1.NM_000039- | CCCAGCUCAUCAGAUAUUAUU | UAAUAUCUGAUGAGCUGGGUU |
| Pr-16′ | (SEQ ID NO. 157) | (SEQ ID NO. 158) |
| APOA1.NM_000039- | CUUAAGAGUUCAAGGAUCAUU | UGAUCCUUGAACUCUUAAGUU |
| Pr-21′ | (SEQ ID NO. 159) | (SEQ ID NO. 160) |
| APOA1.NM_000039- | AGAUAUUGUCCGCCUCCAAUU | UUGGAGGCGGACAAUAUCUUU |
| Pr-23′ | (SEQ ID NO. 161) | (SEQ ID NO. 162) |
| APOA1.NM_000039- | AGAUCGUGGGCAAGUUAUAUU | UAUAACUUGCCCACGAUCUUU |
| Pr-25′ | (SEQ ID NO. 163) | (SEQ ID NO. 164) |
| APOA1.NM_000039- | GGCUGAGAUCUGAGCCGAAUU | UUCGGCUCAGAUCUCAGCCUU |
| Pr-37′ | (SEQ ID NO. 165) | (SEQ ID NO. 166) |

In one embodiment, provided is a method of increasing apoA-I protein levels comprising administering APOA1-saRNA of the present invention, wherein the APOA1-saRNA targets an antisense RNA transcript of APOA1 gene. In one embodiment, the apoA-I protein level is increased by at least 20, 30, 40%, or at least 45, 50, 55, 60, 65, 70, 75%, or at least 80% in the presence of the APOA1-saRNA of the present invention compared to apoA-I protein level in the absence of the APOA1-saRNA of the present invention. In a further embodiment, the apoA-I protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the APOA1-saRNA of the present invention compared to the apoA-I protein level in the absence of the APOA1-saRNA of the present invention.

In another embodiment, provided is a method of modulating HDL levels in a cell or in the blood comprising administering APOA1-saRNA of the present invention, wherein the APOA1-saRNA targets an antisense RNA transcript of APOA1 gene. In one embodiment, HDL level is increased by at least 20, 30, 40%, or at least 45, 50, 55, 60, 65, 70, 75%, or at least 80% in the presence of the APOA1-saRNA of the present invention compared to HDL level in the absence of the APOA1-saRNA of the present invention. In another embodiment, HDL level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the APOA1-saRNA of the present invention compared to HDL level in the absence of the APOA1-saRNA of the present invention.

In another embodiment, provided is a method of modulating cholesterol levels comprising administering APOA1-saRNA of the present invention, wherein the saRNA targets an antisense RNA transcript of APOA1 gene. In one embodiment, cholesterol level is reduced by at least 10%, 20%, 30%, or at least 40%, 50%, 60%, or at least 70%, 80%, 90% in the presence of the APOA1-saRNA of the present invention compared to cholesterol level in the absence of the APOA1-saRNA of the present invention.

In another embodiment, provided is a method of treating familial HDL deficiency or familial visceral amyloidosis comprising administering APOA1-saRNA of the present invention, wherein the APOA1-saRNA targets an antisense RNA transcript of APOA1 gene, and wherein the symptoms of familial HDL deficiency or familial visceral amyloidosis are reduced.

In one embodiment, the APOA1-saRNA of the present invention is an antisense sequence such as, but not limited to, SEQ ID NO: 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158 and 2160. As a non-limiting example, these APOA1-saRNA sequences may be used to increase APOAI protein levels, modulate HDL levels in a cell or the blood, modulate cholesterol levels and/or treat familial HDL deficiency or familial visceral amyloidosis.

In one embodiment, the APOA1-saRNA of the present invention is an saRNA duplex. The saRNA duplex may be a pair of sense and antisense sequences such as, but not limited to, SEQ ID NOs: 91 and 92; SEQ ID NOs: 93 and 94; SEQ ID NOs: 95 and 96; SEQ ID NOs: 97 and 98; SEQ ID NOs: 99 and 100; SEQ ID NOs: 101 and 102; SEQ ID NOs: 103 and 104; SEQ ID NOs: 105 and 106; SEQ ID NOs: 107 and 108; SEQ ID NOs: 109 and 110; SEQ ID NOs: 111 and 112; SEQ ID NOs: 113 and 114; SEQ ID NOs: 115 and 116; SEQ ID NOs: 117 and 118; SEQ ID NOs: 119 and 120; SEQ ID NOs: 121 and 122; SEQ ID NOs: 123 and 124; SEQ ID NOs: 125 and 126; SEQ ID NOs: 127 and 128; SEQ ID NOs: 129 and 130; SEQ ID NOs: 131 and 132; SEQ ID NOs: 133 and 134; SEQ ID NOs: 135 and 136; SEQ ID NOs: 137 and 138; SEQ ID NOs: 139 and 140; SEQ ID NOs: 141 and 142; SEQ ID NOs: 143 and 144; SEQ ID NOs: 145 and 146; SEQ ID NOs: 147 and 148; SEQ ID NOs: 149 and 150; SEQ ID NOs: 151 and 152; SEQ ID NOs: 153 and 154; SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 2061 and 2062; SEQ ID NOs: 2063 and 2064; SEQ ID NOs: 2065 and 2066; SEQ ID NOs: 2067 and 2068; SEQ ID NOs: 2069 and 2070; SEQ ID NOs: 2071 and 2072; SEQ ID NOs: 2073 and 2074; SEQ ID NOs: 2075 and 2076; SEQ ID NOs: 2077 and 2078; SEQ ID NOs: 2079 and 2080; SEQ ID NOs: 2081 and 2082; SEQ ID NOs: 2083 and 2084; SEQ ID NOs: 2085 and 2086; SEQ ID NOs: 2087 and 2088; SEQ ID NOs: 2089 and 2090; SEQ ID NOs: 2091 and 2092; SEQ ID NOs: 2093 and 2094; SEQ ID NOs: 2095 and 2096; SEQ ID NOs: 2097 and 2098; SEQ ID NOs: 2099 and 2100; SEQ ID NOs: 2101 and 2102; SEQ ID NOs: 2103 and 2104; SEQ ID NOs: 2105 and 2106; SEQ ID NOs: 2107 and 2108; SEQ ID NOs: 2109 and 2110; SEQ ID NOs: 2111 and 2112; SEQ ID NOs: 2113 and 2114; SEQ ID NOs: 2115 and 2116; SEQ ID NOs: 2117 and 2118; SEQ ID NOs: 2119 and 2120; SEQ ID NOs: 2121 and 2122; SEQ ID NOs: 2123 and 2124; SEQ ID NOs: 2125 and 2126; SEQ ID NOs: 2127 and 2128; SEQ ID NOs: 2129 and 2130; SEQ ID NOs: 2131 and 2132; SEQ ID NOs: 2133 and 2134; SEQ ID NOs: 2135 and 2136; SEQ ID NOs: 2137 and 2138; SEQ ID NOs: 2139 and 2140; SEQ ID NOs: 2141 and 2142; SEQ ID NOs: 2143 and 2144; SEQ ID NOs: 2145 and 2146; SEQ ID NOs: 2147 and 2148; SEQ ID NOs: 2149 and 2150; SEQ ID NOs: 2151 and 2152; SEQ ID NOs: 2153 and 2154; SEQ ID NOs: 2155 and 2156; SEQ ID NOs: 2157 and 2158; SEQ ID NOs: 2159 and 2160. As a non-limiting example, these APOA1-saRNA sequences which are saRNA duplexes may be used to increase APOAI protein levels, modulate HDL levels in a cell or the blood, modulate cholesterol levels and/or treat familial HDL deficiency or familial visceral amyloidosis.

### LDLR Gene

As another non-limiting example, provided is a method of modulating the expression of LDLR gene comprising administering saRNA of the present invention, wherein the saRNA targets an antisense RNA transcript of LDLR gene. These saRNAs are called LDLR-saRNA. In one embodiment, the expression of LDLR gene is increased by at least 20, 30, 40%, or at least 45, 50, 55, 60, 65, 70, 75%, or at least 80% in the presence of the LDLR-saRNA of the present invention compared to the expression of LDLR gene in the absence of the LDLR-saRNA of the present invention. In a further embodiment, the expression of LDLR is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the LDLR-saRNA of the present invention compared to the expression of LDLR gene in the absence of the LDLR-saRNA of the present invention. The modulation of the expression of LDLR gene may be reflected or determined by the change of LDLR mRNA levels.

LDLR-saRNAs may be single-stranded and comprise 14-30 nucleotides. The sequence of a single-stranded LDLR-saRNA may have at least 60%, 70%, 80% or 90% identity with a sequence selected from the sequences of the antisense strands in Table 4-2. In one embodiment, the single-stranded saRNA comprises a sequence selected from the sequences of the antisense strands in Table 4-2. In one embodiment, the single-stranded LDLR-saRNA may have a 3′ tail. The sequence of a single-stranded LDLR-saRNA with a 3′ tail may have at least 60%, 70%, 80% or 90% identity with a sequence selected from the sequences of the antisense strands in Table 5. In one embodiment, the single-stranded saRNA comprises a sequence selected from the sequences of the antisense strands in Table 5.

LDLR-saRNAs may be double-stranded. The two strands form a duplex and each strand comprises 14-30 nucleotides. The first strand of a double-stranded LDLR-saRNA may have at least 60%, 70%, 80% or 90% identity with a sequence selected from the sequences of the antisense strands in Table 4-2. In one embodiment, the first strand of the double-stranded saRNA comprises a sequence selected from the sequences of the antisense strands in Table 4-2. The second strand of a double-stranded LDLR-saRNA may have at least 60%, 70%, 80% or 90% identity with a sequence selected from the sequences of the sense strands in Table 4-2. In one embodiment, the second strand of the double-stranded saRNA comprises a sequence selected from the sequences of the sense strands in Table 4-2. In one embodiment, the double-stranded LDLR-saRNA may have a 3′ overhang on each strand. The first strand of a double-stranded LDLR-saRNA may have at least 60%, 70%, 80% or 90% identity with a sequence selected from the sequences of the antisense strands in Table 5. In one embodiment, the first strand of the double-stranded saRNA comprises a sequence selected from the sequences of the antisense strands in Table 5. The second strand of a double-stranded LDLR-saRNA may have at least 60%, 70%, 80% or 90% identity with a sequence selected from the sequences of the sense strands in Table 5. In one embodiment, the second strand of the double-stranded saRNA comprises a sequence selected from the sequences of the sense strands in Table 5.

LDLR-saRNAs may be modified or unmodified.

Table 4-1 describes the target gene, the targeted sequence, the genomic location of the targeted sequence and the relative location of LDLR-saRNAs with no 3′ overhang. In Table 4-1, the targeted sequence is defined as a region on the template strand of the target gene which is identical to a region on the target antisense RNA transcript to which the antisense strand of an saRNA hybridizes. In Table 4-1, the relative location is the distance from the 5′ end of the targeted sequence to the TSS. A negative number represents a location upstream of the TSS and a positive number represents a location downstream of the TSS.

TABLE 4-1
Sequences of LDLR-saRNAs (with no 3′ overhang)

| | | | Genomic Location of | Relative |
|---|---|---|---|---|
| saRNA ID | Target gene | Targeted sequence | the Targeted sequence | Location |
| LDLR-Pr-1 | LDLR human | gaaatttctcgatgtttaa | chr19: 111100342 | 324 |
| | | (SEQ ID NO. 167) | minus strand | |
| LDLR-Pr-2 | LDLR human | aacgcatcttctgaaagat | chr19: 111100367 | 349 |
| | | (SEQ ID NO. 168) | minus strand | |
| LDLR-Pr-4 | LDLR human | gcctgtaatcccagctatt | chr19: 111100664 | 646 |
| | | (SEQ ID NO. 169) | minus strand | |
| LDLR-mm10-Pr-1 | LDLR mouse | gtgggaagttcctcaaatt | chr19: 111100418 | 400 |
| | | (SEQ ID NO. 170) | minus strand | |
| LDLR-mm10-Pr-2 | LDLR mouse | gttgtatttctcgaccaaa | chr19: 111100883 | 865 |
| | | (SEQ ID NO. 171) | minus strand | |
| LDLR-mm10-Pr-3 | LDLR mouse | gaaatgttcccgctgcaaa | chr19: 111100678 | 660 |
| | | (SEQ ID NO. 172) | minus strand | |
| LDLR-mm10-Pr-4 | LDLR mouse | tagatttctgacccaagta | chr19: 111100184 | 166 |
| | | (SEQ ID NO. 173) | minus strand | |
| LDLR-rn5-Pr-1 | LDLR rat | gaaatgttcccgctgcaaa | chr19: 111100631 | 613 |
| | | (SEQ ID NO. 174) | minus strand | |
| LDLR-rn5-Pr-2 | LDLR rat | caagttcgcatgagtgatt | chr19: 111100850 | 832 |
| | | (SEQ ID NO. 175) | minus strand | |
| LDLR-rn5-Pr-3 | LDLR rat | tcgagttcctccttcagaa | chr19: 111100329 | 311 |
| | | (SEQ ID NO. 176) | minus strand | |
| LDLR-rn5-Pr-4 | LDLR rat | cacacatagctctgaagaa | chr19: 111100987 | 969 |
| | | (SEQ ID NO. 177) | minus strand | |
| LDLR.NM_000527- | LDLR human | gaaatttctcgatgtttaa | chr19: 11199842 minus | −176 |
| Pr-4 | | (SEQ ID NO. 178) | strand | |
| LDLR.NM_000527- | LDLR human | aacgcatcttctgaaagat | chr19: 11199867 minus | −151 |
| Pr-6 | | (SEQ ID NO. 179) | strand | |
| LDLR.NM_000527- | LDLR human | cctgaaatttctcgatgtt | chr19: 11199845 minus | −173 |
| Pr-7 | | (SEQ ID NO. 180) | strand | |
| LDLR.NM_000527- | LDLR human | ggagtctggacgtaaatat | chr19: 11199468 minus | −550 |
| Pr-13 or LDLR-Pr-3 | | (SEQ ID NO. 181) | strand | |
| LDLR.NM_000527- | LDLR human | cgcctttggctgagtata | chr19: 11199517 minus | −501 |
| Pr-22 | | (SEQ ID NO. 182) | strand | |
| LDLR.NM_000527- | LDLR human | gcctttggctgagtataaa | chr19: 11199516 minus | −502 |
| Pr-23 | | (SEQ ID NO. 183) | strand | |
| LDLR.NM_000527- | LDLR human | aaacgcatcttctgaaaga | chr19: 11199868 minus | −150 |
| Pr-28 | | (SEQ ID NO. 184) | strand | |

TABLE 4-2
Sequences of LDLR-saRNAs (with no 3′ overhang)

| saRNA ID | Sense (passenger) | Antisense (guide) |
|---|---|---|
| LDLR-Pr-1 | GAAAUUUCUCGAUGUUUAA | UUAAACAUCGAGAAAUUUC |
| | (SEQ ID NO. 185) | (SEQ ID NO. 186) |
| LDLR-Pr-2 | AACGCAUCUUCUGAAAGAU | AUCUUUCAGAAGAUGCGUU |
| | (SEQ ID NO. 187) | (SEQ ID NO. 188) |
| LDLR-Pr-4 | GCCUGUAAUCCCAGCUAUU | AAUAGCUGGGAUUACAGGC |
| | (SEQ ID NO. 189) | (SEQ ID NO. 190) |
| LDLR-mm10-Pr-1 | GUGGGAAGUUCCUCAAAUU | AAUUUGAGGAACUUCCCAC |
| | (SEQ ID NO. 191) | (SEQ ID NO. 192) |
| LDLR-mm10-Pr-2 | GUUGUAUUUCUCGACCAAA | UUUGGUCGAGAAAUACAAC |
| | (SEQ ID NO. 193) | (SEQ ID NO. 194) |
| LDLR-mm10-Pr-3 | GAAAUGUUCCCGCUGCAAA | UUUGCAGCGGGAACAUUUC |
| | (SEQ ID NO. 195) | (SEQ ID NO. 196) |
| LDLR-mm10-Pr-4 | UAGAUUUCUGACCCAAGUA | UACUUGGGUCAGAAAUCUA |
| | (SEQ ID NO. 197) | (SEQ ID NO. 198) |
| LDLR-rn5-Pr-1 | GAAAUGUUCCCGCUGCAAA | UUUGCAGCGGGAACAUUUC |
| | (SEQ ID NO. 199) | (SEQ ID NO. 200) |
| LDLR-rn5-Pr-2 | CAAGUUCGCAUGAGUGAUU | AAUCACUCAUGCGAACUUG |
| | (SEQ ID NO. 201) | (SEQ ID NO. 202) |
| LDLR-rn5-Pr-3 | UCGAGUUCCUCCUUCAGAA | UUCUGAAGGAGGAACUCGA |
| | (SEQ ID NO. 203) | (SEQ ID NO. 204) |
| LDLR-rn5-Pr-4 | CACACAUAGCUCUGAAGAA | UUCUUCAGAGCUAUGUGUG |
| | (SEQ ID NO. 205) | (SEQ ID NO. 206) |
| LDLR.NM_000527- | GAAAUUUCUCGAUGUUUAA | UUAAACAUCGAGAAAUUUC |
| Pr-4 | (SEQ ID NO. 207) | (SEQ ID NO. 208) |
| LDLR.NM_000527- | AACGCAUCUUCUGAAAGAU | AUCUUUCAGAAGAUGCGUU |
| Pr-6 | (SEQ ID NO. 209) | (SEQ ID NO. 210) |
| LDLR.NM_000527- | CCUGAAAUUUCUCGAUGUU | AACAUCGAGAAAUUUCAGG |
| Pr-7 | (SEQ ID NO. 211) | (SEQ ID NO. 212) |
| LDLR.NM_000527- | GGAGUCUGGACGUAAAUAU | AUAUUUACGUCCAGACUCC |
| Pr-13 or LDLR-Pr-3 | (SEQ ID NO. 213) | (SEQ ID NO. 214) |
| LDLR.NM_000527- | CGCCUUUGGCUGAGUAUAA | UUAUACUCAGCCAAAGGCG |
| Pr-22 | (SEQ ID NO. 215) | (SEQ ID NO. 216) |
| LDLR.NM_000527- | GCCUUUGGCUGAGUAUAAA | UUUAUACUCAGCCAAAGGC |
| Pr-23 | (SEQ ID NO. 217) | (SEQ ID NO. 218) |
| LDLR.NM_000527- | AAACGCAUCUUCUGAAAGA | UCUUUCAGAAGAUGCGUUU |
| Pr-28 | (SEQ ID NO. 219) | (SEQ ID NO. 220) |

TABLE 5
Sequences of LDLR-saRNAs (with 3′ overhang)

| saRNA ID | Sense (passenger) | Antisense (guide) |
|---|---|---|
| LDLR-Pr-1′ | GAAAUUUCUCGAUGUUUAAUU (SEQ | UUAAACAUCGAGAAAUUUCUU |
| | ID NO. 221) | (SEQ ID NO. 222) |
| LDLR-Pr-2′ | AACGCAUCUUCUGAAAGAUUU (SEQ | AUCUUUCAGAAGAUGCGUUUU |
| | ID NO. 223) | (SEQ ID NO. 224) |
| LDLR-Pr-4′ | GCCUGUAAUCCCAGCUAUUUU (SEQ | AAUAGCUGGGAUUACAGGCUU |
| | ID NO. 225) | (SEQ ID NO. 226) |
| LDLR-mm10-Pr-1′ | GUGGGAAGUUCCUCAAAUUUU (SEQ | AAUUUGAGGAACUUCCCACUU |
| | ID NO. 227) | (SEQ ID NO. 228) |
| LDLR-mm10-Pr-2′ | GUUGUAUUUCUCGACCAAAUU (SEQ | UUUGGUCGAGAAAUACAACUU |
| | ID NO. 229) | (SEQ ID NO. 230) |
| LDLR-mm10-Pr-3′ | GAAAUGUUCCCGCUGCAAAUU (SEQ | UUUGCAGCGGGAACAUUUCUU |
| | ID NO. 231) | (SEQ ID NO. 232) |
| LDLR-mm10-Pr-4′ | UAGAUUUCUGACCCAAGUAUU (SEQ | UACUUGGGUCAGAAAUCUAUU |
| | ID NO. 233) | (SEQ ID NO. 234) |
| LDLR-rn5-Pr-1′ | GAAAUGUUCCCGCUGCAAAUU (SEQ | UUUGCAGCGGGAACAUUUCUU |
| | ID NO. 235) | (SEQ ID NO. 236) |
| LDLR-rn5-Pr-2′ | CAAGUUCGCAUGAGUGAUUUU (SEQ | AAUCACUCAUGCGAACUUGUU |
| | ID NO. 237) | (SEQ ID NO. 238) |
| LDLR-rn5-Pr-3′ | UCGAGUUCCUCCUUCAGAAUU (SEQ | UUCUGAAGGAGGAACUCGAUU |
| | ID NO. 239) | (SEQ ID NO. 240) |
| LDLR-rn5-Pr-4′ | CACACAUAGCUCUGAAGAAUU (SEQ | UUCUUCAGAGCUAUGUGUGUU |
| | ID NO. 241) | (SEQ ID NO. 242) |
| LDLR.NM_000527- | GAAAUUUCUCGAUGUUUAAUU (SEQ | UUAAACAUCGAGAAAUUUCUU |
| Pr-4′ | ID NO. 243) | (SEQ ID NO. 244) |
| LDLR.NM_000527- | AACGCAUCUUCUGAAAGAUUU (SEQ | AUCUUUCAGAAGAUGCGUUUU |
| Pr-6′ | ID NO. 245) | (SEQ ID NO. 246) |
| LDLR.NM_000527- | CCUGAAAUUUCUCGAUGUUUU (SEQ | AACAUCGAGAAAUUUCAGGUU |
| Pr-7′ | ID NO. 247) | (SEQ ID NO. 248) |
| LDLR.NM_000527- | GGAGUCUGGACGUAAAUAUUU (SEQ | AUAUUUACGUCCAGACUCCUU |
| Pr-13′ or LDLR-Pr- | ID NO. 249) | (SEQ ID NO. 250) |
| 3′ | | |
| LDLR.NM_000527- | CGCCUUUGGCUGAGUAUAAUU (SEQ | UUAUACUCAGCCAAAGGCGUU |
| Pr-22′ | ID NO. 251) | (SEQ ID NO. 252) |
| LDLR.NM_000527- | GCCUUUGGCUGAGUAUAAAUU (SEQ | UUUAUACUCAGCCAAAGGCUU |
| Pr-23′ | ID NO. 253) | (SEQ ID NO. 254) |
| LDLR.NM_000527- | AAACGCAUCUUCUGAAAGAUU (SEQ | UCUUUCAGAAGAUGCGUUUUU |
| Pr-28′ | ID NO. 255) | (SEQ ID NO. 256) |

The LDLR gene encodes a protein called a low-density lipoprotein receptor. This receptor binds to particles called low-density lipoproteins (LDLs), which are the primary carriers of cholesterol in the blood. Cholesterol is a waxy, fat-like substance that is produced in the body and obtained from foods that come from animals. Low-density lipoprotein receptors sit on the outer surface of many types of cells, where they pick up low-density lipoproteins circulating in the bloodstream and transport them into the cell. Once inside the cell, the low-density lipoprotein is broken down to release cholesterol. The cholesterol is then used by the cell, stored, or removed from the body. After low-density lipoprotein receptors drop off their cargo, they are recycled back to the cell surface to pick up more low-density lipoproteins. Low-density lipoprotein receptors play a critical role in regulating the amount of cholesterol in the blood. They are particularly abundant in the liver, which is the organ responsible for removing most excess cholesterol from the body. The number of low-density lipoprotein receptors on the surface of liver cells determines how quickly cholesterol (in the form of low-density lipoproteins) is removed from the bloodstream. Mutations of LDLR gene cause various diseases such as hypercholesterolemia, which is characterized by high blood cholesterol levels (Lye et al., PLoS One, vol. 8 (4):e60729 (2013), the contents of which are incorporated herein by reference in their entirety). A buildup of cholesterol in the walls of arteries increases a person's risk of having a heart attack.

In one embodiment, provided is a method of modulating low-density lipoprotein receptor protein levels comprising administering LDLR-saRNA of the present invention, wherein the saRNA targets an antisense RNA transcript of LDLR gene. Low-density lipoprotein receptor protein level may be increased by at least 20, 30, 40%, or at least 45, 50, 55, 60, 65, 70, 75%, or at least 80% in the presence of the LDLR-saRNA of the present invention compared to low-density lipoprotein receptor protein level in the absence of the LDLR-saRNA of the present invention. In a further embodiment, low-density lipoprotein receptor protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the LDLR-saRNA of the present invention compared to low-density lipoprotein receptor protein level in the absence of the LDLR-saRNA of the present invention.

In another embodiment, provided is a method of modulating cholesterol levels comprising administering LDLR-saRNA of the present invention, wherein the LDLR-saRNA targets an antisense RNA transcript of LDLR gene. Cholesterol level may be reduced by at least 10%, 20%, 30%, or at least 40%, 50%, 60%, or 70%, 80%, 90% in the presence of the LDLR-saRNA of the present invention compared to cholesterol level in the absence of the LDLR-saRNA of the present invention.

In another embodiment, provided is a method of treating hypercholesterolemia comprising administering LDLR-saRNA of the present invention, wherein the LDLR-saRNA targets an antisense RNA transcript of LDLR gene, and wherein the symptoms of hypercholesterolemia is reduced.

In one embodiment, the LDLR-saRNA of the present invention is an antisense sequence such as, but not limited to, SEQ ID NO: 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 4610, 4612, 4614, 4616, 4618, 4620, 4622, 4624, 4626, 4628, 4630, 4632, 4634, 4636, 4638, 4640, 4642, 4644, 4646, 4648, 4650, 4652, 4654, 4656, 4658, 4660, 4662, 4664, 4666, 4668, 4670, 4672, 4674, 4676, 4678, 4680, 4682, 4684, 4686, 4688, 4690, 4692, 4694, 4696, 4698, 4700, 4702, 4704, 4706, and 4708. As a non-limiting example, the LDLR-saRNA may be used to modulate low-density lipoprotein receptor protein levels, modulate cholesterol levels and/or treating hypercholesterolemia.

In one embodiment, the APOA1-saRNA of the present invention is an saRNA duplex. The saRNA duplex may be a pair of sense and antisense sequences such as, but not limited to, SEQ ID NOs: 185 and 186; SEQ ID NOs: 187 and 188; SEQ ID NOs: 189 and 190; SEQ ID NOs: 191 and 192; SEQ ID NOs: 193 and 194; SEQ ID NOs: 195 and 196; SEQ ID NOs: 197 and 198; SEQ ID NOs: 199 and 200; SEQ ID NOs: 201 and 202; SEQ ID NOs: 203 and 204; SEQ ID NOs: 205 and 206; SEQ ID NOs: 207 and 208; SEQ ID NOs: 209 and 210; SEQ ID NOs: 211 and 212; SEQ ID NOs: 213 and 214; SEQ ID NOs: 215 and 216; SEQ ID NOs: 217 and 218; SEQ ID NOs: 219 and 220; SEQ ID NOs: 221 and 222; SEQ ID NOs: 223 and 224; SEQ ID NOs: 225 and 226; SEQ ID NOs: 227 and 228; SEQ ID NOs: 229 and 230; SEQ ID NOs: 231 and 232; SEQ ID NOs: 233 and 234; SEQ ID NOs: 235 and 236; SEQ ID NOs: 237 and 238; SEQ ID NOs: 239 and 240; SEQ ID NOs: 241 and 242; SEQ ID NOs: 243 and 244; SEQ ID NOs: 245 and 246; SEQ ID NOs: 247 and 248; SEQ ID NOs: 249 and 250; SEQ ID NOs: 251 and 252; SEQ ID NOs: 253 and 254; SEQ ID NOs: 255 and 256; SEQ ID NOs: 4609 and 4610; SEQ ID NOs: 4611 and 4612; SEQ ID NOs: 4613 and 4614; SEQ ID NOs: 4615 and 4616; SEQ ID NOs: 4617 and 4618; SEQ ID NOs: 4619 and 4620; SEQ ID NOs: 4621 and 4622; SEQ ID NOs: 4623 and 4624; SEQ ID NOs: 4625 and 4626; SEQ ID NOs: 4627 and 4628; SEQ ID NOs: 4629 and 4630; SEQ ID NOs: 4631 and 4632; SEQ ID NOs: 4633 and 4634; SEQ ID NOs: 4635 and 4636; SEQ ID NOs: 4637 and 4638; SEQ ID NOs: 4639 and 4640; SEQ ID NOs: 4641 and 4642; SEQ ID NOs: 4643 and 4644; SEQ ID NOs: 4645 and 4646; SEQ ID NOs: 4647 and 4648; SEQ ID NOs: 4649 and 4650; SEQ ID NOs: 4651 and 4652; SEQ ID NOs: 4653 and 4654; SEQ ID NOs: 4655 and 4656; SEQ ID NOs: 4657 and 4658; SEQ ID NOs: 4659 and 4660; SEQ ID NOs: 4661 and 4662; SEQ ID NOs: 4663 and 4664; SEQ ID NOs: 4665 and 4666; SEQ ID NOs: 4667 and 4668; SEQ ID NOs: 4669 and 4670; SEQ ID NOs: 4671 and 4672; SEQ ID NOs: 4673 and 4674; SEQ ID NOs: 4675 and 4676; SEQ ID NOs: 4677 and 4678; SEQ ID NOs: 4679 and 4680; SEQ ID NOs: 4681 and 4682; SEQ ID NOs: 4683 and 4684; SEQ ID NOs: 4685 and 4686; SEQ ID NOs: 4687 and 4688; SEQ ID NOs: 4689 and 4690; SEQ ID NOs: 4691 and 4692; SEQ ID NOs: 4693 and 4694; SEQ ID NOs: 4695 and 4696; SEQ ID NOs: 4697 and 4698; SEQ ID NOs: 4699 and 4700; SEQ ID NOs: 4701 and 4702; SEQ ID NOs: 4703 and 4704; SEQ ID NOs: 4705 and 4706; SEQ ID NOs: 4707 and 4708. As a non-limiting example, the LDLR-saRNA which are saRNA duplexes may be used to modulate low-density lipoprotein receptor protein levels, modulate cholesterol levels and/or treating hypercholesterolemia.

### DMD Gene

As another non-limiting example, provided is a method of modulating the expression of DMD gene comprising administering saRNA of the present invention, wherein the saRNA targets an antisense RNA transcript of DMD gene. These saRNAs are called DMD-saRNA. In one embodiment, the expression of DMD gene is increased by at least 20, 30, 40%, or at least 45, 50, 55, 60, 65, 70, 75%, or at least 80% in the presence of the DMD-saRNA of the present invention compared to the expression of DMD gene in the absence of the DMD-saRNA of the present invention. In a further embodiment, the expression of DMD gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the DMD-saRNA of the present invention compared to the expression of DMD gene in the absence of the DMD-saRNA of the present invention. The modulation of the expression of DMD gene may be reflected or determined by the change of DMD mRNA levels.

DMD-saRNAs may be single-stranded and comprise 14-30 nucleotides. The sequence of a single-stranded DMD-saRNA may have at least 60%, 70%, 80% or 90% identity with a sequence selected from the sequences of the antisense strands in Table 6-2. In one embodiment, the single-stranded saRNA comprises a sequence selected from the sequences of the antisense strands in Table 6, i.e., SEQ ID NOS 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, and 412. In one embodiment, the single-stranded DMD-saRNA may have a 3′ tail. The sequence of a single-stranded DMD-saRNA with a 3′ tail may have at least 60%, 70%, 80% or 90% identity with a sequence selected from the sequences of the antisense strands in Table 7. In one embodiment, the single-stranded saRNA comprises a sequence selected from the sequences of the antisense strands in Table 7.

DMD-saRNAs may be double-stranded. The two strands form a duplex and each strand comprises 14-30 nucleotides. The first strand of a double-stranded DMD-saRNA may have at least 60%, 70%, 80% or 90% identity with a sequence selected from the sequences of the antisense strands in Table 6-2. In one embodiment, the first strand of the double-stranded saRNA comprises a sequence selected from the sequences of the antisense strands in Table 6-2. The second strand of a double-stranded DMD-saRNA may have at least 60%, 70%, 80% or 90% identity with a sequence selected from the sequences of the sense strands in Table 6-2. In one embodiment, the second strand of the double-stranded saRNA comprises a sequence selected from the sequences of the sense strands in Table 6-2. In one embodiment, the double-stranded DMD-saRNA may have a 3′ overhang on each strand. The first strand of a double-stranded DMD-saRNA may have at least 60%, 70%, 80% or 90% identity with a sequence selected from the sequences of the antisense strands in Table 7. In one embodiment, the first strand of the double-stranded saRNA comprises a sequence selected from the sequences of the antisense strands in Table 7. The second strand of a double-stranded DMD-saRNA may have at least 60%, 70%, 80% or 90% identity with a sequence selected from the sequences of the sense strands in Table 7. In one embodiment, the second strand of the double-stranded saRNA comprises a sequence selected from the sequences of the sense strands in Table 7.

DMD-saRNAs may be modified or unmodified.

Table 6-1 describes the target gene, the targeted sequence, the genomic location of the targeted sequence and the relative location of DMD-saRNAs with no 3′ overhang. In Table 6-1, the targeted sequence is defined as a region on the template strand of the target gene which is identical to a region on the target antisense RNA transcript to which the antisense strand of an saRNA hybridizes. In Table 6-1, the relative location is the distance from the 5′ end of the targeted sequence to the TSS. A negative number represents a location upstream of the TSS and a positive number represents a location downstream of the TSS.

TABLE 6-1
Sequences of DMD-saRNAs (with no 3′ overhang)

| | | | Genomic Location | |
|---|---|---|---|---|
| | | | of the Targeted | Relative |
| saRNA ID | Target gene | Targeted sequence | sequence | Location |
| DMD.NM_000109- | DMD human | ataggttattggtgtcaaa (SEQ | chrX: 33146045 plus | 500 |
| Pr-1 | | ID NO. 257) | strand | |
| DMD.NM_000109- | DMD human | ctacgtccttagaaattta (SEQ | chrX: 33145863 plus | 682 |
| Pr-7 | | ID NO. 258) | strand | |
| DMD.NM_000109- | DMD human | tgcattatcctataggtta (SEQ | chrX: 33146034 plus | 511 |
| Pr-21 | | ID NO. 259) | strand | |
| DMD.NM_000109- | DMD human | gacatctcctcatgaatat (SEQ | chrX: 32431341 plus | −970 |
| Pr-26 | | ID NO. 260) | strand | |
| DMD.NM_000109- | DMD human | gaatcagctccttgtcaaa (SEQ | chrX: 32430478 plus | −107 |
| Pr-35 | | ID NO. 261) | strand | |
| DMD.NM_000109- | DMD human | ctgtaatcctgctgaacaa (SEQ | chrX: 32430084 plus | 287 |
| Pr-36 | | ID NO. 262) | strand | |
| DMD.NM_004006- | DMD human | gggtcatcgtcatcacaat | chrX: 32430403 plus | −32 |
| Pr-3 | | (SEQ ID NO. 263) | strand | |
| DMD.NM_004006- | DMD human | tgcttccaccagtgagata | chrX: 32430442 plus | −71 |
| Pr-10 | | (SEQ ID NO. 264) | strand | |
| DMD.NM_004006- | DMD human | gtagtaatctgtagagtta | chrX: 31525986 plus | 429 |
| Pr-12 | | (SEQ ID NO. 265) | strand | |
| DMD.NM_004006- | DMD human | cgaagtttggtcaaatata | chrX: 31527327 plus | −912 |
| Pr-23 | | (SEQ ID NO. 266) | strand | |
| DMD.NM_004006- | DMD human | ggattgatctgtgtaacta | chrX: 31526871 plus | −456 |
| Pr-30 | | (SEQ ID NO. 267) | strand | |
| DMD.NM_004006- | DMD human | tgagtagtatctctgagta | chrX: 31527220 plus | −805 |
| Pr-40 | | (SEQ ID NO. 268) | strand | |
| DMD.NM_004009- | DMD human | gcatagcaatcctgagaaa | chrX: 31526392 plus | 23 |
| Pr-14 | | (SEQ ID NO. 269) | strand | |
| DMD.NM_004009- | DMD human | cgtagtaatctgtagagtt | chrX: 31525985 plus | 430 |
| Pr-23 | | (SEQ ID NO. 270) | strand | |
| DMD.NM_004009- | DMD human | gaaagtatttctgagtagt | chrX: 31527209 plus | −794 |
| Pr-25 | | (SEQ ID NO. 271) | strand | |
| DMD.NM_004009- | DMD human | ctgacttgctcttagagat | chrX: 31526798 plus | −383 |
| Pr-27 | | (SEQ ID NO. 272) | strand | |
| DMD.NM_004009- | DMD human | gggattcttgcagaacgaa | chrX: 31527312 plus | −897 |
| Pr-34 | | (SEQ ID NO. 273) | strand | |
| DMD.NM_004009- | DMD human | gcagaaataacatgttaga | chrX: 31526962 plus | −547 |
| Pr-38 | | (SEQ ID NO. 274) | strand | |
| DMD.NM_004012- | DMD human | gggaaatactgaaagtatt | chrX: 31527199 plus | −784 |
| Pr-4 | | (SEQ ID NO. 275) | strand | |
| DMD.NM_004012- | DMD human | ggtttgtactggaaggata | chrX: 31527249 plus | −834 |
| Pr-17 | | (SEQ ID NO. 276) | strand | |
| DMD.NM_004012- | DMD human | gcgaacttcttagaaatga | chrX: 31285627 plus | −603 |
| Pr-24 | | (SEQ ID NO. 277) | strand | |
| DMD.NM_004012- | DMD human | agggaaatgtcagttggaa | chrX: 31284424 plus | 600 |
| Pr-26 | | (SEQ ID NO. 278) | strand | |
| DMD.NM_004012- | DMD human | gatttggatttgtgacata | chrX: 31285556 plus | −532 |
| Pr-36 | | (SEQ ID NO. 279) | strand | |
| DMD.NM_004014- | DMD human | ggtatggactctctaaatt | chrX: 31285715 plus | −691 |
| Pr-1 | | (SEQ ID NO. 280) | strand | |
| DMD.NM_004014- | DMD human | ggaaatgtcagttggaatc | chrX: 31284426 plus | 598 |
| Pr-2 | | (SEQ ID NO. 281) | strand | |
| DMD.NM_004014- | DMD human | acaaactttgcttgaagat | chrX: 31284189 plus | 835 |
| Pr-4 | | (SEQ ID NO. 282) | strand | |
| DMD.NM_004014- | DMD human | gcagtatgatgatagaagt | chrX: 31285878 plus | −854 |
| Pr-10 | | (SEQ ID NO. 283) | strand | |
| DMD.NM_004014- | DMD human | gagtaagagcgacgattta | chrX: 31284529 plus | 495 |
| Pr-12 | | (SEQ ID NO. 284) | strand | |
| DMD.NM_004014- | DMD human | gataaagtttcatggtact | chrX: 32172788 plus | 798 |
| Pr-13 | | (SEQ ID NO. 285) | strand | |
| DMD.NM_004014- | DMD human | tggtttaacttgagagcaa | chrX: 32173213 plus | 373 |
| Pr-15 | | (SEQ ID NO. 286) | strand | |
| DMD.NM_004014- | DMD human | acagtcagcacttgataaa | chrX: 32173917 plus | −331 |
| Pr-20 | | (SEQ ID NO. 287) | strand | |
| DMD.NM_004014- | DMD human | ctgtatagctgatgataaa | chrX: 32174134 plus | −548 |
| Pr-24 | | (SEQ ID NO. 288) | strand | |
| DMD.NM_004014- | DMD human | ggtttaacttgagagcaaa | chrX: 32173214 plus | 372 |
| Pr-26 | | (SEQ ID NO. 289) | strand | |
| DMD.NM_004014- | DMD human | gtcagcacttgataaatat | chrX: 32173920 plus | −334 |
| Pr-27 | | (SEQ ID NO. 290) | strand | |
| DMD.NM_004014- | DMD human | caattgatatcaggttcaa | chrX: 32173959 plus | −373 |
| Pr-37 | | (SEQ ID NO. 291) | strand | |
| DMD.NM_004019- | DMD human | aaagaaatcctgggctgaa | chrX: 32173651 plus | −65 |
| Pr-7 | | (SEQ ID NO. 292) | strand | |
| DMD.NM_004019- | DMD human | gcattacctgatggagatt | chrX: 32173718 plus | −132 |
| Pr-14 | | (SEQ ID NO. 293) | strand | |
| DMD.NM_004019- | DMD human | ataggttattggtgtcaaa | chrX: 33146045 plus | 500 |
| Pr-16 | | (SEQ ID NO. 294) | strand | |
| DMD.NM_004019- | DMD human | ctacgtccttagaaattta | chrX: 33145863 plus | 682 |
| Pr-25 | | (SEQ ID NO. 295) | strand | |
| DMD.NM_004019- | DMD human | tgcattatcctataggtta | chrX: 33146034 plus | 511 |
| Pr-29 | | (SEQ ID NO. 296) | strand | |
| DMD.NM_004019- | DMD human | gacatctcctcatgaatat | chrX: 32431341 plus | −970 |
| Pr-33 | | (SEQ ID NO. 297) | strand | |
| DMD.NM_004019- | DMD human | gaatcagctccttgtcaaa | chrX: 32430478 plus | −107 |
| Pr-39 | | (SEQ ID NO. 298) | strand | |
| DMD.NM_004019- | DMD human | ctgtaatcctgctgaacaa | chrX: 32430084 plus | 287 |
| Pr-40 | | (SEQ ID NO. 299) | strand | |
| DMD.NM_004021- | DMD human | gggtcatcgtcatcacaat | chrX: 32430403 plus | −32 |
| Pr-8 | | (SEQ ID NO. 300) | strand | |
| DMD.NM_004021- | DMD human | tgcttccaccagtgagata | chrX: 32430442 plus | −71 |
| Pr-9 | | (SEQ ID NO. 301) | strand | |
| DMD.NM_004021- | DMD human | gtagtaatctgtagagtta | chrX: 31525986 plus | 429 |
| Pr-15 | | (SEQ ID NO. 302) | strand | |
| DMD.NM_004021- | DMD human | cgaagtttggtcaaatata | chrX: 31527327 plus | −912 |
| Pr-20 | | (SEQ ID NO. 303) | strand | |
| DMD.NM_004021- | DMD human | ggattgatctgtgtaacta | chrX: 31526871 plus | −456 |
| Pr-23 | | (SEQ ID NO. 304) | strand | |
| DMD.NM_004021- | DMD human | tgagtagtatctctgagta | chrX: 31527220 plus | −805 |
| Pr-26 | | (SEQ ID NO. 305) | strand | |
| DMD.NM_004021- | DMD human | gcatagcaatcctgagaaa | chrX: 31526392 plus | 23 |
| Pr-27 | | (SEQ ID NO. 306) | strand | |
| DMD.NM_004021- | DMD human | cgtagtaatctgtagagtt | chrX: 31525985 plus | 430 |
| Pr-31 | | (SEQ ID NO. 307) | strand | |
| DMD.NM_004021- | DMD human | gaaagtatttctgagtagt (SEQ | chrX: 31527209 plus | −794 |
| Pr-32 | | ID NO. 308) | strand | |

TABLE 6-2
Sequences of DMD-saRNAs (with no 3′ overhang)

| saRNA ID | Sense (passenger) | Antisense (guide) |
|---|---|---|
| DMD.NM_000109-Pr-1 | AUAGGUUAUUGGUGUCAAA | UUUGACACCAAUAACCUAU |
| | (SEQ ID NO. 309) | (SEQ ID NO. 310) |
| DMD.NM_000109-Pr-7 | CUACGUCCUUAGAAAUUUA | UAAAUUUCUAAGGACGUAG |
| | (SEQ ID NO. 311) | (SEQ ID NO. 312) |
| DMD.NM_000109-Pr-21 | UGCAUUAUCCUAUAGGUUA | UAACCUAUAGGAUAAUGCA |
| | (SEQ ID NO. 313) | (SEQ ID NO. 314) |
| DMD.NM_000109-Pr-26 | GACAUCUCCUCAUGAAUAU | AUAUUCAUGAGGAGAUGUC |
| | (SEQ ID NO. 315) | (SEQ ID NO. 316) |
| DMD.NM_000109-Pr-35 | GAAUCAGCUCCUUGUCAAA | UUUGACAAGGAGCUGAUUC |
| | (SEQ ID NO. 317) | (SEQ ID NO. 318) |
| DMD.NM_000109-Pr-36 | CUGUAAUCCUGCUGAACAA | UUGUUCAGCAGGAUUACAG |
| | (SEQ ID NO. 319) | (SEQ ID NO. 320) |
| DMD.NM_004006-Pr-3 | GGGUCAUCGUCAUCACAAU | AUUGUGAUGACGAUGACCC |
| | (SEQ ID NO. 321) | (SEQ ID NO. 322) |
| DMD.NM_004006-Pr-10 | UGCUUCCACCAGUGAGAUA | UAUCUCACUGGUGGAAGCA |
| | (SEQ ID NO. 323) | (SEQ ID NO. 324) |
| DMD.NM_004006-Pr-12 | GUAGUAAUCUGUAGAGUUA | UAACUCUACAGAUUACUAC |
| | (SEQ ID NO. 325) | (SEQ ID NO. 326) |
| DMD.NM_004006-Pr-23 | CGAAGUUUGGUCAAAUAUA | UAUAUUUGACCAAACUUCG |
| | (SEQ ID NO. 327) | (SEQ ID NO. 328) |
| DMD.NM_004006-Pr-30 | GGAUUGAUCUGUGUAACUA | UAGUUACACAGAUCAAUCC |
| | (SEQ ID NO. 329) | (SEQ ID NO. 330) |
| DMD.NM_004006-Pr-40 | UGAGUAGUAUCUCUGAGUA | UACUCAGAGAUACUACUCA |
| | (SEQ ID NO. 331) | (SEQ ID NO. 332) |
| DMD.NM_004009-Pr-14 | GCAUAGCAAUCCUGAGAAA | UUUCUCAGGAUUGCUAUGC |
| | (SEQ ID NO. 333) | (SEQ ID NO. 334) |
| DMD.NM_004009-Pr-23 | CGUAGUAAUCUGUAGAGUU | AACUCUACAGAUUACUACG |
| | (SEQ ID NO. 335) | (SEQ ID NO. 336) |
| DMD.NM_004009-Pr-25 | GAAAGUAUUUCUGAGUAGU | ACUACUCAGAAAUACUUUC |
| | (SEQ ID NO. 337) | (SEQ ID NO. 338) |
| DMD.NM_004009-Pr-27 | CUGACUUGCUCUUAGAGAU | AUCUCUAAGAGCAAGUCAG |
| | (SEQ ID NO. 339) | (SEQ ID NO. 340) |
| DMD.NM_004009-Pr-34 | GGGAUUCUUGCAGAACGAA | UUCGUUCUGCAAGAAUCCC |
| | (SEQ ID NO. 341) | (SEQ ID NO. 342) |
| DMD.NM_004009-Pr-38 | GCAGAAAUAACAUGUUAGA | UCUAACAUGUUAUUUCUGC |
| | (SEQ ID NO. 343) | (SEQ ID NO. 344) |
| DMD.NM_004012-Pr-4 | GGGAAAUACUGAAAGUAUU | AAUACUUUCAGUAUUUCCC |
| | (SEQ ID NO. 345) | (SEQ ID NO. 346) |
| DMD.NM_004012-Pr-17 | GGUUUGUACUGGAAGGAUA | UAUCCUUCCAGUACAAACC |
| | (SEQ ID NO. 347) | (SEQ ID NO. 348) |
| DMD.NM_004012-Pr-24 | GCGAACUUCUUAGAAAUGA | UCAUUUCUAAGAAGUUCGC |
| | (SEQ ID NO. 349) | (SEQ ID NO. 350) |
| DMD.NM_004012-Pr-26 | AGGGAAAUGUCAGUUGGAA | UUCCAACUGACAUUUCCCU |
| | (SEQ ID NO. 351) | (SEQ ID NO. 352) |
| DMD.NM_004012-Pr-36 | GAUUUGGAUUUGUGACAUA | UAUGUCACAAAUCCAAAUC |
| | (SEQ ID NO. 353) | (SEQ ID NO. 354) |
| DMD.NM_004014-Pr-1 | GGUAUGGACUCUCUAAAUU | AAUUUAGAGAGUCCAUACC |
| | (SEQ ID NO. 355) | (SEQ ID NO. 356) |
| DMD.NM_004014-Pr-2 | GGAAAUGUCAGUUGGAAUC | GAUUCCAACUGACAUUUCC |
| | (SEQ ID NO. 357) | (SEQ ID NO. 358) |
| DMD.NM_004014-Pr-4 | ACAAACUUUGCUUGAAGAU | AUCUUCAAGCAAAGUUUGU |
| | (SEQ ID NO. 359) | (SEQ ID NO. 360) |
| DMD.NM_004014-Pr-10 | GCAGUAUGAUGAUAGAAGU | ACUUCUAUCAUCAUACUGC |
| | (SEQ ID NO. 361) | (SEQ ID NO. 362) |
| DMD.NM_004014-Pr-12 | GAGUAAGAGCGACGAUUUA | UAAAUCGUCGCUCUUACUC |
| | (SEQ ID NO. 363) | (SEQ ID NO. 364) |
| DMD.NM_004014-Pr-13 | GAUAAAGUUUCAUGGUACU | AGUACCAUGAAACUUUAUC |
| | (SEQ ID NO. 365) | (SEQ ID NO. 366) |
| DMD.NM_004014-Pr-15 | UGGUUUAACUUGAGAGCAA | UUGCUCUCAAGUUAAACCA |
| | (SEQ ID NO. 367) | (SEQ ID NO. 368) |
| DMD.NM_004014-Pr-20 | ACAGUCAGCACUUGAUAAA | UUUAUCAAGUGCUGACUGU |
| | (SEQ ID NO. 369) | (SEQ ID NO. 370) |
| DMD.NM_004014-Pr-24 | CUGUAUAGCUGAUGAUAAA | UUUAUCAUCAGCUAUACAG |
| | (SEQ ID NO. 371) | (SEQ ID NO. 372) |
| DMD.NM_004014-Pr-26 | GGUUUAACUUGAGAGCAAA | UUUGCUCUCAAGUUAAACC |
| | (SEQ ID NO. 373) | (SEQ ID NO. 374) |
| DMD.NM_004014-Pr-27 | GUCAGCACUUGAUAAAUAU | AUAUUUAUCAAGUGCUGAC |
| | (SEQ ID NO. 375) | (SEQ ID NO. 376) |
| DMD.NM_004014-Pr-37 | CAAUUGAUAUCAGGUUCAA | UUGAACCUGAUAUCAAUUG |
| | (SEQ ID NO. 377) | (SEQ ID NO. 378) |
| DMD.NM_004019-Pr-7 | AAAGAAAUCCUGGGCUGAA | UUCAGCCCAGGAUUUCUUU |
| | (SEQ ID NO. 379) | (SEQ ID NO. 380) |
| DMD.NM_004019-Pr-14 | GCAUUACCUGAUGGAGAUU | AAUCUCCAUCAGGUAAUGC |
| | (SEQ ID NO. 381) | (SEQ ID NO. 382) |
| DMD.NM_004019-Pr-16 | AUAGGUUAUUGGUGUCAAA | UUUGACACCAAUAACCUAU |
| | (SEQ ID NO. 383) | (SEQ ID NO. 384) |
| DMD.NM_004019-Pr-25 | CUACGUCCUUAGAAAUUUA | UAAAUUUCUAAGGACGUAG |
| | (SEQ ID NO. 385) | (SEQ ID NO. 386) |
| DMD.NM_004019-Pr-29 | UGCAUUAUCCUAUAGGUUA | UAACCUAUAGGAUAAUGCA |
| | (SEQ ID NO. 387) | (SEQ ID NO. 388) |
| DMD.NM_004019-Pr-33 | GACAUCUCCUCAUGAAUAU | AUAUUCAUGAGGAGAUGUC |
| | (SEQ ID NO. 389) | (SEQ ID NO. 390) |
| DMD.NM_004019-Pr-39 | GAAUCAGCUCCUUGUCAAA | UUUGACAAGGAGCUGAUUC |
| | (SEQ ID NO. 391) | (SEQ ID NO. 392) |
| DMD.NM_004019-Pr-40 | CUGUAAUCCUGCUGAACAA | UUGUUCAGCAGGAUUACAG |
| | (SEQ ID NO. 393) | (SEQ ID NO. 394) |
| DMD.NM_004021-Pr-8 | GGGUCAUCGUCAUCACAAU | AUUGUGAUGACGAUGACCC |
| | (SEQ ID NO. 395) | (SEQ ID NO. 396) |
| DMD.NM_004021-Pr-9 | UGCUUCCACCAGUGAGAUA | UAUCUCACUGGUGGAAGCA |
| | (SEQ ID NO. 397) | (SEQ ID NO. 398) |
| DMD.NM_004021-Pr-15 | GUAGUAAUCUGUAGAGUUA | UAACUCUACAGAUUACUAC |
| | (SEQ ID NO. 399) | (SEQ ID NO. 400) |
| DMD.NM_004021-Pr-20 | CGAAGUUUGGUCAAAUAUA | UAUAUUUGACCAAACUUCG |
| | (SEQ ID NO. 401) | (SEQ ID NO. 402) |
| DMD.NM_004021-Pr-23 | GGAUUGAUCUGUGUAACUA | UAGUUACACAGAUCAAUCC |
| | (SEQ ID NO. 403) | (SEQ ID NO. 404) |
| DMD.NM_004021-Pr-26 | UGAGUAGUAUCUCUGAGUA | UACUCAGAGAUACUACUCA |
| | (SEQ ID NO. 405) | (SEQ ID NO. 406) |
| DMD.NM_004021-Pr-27 | GCAUAGCAAUCCUGAGAAA | UUUCUCAGGAUUGCUAUGC |
| | (SEQ ID NO. 407) | (SEQ ID NO. 408) |
| DMD.NM_004021-Pr-31 | CGUAGUAAUCUGUAGAGUU | AACUCUACAGAUUACUACG |
| | (SEQ ID NO. 409) | (SEQ ID NO. 410) |
| DMD.NM_004021-Pr-32 | GAAAGUAUUUCUGAGUAGU | ACUACUCAGAAAUACUUUC |
| | (SEQ ID NO. 411) | (SEQ ID NO. 412) |

TABLE 7
Sequences of DMD-saRNAs (with 3′ overhang)

| saRNA ID | Sense (passenger) | Antisense (guide) |
|---|---|---|
| DMD.NM_000109- | AUAGGUUAUUGGUGUCAAAUU (SEQ | UUUGACACCAAUAACCUAUUU |
| Pr-1′ | ID NO. 413) | (SEQ ID NO. 414) |
| DMD.NM_000109- | CUACGUCCUUAGAAAUUUAUU (SEQ | UAAAUUUCUAAGGACGUAGUU |
| Pr-7′ | ID NO. 415) | (SEQ ID NO. 416) |
| DMD.NM_000109- | UGCAUUAUCCUAUAGGUUAUU (SEQ | UAACCUAUAGGAUAAUGCAUU |
| Pr-21′ | ID NO. 417) | (SEQ ID NO. 418) |
| DMD.NM_000109- | GACAUCUCCUCAUGAAUAUUU (SEQ | AUAUUCAUGAGGAGAUGUCUU |
| Pr-26′ | ID NO. 419) | (SEQ ID NO. 420) |
| DMD.NM_000109- | GAAUCAGCUCCUUGUCAAAUU (SEQ | UUUGACAAGGAGCUGAUUCUU |
| Pr-35′ | ID NO. 421) | (SEQ ID NO. 422) |
| DMD.NM_000109- | CUGUAAUCCUGCUGAACAAUU (SEQ | UUGUUCAGCAGGAUUACAGUU |
| Pr-36′ | ID NO. 423) | (SEQ ID NO. 424) |
| DMD.NM_004006- | GGGUCAUCGUCAUCACAAUUU (SEQ | AUUGUGAUGACGAUGACCCUU |
| Pr-3′ | ID NO. 425) | (SEQ ID NO. 426) |
| DMD.NM_004006- | UGCUUCCACCAGUGAGAUAUU (SEQ | UAUCUCACUGGUGGAAGCAUU |
| Pr-10′ | ID NO. 427) | (SEQ ID NO. 428) |
| DMD.NM_004006- | GUAGUAAUCUGUAGAGUUAUU (SEQ | UAACUCUACAGAUUACUACUU |
| Pr-12′ | ID NO. 429) | (SEQ ID NO. 430) |
| DMD.NM_004006- | CGAAGUUUGGUCAAAUAUAUU (SEQ | UAUAUUUGACCAAACUUCGUU |
| Pr-23′ | ID NO. 431) | (SEQ ID NO. 432) |
| DMD.NM_004006- | GGAUUGAUCUGUGUAACUAUU (SEQ | UAGUUACACAGAUCAAUCCUU |
| Pr-30′ | ID NO. 433) | (SEQ ID NO. 434) |
| DMD.NM_004006- | UGAGUAGUAUCUCUGAGUAUU (SEQ | UACUCAGAGAUACUACUCAUU |
| Pr-40′ | ID NO. 435) | (SEQ ID NO. 436) |
| DMD.NM_004009- | GCAUAGCAAUCCUGAGAAAUU (SEQ | UUUCUCAGGAUUGCUAUGCUU |
| Pr-14′ | ID NO. 437) | (SEQ ID NO. 438) |
| DMD.NM_004009- | CGUAGUAAUCUGUAGAGUUUU (SEQ | AACUCUACAGAUUACUACGUU |
| Pr-23′ | ID NO. 439) | (SEQ ID NO. 440) |
| DMD.NM_004009- | GAAAGUAUUUCUGAGUAGUUU (SEQ | ACUACUCAGAAAUACUUUCUU |
| Pr-25′ | ID NO. 441) | (SEQ ID NO. 442) |
| DMD.NM_004009- | CUGACUUGCUCUUAGAGAUUU (SEQ | AUCUCUAAGAGCAAGUCAGUU |
| Pr-27′ | ID NO. 443) | (SEQ ID NO. 444) |
| DMD.NM_004009- | GGGAUUCUUGCAGAACGAAUU (SEQ | UUCGUUCUGCAAGAAUCCCUU |
| Pr-34′ | ID NO. 445) | (SEQ ID NO. 446) |
| DMD.NM_004009- | GCAGAAAUAACAUGUUAGAUU (SEQ | UCUAACAUGUUAUUUCUGCUU |
| Pr-38′ | ID NO. 447) | (SEQ ID NO. 448) |
| DMD.NM_004012- | GGGAAAUACUGAAAGUAUUUU (SEQ | AAUACUUUCAGUAUUUCCCUU |
| Pr-4′ | ID NO. 449) | (SEQ ID NO. 450) |
| DMD.NM_004012- | GGUUUGUACUGGAAGGAUAUU (SEQ | UAUCCUUCCAGUACAAACCUU |
| Pr-17′ | ID NO. 451) | (SEQ ID NO. 452) |
| DMD.NM_004012- | GCGAACUUCUUAGAAAUGAUU (SEQ | UCAUUUCUAAGAAGUUCGCUU |
| Pr-24′ | ID NO. 453) | (SEQ ID NO. 454) |
| DMD.NM_004012- | AGGGAAAUGUCAGUUGGAAUU (SEQ | UUCCAACUGACAUUUCCCUUU |
| Pr-26′ | ID NO. 455) | (SEQ ID NO. 456) |
| DMD.NM_004012- | GAUUUGGAUUUGUGACAUAUU (SEQ | UAUGUCACAAAUCCAAAUCUU |
| Pr-36′ | ID NO. 457) | (SEQ ID NO. 458) |
| DMD.NM_004014- | GGUAUGGACUCUCUAAAUUUU (SEQ | AAUUUAGAGAGUCCAUACCUU |
| Pr-1′ | ID NO. 459) | (SEQ ID NO. 460) |
| DMD.NM_004014- | GGAAAUGUCAGUUGGAAUCUU (SEQ | GAUUCCAACUGACAUUUCCUU |
| Pr-2′ | ID NO. 461) | (SEQ ID NO. 462) |
| DMD.NM_004014- | ACAAACUUUGCUUGAAGAUUU (SEQ | AUCUUCAAGCAAAGUUUGUUU |
| Pr-4′ | ID NO. 463) | (SEQ ID NO. 464) |
| DMD.NM_004014- | GCAGUAUGAUGAUAGAAGUUU (SEQ | ACUUCUAUCAUCAUACUGCUU |
| Pr-10′ | ID NO. 465) | (SEQ ID NO. 466) |
| DMD.NM_004014- | GAGUAAGAGCGACGAUUUAUU (SEQ | UAAAUCGUCGCUCUUACUCUU |
| Pr-12′ | ID NO. 467) | (SEQ ID NO. 468) |
| DMD.NM_004014- | GAUAAAGUUUCAUGGUACUUU (SEQ | AGUACCAUGAAACUUUAUCUU |
| Pr-13′ | ID NO. 469) | (SEQ ID NO. 470) |
| DMD.NM_004014- | UGGUUUAACUUGAGAGCAAUU (SEQ | UUGCUCUCAAGUUAAACCAUU |
| Pr-15′ | ID NO. 471) | (SEQ ID NO. 472) |
| DMD.NM_004014- | ACAGUCAGCACUUGAUAAAUU (SEQ | UUUAUCAAGUGCUGACUGUUU |
| Pr-20′ | ID NO. 473) | (SEQ ID NO. 474) |
| DMD.NM_004014- | CUGUAUAGCUGAUGAUAAAUU (SEQ | UUUAUCAUCAGCUAUACAGUU |
| Pr-24′ | ID NO. 475) | (SEQ ID NO. 476) |
| DMD.NM_004014- | GGUUUAACUUGAGAGCAAAUU (SEQ | UUUGCUCUCAAGUUAAACCUU |
| Pr-26′ | ID NO. 477) | (SEQ ID NO. 478) |
| DMD.NM_004014- | GUCAGCACUUGAUAAAUAUUU (SEQ | AUAUUUAUCAAGUGCUGACUU |
| Pr-27′ | ID NO. 479) | (SEQ ID NO. 480) |
| DMD.NM_004014- | CAAUUGAUAUCAGGUUCAAUU (SEQ | UUGAACCUGAUAUCAAUUGUU |
| Pr-37′ | ID NO. 481) | (SEQ ID NO. 482) |
| DMD.NM_004019- | AAAGAAAUCCUGGGCUGAAUU (SEQ | UUCAGCCCAGGAUUUCUUUUU |
| Pr-7′ | ID NO. 483) | (SEQ ID NO. 484) |
| DMD.NM_004019- | GCAUUACCUGAUGGAGAUUUU (SEQ | AAUCUCCAUCAGGUAAUGCUU |
| Pr-14′ | ID NO. 485) | (SEQ ID NO. 486) |
| DMD.NM_004019- | AUAGGUUAUUGGUGUCAAAUU (SEQ | UUUGACACCAAUAACCUAUUU |
| Pr-16′ | ID NO. 487) | (SEQ ID NO. 488) |
| DMD.NM_004019- | CUACGUCCUUAGAAAUUUAUU (SEQ | UAAAUUUCUAAGGACGUAGUU |
| Pr-25′ | ID NO. 489) | (SEQ ID NO. 490) |
| DMD.NM_004019- | UGCAUUAUCCUAUAGGUUAUU (SEQ | UAACCUAUAGGAUAAUGCAUU |
| Pr-29′ | ID NO. 491) | (SEQ ID NO. 492) |
| DMD.NM_004019- | GACAUCUCCUCAUGAAUAUUU (SEQ | AUAUUCAUGAGGAGAUGUCUU |
| Pr-33′ | ID NO. 493) | (SEQ ID NO. 494) |
| DMD.NM_004019- | GAAUCAGCUCCUUGUCAAAUU (SEQ | UUUGACAAGGAGCUGAUUCUU |
| Pr-39′ | ID NO. 495) | (SEQ ID NO. 496) |
| DMD.NM_004019- | CUGUAAUCCUGCUGAACAAUU (SEQ | UUGUUCAGCAGGAUUACAGUU |
| Pr-40′ | ID NO. 497) | (SEQ ID NO. 498) |
| DMD.NM_004021- | GGGUCAUCGUCAUCACAAUUU (SEQ | AUUGUGAUGACGAUGACCCUU |
| Pr-8′ | ID NO. 499) | (SEQ ID NO. 500) |
| DMD.NM_004021- | UGCUUCCACCAGUGAGAUAUU (SEQ | UAUCUCACUGGUGGAAGCAUU |
| Pr-9′ | ID NO. 501) | (SEQ ID NO. 502) |
| DMD.NM_004021- | GUAGUAAUCUGUAGAGUUAUU (SEQ | UAACUCUACAGAUUACUACUU |
| Pr-15′ | ID NO. 503) | (SEQ ID NO. 504) |
| DMD.NM_004021- | CGAAGUUUGGUCAAAUAUAUU (SEQ | UAUAUUUGACCAAACUUCGUU |
| Pr-20′ | ID NO. 505) | (SEQ ID NO. 506) |
| DMD.NM_004021- | GGAUUGAUCUGUGUAACUAUU (SEQ | UAGUUACACAGAUCAAUCCUU |
| Pr-23′ | ID NO. 507) | (SEQ ID NO. 508) |
| DMD.NM_004021- | UGAGUAGUAUCUCUGAGUAUU (SEQ | UACUCAGAGAUACUACUCAUU |
| Pr-26′ | ID NO. 509) | (SEQ ID NO. 510) |
| DMD.NM_004021- | GCAUAGCAAUCCUGAGAAAUU (SEQ | UUUCUCAGGAUUGCUAUGCUU |
| Pr-27′ | ID NO. 511) | (SEQ ID NO. 512) |
| DMD.NM_004021- | CGUAGUAAUCUGUAGAGUUUU (SEQ | AACUCUACAGAUUACUACGUU |
| Pr-31′ | ID NO. 513) | (SEQ ID NO. 514) |
| DMD.NM_004021- | GAAAGUAUUUCUGAGUAGUUU (SEQ | ACUACUCAGAAAUACUUUCUU |
| Pr-32′ | ID NO. 515) | (SEQ ID NO. 516) |

DMD gene encodes a protein called dystrophin. This protein is located primarily in muscles used for movement (skeletal muscles) and in heart (cardiac) muscle. Small amounts of dystrophin are present in nerve cells in the brain. In skeletal and cardiac muscles, dystrophin is part of a group of proteins (a protein complex) that work together to strengthen muscle fibers and protect them from injury as muscles contract and relax. The dystrophin complex acts as an anchor, connecting each muscle cell's structural framework (cytoskeleton) with the lattice of proteins and other molecules outside the cell (extracellular matrix). The dystrophin complex may also play a role in cell signaling by interacting with proteins that send and receive chemical signals. Research also suggests that dystrophin is important for the normal structure and function of synapses, which are specialized connections between nerve cells where cell-to-cell communication occurs. Mutations in DMD gene cause diseases such as a heart disease called DMD-associated dilated cardiomyopathy and Duchenne and Becker muscular dystrophy (Bovolenta et al., PLoS One, vol. 7(9):e45328 (2012); Narayanan et al., PLoS One, vol. 8(6):e67237 (2013), the contents of each of which are incorporated herein by reference in their entirety). Patients with DMD-associated dilated cardiomyopathy have little or no functional dystrophin in the heart and reduced amounts of dystrophin in skeletal muscle cells. The cardiac muscle is enlarged and weakened, preventing the heart from pumping blood efficiently. Duchenne and Becker muscular dystrophy are characterized by progressive muscle weakness and wasting (atrophy). Skeletal and cardiac muscle cells without enough functional dystrophin become damaged as the muscles repeatedly contract and relax with use. The damaged cells weaken and die over time, causing the characteristic muscle weakness and heart problems seen in Duchenne and Becker muscular dystrophy.

In one embodiment, provided is a method of modulating dystrophin protein levels comprising administering DMD-saRNA of the present invention, wherein the DMD-saRNA targets an antisense RNA transcript of DMD gene. The dystrophin protein levels may be dystrophin protein levels in skeletal muscle cells or in cardiac muscle cells. In one embodiment, dystrophin protein level is increased by at least 20, 30, 40%, or at least 45, 50, 55, 60, 65, 70, 75%, or at least 80% in the presence of the DMD-saRNA of the present invention compared to dystrophin protein level in the absence of the DMD-saRNA of the present invention. In a further embodiment, dystrophin protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the DMD-saRNA of the present invention compared to dystrophin protein level in the absence of the DMD-saRNA of the present invention.

In another embodiment, provided is a method of treating DMD-associated dilated cardiomyopathy or Duchenne and Becker muscular dystrophy comprising administering DMD-saRNA of the present invention, wherein the DMD-saRNA targets an antisense RNA transcript of DMD gene, and wherein the symptoms of DMD-associated dilated cardiomyopathy or Duchenne and Becker muscular dystrophy are reduced.

In one embodiment, the DMD-saRNA of the present invention is an antisense sequence such as, but not limited to, SEQ ID NO: 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 2808, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, 3058, 3060, 3062, 3064, 3066, 3068, 3070, 3072, 3074, 3076, 3078, 3080, 3082, 3084, 3086, 3088, 3090, 3092, 3094, 3096, 3098, 3100, 3102, 3104, 3106, 3108, 3110, 3112, 3114, 3116, 3118, 3120, 3122, 3124, 3126, 3128, 3130, 3132, 3134, 3136 and 3138. As a non-limiting example, the DMD-saRNA may be used to modulate dystrophin proteins levels and/or treat DMD-associated dilated cardiomyopathy or Duchenne and Becker muscular dystrophy.

In one embodiment, the DMD-saRNA of the present invention is an saRNA duplex. The saRNA duplex may be a pair of sense and antisense sequences such as, but not limited to, SEQ ID NOs: 309 and 310; SEQ ID NOs: 311 and 312; SEQ ID NOs: 313 and 314; SEQ ID NOs: 315 and 316; SEQ ID NOs: 317 and 318; SEQ ID NOs: 319 and 320; SEQ ID NOs: 321 and 322; SEQ ID NOs: 323 and 324; SEQ ID NOs: 325 and 326; SEQ ID NOs: 327 and 328; SEQ ID NOs: 329 and 330; SEQ ID NOs: 331 and 332; SEQ ID NOs: 333 and 334; SEQ ID NOs: 335 and 336; SEQ ID NOs: 337 and 338; SEQ ID NOs: 339 and 340; SEQ ID NOs: 341 and 342; SEQ ID NOs: 343 and 344; SEQ ID NOs: 345 and 346; SEQ ID NOs: 347 and 348; SEQ ID NOs: 349 and 350; SEQ ID NOs: 351 and 352; SEQ ID NOs: 353 and 354; SEQ ID NOs: 355 and 356; SEQ ID NOs: 357 and 358; SEQ ID NOs: 359 and 360; SEQ ID NOs: 361 and 362; SEQ ID NOs: 363 and 364; SEQ ID NOs: 365 and 366; SEQ ID NOs: 367 and 368; SEQ ID NOs: 369 and 370; SEQ ID NOs: 371 and 372; SEQ ID NOs: 373 and 374; SEQ ID NOs: 375 and 376; SEQ ID NOs: 377 and 378; SEQ ID NOs: 379 and 380; SEQ ID NOs: 381 and 382; SEQ ID NOs: 383 and 384; SEQ ID NOs: 385 and 386; SEQ ID NOs: 387 and 388; SEQ ID NOs: 389 and 390; SEQ ID NOs: 391 and 392; SEQ ID NOs: 393 and 394; SEQ ID NOs: 395 and 396; SEQ ID NOs: 397 and 398; SEQ ID NOs: 399 and 400; SEQ ID NOs: 401 and 402; SEQ ID NOs: 403 and 404; SEQ ID NOs: 405 and 406; SEQ ID NOs: 407 and 408; SEQ ID NOs: 409 and 410; SEQ ID NOs: 411 and 412; SEQ ID NOs: 413 and 414; SEQ ID NOs: 415 and 416; SEQ ID NOs: 417 and 418; SEQ ID NOs: 419 and 420; SEQ ID NOs: 421 and 422; SEQ ID NOs: 423 and 424; SEQ ID NOs: 425 and 426; SEQ ID NOs: 427 and 428; SEQ ID NOs: 429 and 430; SEQ ID NOs: 431 and 432; SEQ ID NOs: 433 and 434; SEQ ID NOs: 435 and 436; SEQ ID NOs: 437 and 438; SEQ ID NOs: 439 and 440; SEQ ID NOs: 441 and 442; SEQ ID NOs: 443 and 444; SEQ ID NOs: 445 and 446; SEQ ID NOs: 447 and 448; SEQ ID NOs: 449 and 450; SEQ ID NOs: 451 and 452; SEQ ID NOs: 453 and 454; SEQ ID NOs: 455 and 456; SEQ ID NOs: 457 and 458; SEQ ID NOs: 459 and 460; SEQ ID NOs: 461 and 462; SEQ ID NOs: 463 and 464; SEQ ID NOs: 465 and 466; SEQ ID NOs: 467 and 468; SEQ ID NOs: 469 and 470; SEQ ID NOs: 471 and 472; SEQ ID NOs: 473 and 474; SEQ ID NOs: 475 and 476; SEQ ID NOs: 477 and 478; SEQ ID NOs: 479 and 480; SEQ ID NOs: 481 and 482; SEQ ID NOs: 483 and 484; SEQ ID NOs: 485 and 486; SEQ ID NOs: 487 and 488; SEQ ID NOs: 489 and 490; SEQ ID NOs: 491 and 492; SEQ ID NOs: 493 and 494; SEQ ID NOs: 495 and 496; SEQ ID NOs: 497 and 498; SEQ ID NOs: 499 and 500; SEQ ID NOs: 501 and 502; SEQ ID NOs: 503 and 504; SEQ ID NOs: 505 and 506; SEQ ID NOs: 507 and 508; SEQ ID NOs: 509 and 510; SEQ ID NOs: 511 and 512; SEQ ID NOs: 513 and 514; SEQ ID NOs: 515 and 516; SEQ ID NOs: 2361 and 2362; SEQ ID NOs: 2363 and 2364; SEQ ID NOs: 2365 and 2366; SEQ ID NOs: 2367 and 2368; SEQ ID NOs: 2369 and 2370; SEQ ID NOs: 2371 and 2372; SEQ ID NOs: 2373 and 2374; SEQ ID NOs: 2375 and 2376; SEQ ID NOs: 2377 and 2378; SEQ ID NOs: 2379 and 2380; SEQ ID NOs: 2381 and 2382; SEQ ID NOs: 2383 and 2384; SEQ ID NOs: 2385 and 2386; SEQ ID NOs: 2387 and 2388; SEQ ID NOs: 2389 and 2390; SEQ ID NOs: 2391 and 2392; SEQ ID NOs: 2393 and 2394; SEQ ID NOs: 2395 and 2396; SEQ ID NOs: 2397 and 2398; SEQ ID NOs: 2399 and 2400; SEQ ID NOs: 2401 and 2402; SEQ ID NOs: 2403 and 2404; SEQ ID NOs: 2405 and 2406; SEQ ID NOs: 2407 and 2408; SEQ ID NOs: 2409 and 2410; SEQ ID NOs: 2411 and 2412; SEQ ID NOs: 2413 and 2414; SEQ ID NOs: 2415 and 2416; SEQ ID NOs: 2417 and 2418; SEQ ID NOs: 2419 and 2420; SEQ ID NOs: 2421 and 2422; SEQ ID NOs: 2423 and 2424; SEQ ID NOs: 2425 and 2426; SEQ ID NOs: 2427 and 2428; SEQ ID NOs: 2429 and 2430; SEQ ID NOs: 2431 and 2432; SEQ ID NOs: 2433 and 2434; SEQ ID NOs: 2435 and 2436; SEQ ID NOs: 2437 and 2438; SEQ ID NOs: 2439 and 2440; SEQ ID NOs: 2441 and 2442; SEQ ID NOs: 2443 and 2444; SEQ ID NOs: 2445 and 2446; SEQ ID NOs: 2447 and 2448; SEQ ID NOs: 2449 and 2450; SEQ ID NOs: 2451 and 2452; SEQ ID NOs: 2453 and 2454; SEQ ID NOs: 2455 and 2456; SEQ ID NOs: 2457 and 2458; SEQ ID NOs: 2459 and 2460; SEQ ID NOs: 2461 and 2462; SEQ ID NOs: 2463 and 2464; SEQ ID NOs: 2465 and 2466; SEQ ID NOs: 2467 and 2468; SEQ ID NOs: 2469 and 2470; SEQ ID NOs: 2471 and 2472; SEQ ID NOs: 2473 and 2474; SEQ ID NOs: 2475 and 2476; SEQ ID NOs: 2477 and 2478; SEQ ID NOs: 2479 and 2480; SEQ ID NOs: 2481 and 2482; SEQ ID NOs: 2483 and 2484; SEQ ID NOs: 2485 and 2486; SEQ ID NOs: 2487 and 2488; SEQ ID NOs: 2489 and 2490; SEQ ID NOs: 2491 and 2492; SEQ ID NOs: 2493 and 2494; SEQ ID NOs: 2495 and 2496; SEQ ID NOs: 2497 and 2498; SEQ ID NOs: 2499 and 2500; SEQ ID NOs: 2501 and 2502; SEQ ID NOs: 2503 and 2504; SEQ ID NOs: 2505 and 2506; SEQ ID NOs: 2507 and 2508; SEQ ID NOs: 2509 and 2510; SEQ ID NOs: 2511 and 2512; SEQ ID NOs: 2513 and 2514; SEQ ID NOs: 2515 and 2516; SEQ ID NOs: 2517 and 2518; SEQ ID NOs: 2519 and 2520; SEQ ID NOs: 2521 and 2522; SEQ ID NOs: 2523 and 2524; SEQ ID NOs: 2525 and 2526; SEQ ID NOs: 2527 and 2528; SEQ ID NOs: 2529 and 2530; SEQ ID NOs: 2531 and 2532; SEQ ID NOs: 2533 and 2534; SEQ ID NOs: 2535 and 2536; SEQ ID NOs: 2537 and 2538; SEQ ID NOs: 2539 and 2540; SEQ ID NOs: 2541 and 2542; SEQ ID NOs: 2543 and 2544; SEQ ID NOs: 2545 and 2546; SEQ ID NOs: 2547 and 2548; SEQ ID NOs: 2549 and 2550; SEQ ID NOs: 2551 and 2552; SEQ ID NOs: 2553 and 2554; SEQ ID NOs: 2555 and 2556; SEQ ID NOs: 2557 and 2558; SEQ ID NOs: 2559 and 2560; SEQ ID NOs: 2561 and 2562; SEQ ID NOs: 2563 and 2564; SEQ ID NOs: 2565 and 2566; SEQ ID NOs: 2567 and 2568; SEQ ID NOs: 2569 and 2570; SEQ ID NOs: 2571 and 2572; SEQ ID NOs: 2573 and 2574; SEQ ID NOs: 2575 and 2576; SEQ ID NOs: 2577 and 2578; SEQ ID NOs: 2579 and 2580; SEQ ID NOs: 2581 and 2582; SEQ ID NOs: 2583 and 2584; SEQ ID NOs: 2585 and 2586; SEQ ID NOs: 2587 and 2588; SEQ ID NOs: 2589 and 2590; SEQ ID NOs: 2591 and 2592; SEQ ID NOs: 2593 and 2594; SEQ ID NOs: 2595 and 2596; SEQ ID NOs: 2597 and 2598; SEQ ID NOs: 2599 and 2600; SEQ ID NOs: 2601 and 2602; SEQ ID NOs: 2603 and 2604; SEQ ID NOs: 2605 and 2606; SEQ ID NOs: 2607 and 2608; SEQ ID NOs: 2609 and 2610; SEQ ID NOs: 2611 and 2612; SEQ ID NOs: 2613 and 2614; SEQ ID NOs: 2615 and 2616; SEQ ID NOs: 2617 and 2618; SEQ ID NOs: 2619 and 2620; SEQ ID NOs: 2621 and 2622; SEQ ID NOs: 2623 and 2624; SEQ ID NOs: 2625 and 2626; SEQ ID NOs: 2627 and 2628; SEQ ID NOs: 2629 and 2630; SEQ ID NOs: 2631 and 2632; SEQ ID NOs: 2633 and 2634; SEQ ID NOs: 2635 and 2636; SEQ ID NOs: 2637 and 2638; SEQ ID NOs: 2639 and 2640; SEQ ID NOs: 2641 and 2642; SEQ ID NOs: 2643 and 2644; SEQ ID NOs: 2645 and 2646; SEQ ID NOs: 2647 and 2648; SEQ ID NOs: 2649 and 2650; SEQ ID NOs: 2651 and 2652; SEQ ID NOs: 2653 and 2654; SEQ ID NOs: 2655 and 2656; SEQ ID NOs: 2657 and 2658; SEQ ID NOs: 2659 and 2660; SEQ ID NOs: 2661 and 2662; SEQ ID NOs: 2663 and 2664; SEQ ID NOs: 2665 and 2666; SEQ ID NOs: 2667 and 2668; SEQ ID NOs: 2669 and 2670; SEQ ID NOs: 2671 and 2672; SEQ ID NOs: 2673 and 2674; SEQ ID NOs: 2675 and 2676; SEQ ID NOs: 2677 and 2678; SEQ ID NOs: 2679 and 2680; SEQ ID NOs: 2681 and 2682; SEQ ID NOs: 2683 and 2684; SEQ ID NOs: 2685 and 2686; SEQ ID NOs: 2687 and 2688; SEQ ID NOs: 2689 and 2690; SEQ ID NOs: 2691 and 2692; SEQ ID NOs: 2693 and 2694; SEQ ID NOs: 2695 and 2696; SEQ ID NOs: 2697 and 2698; SEQ ID NOs: 2699 and 2700; SEQ ID NOs: 2701 and 2702; SEQ ID NOs: 2703 and 2704; SEQ ID NOs: 2705 and 2706; SEQ ID NOs: 2707 and 2708; SEQ ID NOs: 2709 and 2710; SEQ ID NOs: 2711 and 2712; SEQ ID NOs: 2713 and 2714; SEQ ID NOs: 2715 and 2716; SEQ ID NOs: 2717 and 2718; SEQ ID NOs: 2719 and 2720; SEQ ID NOs: 2721 and 2722; SEQ ID NOs: 2723 and 2724; SEQ ID NOs: 2725 and 2726; SEQ ID NOs: 2727 and 2728; SEQ ID NOs: 2729 and 2730; SEQ ID NOs: 2731 and 2732; SEQ ID NOs: 2733 and 2734; SEQ ID NOs: 2735 and 2736; SEQ ID NOs: 2737 and 2738; SEQ ID NOs: 2739 and 2740; SEQ ID NOs: 2741 and 2742; SEQ ID NOs: 2743 and 2744; SEQ ID NOs: 2745 and 2746; SEQ ID NOs: 2747 and 2748; SEQ ID NOs: 2749 and 2750; SEQ ID NOs: 2751 and 2752; SEQ ID NOs: 2753 and 2754; SEQ ID NOs: 2755 and 2756; SEQ ID NOs: 2757 and 2758; SEQ ID NOs: 2759 and 2760; SEQ ID NOs: 2761 and 2762; SEQ ID NOs: 2763 and 2764; SEQ ID NOs: 2765 and 2766; SEQ ID NOs: 2767 and 2768; SEQ ID NOs: 2769 and 2770; SEQ ID NOs: 2771 and 2772; SEQ ID NOs: 2773 and 2774; SEQ ID NOs: 2775 and 2776; SEQ ID NOs: 2777 and 2778; SEQ ID NOs: 2779 and 2780; SEQ ID NOs: 2781 and 2782; SEQ ID NOs: 2783 and 2784; SEQ ID NOs: 2785 and 2786; SEQ ID NOs: 2787 and 2788; SEQ ID NOs: 2789 and 2790; SEQ ID NOs: 2791 and 2792; SEQ ID NOs: 2793 and 2794; SEQ ID NOs: 2795 and 2796; SEQ ID NOs: 2797 and 2798; SEQ ID NOs: 2799 and 2800; SEQ ID NOs: 2801 and 2802; SEQ ID NOs: 2803 and 2804; SEQ ID NOs: 2805 and 2806; SEQ ID NOs: 2807 and 2808; SEQ ID NOs: 2809 and 2810; SEQ ID NOs: 2811 and 2812; SEQ ID NOs: 2813 and 2814; SEQ ID NOs: 2815 and 2816; SEQ ID NOs: 2817 and 2818; SEQ ID NOs: 2819 and 2820; SEQ ID NOs: 2821 and 2822; SEQ ID NOs: 2823 and 2824; SEQ ID NOs: 2825 and 2826; SEQ ID NOs: 2827 and 2828; SEQ ID NOs: 2829 and 2830; SEQ ID NOs: 2831 and 2832; SEQ ID NOs: 2833 and 2834; SEQ ID NOs: 2835 and 2836; SEQ ID NOs: 2837 and 2838; SEQ ID NOs: 2839 and 2840; SEQ ID NOs: 2841 and 2842; SEQ ID NOs: 2843 and 2844; SEQ ID NOs: 2845 and 2846; SEQ ID NOs: 2847 and 2848; SEQ ID NOs: 2849 and 2850; SEQ ID NOs: 2851 and 2852; SEQ ID NOs: 2853 and 2854; SEQ ID NOs: 2855 and 2856; SEQ ID NOs: 2857 and 2858; SEQ ID NOs: 2859 and 2860; SEQ ID NOs: 2861 and 2862; SEQ ID NOs: 2863 and 2864; SEQ ID NOs: 2865 and 2866; SEQ ID NOs: 2867 and 2868; SEQ ID NOs: 2869 and 2870; SEQ ID NOs: 2871 and 2872; SEQ ID NOs: 2873 and 2874; SEQ ID NOs: 2875 and 2876; SEQ ID NOs: 2877 and 2878; SEQ ID NOs: 2879 and 2880; SEQ ID NOs: 2881 and 2882; SEQ ID NOs: 2883 and 2884; SEQ ID NOs: 2885 and 2886; SEQ ID NOs: 2887 and 2888; SEQ ID NOs: 2889 and 2890; SEQ ID NOs: 2891 and 2892; SEQ ID NOs: 2893 and 2894; SEQ ID NOs: 2895 and 2896; SEQ ID NOs: 2897 and 2898; SEQ ID NOs: 2899 and 2900; SEQ ID NOs: 2901 and 2902; SEQ ID NOs: 2903 and 2904; SEQ ID NOs: 2905 and 2906; SEQ ID NOs: 2907 and 2908; SEQ ID NOs: 2909 and 2910; SEQ ID NOs: 2911 and 2912; SEQ ID NOs: 2913 and 2914; SEQ ID NOs: 2915 and 2916; SEQ ID NOs: 2917 and 2918; SEQ ID NOs: 2919 and 2920; SEQ ID NOs: 2921 and 2922; SEQ ID NOs: 2923 and 2924; SEQ ID NOs: 2925 and 2926; SEQ ID NOs: 2927 and 2928; SEQ ID NOs: 2929 and 2930; SEQ ID NOs: 2931 and 2932; SEQ ID NOs: 2933 and 2934; SEQ ID NOs: 2935 and 2936; SEQ ID NOs: 2937 and 2938; SEQ ID NOs: 2939 and 2940; SEQ ID NOs: 2941 and 2942; SEQ ID NOs: 2943 and 2944; SEQ ID NOs: 2945 and 2946; SEQ ID NOs: 2947 and 2948; SEQ ID NOs: 2949 and 2950; SEQ ID NOs: 2951 and 2952; SEQ ID NOs: 2953 and 2954; SEQ ID NOs: 2955 and 2956; SEQ ID NOs: 2957 and 2958; SEQ ID NOs: 2959 and 2960; SEQ ID NOs: 2961 and 2962; SEQ ID NOs: 2963 and 2964; SEQ ID NOs: 2965 and 2966; SEQ ID NOs: 2967 and 2968; SEQ ID NOs: 2969 and 2970; SEQ ID NOs: 2971 and 2972; SEQ ID NOs: 2973 and 2974; SEQ ID NOs: 2975 and 2976; SEQ ID NOs: 2977 and 2978; SEQ ID NOs: 2979 and 2980; SEQ ID NOs: 2981 and 2982; SEQ ID NOs: 2983 and 2984; SEQ ID NOs: 2985 and 2986; SEQ ID NOs: 2987 and 2988; SEQ ID NOs: 2989 and 2990; SEQ ID NOs: 2991 and 2992; SEQ ID NOs: 2993 and 2994; SEQ ID NOs: 2995 and 2996; SEQ ID NOs: 2997 and 2998; SEQ ID NOs: 2999 and 3000; SEQ ID NOs: 3001 and 3002; SEQ ID NOs: 3003 and 3004; SEQ ID NOs: 3005 and 3006; SEQ ID NOs: 3007 and 3008; SEQ ID NOs: 3009 and 3010; SEQ ID NOs: 3011 and 3012; SEQ ID NOs: 3013 and 3014; SEQ ID NOs: 3015 and 3016; SEQ ID NOs: 3017 and 3018; SEQ ID NOs: 3019 and 3020; SEQ ID NOs: 3021 and 3022; SEQ ID NOs: 3023 and 3024; SEQ ID NOs: 3025 and 3026; SEQ ID NOs: 3027 and 3028; SEQ ID NOs: 3029 and 3030; SEQ ID NOs: 3031 and 3032; SEQ ID NOs: 3033 and 3034; SEQ ID NOs: 3035 and 3036; SEQ ID NOs: 3037 and 3038; SEQ ID NOs: 3039 and 3040; SEQ ID NOs: 3041 and 3042; SEQ ID NOs: 3043 and 3044; SEQ ID NOs: 3045 and 3046; SEQ ID NOs: 3047 and 3048; SEQ ID NOs: 3049 and 3050; SEQ ID NOs: 3051 and 3052; SEQ ID NOs: 3053 and 3054; SEQ ID NOs: 3055 and 3056; SEQ ID NOs: 3057 and 3058; SEQ ID NOs: 3059 and 3060; SEQ ID NOs: 3061 and 3062; SEQ ID NOs: 3063 and 3064; SEQ ID NOs: 3065 and 3066; SEQ ID NOs: 3067 and 3068; SEQ ID NOs: 3069 and 3070; SEQ ID NOs: 3071 and 3072; SEQ ID NOs: 3073 and 3074; SEQ ID NOs: 3075 and 3076; SEQ ID NOs: 3077 and 3078; SEQ ID NOs: 3079 and 3080; SEQ ID NOs: 3081 and 3082; SEQ ID NOs: 3083 and 3084; SEQ ID NOs: 3085 and 3086; SEQ ID NOs: 3087 and 3088; SEQ ID NOs: 3089 and 3090; SEQ ID NOs: 3091 and 3092; SEQ ID NOs: 3093 and 3094; SEQ ID NOs: 3095 and 3096; SEQ ID NOs: 3097 and 3098; SEQ ID NOs: 3099 and 3100; SEQ ID NOs: 3101 and 3102; SEQ ID NOs: 3103 and 3104; SEQ ID NOs: 3105 and 3106; SEQ ID NOs: 3107 and 3108; SEQ ID NOs: 3109 and 3110; SEQ ID NOs: 3111 and 3112; SEQ ID NOs: 3113 and 3114; SEQ ID NOs: 3115 and 3116; SEQ ID NOs: 3117 and 3118; SEQ ID NOs: 3119 and 3120; SEQ ID NOs: 3121 and 3122; SEQ ID NOs: 3123 and 3124; SEQ ID NOs: 3125 and 3126; SEQ ID NOs: 3127 and 3128; SEQ ID NOs: 3129 and 3130; SEQ ID NOs: 3131 and 3132; SEQ ID NOs: 3133 and 3134; SEQ ID NOs: 3135 and 3136; SEQ ID NOs: 3137 and 3138. As a non-limiting example, the DMD-saRNA which are saRNA duplexes may be used to modulate dystrophin proteins levels and/or treat DMD-associated dilated cardiomyopathy or Duchenne and Becker muscular dystrophy.

### PAX5 Gene

As another non-limiting example, provided is a method of modulating the expression of PAX5 gene comprising administering saRNA of the present invention, wherein the saRNA targets an antisense RNA transcript of PAX5 gene. These saRNAs are called PAX5-saRNA. In one embodiment, the expression of PAX5 gene is increased by at least 20, 30, 40%, or at least 45, 50, 55, 60, 65, 70, 75%, or at least 80% in the presence of the saRNA of the present invention compared to the expression of PAX5 gene in the absence of the saRNA of the present invention. In a further embodiment, the expression of PAX5 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present invention compared to the expression of PAX5 gene in the absence of the saRNA of the present invention. The modulation of the expression of PAX5 gene may be reflected or determined by the change of PAX5 mRNA levels.

PAX5-saRNAs may be single-stranded and comprise 14-30 nucleotides. The sequence of a single-stranded PAX5-saRNA may have at least 60%, 70%, 80% or 90% identity with a sequence selected from the sequences of the antisense strands in Table 8-2. In one embodiment, the single-stranded saRNA comprises a sequence selected from the sequences of the antisense strands in Table 8-2. In one embodiment, the single-stranded PAX5-saRNA may have a 3′ tail. The sequence of a single-stranded PAX5-saRNA with a 3′ tail may have at least 60%, 70%, 80% or 90% identity with a sequence selected from the sequences of the antisense strands in Table 9. In one embodiment, the single-stranded saRNA comprises a sequence selected from the sequences of the antisense strands in Table 9.

PAX5-saRNAs may be double-stranded. The two strands form a duplex and each strand comprises 14-30 nucleotides. The first strand of a double-stranded PAX5-saRNA may have at least 60%, 70%, 80% or 90% identity with a sequence selected from the sequences of the antisense strands in Table 8-2. In one embodiment, the first strand of the double-stranded saRNA comprises a sequence selected from the sequences of the antisense strands in Table 8-2. The second strand of a double-stranded PAX5-saRNA may have at least 60%, 70%, 80% or 90% identity with a sequence selected from the sequences of the sense strands in Table 8-2. In one embodiment, the second strand of the double-stranded saRNA comprises a sequence selected from the sequences of the sense strands in Table 8-2. In one embodiment, the double-stranded PAX5-saRNA may have a 3′ overhang on each strand. The first strand of a double-stranded PAX5-saRNA may have at least 60%, 70%, 80% or 90% identity with a sequence selected from the sequences of the antisense strands in Table 9. In one embodiment, the first strand of the double-stranded saRNA comprises a sequence selected from the sequences of the antisense strands in Table 9. The second strand of a double-stranded PAX5-saRNA may have at least 60%, 70%, 80% or 90% identity with a sequence selected from the sequences of the sense strands in Table 9. In one embodiment, the second strand of the double-stranded saRNA comprises a sequence selected from the sequences of the sense strands in Table 9, i.e., SEQ ID NOS 544, 546, 548, 550, 552, 554, 556, 558, and 560.

PAX-saRNAs may be modified or unmodified.

Table 8-1 describes the target gene, the targeted sequence, the genomic location of the targeted sequence and the relative location of PAX5-saRNAs with no 3′ overhang. In Table 8-1, the targeted sequence is defined as a region on the template strand of the target gene which is identical to a region on the target antisense RNA transcript to which the antisense strand of an saRNA hybridizes. In Table 8-1, the relative location is the distance from the 5′ end of the targeted sequence to the TSS. A negative number represents a location upstream of the TSS and a positive number represents a location downstream of the TSS.

TABLE 8-1
Sequences of PAX5-saRNAs (with no 3′ overhang)

| | Target | | Genomic Location of the | Relative |
|---|---|---|---|---|
| saRNA ID | Gene | Targeted sequence | Targeted sequence | Location |
| PAX.NM_016734-Pr-2 | PAX | ctcagatatttccgaatta | chr9: 37033476 plus | 1000 |
| | human | (SEQ ID NO. 517) | strand | |
| PAX.NM_016734-Pr-3 | PAX | gaggttaagctgtgaaata | chr9: 37035021 plus | −545 |
| | human | (SEQ ID NO. 518) | strand | |
| PAX.NM_016734-Pr- | PAX | ggttaagctgtgaaatact | chr9: 37035023 plus | −547 |
| 20 | human | (SEQ ID NO. 519) | strand | |
| PAX.NM_016734-Pr- | PAX | tggaggttaagctgtgaaa | chr9: 37035019 plus | −543 |
| 25 | human | (SEQ ID NO. 520) | strand | |
| PAX.NM_016734-Pr- | PAX | tccgaggagtcggataatt | chr9: 37033994 plus | 482 |
| 27 | human | (SEQ ID NO. 521) | strand | |
| PAX.NM_016734-Pr- | PAX | tcagatatttccgaattac | chr9: 37033477 plus | 999 |
| 31 | human | (SEQ ID NO. 522) | strand | |
| PAX.NM_016734-Pr- | PAX | agatatttggactgatgca | chr9: 37033796 plus | 680 |
| 36 | human | (SEQ ID NO. 523) | strand | |
| PAX.NM_016734-Pr- | PAX | gtccgaggagtcggataat | chr9: 37033993 plus | 483 |
| 37 | human | (SEQ ID NO. 524) | strand | |
| PAX.NM_016734-Pr- | PAX | cacagatatttggactgat | chr9: 37033793 plus | 683 |
| 39 | human | (SEQ ID NO. 525) | strand | |

TABLE 8-2
Sequences of PAX5-saRNAs
(with no 3′ overhang)

| | | Antisense |
|---|---|---|
| saRNA ID | Sense (passenger) | (guide) |
| PAX.NM_016734- | CUCAGAUAUUUCCGAAUUA | UAAUUCGGAAAUAUCU |
| Pr-2 | (SEQ ID NO. 526) | GAG |
| | | (SEQ ID NO. 527) |
| PAX.NM_016734- | GAGGUUAAGCUGUGAAAUA | UAUUUCACAGCUUAAC |
| Pr-3 | (SEQ ID NO. 528) | CUC |
| | | (SEQ ID NO. 529) |
| PAX.NM_016734- | GGUUAAGCUGUGAAAUACU | AGUAUUUCACAGCUUA |
| Pr-20 | (SEQ ID NO. 530) | ACC |
| | | (SEQ ID NO. 531) |
| PAX.NM_016734- | UGGAGGUUAAGCUGUGAAA | UUUCACAGCUUAACCU |
| Pr-25 | (SEQ ID NO. 532) | CCA |
| | | (SEQ ID NO. 533) |
| PAX.NM_016734- | UCCGAGGAGUCGGAUAAUU | AAUUAUCCGACUCCUC |
| Pr-27 | (SEQ ID NO. 534) | GGA |
| | | (SEQ ID NO. 535) |
| PAX.NM_016734- | UCAGAUAUUUCCGAAUUAC | GUAAUUCGGAAAUAUC |
| Pr-31 | (SEQ ID NO. 536) | UGA |
| | | (SEQ ID NO. 537) |
| PAX.NM_016734- | AGAUAUUUGGACUGAUGCA | UGCAUCAGUCCAAAUA |
| Pr-36 | (SEQ ID NO. 538) | UCU |
| | | (SEQ ID NO. 539) |
| PAX.NM_016734- | GUCCGAGGAGUCGGAUAAU | AUUAUCCGACUCCUCG |
| Pr-37 | (SEQ ID NO. 540) | GAC |
| | | (SEQ ID NO. 541) |
| PAX.NM_016734- | CACAGAUAUUUGGACUGAU | AUCAGUCCAAAUAUCU |
| Pr-39 | (SEQ ID NO. 542) | GUG |
| | | (SEQ ID NO. 543) |

TABLE 9
Sequences of PAX5-saRNAs (with 3′ overhang)

| saRNA ID | Sense (passenger) | Antisense (guide) |
|---|---|---|
| PAX.NM_016734- | CUCAGAUAUUUCCGAAUUAUU (SEQ | UAAUUCGGAAAUAUCUGAGUU |
| Pr-2′ | ID NO. 544) | (SEQ ID NO. 545) |
| PAX.NM_016734- | GAGGUUAAGCUGUGAAAUAUU | UAUUUCACAGCUUAACCUCUU (SEQ |
| Pr-3′ | (SEQ ID NO. 546) | ID NO. 547) |
| PAX.NM_016734- | GGUUAAGCUGUGAAAUACUUU | AGUAUUUCACAGCUUAACCUU (SEQ |
| Pr-20′ | (SEQ ID NO. 548) | ID NO. 549) |
| PAX.NM_016734- | UGGAGGUUAAGCUGUGAAAUU | UUUCACAGCUUAACCUCCAUU (SEQ |
| Pr-25′ | (SEQ ID NO. 550) | ID NO. 551) |
| PAX.NM_016734- | UCCGAGGAGUCGGAUAAUUUU | AAUUAUCCGACUCCUCGGAUU (SEQ |
| Pr-27′ | (SEQ ID NO. 552) | ID NO. 553) |
| PAX.NM_016734- | UCAGAUAUUUCCGAAUUACUU (SEQ | GUAAUUCGGAAAUAUCUGAUU |
| Pr-31′ | ID NO. 554) | (SEQ ID NO. 555) |
| PAX.NM_016734- | AGAUAUUUGGACUGAUGCAUU | UGCAUCAGUCCAAAUAUCUUU (SEQ |
| Pr-36′ | (SEQ ID NO. 556) | ID NO. 557) |
| PAX.NM_016734- | GUCCGAGGAGUCGGAUAAUUU | AUUAUCCGACUCCUCGGACUU (SEQ |
| Pr-37′ | (SEQ ID NO. 558) | ID NO. 559) |
| PAX.NM_016734- | CACAGAUAUUUGGACUGAUUU | AUCAGUCCAAAUAUCUGUGUU (SEQ |
| Pr-39′ | (SEQ ID NO. 560) | ID NO. 561) |

PAX5 gene encodes a protein called B-cell lineage specific activation protein (BSAP), a member of the paired box (PAX) family of transcription factors. Paired box transcription factors are important regulators in early development, and alterations in the expression of their genes are thought to contribute to neoplastic transformation. BSAP is expressed at early, but not late stages of B-cell differentiation. Its expression has also been detected in developing CNS and testis and so the encoded protein may also play a role in neural development and spermatogenesis (Adams et al., Genes Dev., vol. 6:1589 (1992), the contents of which are incorporated herein by reference in their entirety). PAX5 gene plays an important role in B-cell differentiation as well as neural development and spermatogenesis. It is also involved in the regulation of the CD19 gene, a B-lymphoid-specific target gene. Diseases associated with PAX5 include lymphoplasmacytic lymphoma and diffuse large B-cell lymphoma of the central nervous system.

In one embodiment, provided is a method of modulating BSAP protein levels comprising administering PAX5-saRNA of the present invention, wherein the PAX5-saRNA targets an antisense RNA transcript of PAX5 gene. In one embodiment, BSAP protein level is increased by at least 20, 30, 40%, or at least 45, 50, 55, 60, 65, 70, 75%, or at least 80% in the presence of the PAX5-saRNA of the present invention compared to B SAP protein level in the absence of the PAX5-saRNA of the present invention. In a further embodiment, BSAP protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the PAX5-saRNA of the present invention compared to BSAP protein level in the absence of the PAX5-saRNA of the present invention.

In another embodiment, provided is a method of modulating B-cell differentiation comprising administering the PAX5-saRNA of the present invention, wherein the PAX5-saRNA targets an antisense RNA transcript of PAX5 gene.

In another embodiment, provided is a method of modulating neural development and spermatogenesis comprising administering the PAX5-saRNA of the present invention, wherein the PAX5-saRNA targets an antisense RNA transcript of PAX5 gene.

In another embodiment, provided is a method of treating lymphoplasmacytic lymphoma or diffuse large B-cell lymphoma of the central nervous system comprising administering the PAX5-saRNA of the present invention, wherein the PAX5-saRNA targets an antisense RNA transcript of PAX5 gene, and wherein the symptoms of treating lymphoplasmacytic lymphoma or diffuse large B-cell lymphoma are reduced.

In one embodiment, the PAX5-saRNA of the present invention is an antisense sequence such as, but not limited to, SEQ ID NO: 527, 529, 531, 533, 535, 537, 539, 541, 543, 545, 547, 549, 551, 553, 555, 557, 559, 561, 5216, 5218, 5220, 5222, 5224, 5226, 5228, 5230, 5232, 5234, 5236, 5238, 5240, 5242, 5244, 5246, 5248, 5250, 5252, 5254, 5256, 5258, 5260, 5262, 5264, 5266, 5268, 5270, 5272, 5274, 5276, 5278, 5280, 5282, 5284, 5286, 5288, 5290, 5292, 5294, 5296, 5298, 5300, 5302, 5304, 5306, 5308, 5310, 5312 and 5314. As a non-limiting example, these PAX5-saRNA sequences may be used to modulate BSAP protein levels, and/or treat lymphoplasmacytic lymphoma or diffuse large B-cell lymphoma of the central nervous system.

In one embodiment, the PAX5-saRNA of the present invention is an saRNA duplex. The saRNA duplex may be a pair of sense and antisense sequences such as, but not limited to, SEQ ID NOs: 526 and 527; SEQ ID NOs: 528 and 529; SEQ ID NOs: 530 and 531; SEQ ID NOs: 532 and 533; SEQ ID NOs: 534 and 535; SEQ ID NOs: 536 and 537; SEQ ID NOs: 538 and 539; SEQ ID NOs: 540 and 541; SEQ ID NOs: 542 and 543; SEQ ID NOs: 544 and 545; SEQ ID NOs: 546 and 547; SEQ ID NOs: 548 and 549; SEQ ID NOs: 550 and 551; SEQ ID NOs: 552 and 553; SEQ ID NOs: 554 and 555; SEQ ID NOs: 556 and 557; SEQ ID NOs: 558 and 559; SEQ ID NOs: 560 and 561; SEQ ID NOs: 5215 and 5216; SEQ ID NOs: 5217 and 5218; SEQ ID NOs: 5219 and 5220; SEQ ID NOs: 5221 and 5222; SEQ ID NOs: 5223 and 5224; SEQ ID NOs: 5225 and 5226; SEQ ID NOs: 5227 and 5228; SEQ ID NOs: 5229 and 5230; SEQ ID NOs: 5231 and 5232; SEQ ID NOs: 5233 and 5234; SEQ ID NOs: 5235 and 5236; SEQ ID NOs: 5237 and 5238; SEQ ID NOs: 5239 and 5240; SEQ ID NOs: 5241 and 5242; SEQ ID NOs: 5243 and 5244; SEQ ID NOs: 5245 and 5246; SEQ ID NOs: 5247 and 5248; SEQ ID NOs: 5249 and 5250; SEQ ID NOs: 5251 and 5252; SEQ ID NOs: 5253 and 5254; SEQ ID NOs: 5255 and 5256; SEQ ID NOs: 5257 and 5258; SEQ ID NOs: 5259 and 5260; SEQ ID NOs: 5261 and 5262; SEQ ID NOs: 5263 and 5264; SEQ ID NOs: 5265 and 5266; SEQ ID NOs: 5267 and 5268; SEQ ID NOs: 5269 and 5270; SEQ ID NOs: 5271 and 5272; SEQ ID NOs: 5273 and 5274; SEQ ID NOs: 5275 and 5276; SEQ ID NOs: 5277 and 5278; SEQ ID NOs: 5279 and 5280; SEQ ID NOs: 5281 and 5282; SEQ ID NOs: 5283 and 5284; SEQ ID NOs: 5285 and 5286; SEQ ID NOs: 5287 and 5288; SEQ ID NOs: 5289 and 5290; SEQ ID NOs: 5291 and 5292; SEQ ID NOs: 5293 and 5294; SEQ ID NOs: 5295 and 5296; SEQ ID NOs: 5297 and 5298; SEQ ID NOs: 5299 and 5300; SEQ ID NOs: 5301 and 5302; SEQ ID NOs: 5303 and 5304; SEQ ID NOs: 5305 and 5306; SEQ ID NOs: 5307 and 5308; SEQ ID NOs: 5309 and 5310; SEQ ID NOs: 5311 and 5312; SEQ ID NOs: 5313 and 5314. As a non-limiting example, these PAX5-saRNA sequences which are saRNA duplexes may be used to modulate BSAP protein levels, and/or treat lymphoplasmacytic lymphoma or diffuse large B-cell lymphoma of the central nervous system.

### SCN1A Gene

As another non-limiting example, provided is a method of modulating the expression of SCN1A gene comprising administering saRNA of the present invention, wherein the saRNA targets an antisense RNA transcript of SCN1A gene. These saRNAs are called SCN1A-saRNA. In one embodiment, the expression of SCN1A gene is increased by at least 20, 30, 40%, or at least 45, 50, 55, 60, 65, 70, 75%, or at least 80% in the presence of the saRNA of the present invention compared to the expression of SCN1A gene in the absence of the saRNA of the present invention. In a further embodiment, the expression of SCN1A gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present invention compared to the expression of SCN1A gene in the absence of the saRNA of the present invention. The modulation of the expression of SCN1A gene may be reflected or determined by the change of SCN1A mRNA levels.

SCN1A-saRNAs may be single-stranded and comprise 14-30 nucleotides. The sequence of a single-stranded SCN1A-saRNA may have at least 60%, 70%, 80% or 90% identity with a sequence selected from the sequences of the antisense strands in Table 10-2. In one embodiment, the single-stranded saRNA comprises a sequence selected from the sequences of the antisense strands in Table 10-2. In one embodiment, the single-stranded SCN1A-saRNA may have a 3′ tail. The sequence of a single-stranded SCN1A-saRNA with a 3′ tail may have at least 60%, 70%, 80% or 90% identity with a sequence selected from the sequences of the antisense strands in Table 11. In one embodiment, the single-stranded saRNA comprises a sequence selected from the sequences of the antisense strands in Table 11.

SCN1A-saRNAs may be double-stranded. The two strands form a duplex and each strand comprises 14-30 nucleotides. The first strand of a double-stranded SCN1A-saRNA may have at least 60%, 70%, 80% or 90% identity with a sequence selected from the sequences of the antisense strands in Table 10-2. In one embodiment, the first strand of the double-stranded saRNA comprises a sequence selected from the sequences of the antisense strands in Table 10-2. The second strand of a double-stranded SCN1A-saRNA may have at least 60%, 70%, 80% or 90% identity with a sequence selected from the sequences of the sense strands in Table 10-2. In one embodiment, the second strand of the double-stranded saRNA comprises a sequence selected from the sequences of the sense strands in Table 10-2. In one embodiment, the double-stranded SCN1A-saRNA may have a 3′ overhang on each strand. The first strand of a double-stranded SCN1A-saRNA may have at least 60%, 70%, 80% or 90% identity with a sequence selected from the sequences of the antisense strands in Table 11. In one embodiment, the first strand of the double-stranded saRNA comprises a sequence selected from the sequences of the antisense strands in Table 11. The second strand of a double-stranded SCN1A-saRNA may have at least 60%, 70%, 80% or 90% identity with a sequence selected from the sequences of the sense strands in Table 11. In one embodiment, the second strand of the double-stranded saRNA comprises a sequence selected from the sequences of the sense strands in Table 11.

SCN1A-saRNAs may be modified or unmodified. Table 10-1 describes the target gene, the targeted sequence, the genomic location of the targeted sequence and the relative location of SCN1A-saRNAs with no 3′ overhang. In Table 10-1, the targeted sequence is defined as a region on the template strand of the target gene which is identical to a region on the target antisense RNA transcript to which the antisense strand of an saRNA hybridizes. In Table 10-1, the relative location is the distance from the 5′ end of the targeted sequence to the TSS. A negative number represents a location upstream of the TSS and a positive number represents a location downstream of the TSS.

TABLE 10-1
Sequences of SCN1A-saRNAs (with no 3′ overhang)

| | Target | | Genomic Location of the | Relative |
|---|---|---|---|---|
| saRNA ID | Gene | Targeted sequence | Targeted sequence | Location |
| SCN1A.NM_001202435- | SCN1A | tgagataatctgtgttaga | chr2: 167006477 plus | −835 |
| Pr-1 | human | (SEQ ID NO. 562) | strand | |
| SCN1A.NM_001202435- | SCN1A | gatgatgtccgataagcaa | chr2: 167005347 plus | 295 |
| Pr-8 | human | (SEQ ID NO. 563) | strand | |
| SCN1A.NM_001202435- | SCN1A | tagacttactcctagatat | chr2: 167006492 plus | −850 |
| Pr-10 | human | (SEQ ID NO. 564) | strand | |
| SCN1A.NM_001202435- | SCN1A | gtgttaatctggaggaaca | chr2: 167005609 plus | 33 |
| Pr-17 | human | (SEQ ID NO. 565) | strand | |
| SCN1A.NM_001202435- | SCN1A | agtaaatctctttgagcta | chr2: 167005229 plus | 413 |
| Pr-21 | human | (SEQ ID NO. 566) | strand | |
| SCN1A.NM_001202435- | SCN1A | atgaattgctatcagtaaa | chr2: 167005965 plus | −323 |
| Pr-24 | human | (SEQ ID NO. 567) | strand | |
| SCN1A.NM_001202435- | SCN1A | agacttactcctagatatc | chr2: 167006493 plus | −851 |
| Pr-26 | human | (SEQ ID NO. 568) | strand | |
| SCN1A.NM_001202435- | SCN1A | ggagtttgcatggaaatca | chr2: 167005778 plus | −136 |
| Pr-29 | human | (SEQ ID NO. 569) | strand | |
| SCN1A.NM_001202435- | SCN1A | tggtgggttgccgataaat | chr2: 167006317 plus | −675 |
| Pr-31 | human | (SEQ ID NO. 570) | strand | |
| SCN1A.NM_001202435- | SCN1A | gtgggttgccgataaatgt | chr2: 167006319 plus | −677 |
| Pr-32 | human | (SEQ ID NO. 571) | strand | |
| SCN1A.NM_001202435- | SCN1A | aaatgtcagatgtgttaat | chr2: 167006332 plus | −690 |
| Pr-34 | human | (SEQ ID NO. 572) | strand | |
| SCN1A.NM_006920- | SCN1A | atgtcatttatgtgagtaa | chr2: 166930813 plus | −664 |
| Pr-30 | human | (SEQ ID NO. 573) | strand | |
| SCN1A.NM_006920- | SCN1A | gagagaatctgtcaatttg | chr2: 166929259 plus | 890 |
| Pr-34 | human | (SEQ ID NO. 574) | strand | |
| SCN1A.NM_006920- | SCN1A | ctatatcactaatgagtta | chr2: 166930465 plus | −316 |
| Pr-35 | human | (SEQ ID NO. 575) | strand | |
| SCN1A.NM_006920- | SCN1A | tggactaagagatgttaat | chr2: 166930213 plus | −64 |
| Pr-37 | human | (SEQ ID NO. 576) | strand | |

TABLE 10-2
Sequences of SCN1A-saRNAs (with no 3′ overhang)

| saRNA ID | Sense (passenger) | Antisense (guide) |
|---|---|---|
| SCN1A.NM_001202435- | UGAGAUAAUCUGUGUU | UCUAACACAGAUUAUCUCA |
| Pr-1 | AGA (SEQ ID NO. 577) | (SEQ ID NO. 578) |
| SCN1A.NM_001202435- | GAUGAUGUCCGAUAAG | UUGCUUAUCGGACAUCAUC |
| Pr-8 | CAA (SEQ ID NO. 579) | (SEQ ID NO. 580) |
| SCN1A.NM_001202435- | UAGACUUACUCCUAGA | AUAUCUAGGAGUAAGUCUA |
| Pr-10 | UAU (SEQ ID NO. 581) | (SEQ ID NO. 582) |
| SCN1A.NM_001202435- | GUGUUAAUCUGGAGGA | UGUUCCUCCAGAUUAACAC |
| Pr-17 | ACA (SEQ ID NO. 583) | (SEQ ID NO. 584) |
| SCN1A.NM_001202435- | AGUAAAUCUCUUUGAG | UAGCUCAAAGAGAUUUACU |
| Pr-21 | CUA (SEQ ID NO. 585) | (SEQ ID NO. 586) |
| SCN1A.NM_001202435- | AUGAAUUGCUAUCAGU | UUUACUGAUAGCAAUUCAU |
| Pr-24 | AAA (SEQ ID NO. 587) | (SEQ ID NO. 588) |
| SCN1A.NM_001202435- | AGACUUACUCCUAGAU | GAUAUCUAGGAGUAAGUCU |
| Pr-26 | AUC (SEQ ID NO. 589) | (SEQ ID NO. 590) |
| SCN1A.NM_001202435- | GGAGUUUGCAUGGAAA | UGAUUUCCAUGCAAACUCC |
| Pr-29 | UCA (SEQ ID NO. 591) | (SEQ ID NO. 592) |
| SCN1A.NM_001202435- | UGGUGGGUUGCCGAUA | AUUUAUCGGCAACCCACCA |
| Pr-31 | AAU (SEQ ID NO. 593) | (SEQ ID NO. 594) |
| SCN1A.NM_001202435- | GUGGGUUGCCGAUAAA | ACAUUUAUCGGCAACCCAC |
| Pr-32 | UGU (SEQ ID NO. 595) | (SEQ ID NO. 596) |
| SCN1A.NM_001202435- | AAAUGUCAGAUGUGUU | AUUAACACAUCUGACAUUU |
| Pr-34 | AAU (SEQ ID NO. 597) | (SEQ ID NO. 598) |
| SCN1A.NM_006920- | AUGUCAUUUAUGUGAG | UUACUCACAUAAAUGACAU |
| Pr-30 | UAA (SEQ ID NO. 599) | (SEQ ID NO. 600) |
| SCN1A.NM_006920- | GAGAGAAUCUGUCAAU | CAAAUUGACAGAUUCUCUC |
| Pr-34 | UUG (SEQ ID NO. 601) | (SEQ ID NO. 602) |
| SCN1A.NM_006920- | CUAUAUCACUAAUGAG | UAACUCAUUAGUGAUAUAG |
| Pr-35 | UUA (SEQ ID NO. 603) | (SEQ ID NO. 604) |
| SCN1A.NM_006920- | UGGACUAAGAGAUGUU | AUUAACAUCUCUUAGUCCA |
| Pr-37 | AAU (SEQ ID NO. 605) | (SEQ ID NO. 606) |

TABLE 11
Sequences of SCN1A-saRNAs (with 3′ overhang)

| saRNA ID | Sense (passenger) | Antisense (guide) |
|---|---|---|
| SCN1A.NM_001202435- | UGAGAUAAUCUGUGUUAGAUU | UCUAACACAGAUUAUCUCAUU |
| Pr-1′ | (SEQ ID NO. 607) | (SEQ ID NO. 608) |
| SCN1A.NM_001202435- | GAUGAUGUCCGAUAAGCAAUU | UUGCUUAUCGGACAUCAUCUU |
| Pr-8′ | (SEQ ID NO. 609) | (SEQ ID NO. 610) |
| SCN1A.NM_001202435- | UAGACUUACUCCUAGAUAUUU | AUAUCUAGGAGUAAGUCUAUU |
| Pr-10′ | (SEQ ID NO. 611) | (SEQ ID NO. 612) |
| SCN1A.NM_001202435- | GUGUUAAUCUGGAGGAACAUU | UGUUCCUCCAGAUUAACACUU |
| Pr-17′ | (SEQ ID NO. 613) | (SEQ ID NO. 614) |
| SCN1A.NM_001202435- | AGUAAAUCUCUUUGAGCUAUU | UAGCUCAAAGAGAUUUACUUU |
| Pr-21′ | (SEQ ID NO. 615) | (SEQ ID NO. 616) |
| SCN1A.NM_001202435- | AUGAAUUGCUAUCAGUAAAUU | UUUACUGAUAGCAAUUCAUUU |
| Pr-24′ | (SEQ ID NO. 617) | (SEQ ID NO. 618) |
| SCN1A.NM_001202435- | AGACUUACUCCUAGAUAUCUU | GAUAUCUAGGAGUAAGUCUUU |
| Pr-26′ | (SEQ ID NO. 619) | (SEQ ID NO. 620) |
| SCN1A.NM_001202435- | GGAGUUUGCAUGGAAAUCAUU | UGAUUUCCAUGCAAACUCCUU |
| Pr-29′ | (SEQ ID NO. 621) | (SEQ ID NO. 622) |
| SCN1A.NM_001202435- | UGGUGGGUUGCCGAUAAAUUU | AUUUAUCGGCAACCCACCAUU |
| Pr-31′ | (SEQ ID NO. 623) | (SEQ ID NO. 624) |
| SCN1A.NM_001202435- | GUGGGUUGCCGAUAAAUGUUU | ACAUUUAUCGGCAACCCACUU |
| Pr-32′ | (SEQ ID NO. 625) | (SEQ ID NO. 626) |
| SCN1A.NM_001202435- | AAAUGUCAGAUGUGUUAAUUU | AUUAACACAUCUGACAUUUUU |
| Pr-34′ | (SEQ ID NO. 627) | (SEQ ID NO. 628) |
| SCN1A.NM_006920-Pr- | AUGUCAUUUAUGUGAGUAAUU | UUACUCACAUAAAUGACAUUU |
| 30′ | (SEQ ID NO. 629) | (SEQ ID NO. 630) |
| SCN1A.NM_006920-Pr- | GAGAGAAUCUGUCAAUUUGUU | CAAAUUGACAGAUUCUCUCUU |
| 34′ | (SEQ ID NO. 631) | (SEQ ID NO. 632) |
| SCN1A.NM_006920-Pr- | CUAUAUCACUAAUGAGUUAUU | UAACUCAUUAGUGAUAUAGUU |
| 35′ | (SEQ ID NO. 633) | (SEQ ID NO. 634) |
| SCN1A.NM_006920-Pr- | UGGACUAAGAGAUGUUAAUUU | AUUAACAUCUCUUAGUCCAUU |
| 37′ | (SEQ ID NO. 635) | (SEQ ID NO. 636) |

The SCN1A gene encodes the alpha subunit of a sodium channel called NaV1.1. These channels are found in the brain and muscles, where they control the flow of sodium ions into cells. In the brain, NaV1.1 channels are involved in transmitting signals from one nerve cell (neuron) to another. Communication between neurons depends on chemicals called neurotransmitters, which are released from one neuron and taken up by neighboring neurons. The flow of sodium ions through NaV1.1 channels helps determine when neurotransmitters will be released. Mutations in the SCN1A gene change building blocks in the NaV1.1 channel and change the channel's structure. The abnormal channels stay open longer than usual, which increases the flow of sodium ions into neurons. This increase triggers the cell to release more neurotransmitters. The resulting changes in signaling between neurons make people more susceptible to developing these severe headaches. Diseases associated with SCN1A mutations include, but not limited to, familial hemiplegic migraine and seizure disorders (Martin et al., J Biol Chem, vol. 285 (13):9823 (2010), the contents of which are incorporated herein by reference in their entirety).

In one embodiment, provided is a method of modulating the sodium channel NaV1.1 comprising administering SCN1A-saRNA of the present invention, wherein the SCN1A-saRNA targets an antisense RNA transcript of SCN1A gene.

In another embodiment, provided is a method of treating familial hemiplegic migraine or seizure disorders comprising administering SCN1A-saRNA of the present invention, wherein the SCN1A-saRNA targets an antisense RNA transcript of SCN1A gene.

In one embodiment, the APOA1-saRNA of the present invention is an antisense sequence such as, but not limited to, SEQ ID NO: 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 5316, 5318, 5320, 5322, 5324, 5326, 5328, 5330, 5332, 5334, 5336, 5338, 5340, 5342, 5344, 5346, 5348, 5350, 5352, 5354, 5356, 5358, 5360, 5362, 5364, 5366, 5368, 5370, 5372, 5374, 5376, 5378, 5380, 5382, 5384, 5386, 5388, 5390, 5392, 5394, 5396, 5398, 5400, 5402, 5404, 5406, 5408, 5410, 5412, 5414, 5416, 5418, 5420, 5422, 5424, 5426, 5428, 5430, 5432, 5434, 5436, 5438, 5440, 5442, 5444, 5446, 5448, 5450, 5452, 5454, 5456, 5458, 5460, 5462, 5464, 5466, 5468, 5470, 5472, 5474, 5476, 5478, 5480, 5482, 5484, 5486, 5488, 5490, 5492, 5494, 5496, 5498, 5500, 5502, 5504, 5506, 5508, 5510, 5512, 5514, 5516 and 5518. As a non-limiting example, these SCN1A-saRNA sequences may be used to modulate the sodium channel NaV1.1 and/or treat familial hemiplegic migraine or seizure disorders.

In one embodiment, the SCN1A-saRNA of the present invention is an saRNA duplex. The saRNA duplex may be a pair of sense and antisense sequences such as, but not limited to, SEQ ID NOs: 577 and 578; SEQ ID NOs: 579 and 580; SEQ ID NOs: 581 and 582; SEQ ID NOs: 583 and 584; SEQ ID NOs: 585 and 586; SEQ ID NOs: 587 and 588; SEQ ID NOs: 589 and 590; SEQ ID NOs: 591 and 592; SEQ ID NOs: 593 and 594; SEQ ID NOs: 595 and 596; SEQ ID NOs: 597 and 598; SEQ ID NOs: 599 and 600; SEQ ID NOs: 601 and 602; SEQ ID NOs: 603 and 604; SEQ ID NOs: 605 and 606; SEQ ID NOs: 607 and 608; SEQ ID NOs: 609 and 610; SEQ ID NOs: 611 and 612; SEQ ID NOs: 613 and 614; SEQ ID NOs: 615 and 616; SEQ ID NOs: 617 and 618; SEQ ID NOs: 619 and 620; SEQ ID NOs: 621 and 622; SEQ ID NOs: 623 and 624; SEQ ID NOs: 625 and 626; SEQ ID NOs: 627 and 628; SEQ ID NOs: 629 and 630; SEQ ID NOs: 631 and 632; SEQ ID NOs: 633 and 634; SEQ ID NOs: 635 and 636; SEQ ID NOs: 5315 and 5316; SEQ ID NOs: 5317 and 5318; SEQ ID NOs: 5319 and 5320; SEQ ID NOs: 5321 and 5322; SEQ ID NOs: 5323 and 5324; SEQ ID NOs: 5325 and 5326; SEQ ID NOs: 5327 and 5328; SEQ ID NOs: 5329 and 5330; SEQ ID NOs: 5331 and 5332; SEQ ID NOs: 5333 and 5334; SEQ ID NOs: 5335 and 5336; SEQ ID NOs: 5337 and 5338; SEQ ID NOs: 5339 and 5340; SEQ ID NOs: 5341 and 5342; SEQ ID NOs: 5343 and 5344; SEQ ID NOs: 5345 and 5346; SEQ ID NOs: 5347 and 5348; SEQ ID NOs: 5349 and 5350; SEQ ID NOs: 5351 and 5352; SEQ ID NOs: 5353 and 5354; SEQ ID NOs: 5355 and 5356; SEQ ID NOs: 5357 and 5358; SEQ ID NOs: 5359 and 5360; SEQ ID NOs: 5361 and 5362; SEQ ID NOs: 5363 and 5364; SEQ ID NOs: 5365 and 5366; SEQ ID NOs: 5367 and 5368; SEQ ID NOs: 5369 and 5370; SEQ ID NOs: 5371 and 5372; SEQ ID NOs: 5373 and 5374; SEQ ID NOs: 5375 and 5376; SEQ ID NOs: 5377 and 5378; SEQ ID NOs: 5379 and 5380; SEQ ID NOs: 5381 and 5382; SEQ ID NOs: 5383 and 5384; SEQ ID NOs: 5385 and 5386; SEQ ID NOs: 5387 and 5388; SEQ ID NOs: 5389 and 5390; SEQ ID NOs: 5391 and 5392; SEQ ID NOs: 5393 and 5394; SEQ ID NOs: 5395 and 5396; SEQ ID NOs: 5397 and 5398; SEQ ID NOs: 5399 and 5400; SEQ ID NOs: 5401 and 5402; SEQ ID NOs: 5403 and 5404; SEQ ID NOs: 5405 and 5406; SEQ ID NOs: 5407 and 5408; SEQ ID NOs: 5409 and 5410; SEQ ID NOs: 5411 and 5412; SEQ ID NOs: 5413 and 5414; SEQ ID NOs: 5415 and 5416; SEQ ID NOs: 5417 and 5418; SEQ ID NOs: 5419 and 5420; SEQ ID NOs: 5421 and 5422; SEQ ID NOs: 5423 and 5424; SEQ ID NOs: 5425 and 5426; SEQ ID NOs: 5427 and 5428; SEQ ID NOs: 5429 and 5430; SEQ ID NOs: 5431 and 5432; SEQ ID NOs: 5433 and 5434; SEQ ID NOs: 5435 and 5436; SEQ ID NOs: 5437 and 5438; SEQ ID NOs: 5439 and 5440; SEQ ID NOs: 5441 and 5442; SEQ ID NOs: 5443 and 5444; SEQ ID NOs: 5445 and 5446; SEQ ID NOs: 5447 and 5448; SEQ ID NOs: 5449 and 5450; SEQ ID NOs: 5451 and 5452; SEQ ID NOs: 5453 and 5454; SEQ ID NOs: 5455 and 5456; SEQ ID NOs: 5457 and 5458; SEQ ID NOs: 5459 and 5460; SEQ ID NOs: 5461 and 5462; SEQ ID NOs: 5463 and 5464; SEQ ID NOs: 5465 and 5466; SEQ ID NOs: 5467 and 5468; SEQ ID NOs: 5469 and 5470; SEQ ID NOs: 5471 and 5472; SEQ ID NOs: 5473 and 5474; SEQ ID NOs: 5475 and 5476; SEQ ID NOs: 5477 and 5478; SEQ ID NOs: 5479 and 5480; SEQ ID NOs: 5481 and 5482; SEQ ID NOs: 5483 and 5484; SEQ ID NOs: 5485 and 5486; SEQ ID NOs: 5487 and 5488; SEQ ID NOs: 5489 and 5490; SEQ ID NOs: 5491 and 5492; SEQ ID NOs: 5493 and 5494; SEQ ID NOs: 5495 and 5496; SEQ ID NOs: 5497 and 5498; SEQ ID NOs: 5499 and 5500; SEQ ID NOs: 5501 and 5502; SEQ ID NOs: 5503 and 5504; SEQ ID NOs: 5505 and 5506; SEQ ID NOs: 5507 and 5508; SEQ ID NOs: 5509 and 5510; SEQ ID NOs: 5511 and 5512; SEQ ID NOs: 5513 and 5514; SEQ ID NOs: 5515 and 5516; SEQ ID NOs: 5517 and 5518. As a non-limiting example, these SCN1A-saRNA sequences which are saRNA duplexes may be used to modulate the sodium channel NaV1.1 and/or treat familial hemiplegic migraine or seizure disorders.

### IDUA Gene

As another non-limiting example, provided is a method of modulating the expression of IDUA gene comprising administering saRNA of the present invention, wherein the saRNA targets an antisense RNA transcript of IDUA gene. These saRNAs are called IDUA-saRNA. In one embodiment, the expression of IDUA gene is increased by at least 20, 30, 40%, or at least 45, 50, 55, 60, 65, 70, 75%, or at least 80% in the presence of the IDUA-saRNA of the present invention compared to the expression of IDUA gene in the absence of the saRNA of the present invention. In a further embodiment, the expression of IDUA gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IDUA-saRNA of the present invention compared to the expression of IDUA gene in the absence of the IDUA-saRNA of the present invention. The modulation of the expression of IDUA gene may be reflected or determined by the change of IDUA mRNA levels.

IDUA-saRNAs may be single-stranded and comprise 14-30 nucleotides. The sequence of a single-stranded IDUA-saRNA may have at least 60%, 70%, 80% or 90% identity with a sequence selected from the sequences of the antisense strands in Table 12-2. In one embodiment, the single-stranded saRNA comprises a sequence selected from the sequences of the antisense strands in Table 12-2. In one embodiment, the single-stranded IDUA-saRNA may have a 3′ tail. The sequence of a single-stranded IDUA-saRNA with a 3′ tail may have at least 60%, 70%, 80% or 90% identity with a sequence selected from the sequences of the antisense strands in Table 13. In one embodiment, the single-stranded saRNA comprises a sequence selected from the sequences of the antisense strands in Table 13.

IDUA-saRNAs may be double-stranded. The two strands form a duplex and each strand comprises 14-30 nucleotides. The first strand of a double-stranded IDUA-saRNA may have at least 60%, 70%, 80% or 90% identity with a sequence selected from the sequences of the antisense strands in Table 12-2. In one embodiment, the first strand of the double-stranded saRNA comprises a sequence selected from the sequences of the antisense strands in Table 12-2. The second strand of a double-stranded IDUA-saRNA may have at least 60%, 70%, 80% or 90% identity with a sequence selected from the sequences of the sense strands in Table 12-2. In one embodiment, the second strand of the double-stranded saRNA comprises a sequence selected from the sequences of the sense strands in Table 12-2. In one embodiment, the double-stranded IDUA-saRNA may have a 3′ overhang on each strand. The first strand of a double-stranded IDUA-saRNA may have at least 60%, 70%, 80% or 90% identity with a sequence selected from the sequences of the antisense strands in Table 13. In one embodiment, the first strand of the double-stranded saRNA comprises a sequence selected from the sequences of the antisense strands in Table 13. The second strand of a double-stranded IDUA-saRNA may have at least 60%, 70%, 80% or 90% identity with a sequence selected from the sequences of the sense strands in Table 13. In one embodiment, the second strand of the double-stranded saRNA comprises a sequence selected from the sequences of the sense strands in Table 13.

IDUA-saRNAs may be modified or unmodified.

Table 12-1 describes the target gene, the targeted sequence, the genomic location of the targeted sequence and the relative location of IDUA-saRNAs with no 3′ overhang. In Table 12-1, the targeted sequence is defined as a region on the template strand of the target gene which is identical to a region on the target antisense RNA transcript to which the antisense strand of an saRNA hybridizes. In Table 12-1, the relative location is the distance from the 5′ end of the targeted sequence to the TSS. A negative number represents a location upstream of the TSS and a positive number represents a location downstream of the TSS.

TABLE 12-1
Sequences of IDUA-saRNAs (with no 3′ overhang)

| | | | Genomic Location of | Relative |
|---|---|---|---|---|
| saRNA ID | Target Gene | Targeted sequence | the Targeted sequence | Location |
| IDUA.NM_000203- | IDUA human | atgtgtgaatccaagcaaa | chr4: 980039 minus | −726 |
| Pr-8 | | (SEQ ID NO. 637) | strand | |
| IDUA.NM_000203- | IDUA human | agccagatgcgatggtata | chr4: 979853 minus | −912 |
| Pr-15 | | (SEQ ID NO. 638) | strand | |
| IDUA.NM_000203- | IDUA human | tgtgaatccaagcaaacat | chr4: 980036 minus | −729 |
| Pr-22 | | (SEQ ID NO. 639) | strand | |
| IDUA.NM_000203- | IDUA human | gagtaaagcgtgcgcttta | chr4: 980418 minus | −347 |
| Pr-25 | | (SEQ ID NO. 640) | strand | |
| IDUA.NM_000203- | IDUA human | tgggagattgcttgagctt | chr4: 979800 minus | −965 |
| Pr-29 | | (SEQ ID NO. 641) | strand | |
| IDUA.NM_000203- | IDUA human | cgtaggtaatgtgcaaata | chr4: 980178 minus | −587 |
| Pr-40 | | (SEQ ID NO. 642) | strand | |

TABLE 12-2
Sequences of IDUA-saRNAs (with no 3′ overhang)

| saRNA ID | Sense (passenger) | Antisense (guide) |
|---|---|---|
| IDUA.NM_000203-Pr-8 | AUGUGUGAAUCCAAGCAAA | UUUGCUUGGAUUCACACAU |
| | (SEQ ID NO. 643) | (SEQ ID NO. 644) |
| IDUA.NM_000203-Pr-15 | AGCCAGAUGCGAUGGUAUA | UAUACCAUCGCAUCUGGCU |
| | (SEQ ID NO. 645) | (SEQ ID NO. 646) |
| IDUA.NM_000203-Pr-22 | UGUGAAUCCAAGCAAACAU | AUGUUUGCUUGGAUUCACA |
| | (SEQ ID NO. 647) | (SEQ ID NO. 648) |
| IDUA.NM_000203-Pr-25 | GAGUAAAGCGUGCGCUUUA | UAAAGCGCACGCUUUACUC |
| | (SEQ ID NO. 649) | (SEQ ID NO. 650) |
| IDUA.NM_000203-Pr-29 | UGGGAGAUUGCUUGAGCUU | AAGCUCAAGCAAUCUCCCA |
| | (SEQ ID NO. 651) | (SEQ ID NO. 652) |
| IDUA.NM_000203-Pr-40 | CGUAGGUAAUGUGCAAAUA | UAUUUGCACAUUACCUACG |
| | (SEQ ID NO. 653) | (SEQ ID NO. 654) |

TABLE 13
Sequences of IDUA-saRNAs (with 3′ overhang)

| saRNA ID | Sense (passenger) | Antisense (guide) |
|---|---|---|
| IDUA.NM_000203-Pr- | AUGUGUGAAUCCAAGCAAAUU | UUUGCUUGGAUUCACACAUUU |
| 8′ | (SEQ ID NO. 655) | (SEQ ID NO. 656) |
| IDUA.NM_000203-Pr- | AGCCAGAUGCGAUGGUAUAUU | UAUACCAUCGCAUCUGGCUUU |
| 15′ | (SEQ ID NO. 657) | (SEQ ID NO. 658) |
| IDUA.NM_000203-Pr- | UGUGAAUCCAAGCAAACAUUU | AUGUUUGCUUGGAUUCACAUU |
| 22′ | (SEQ ID NO. 659) | (SEQ ID NO. 660) |
| IDUA.NM_000203-Pr- | GAGUAAAGCGUGCGCUUUAUU | UAAAGCGCACGCUUUACUCUU |
| 25′ | (SEQ ID NO. 661) | (SEQ ID NO. 662) |
| IDUA.NM_000203-Pr- | UGGGAGAUUGCUUGAGCUUUU | AAGCUCAAGCAAUCUCCCAUU |
| 29′ | (SEQ ID NO. 663) | (SEQ ID NO. 664) |
| IDUA.NM_000203-Pr- | CGUAGGUAAUGUGCAAAUAUU | UAUUUGCACAUUACCUACGUU |
| 40′ | (SEQ ID NO. 665) | (SEQ ID NO. 666) |

The IDUA gene encodes an enzyme called alpha-L-iduronidase, which is essential for the breakdown of large sugar molecules called glycosaminoglycans (GAGs). Through a process called hydrolysis, alpha-L-iduronidase uses water molecules to break down a molecule known as unsulfated alpha-L-iduronic acid, which is present in two GAGs called heparan sulfate and dermatan sulfate. Alpha-L-iduronidase is located in lysosomes, compartments within cells that digest and recycle different types of molecules. Mutations of IDUA gene cause mucopolysaccharidosis type I (MPS I) (Chkioua et al., Diagn Pathol. vol. 6:47 (2011), the contents of which are incorporated herein by reference in their entirety). The lack of alpha-L-iduronidase enzyme activity leads to the accumulation of heparan sulfate and dermatan sulfate within the lysosomes. The buildup of these GAGs increases the size of the lysosomes, which is why many tissues and organs are enlarged in MPS I.

In one embodiment, provided is a method of modulating alpha-L-iduronidase protein levels comprising administering IDUA-saRNA of the present invention, wherein the IDUA-saRNA targets an antisense RNA transcript of IDUA gene. In one embodiment, alpha-L-iduronidase protein level is increased by at least 20, 30, 40%, or at least 45, 50, 55, 60, 65, 70, 75%, or at least 80% in the presence of the IDUA-saRNA of the present invention compared to alpha-L-iduronidase protein level in the absence of the IDUA-saRNA of the present invention. In a further embodiment, alpha-L-iduronidase protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IDUA-saRNA of the present invention compared to alpha-L-iduronidase protein level in the absence of the IDUA-saRNA of the present invention.

In another embodiment, provided is a method of modulating the levels of unsulfated alpha-L-iduronic acid, heparan sulfate or dermatan sulfate, comprising administering IDUA-saRNA of the present invention, wherein the IDUA-saRNA targets an antisense RNA transcript of IDUA gene. In one embodiment, the level of unsulfated alpha-L-iduronic acid, heparan sulfate or dermatan sulfate is reduced by at least 10%, 20%, 30%, or at least 40%, 50%, 60%, or 70%, 80%, 90% in the presence of the IDUA-saRNA of the present invention compared to the level of unsulfated alpha-L-iduronic acid, heparan sulfate or dermatan sulfate in the absence of the IDUA-saRNA of the present invention.

In another embodiment, provided is a method of treating MPS I comprising administering IDUA-saRNA of the present invention, wherein the IDUA-saRNA targets an antisense RNA transcript of IDUA gene, and wherein the symptoms of MPS I are reduced.

In one embodiment, the IDUA-saRNA of the present invention is an antisense sequence such as, but not limited to, SEQ ID NO: 644, 646, 648, 650, 652, 654, 656, 658, 660, 662, 664, 666, 4042, 4044, 4046, 4048, 4050, 4052, 4054, 4056, 4058, 4060, 4062, 4064, 4066, 4068, 4070, 4072, 4074, 4076, 4078, 4080, 4082, 4084, 4086, 4088, 4090, 4092, 4094, 4096, 4098, 4100, 4102, 4104, 4106 and 4108. As a non-limiting example, these IDUA-saRNA sequences may be used to modulate alpha-L-iduronidase protein levels, modulate the level of unsulfated alpha-L-iduronic acid, heparan sulfate or dermatan sulfate and/or treat MPS I.

In one embodiment, the IDUA-saRNA of the present invention is an saRNA duplex. The saRNA duplex may be a pair of sense and antisense sequences such as, but not limited to, SEQ ID NOs: 643 and 644; SEQ ID NOs: 645 and 646; SEQ ID NOs: 647 and 648; SEQ ID NOs: 649 and 650; SEQ ID NOs: 651 and 652; SEQ ID NOs: 653 and 654; SEQ ID NOs: 655 and 656; SEQ ID NOs: 657 and 658; SEQ ID NOs: 659 and 660; SEQ ID NOs: 661 and 662; SEQ ID NOs: 663 and 664; SEQ ID NOs: 665 and 666; SEQ ID NOs: 4041 and 4042; SEQ ID NOs: 4043 and 4044; SEQ ID NOs: 4045 and 4046; SEQ ID NOs: 4047 and 4048; SEQ ID NOs: 4049 and 4050; SEQ ID NOs: 4051 and 4052; SEQ ID NOs: 4053 and 4054; SEQ ID NOs: 4055 and 4056; SEQ ID NOs: 4057 and 4058; SEQ ID NOs: 4059 and 4060; SEQ ID NOs: 4061 and 4062; SEQ ID NOs: 4063 and 4064; SEQ ID NOs: 4065 and 4066; SEQ ID NOs: 4067 and 4068; SEQ ID NOs: 4069 and 4070; SEQ ID NOs: 4071 and 4072; SEQ ID NOs: 4073 and 4074; SEQ ID NOs: 4075 and 4076; SEQ ID NOs: 4077 and 4078; SEQ ID NOs: 4079 and 4080; SEQ ID NOs: 4081 and 4082; SEQ ID NOs: 4083 and 4084; SEQ ID NOs: 4085 and 4086; SEQ ID NOs: 4087 and 4088; SEQ ID NOs: 4089 and 4090; SEQ ID NOs: 4091 and 4092; SEQ ID NOs: 4093 and 4094; SEQ ID NOs: 4095 and 4096; SEQ ID NOs: 4097 and 4098; SEQ ID NOs: 4099 and 4100; SEQ ID NOs: 4101 and 4102; SEQ ID NOs: 4103 and 4104; SEQ ID NOs: 4105 and 4106; SEQ ID NOs: 4107 and 4108. As a non-limiting example, these IDUA-saRNA sequences which are saRNA duplexes may be used to modulate alpha-L-iduronidase protein levels, modulate the level of unsulfated alpha-L-iduronic acid, heparan sulfate or dermatan sulfate and/or treat MPS I.

### FNDC5 Gene

As another non-limiting example, provided is a method of modulating the expression of FNDC5 gene comprising administering saRNA of the present invention, wherein the saRNA targets an antisense RNA transcript of FNDC5 gene. These saRNAs are called FNDC5-saRNA. In one embodiment, the expression of FNDC5 gene is increased by at least 20, 30, 40%, or at least 45, 50, 55, 60, 65, 70, 75%, or at least 80% in the presence of the FNDC5-saRNA of the present invention compared to the expression of FNDC5 gene in the absence of the FNDC5-saRNA of the present invention. In a further embodiment, the expression of FNDC5 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FNDC5-saRNA of the present invention compared to the expression of FNDC5 gene in the absence of the FNDC5-saRNA of the present invention. The modulation of the expression of FNDC5 gene may be reflected or determined by the change of FNDC5 mRNA levels.

FNDC5-saRNAs may be single-stranded and comprise 14-30 nucleotides. The sequence of a single-stranded FNDC5-saRNA may have at least 60%, 70%, 80% or 90% identity with a sequence selected from the sequences of the antisense strands in Table 14-2. In one embodiment, the single-stranded saRNA comprises a sequence selected from the sequences of the antisense strands in Table 14-2. In one embodiment, the single-stranded FNDC5-saRNA may have a 3′ tail. The sequence of a single-stranded FNDC5-saRNA with a 3′ tail may have at least 60%, 70%, 80% or 90% identity with a sequence selected from the sequences of the antisense strands in Table 15. In one embodiment, the single-stranded saRNA comprises a sequence selected from the sequences of the antisense strands in Table 15.

FNDC5-saRNAs may be double-stranded. The two strands form a duplex and each strand comprises 14-30 nucleotides. The first strand of a double-stranded FNDC5-saRNA may have at least 60%, 70%, 80% or 90% identity with a sequence selected from the sequences of the antisense strands in Table 14-2. In one embodiment, the first strand of the double-stranded saRNA comprises a sequence selected from the sequences of the antisense strands in Table 14-2. The second strand of a double-stranded FNDC5-saRNA may have at least 60%, 70%, 80% or 90% identity with a sequence selected from the sequences of the sense strands in Table 14-2. In one embodiment, the second strand of the double-stranded saRNA comprises a sequence selected from the sequences of the sense strands in Table 14-2. In one embodiment, the double-stranded FNDC5-saRNA may have a 3′ overhang on each strand. The first strand of a double-stranded FNDC5-saRNA may have at least 60%, 70%, 80% or 90% identity with a sequence selected from the sequences of the antisense strands in Table 15. In one embodiment, the first strand of the double-stranded saRNA comprises a sequence selected from the sequences of the antisense strands in Table 15. The second strand of a double-stranded FNDC5-saRNA may have at least 60%, 70%, 80% or 90% identity with a sequence selected from the sequences of the sense strands in Table 15. In one embodiment, the second strand of the double-stranded saRNA comprises a sequence selected from the sequences of the sense strands in Table 15.

FNDC5-saRNAs may be modified or unmodified.

Table 14-1 describes the target gene, the targeted sequence, the genomic location of the targeted sequence and the relative location of FNDC5-saRNAs with no 3′ overhang. In Table 14-1, the targeted sequence is defined as a region on the template strand of the target gene which is identical to a region on the target antisense RNA transcript to which the antisense strand of an saRNA hybridizes. In Table 14-1, the relative location is the distance from the 5′ end of the targeted sequence to the TSS. A negative number represents a location upstream of the TSS and a positive number represents a location downstream of the TSS.

TABLE 14-1
Sequences of FNDC5-saRNAs (with no 3′ overhang)

| | Target | | Genomic Location of the | Relative |
|---|---|---|---|---|
| saRNA ID | Gene | Targeted sequence | Targeted sequence | Location |
| FNDC5.NM_001171941- | FNDC5 | gcaaagttctgccacagaa | chr1: 33337658 plus strand | 435 |
| Pr-5 | human | (SEQ ID NO. 667) | | |
| FNDC5.NM_001171941- | FNDC5 | ggagacgtcttgcaaagtt | chr1: 33337647 plus strand | 446 |
| Pr-7 | human | (SEQ ID NO. 668) | | |
| FNDC5.NM_001171941- | FNDC5 | tccggagattcgtgagact | chr1: 33337347 plus strand | 746 |
| Pr-8 | human | (SEQ ID NO. 669) | | |
| FNDC5.NM_001171941- | FNDC5 | cggagattcgtgagactct | chr1: 33337349 plus strand | 744 |
| Pr-13 | human | (SEQ ID NO. 670) | | |
| FNDC5.NM_001171941- | FNDC5 | ggagattcgtgagactcta | chr1: 33337350 plus strand | 743 |
| Pr-14 | human | (SEQ ID NO. 671) | | |
| FNDC5.NM_001171941- | FNDC5 | tcgtgagactctagaattg | chr1: 33337356 plus strand | 737 |
| Pr-16 | human | (SEQ ID NO. 672) | | |
| FNDC5.NM_001171941- | FNDC5 | cagcaatctctaagagcat | chr1: 33337412 plus strand | 681 |
| Pr-17 | human | (SEQ ID NO. 673) | | |
| FNDC5.NM_001171941- | FNDC5 | aggagacgtcttgcaaagt | chr1: 33337646 plus strand | 447 |
| Pr-19 | human | (SEQ ID NO. 674) | | |
| FNDC5.NM_001171941- | FNDC5 | gcagcaatctctaagagca | chr1: 33337411 plus strand | 682 |
| Pr-22 | human | (SEQ ID NO. 675) | | |
| FNDC5.NM_001171941- | FNDC5 | ggaagtgaggtctaccgat | chr1: 33337721 plus strand | 372 |
| Pr-36 | human | (SEQ ID NO. 676) | | |
| FNDC5.NM_001171941- | FNDC5 | tgcattatgtggcttaatt | chr1: 33337148 plus strand | 945 |
| Pr-38 | human | (SEQ ID NO. 677) | | |
| FNDC5.NM_001171941- | FNDC5 | cccacgactatccgaaatt | chr1: 33337553 plus strand | 540 |
| Pr-39 | human | (SEQ ID NO. 678) | | |
| FNDC5.NM_153756- | FNDC5 | tccggagattcgtgagact | chr1: 33337347 plus strand | −933 |
| Pr-3 | human | (SEQ ID NO. 679) | | |
| FNDC5.NM_153756- | FNDC5 | cggagattcgtgagactct | chr1: 33337349 plus strand | −935 |
| Pr-6 | human | (SEQ ID NO. 680) | | |
| FNDC5.NM_153756- | FNDC5 | ggagattcgtgagactcta | chr1: 33337350 plus strand | −936 |
| Pr-7 | human | (SEQ ID NO. 681) | | |
| FNDC5.NM_153756- | FNDC5 | tcgtgagactctagaattg | chr1: 33337356 plus strand | −942 |
| Pr-9 | human | (SEQ ID NO. 682) | | |
| FNDC5.NM_153756- | FNDC5 | cagtgtttgggccgagaca | chr1: 33336134 plus strand | 280 |
| Pr-12 | human | (SEQ ID NO. 683) | | |
| FNDC5.NM_153756- | FNDC5 | tgcattatgtggcttaatt | chr1: 33337148 plus strand | −734 |
| Pr-16 | human | (SEQ ID NO. 684) | | |
| FNDC5.NM_153756- | FNDC5 | ggacaagtgccaccgagat | chr1: 33336810 plus strand | −396 |
| Pr-20 | human | (SEQ ID NO. 685) | | |
| FNDC5.NM_153756- | FNDC5 | ctcccaaagctgtgcttaa | chr1: 33337293 plus strand | −879 |
| Pr-24 | human | (SEQ ID NO. 686) | | |
| FNDC5.NM_153756- | FNDC5 | caaagctgtgcttaactga | chr1: 33337297 plus strand | −883 |
| Pr-27 | human | (SEQ ID NO. 687) | | |
| FNDC5.NM_153756- | FNDC5 | gcaaccctgtaaagtcata | chr1: 33337173 plus strand | −759 |
| Pr-30 | human | (SEQ ID NO. 688) | | |

TABLE 14-2
Sequences of FNDC5-saRNAs (with no 3′ overhang)

| saRNA ID | Sense (passenger) | Antisense (guide) |
|---|---|---|
| FNDC5.NM_001171941- | GCAAAGUUCUGCCACAGAA | UUCUGUGGCAGAACUUUGC (SEQ |
| Pr-5 | (SEQ ID NO. 689) | ID NO. 690) |
| FNDC5.NM_001171941- | GGAGACGUCUUGCAAAGUU | AACUUUGCAAGACGUCUCC (SEQ |
| Pr-7 | (SEQ ID NO. 691) | ID NO. 692) |
| FNDC5.NM_001171941- | UCCGGAGAUUCGUGAGACU | AGUCUCACGAAUCUCCGGA (SEQ |
| Pr-8 | (SEQ ID NO. 693) | ID NO. 694) |
| FNDC5.NM_001171941- | CGGAGAUUCGUGAGACUCU | AGAGUCUCACGAAUCUCCG (SEQ |
| Pr-13 | (SEQ ID NO. 695) | ID NO. 696) |
| FNDC5.NM_001171941- | GGAGAUUCGUGAGACUCUA | UAGAGUCUCACGAAUCUCC (SEQ |
| Pr-14 | (SEQ ID NO. 697) | ID NO. 698) |
| FNDC5.NM_001171941- | UCGUGAGACUCUAGAAUUG | CAAUUCUAGAGUCUCACGA (SEQ |
| Pr-16 | (SEQ ID NO. 699) | ID NO. 700) |
| FNDC5.NM_001171941- | CAGCAAUCUCUAAGAGCAU | AUGCUCUUAGAGAUUGCUG (SEQ |
| Pr-17 | (SEQ ID NO. 701) | ID NO. 702) |
| FNDC5.NM_001171941- | AGGAGACGUCUUGCAAAGU | ACUUUGCAAGACGUCUCCU (SEQ |
| Pr-19 | (SEQ ID NO. 703) | ID NO. 704) |
| FNDC5.NM_001171941- | GCAGCAAUCUCUAAGAGCA | UGCUCUUAGAGAUUGCUGC (SEQ |
| Pr-22 | (SEQ ID NO. 705) | ID NO. 706) |
| FNDC5.NM_001171941- | GGAAGUGAGGUCUACCGAU | AUCGGUAGACCUCACUUCC (SEQ |
| Pr-36 | (SEQ ID NO. 707) | ID NO. 708) |
| FNDC5.NM_001171941- | UGCAUUAUGUGGCUUAAUU | AAUUAAGCCACAUAAUGCA (SEQ |
| Pr-38 | (SEQ ID NO. 709) | ID NO. 710) |
| FNDC5.NM_001171941- | CCCACGACUAUCCGAAAUU | AAUUUCGGAUAGUCGUGGG (SEQ |
| Pr-39 | (SEQ ID NO. 711) | ID NO. 712) |
| FNDC5.NM_153756- | UCCGGAGAUUCGUGAGACU | AGUCUCACGAAUCUCCGGA (SEQ |
| Pr-3 | (SEQ ID NO. 713) | ID NO. 714) |
| FNDC5.NM_153756- | CGGAGAUUCGUGAGACUCU | AGAGUCUCACGAAUCUCCG (SEQ |
| Pr-6 | (SEQ ID NO. 715) | ID NO. 716) |
| FNDC5.NM_153756- | GGAGAUUCGUGAGACUCUA | UAGAGUCUCACGAAUCUCC (SEQ |
| Pr-7 | (SEQ ID NO. 717) | ID NO. 718) |
| FNDC5.NM_153756- | UCGUGAGACUCUAGAAUUG | CAAUUCUAGAGUCUCACGA (SEQ |
| Pr-9 | (SEQ ID NO. 719) | ID NO. 720) |
| FNDC5.NM_153756- | CAGUGUUUGGGCCGAGACA | UGUCUCGGCCCAAACACUG (SEQ |
| Pr-12 | (SEQ ID NO. 721) | ID NO. 722) |
| FNDC5.NM_153756- | UGCAUUAUGUGGCUUAAUU | AAUUAAGCCACAUAAUGCA (SEQ |
| Pr-16 | (SEQ ID NO. 723) | ID NO. 724) |
| FNDC5.NM_153756- | GGACAAGUGCCACCGAGAU | AUCUCGGUGGCACUUGUCC (SEQ |
| Pr-20 | (SEQ ID NO. 725) | ID NO. 726) |
| FNDC5.NM_153756- | CUCCCAAAGCUGUGCUUAA | UUAAGCACAGCUUUGGGAG (SEQ |
| Pr-24 | (SEQ ID NO. 727) | ID NO. 728) |
| FNDC5.NM_153756- | CAAAGCUGUGCUUAACUGA | UCAGUUAAGCACAGCUUUG (SEQ |
| Pr-27 | (SEQ ID NO. 729) | ID NO. 730) |
| FNDC5.NM_153756- | GCAACCCUGUAAAGUCAUA | UAUGACUUUACAGGGUUGC (SEQ |
| Pr-30 | (SEQ ID NO. 731) | ID NO. 732) |

TABLE 15
Sequences of FNDC5-saRNAs (with 3′ overhang)

| saRNA ID | Sense (passenger) | Antisense (guide) |
|---|---|---|
| FNDC5.NM_001171941-Pr-5′ | GCAAAGUUCUGCCACAGAAU | UUCUGUGGCAGAACUUUGCU |
| | U (SEQ ID NO. 733) | U (SEQ ID NO. 734) |
| FNDC5.NM_001171941-Pr-7′ | GGAGACGUCUUGCAAAGUUU | AACUUUGCAAGACGUCUCCU |
| | U (SEQ ID NO. 735) | U (SEQ ID NO. 736) |
| FNDC5.NM_001171941-Pr-8′ | UCCGGAGAUUCGUGAGACUU | AGUCUCACGAAUCUCCGGAU |
| | U (SEQ ID NO. 737) | U (SEQ ID NO. 738) |
| FNDC5.NM_001171941-Pr- | CGGAGAUUCGUGAGACUCUU | AGAGUCUCACGAAUCUCCGU |
| 13′ | U (SEQ ID NO. 739) | U (SEQ ID NO. 740) |
| FNDC5.NM_001171941-Pr- | GGAGAUUCGUGAGACUCUAU | UAGAGUCUCACGAAUCUCCU |
| 14′ | U (SEQ ID NO. 741) | U (SEQ ID NO. 742) |
| FNDC5.NM_001171941-Pr- | UCGUGAGACUCUAGAAUUGU | CAAUUCUAGAGUCUCACGAU |
| 16′ | U (SEQ ID NO. 743) | U (SEQ ID NO. 744) |
| FNDC5.NM_001171941-Pr- | CAGCAAUCUCUAAGAGCAUU | AUGCUCUUAGAGAUUGCUGU |
| 17′ | U (SEQ ID NO. 745) | U (SEQ ID NO. 746) |
| FNDC5.NM_001171941-Pr- | AGGAGACGUCUUGCAAAGUU | ACUUUGCAAGACGUCUCCUU |
| 19′ | U (SEQ ID NO. 747) | U (SEQ ID NO. 748) |
| FNDC5.NM_001171941-Pr- | GCAGCAAUCUCUAAGAGCAU | UGCUCUUAGAGAUUGCUGCU |
| 22′ | U (SEQ ID NO. 749) | U (SEQ ID NO. 750) |
| FNDC5.NM_001171941-Pr- | GGAAGUGAGGUCUACCGAUU | AUCGGUAGACCUCACUUCCU |
| 36′ | U (SEQ ID NO. 751) | U (SEQ ID NO. 752) |
| FNDC5.NM_001171941-Pr- | UGCAUUAUGUGGCUUAAUUU | AAUUAAGCCACAUAAUGCAU |
| 38′ | U (SEQ ID NO. 753) | U (SEQ ID NO. 754) |
| FNDC5.NM_001171941-Pr- | CCCACGACUAUCCGAAAUUU | AAUUUCGGAUAGUCGUGGGU |
| 39′ | U (SEQ ID NO. 755) | U (SEQ ID NO. 756) |
| FNDC5.NM_153756-Pr-3′ | UCCGGAGAUUCGUGAGACUU | AGUCUCACGAAUCUCCGGAU |
| | U (SEQ ID NO. 757) | U (SEQ ID NO. 758) |
| FNDC5.NM_153756-Pr-6′ | CGGAGAUUCGUGAGACUCUU | AGAGUCUCACGAAUCUCCGU |
| | U (SEQ ID NO. 759) | U (SEQ ID NO. 760) |
| FNDC5.NM_153756-Pr-7′ | GGAGAUUCGUGAGACUCUAU | UAGAGUCUCACGAAUCUCCU |
| | U (SEQ ID NO. 761) | U (SEQ ID NO. 762) |
| FNDC5.NM_153756-Pr-9′ | UCGUGAGACUCUAGAAUUGU | CAAUUCUAGAGUCUCACGAU |
| | U (SEQ ID NO. 763) | U (SEQ ID NO. 764) |
| FNDC5.NM_153756-Pr-12′ | CAGUGUUUGGGCCGAGACAU | UGUCUCGGCCCAAACACUGU |
| | U (SEQ ID NO. 765) | U (SEQ ID NO. 766) |
| FNDC5.NM_153756-Pr-16′ | UGCAUUAUGUGGCUUAAUUU | AAUUAAGCCACAUAAUGCAU |
| | U (SEQ ID NO. 767) | U (SEQ ID NO. 768) |
| FNDC5.NM_153756-Pr-20′ | GGACAAGUGCCACCGAGAUU | AUCUCGGUGGCACUUGUCCU |
| | U (SEQ ID NO. 769) | U (SEQ ID NO. 770) |
| FNDC5.NM_153756-Pr-24′ | CUCCCAAAGCUGUGCUUAAU | UUAAGCACAGCUUUGGGAGU |
| | U (SEQ ID NO. 771) | U (SEQ ID NO. 772) |
| FNDC5.NM_153756-Pr-27′ | CAAAGCUGUGCUUAACUGAU | UCAGUUAAGCACAGCUUUGU |
| | U (SEQ ID NO. 773) | U (SEQ ID NO. 774) |
| FNDC5.NM_153756-Pr-30′ | GCAACCCUGUAAAGUCAUAU | UAUGACUUUACAGGGUUGCU |
| | U (SEQ ID NO. 775) | U (SEQ ID NO. 776) |

FNDC5 gene, also called FRCP2, is a gene in the fibronectin type III domain containing family. It encodes a transmembrane protein FNDC5. Irisin is a cleaved and secreted fragment of FNDC5 protein. Irisin is released from muscle cells during exercise and induces browning of white adipose tissue (Novelle, et al., Int J Endocrinol, vol. 2013:746281 (2013), the contents of which are incorporated herein by reference in their entirety). FNDC5 and irisin have great therapeutic potential in diabetes and obesity. They also involve in energy and metabolic homeostasis and affect the central nervous system (CNS).

In one embodiment, provided is a method of modulating FNDC5 or irisin protein levels, comprising administering FNDC5-saRNA of the present invention, wherein the FNDC5-saRNA targets an antisense RNA transcript of FNDC5 gene. In one embodiment, FNDC5 or irisin protein level is increased by at least 20, 30, 40%, or at least 45, 50, 55, 60, 65, 70, 75%, or at least 80% in the presence of the FNDC5-saRNA of the present invention compared to FNDC5 or irisin protein level in the absence of the FNDC5-saRNA of the present invention. In a further embodiment, FNDC5 or irisin protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FNDC5-saRNA of the present invention compared to FNDC5 or irisin protein level in the absence of the FNDC5-saRNA of the present invention.

In another embodiment, provided is a method of modulating browning of white adipose tissue comprising administering FNDC5-saRNA of the present invention, wherein the FNDC5-saRNA targets an antisense RNA transcript of FNDC5 gene.

In another embodiment, provided is a method of treating diabetes and obesity comprising administering FNDC5-saRNA of the present invention, wherein the FNDC5-saRNA targets an antisense RNA transcript of FNDC5 gene, wherein the symptoms of diabetes are reduced and the weight of the patient is reduced.

In one embodiment, the FNDC5-saRNA of the present invention is an antisense sequence such as, but not limited to, SEQ ID NO: 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 3398, 3400, 3402, 3404, 3406, 3408, 3410, 3412, 3414, 3416, 3418, 3420, 3422, 3424, 3426, 3428, 3430, 3432, 3434, 3436, 3438, 3440, 3442, 3444, 3446, 3448, 3450, 3452, 3454, 3456, 3458, 3460, 3462, 3464, 3466, 3468, 3470, 3472, 3474, 3476, 3478, 3480, 3482, 3484, 3486, 3488, 3490, 3492, 3494, 3496, 3498, 3500, 3502, 3504, 3506, 3508, 3510, 3512, 3514, 3516, 3518, 3520, 3522, 3524, 3526, 3528, 3530, 3532, 3534, 3536, 3538, 3540, 3542, 3544, 3546, 3548, 3550, 3552, 3554, 3556, 3558, 3560, 3562, 3564, 3566, 3568, 3570, 3572, 3574, 3576, 3578, 3580, 3582, 3584, 3586, 3588, 3590, 3592, 3594, 3596, and 3598. As a non-limiting example, these FNDC5-saRNA sequences may be used to modulate FNDC5 or irisin protein levels, modulate browning of white adipose tissue and/or treat diabetes and obesity.

In one embodiment, the APOA1-saRNA of the present invention is an saRNA duplex. The saRNA duplex may be a pair of sense and antisense sequences such as, but not limited to, SEQ ID NOs: 689 and 690; SEQ ID NOs: 691 and 692; SEQ ID NOs: 693 and 694; SEQ ID NOs: 695 and 696; SEQ ID NOs: 697 and 698; SEQ ID NOs: 699 and 700; SEQ ID NOs: 701 and 702; SEQ ID NOs: 703 and 704; SEQ ID NOs: 705 and 706; SEQ ID NOs: 707 and 708; SEQ ID NOs: 709 and 710; SEQ ID NOs: 711 and 712; SEQ ID NOs: 713 and 714; SEQ ID NOs: 715 and 716; SEQ ID NOs: 717 and 718; SEQ ID NOs: 719 and 720; SEQ ID NOs: 721 and 722; SEQ ID NOs: 723 and 724; SEQ ID NOs: 725 and 726; SEQ ID NOs: 727 and 728; SEQ ID NOs: 729 and 730; SEQ ID NOs: 731 and 732; SEQ ID NOs: 733 and 734; SEQ ID NOs: 735 and 736; SEQ ID NOs: 737 and 738; SEQ ID NOs: 739 and 740; SEQ ID NOs: 741 and 742; SEQ ID NOs: 743 and 744; SEQ ID NOs: 745 and 746; SEQ ID NOs: 747 and 748; SEQ ID NOs: 749 and 750; SEQ ID NOs: 751 and 752; SEQ ID NOs: 753 and 754; SEQ ID NOs: 755 and 756; SEQ ID NOs: 757 and 758; SEQ ID NOs: 759 and 760; SEQ ID NOs: 761 and 762; SEQ ID NOs: 763 and 764; SEQ ID NOs: 765 and 766; SEQ ID NOs: 767 and 768; SEQ ID NOs: 769 and 770; SEQ ID NOs: 771 and 772; SEQ ID NOs: 773 and 774; SEQ ID NOs: 775 and 776; SEQ ID NOs: 3397 and 3398; SEQ ID NOs: 3399 and 3400; SEQ ID NOs: 3401 and 3402; SEQ ID NOs: 3403 and 3404; SEQ ID NOs: 3405 and 3406; SEQ ID NOs: 3407 and 3408; SEQ ID NOs: 3409 and 3410; SEQ ID NOs: 3411 and 3412; SEQ ID NOs: 3413 and 3414; SEQ ID NOs: 3415 and 3416; SEQ ID NOs: 3417 and 3418; SEQ ID NOs: 3419 and 3420; SEQ ID NOs: 3421 and 3422; SEQ ID NOs: 3423 and 3424; SEQ ID NOs: 3425 and 3426; SEQ ID NOs: 3427 and 3428; SEQ ID NOs: 3429 and 3430; SEQ ID NOs: 3431 and 3432; SEQ ID NOs: 3433 and 3434; SEQ ID NOs: 3435 and 3436; SEQ ID NOs: 3437 and 3438; SEQ ID NOs: 3439 and 3440; SEQ ID NOs: 3441 and 3442; SEQ ID NOs: 3443 and 3444; SEQ ID NOs: 3445 and 3446; SEQ ID NOs: 3447 and 3448; SEQ ID NOs: 3449 and 3450; SEQ ID NOs: 3451 and 3452; SEQ ID NOs: 3453 and 3454; SEQ ID NOs: 3455 and 3456; SEQ ID NOs: 3457 and 3458; SEQ ID NOs: 3459 and 3460; SEQ ID NOs: 3461 and 3462; SEQ ID NOs: 3463 and 3464; SEQ ID NOs: 3465 and 3466; SEQ ID NOs: 3467 and 3468; SEQ ID NOs: 3469 and 3470; SEQ ID NOs: 3471 and 3472; SEQ ID NOs: 3473 and 3474; SEQ ID NOs: 3475 and 3476; SEQ ID NOs: 3477 and 3478; SEQ ID NOs: 3479 and 3480; SEQ ID NOs: 3481 and 3482; SEQ ID NOs: 3483 and 3484; SEQ ID NOs: 3485 and 3486; SEQ ID NOs: 3487 and 3488; SEQ ID NOs: 3489 and 3490; SEQ ID NOs: 3491 and 3492; SEQ ID NOs: 3493 and 3494; SEQ ID NOs: 3495 and 3496; SEQ ID NOs: 3497 and 3498; SEQ ID NOs: 3499 and 3500; SEQ ID NOs: 3501 and 3502; SEQ ID NOs: 3503 and 3504; SEQ ID NOs: 3505 and 3506; SEQ ID NOs: 3507 and 3508; SEQ ID NOs: 3509 and 3510; SEQ ID NOs: 3511 and 3512; SEQ ID NOs: 3513 and 3514; SEQ ID NOs: 3515 and 3516; SEQ ID NOs: 3517 and 3518; SEQ ID NOs: 3519 and 3520; SEQ ID NOs: 3521 and 3522; SEQ ID NOs: 3523 and 3524; SEQ ID NOs: 3525 and 3526; SEQ ID NOs: 3527 and 3528; SEQ ID NOs: 3529 and 3530; SEQ ID NOs: 3531 and 3532; SEQ ID NOs: 3533 and 3534; SEQ ID NOs: 3535 and 3536; SEQ ID NOs: 3537 and 3538; SEQ ID NOs: 3539 and 3540; SEQ ID NOs: 3541 and 3542; SEQ ID NOs: 3543 and 3544; SEQ ID NOs: 3545 and 3546; SEQ ID NOs: 3547 and 3548; SEQ ID NOs: 3549 and 3550; SEQ ID NOs: 3551 and 3552; SEQ ID NOs: 3553 and 3554; SEQ ID NOs: 3555 and 3556; SEQ ID NOs: 3557 and 3558; SEQ ID NOs: 3559 and 3560; SEQ ID NOs: 3561 and 3562; SEQ ID NOs: 3563 and 3564; SEQ ID NOs: 3565 and 3566; SEQ ID NOs: 3567 and 3568; SEQ ID NOs: 3569 and 3570; SEQ ID NOs: 3571 and 3572; SEQ ID NOs: 3573 and 3574; SEQ ID NOs: 3575 and 3576; SEQ ID NOs: 3577 and 3578; SEQ ID NOs: 3579 and 3580; SEQ ID NOs: 3581 and 3582; SEQ ID NOs: 3583 and 3584; SEQ ID NOs: 3585 and 3586; SEQ ID NOs: 3587 and 3588; SEQ ID NOs: 3589 and 3590; SEQ ID NOs: 3591 and 3592; SEQ ID NOs: 3593 and 3594; SEQ ID NOs: 3595 and 3596; SEQ ID NOs: 3597 and 3598. As a non-limiting example, these FNDC5-saRNA sequences which are saRNA duplexes may be used to modulate FNDC5 or irisin protein levels, modulate browning of white adipose tissue and/or treat diabetes and obesity.

### FOXA2 Gene

As another non-limiting example, provided is a method of modulating the expression of FOXA2 gene comprising administering saRNA of the present invention, wherein the saRNA targets an antisense RNA transcript of FOXA2 gene. These saRNAs are called FOXA2-saRNA. In one embodiment, the expression of FOXA2 gene is increased by at least 20, 30, 40%, or at least 45, 50, 55, 60, 65, 70, 75%, or at least 80% in the presence of the FOXA2-saRNA of the present invention compared to the expression of FOXA2 gene in the absence of the FOXA2-saRNA of the present invention. In a further embodiment, the expression of FOXA2 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FOXA2-saRNA of the present invention compared to the expression of FOXA2 gene in the absence of the FOXA2-saRNA of the present invention. The modulation of the expression of FOXA2 gene may be reflected or determined by the change of FOXA2 mRNA levels.

FOXA2-saRNAs may be single-stranded and comprise 14-30 nucleotides. The sequence of a single-stranded FOXA2-saRNA may have at least 60%, 70%, 80% or 90% identity with a sequence selected from the sequences of the antisense strands in Table 16-2. In one embodiment, the single-stranded saRNA comprises a sequence selected from the sequences of the antisense strands in Table 16-2. In one embodiment, the single-stranded FOXA2-saRNA may have a 3′ tail. The sequence of a single-stranded FOXA2-saRNA with a 3′ tail may have at least 60%, 70%, 80% or 90% identity with a sequence selected from the sequences of the antisense strands in Table 17. In one embodiment, the single-stranded saRNA comprises a sequence selected from the sequences of the antisense strands in Table 17.

FOXA2-saRNAs may be double-stranded. The two strands form a duplex and each strand comprises 14-30 nucleotides. The first strand of a double-stranded FOXA2-saRNA may have at least 60%, 70%, 80% or 90% identity with a sequence selected from the sequences of the antisense strands in Table 16-2. In one embodiment, the first strand of the double-stranded saRNA comprises a sequence selected from the sequences of the antisense strands in Table 16-2. The second strand of a double-stranded FOXA2-saRNA may have at least 60%, 70%, 80% or 90% identity with a sequence selected from the sequences of the sense strands in Table 16-2. In one embodiment, the second strand of the double-stranded saRNA comprises a sequence selected from the sequences of the sense strands in Table 16-2. In one embodiment, the double-stranded FOXA2-saRNA may have a 3′ overhang on each strand. The first strand of a double-stranded FOXA2-saRNA may have at least 60%, 70%, 80% or 90% identity with a sequence selected from the sequences of the antisense strands in Table 17. In one embodiment, the first strand of the double-stranded saRNA comprises a sequence selected from the sequences of the antisense strands in Table 17. The second strand of a double-stranded FOXA2-saRNA may have at least 60%, 70%, 80% or 90% identity with a sequence selected from the sequences of the sense strands in Table 17. In one embodiment, the second strand of the double-stranded saRNA comprises a sequence selected from the sequences of the sense strands in Table 17.

FOXA2-saRNA may be modified or unmodified.

Table 16-1 describes the target gene, the targeted sequence, the genomic location of the targeted sequence and the relative location of FOXA2-saRNAs with no 3′ overhang. In Table 16-1, the targeted sequence is defined as a region on the template strand of the target gene which is identical to a region on the target antisense RNA transcript to which the antisense strand of an saRNA hybridizes. In Table 16-1, the relative location is the distance from the 5′ end of the targeted sequence to the TSS. A negative number represents a location upstream of the TSS and a positive number represents a location downstream of the TSS.

TABLE 16-1
Sequences of FOXA2-saRNAs (with no 3′ overhang)

| | Target | | Genomic Location of the | Relative |
|---|---|---|---|---|
| saRNA ID | Gene | Targeted sequence | Targeted sequence | Location |
| FOXA2.NM_021784- | FOXA2 | gcgctgtcctatttagaaa | chr20: 22565879 plus strand | −778 |
| Pr-1 | human | (SEQ ID NO. 777) | | |
| FOXA2.NM_021784- | FOXA2 | ccggcagatccctatgaaa | chr20: 22565776 plus strand | −675 |
| Pr-3 | human | (SEQ ID NO. 778) | | |
| FOXA2.NM_021784- | FOXA2 | ctatgaaagccggatttat | chr20: 22565787 plus strand | −686 |
| Pr-4 | human | (SEQ ID NO. 779) | | |
| FOXA2.NM_021784- | FOXA2 | acataactctgttaggata | chr20: 22564549 plus strand | 552 |
| Pr-6 | human | (SEQ ID NO. 780) | | |
| FOXA2.NM_021784- | FOXA2 | agcgctgtcctatttagaa | chr20: 22565878 plus strand | −777 |
| Pr-7 | human | (SEQ ID NO. 781) | | |
| FOXA2.NM_021784- | FOXA2 | aatattatctcaaggctaa | chr20: 22564631 plus strand | 470 |
| Pr-13 | human | (SEQ ID NO. 782) | | |
| FOXA2.NM_021784- | FOXA2 | ctaacagtgtgtcgtataa | chr20: 22564646 plus strand | 455 |
| Pr-16 | human | (SEQ ID NO. 783) | | |
| FOXA2.NM_021784- | FOXA2 | gccgcaagtcaatatttga | chr20: 22564602 plus strand | 499 |
| Pr-23 | human | (SEQ ID NO. 784) | | |
| FOXA2.NM_021784- | FOXA2 | cctatgaaagccggattta | chr20: 22565786 plus strand | −685 |
| Pr-29 | human | (SEQ ID NO. 785) | | |
| FOXA2.NM_021784- | FOXA2 | aagtcaatatttgatcaca | chr20: 22564607 plus strand | 494 |
| Pr-30 | human | (SEQ ID NO. 786) | | |
| FOXA2.NM_021784- | FOXA2 | gctgagctttgcaggcaaa | chr20: 22564394 plus strand | 707 |
| Pr-33 | human | (SEQ ID NO. 787) | | |
| FOXA2.NM_021784- | FOXA2 | ggcagatccctatgaaagc | chr20: 22565778 plus strand | −677 |
| Pr-35 | human | (SEQ ID NO. 788) | | |
| FOXA2.NM_021784- | FOXA2 | gggtttcttcgctctcagt | chr20: 22565813 plus strand | −712 |
| Pr-36 | human | (SEQ ID NO. 789) | | |
| FOXA2.NM_021784- | FOXA2 | tgcaaagcgctgtcctatt | chr20: 22565873 plus strand | −772 |
| Pr-37 | human | (SEQ ID NO. 790) | | |
| FOXA2.NM_021784- | FOXA2 | gaaagccggatttatttat | chr20: 22565791 plus strand | −690 |
| Pr-39 | human | (SEQ ID NO. 791) | | |
| FOXA2.NM_153675- | FOXA2 | tgagatttgtctctgatat | chr20: 22566114 plus strand | −13 |
| Pr-5 | human | (SEQ ID NO. 792) | | |
| FOXA2.NM_153675- | FOXA2 | gcgctgtcctatttagaaa | chr20: 22565879 plus strand | 222 |
| Pr-6 | human | (SEQ ID NO. 793) | | |
| FOXA2.NM_153675- | FOXA2 | ccggcagatccctatgaaa | chr20: 22565776 plus strand | 325 |
| Pr-8 | human | (SEQ ID NO. 794) | | |
| FOXA2.NM_153675- | FOXA2 | ctatgaaagccggatttat | chr20: 22565787 plus strand | 314 |
| Pr-9 | human | (SEQ ID NO. 795) | | |
| FOXA2.NM_153675- | FOXA2 | ggactgcttaatagaaatt | chr20: 22566315 plus strand | −214 |
| Pr-12 | human | (SEQ ID NO. 796) | | |
| FOXA2.NM_153675- | FOXA2 | agcgctgtcctatttagaa | chr20: 22565878 plus strand | 223 |
| Pr-13 | human | (SEQ ID NO. 797) | | |
| FOXA2.NM_153675- | FOXA2 | agaagaatccaggaatcaa | chr20: 22566239 plus strand | −138 |
| Pr-19 | human | (SEQ ID NO. 798) | | |
| FOXA2.NM_153675- | FOXA2 | ggcgggattctcccacaaa | chr20: 22566838 plus strand | −737 |
| Pr-21 | human | (SEQ ID NO. 799) | | |
| FOXA2.NM_153675- | FOXA2 | cacagcatttcgtaactaa | chr20: 22566181 plus strand | −80 |
| Pr-26 | human | (SEQ ID NO. 800) | | |
| FOXA2.NM_153675- | FOXA2 | agagggactgcttaataga | chr20: 22566311 plus strand | −210 |
| Pr-32 | human | (SEQ ID NO. 801) | | |
| FOXA2.NM_153675- | FOXA2 | agatttgtctctgatattc | chr20: 22566116 plus strand | −15 |
| Pr-40 | human | (SEQ ID NO. 802) | | |

TABLE 16-2
Sequences of FOXA2-saRNAs (with no 3′ overhang)

| | | Sense | | Antisense |
|---|---|---|---|---|
| saRNA ID | Sense (passenger) | SEQ ID | Antisense (guide) | SEQ ID |
| FOXA2.NM_021784- | GCGCUGUCCUAUUUAG | 803 | UUUCUAAAUAGGACAG | 804 |
| Pr-1 | AAA | | CGC | |
| FOXA2.NM_021784- | CCGGCAGAUCCCUAUG | 805 | UUUCAUAGGGAUCUGCC | 806 |
| Pr-3 | AAA | | GG | |
| FOXA2.NM_021784- | CUAUGAAAGCCGGAUU | 807 | AUAAAUCCGGCUUUCAU | 808 |
| Pr-4 | UAU | | AG | |
| FOXA2.NM_021784- | ACAUAACUCUGUUAGG | 809 | UAUCCUAACAGAGUUA | 810 |
| Pr-6 | AUA | | UGU | |
| FOXA2.NM_021784- | AGCGCUGUCCUAUUUA | 811 | UUCUAAAUAGGACAGC | 812 |
| Pr-7 | GAA | | GCU | |
| FOXA2.NM_021784- | AAUAUUAUCUCAAGGC | 813 | UUAGCCUUGAGAUAAU | 814 |
| Pr-13 | UAA | | AUU | |
| FOXA2.NM_021784- | CUAACAGUGUGUCGUA | 815 | UUAUACGACACACUGUU | 816 |
| Pr-16 | UAA | | AG | |
| FOXA2.NM_021784- | GCCGCAAGUCAAUAUU | 817 | UCAAAUAUUGACUUGC | 818 |
| Pr-23 | UGA | | GGC | |
| FOXA2.NM_021784- | CCUAUGAAAGCCGGAU | 819 | UAAAUCCGGCUUUCAUA | 820 |
| Pr-29 | UUA | | GG | |
| FOXA2.NM_021784- | AAGUCAAUAUUUGAUC | 821 | UGUGAUCAAAUAUUGA | 822 |
| Pr-30 | ACA | | CUU | |
| FOXA2.NM_021784- | GCUGAGCUUUGCAGGC | 823 | UUUGCCUGCAAAGCUCA | 824 |
| Pr-33 | AAA | | GC | |
| FOXA2.NM_021784- | GGCAGAUCCCUAUGAA | 825 | GCUUUCAUAGGGAUCU | 826 |
| Pr-35 | AGC | | GCC | |
| FOXA2.NM_021784- | GGGUUUCUUCGCUCUC | 827 | ACUGAGAGCGAAGAAA | 828 |
| Pr-36 | AGU | | CCC | |
| FOXA2.NM_021784- | UGCAAAGCGCUGUCCU | 829 | AAUAGGACAGCGCUUU | 830 |
| Pr-37 | AUU | | GCA | |
| FOXA2.NM_021784- | GAAAGCCGGAUUUAUU | 831 | AUAAAUAAAUCCGGCU | 832 |
| Pr-39 | UAU | | UUC | |
| FOXA2.NM_153675- | UGAGAUUUGUCUCUGA | 833 | AUAUCAGAGACAAAUC | 834 |
| Pr-5 | UAU | | UCA | |
| FOXA2.NM_153675- | GCGCUGUCCUAUUUAG | 835 | UUUCUAAAUAGGACAG | 836 |
| Pr-6 | AAA | | CGC | |
| FOXA2.NM_153675- | CCGGCAGAUCCCUAUG | 837 | UUUCAUAGGGAUCUGCC | 838 |
| Pr-8 | AAA | | GG | |
| FOXA2.NM_153675- | CUAUGAAAGCCGGAUU | 839 | AUAAAUCCGGCUUUCAU | 840 |
| Pr-9 | UAU | | AG | |
| FOXA2.NM_153675- | GGACUGCUUAAUAGAA | 841 | AAUUUCUAUUAAGCAG | 842 |
| Pr-12 | AUU | | UCC | |
| FOXA2.NM_153675- | AGCGCUGUCCUAUUUA | 843 | UUCUAAAUAGGACAGC | 844 |
| Pr-13 | GAA | | GCU | |
| FOXA2.NM_153675- | AGAAGAAUCCAGGAAU | 845 | UUGAUUCCUGGAUUCU | 846 |
| Pr-19 | CAA | | UCU | |
| FOXA2.NM_153675- | GGCGGGAUUCUCCCAC | 847 | UUUGUGGGAGAAUCCC | 848 |
| Pr-21 | AAA | | GCC | |
| FOXA2.NM_153675- | CACAGCAUUUCGUAAC | 849 | UUAGUUACGAAAUGCU | 850 |
| Pr-26 | UAA | | GUG | |
| FOXA2.NM_153675- | AGAGGGACUGCUUAAU | 851 | UCUAUUAAGCAGUCCCU | 852 |
| Pr-32 | AGA | | CU | |
| FOXA2.NM_153675- | AGAUUUGUCUCUGAUA | 853 | GAAUAUCAGAGACAAA | 854 |
| Pr-40 | UUC | | UCU | |

TABLE 17
Sequences of FOXA2-saRNAs (with 3′ overhang)

| | | Sense | | Antisense |
|---|---|---|---|---|
| saRNA ID | Sense (passenger) | SEQ ID | Antisense (guide) | SEQ ID |
| FOXA2.NM_021784- | GCGCUGUCCUAUUUA | 855 | UUUCUAAAUAGGACAGC | 856 |
| Pr-1′ | GAAAUU | | GCUU | |
| FOXA2.NM_021784- | CCGGCAGAUCCCUAU | 857 | UUUCAUAGGGAUCUGCC | 858 |
| Pr-3′ | GAAAUU | | GGUU | |
| FOXA2.NM_021784- | CUAUGAAAGCCGGAU | 859 | AUAAAUCCGGCUUUCAU | 860 |
| Pr-4′ | UUAUUU | | AGUU | |
| FOXA2.NM_021784- | ACAUAACUCUGUUAG | 861 | UAUCCUAACAGAGUUAU | 862 |
| Pr-6′ | GAUAUU | | GUUU | |
| FOXA2.NM_021784- | AGCGCUGUCCUAUUU | 863 | UUCUAAAUAGGACAGCG | 864 |
| Pr-7′ | AGAAUU | | CUUU | |
| FOXA2.NM_021784- | AAUAUUAUCUCAAGG | 865 | UUAGCCUUGAGAUAAUA | 866 |
| Pr-13′ | CUAAUU | | UUUU | |
| FOXA2.NM_021784- | CUAACAGUGUGUCGU | 867 | UUAUACGACACACUGUU | 868 |
| Pr-16′ | AUAAUU | | AGUU | |
| FOXA2.NM_021784- | GCCGCAAGUCAAUAU | 869 | UCAAAUAUUGACUUGCG | 870 |
| Pr-23′ | UUGAUU | | GCUU | |
| FOXA2.NM_021784- | CCUAUGAAAGCCGGA | 871 | UAAAUCCGGCUUUCAUA | 872 |
| Pr-29′ | UUUAUU | | GGUU | |
| FOXA2.NM_021784- | AAGUCAAUAUUUGAU | 873 | UGUGAUCAAAUAUUGAC | 874 |
| Pr-30′ | CACAUU | | UUUU | |
| FOXA2.NM_021784- | GCUGAGCUUUGCAGG | 875 | UUUGCCUGCAAAGCUCA | 876 |
| Pr-33′ | CAAAUU | | GCUU | |
| FOXA2.NM_021784- | GGCAGAUCCCUAUGA | 877 | GCUUUCAUAGGGAUCUG | 878 |
| Pr-35′ | AAGCUU | | CCUU | |
| FOXA2.NM_021784- | GGGUUUCUUCGCUCU | 879 | ACUGAGAGCGAAGAAAC | 880 |
| Pr-36′ | CAGUUU | | CCUU | |
| FOXA2.NM_021784- | UGCAAAGCGCUGUCC | 881 | AAUAGGACAGCGCUUUG | 882 |
| Pr-37′ | UAUUUU | | CAUU | |
| FOXA2.NM_021784- | GAAAGCCGGAUUUAU | 883 | AUAAAUAAAUCCGGCUU | 884 |
| Pr-39′ | UUAUUU | | UCUU | |
| FOXA2.NM_153675- | UGAGAUUUGUCUCUG | 885 | AUAUCAGAGACAAAUCU | 886 |
| Pr-5′ | AUAUUU | | CAUU | |
| FOXA2.NM_153675- | GCGCUGUCCUAUUUA | 887 | UUUCUAAAUAGGACAGC | 888 |
| Pr-6′ | GAAAUU | | GCUU | |
| FOXA2.NM_153675- | CCGGCAGAUCCCUAU | 889 | UUUCAUAGGGAUCUGCC | 890 |
| Pr-8′ | GAAAUU | | GGUU | |
| FOXA2.NM_153675- | CUAUGAAAGCCGGAU | 891 | AUAAAUCCGGCUUUCAU | 892 |
| Pr-9′ | UUAUUU | | AGUU | |
| FOXA2.NM_153675- | GGACUGCUUAAUAGA | 893 | AAUUUCUAUUAAGCAGU | 894 |
| Pr-12′ | AAUUUU | | CCUU | |
| FOXA2.NM_153675- | AGCGCUGUCCUAUUU | 895 | UUCUAAAUAGGACAGCG | 896 |
| Pr-13′ | AGAAUU | | CUUU | |
| FOXA2.NM_153675- | AGAAGAAUCCAGGAA | 897 | UUGAUUCCUGGAUUCUU | 898 |
| Pr-19′ | UCAAUU | | CUUU | |
| FOXA2.NM_153675- | GGCGGGAUUCUCCCA | 899 | UUUGUGGGAGAAUCCCG | 900 |
| Pr-21′ | CAAAUU | | CCUU | |
| FOXA2.NM_153675- | CACAGCAUUUCGUAA | 901 | UUAGUUACGAAAUGCUG | 902 |
| Pr-26′ | CUAAUU | | UGUU | |
| FOXA2.NM_153675- | AGAGGGACUGCUUAA | 903 | UCUAUUAAGCAGUCCCU | 904 |
| Pr-32′ | UAGAUU | | CUUU | |
| FOXA2.NM_153675- | AGAUUUGUCUCUGAU | 905 | GAAUAUCAGAGACAAAU | 906 |
| Pr-40′ | AUUCUU | | CUUU | |

FOXA2 gene, also called HNF3G, encodes a member of the forkhead class of DNA-binding proteins. It is a transcription factor that is involved in embryonic development, establishment of tissue-specific gene expression and regulation of gene expression in differentiated tissues. FOXA2 is a transcriptional activator for liver-specific genes such as albumin, transthyretin, and tyrosine aminotransferase. It also interacts with chromatin and regulates the expression of genes for glucose sensing in pancreatic beta-cells and glucose homeostasis (Wang et al., J. Biol. Chem., vol. 277:17564 (2002), the contents of which are incorporated herein by reference in their entirety). It also regulates fat metabolism, binds to fibrinogen beta promoter and is involved in IL6-induced fibrinogen beta transcriptional activation. Diseases associated with FOXA2 include Meckel's diverticulum, and maturity-onset diabetes of the young.

In one embodiment, provided is a method of modulating FOXA2 protein levels comprising administering FOXA2-saRNA of the present invention, wherein the FOXA2-saRNA targets an antisense RNA transcript of FOXA2 gene. In one embodiment, FOXA2 protein level is increased by at least 20, 30, 40%, or at least 45, 50, 55, 60, 65, 70, 75%, or at least 80% in the presence of the FOXA2-saRNA of the present invention compared to FOXA2 protein level in the absence of the FOXA2-saRNA of the present invention. In a further embodiment, FOXA2 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FOXA2-saRNA of the present invention compared to FOXA2 protein level in the absence of the FOXA2-saRNA of the present invention.

In another embodiment, provided is a method of modulating glucose sensing or glucose homeostasis, comprising administering FOXA2-saRNA of the present invention, wherein the FOXA2-saRNA targets an antisense RNA transcript of FOXA2 gene.

In another embodiment, provided is a method of treating Meckel's diverticulum or maturity-onset diabetes of the young, comprising administering FOXA2-saRNA of the present invention, wherein the FOXA2-saRNA targets an antisense RNA transcript of FOXA2 gene.

In one embodiment, the FOXA2-saRNA of the present invention is an antisense sequence such as, but not limited to, SEQ ID NO: 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 3600, 3602, 3604, 3606, 3608, 3610, 3612, 3614, 3616, 3618, 3620, 3622, 3624, 3626, 3628, 3630, 3632, 3634, 3636, 3638, 3640, 3642, 3644, 3646, 3648, 3650, 3652, 3654, 3656, 3658, 3660, 3662, 3664, 3666, 3668, 3670, 3672, 3674, 3676, 3678, 3680, 3682, 3684, 3686, 3688, 3690, 3692, 3694, 3696, 3698, 3700, 3702, 3704, 3706, 3708, 3710, 3712, 3714, 3716, 3718, 3720, 3722, 3724, 3726, 3728, 3730, 3732, 3734, 3736, 3738, 3740, 3742, 3744, 3746, 3748, 3750, 3752, 3754, 3756, 3758 and 3760. As a non-limiting example, these FOXA2-saRNA sequences may be used to modulate FOXA2 protein levels, modulating glucose sensing or glucose homeostasis and/or treating Meckel's diverticulum or maturity-onset diabetes of the young.

In one embodiment, the FOXA2-saRNA of the present invention is an saRNA duplex. The saRNA duplex may be a pair of sense and antisense sequences such as, but not limited to, SEQ ID NOs: 803 and 804; SEQ ID NOs: 805 and 806; SEQ ID NOs: 807 and 808; SEQ ID NOs: 809 and 810; SEQ ID NOs: 811 and 812; SEQ ID NOs: 813 and 814; SEQ ID NOs: 815 and 816; SEQ ID NOs: 817 and 818; SEQ ID NOs: 819 and 820; SEQ ID NOs: 821 and 822; SEQ ID NOs: 823 and 824; SEQ ID NOs: 825 and 826; SEQ ID NOs: 827 and 828; SEQ ID NOs: 829 and 830; SEQ ID NOs: 831 and 832; SEQ ID NOs: 833 and 834; SEQ ID NOs: 835 and 836; SEQ ID NOs: 837 and 838; SEQ ID NOs: 839 and 840; SEQ ID NOs: 841 and 842; SEQ ID NOs: 843 and 844; SEQ ID NOs: 845 and 846; SEQ ID NOs: 847 and 848; SEQ ID NOs: 849 and 850; SEQ ID NOs: 851 and 852; SEQ ID NOs: 853 and 854; SEQ ID NOs: 855 and 856; SEQ ID NOs: 857 and 858; SEQ ID NOs: 859 and 860; SEQ ID NOs: 861 and 862; SEQ ID NOs: 863 and 864; SEQ ID NOs: 865 and 866; SEQ ID NOs: 867 and 868; SEQ ID NOs: 869 and 870; SEQ ID NOs: 871 and 872; SEQ ID NOs: 873 and 874; SEQ ID NOs: 875 and 876; SEQ ID NOs: 877 and 878; SEQ ID NOs: 879 and 880; SEQ ID NOs: 881 and 882; SEQ ID NOs: 883 and 884; SEQ ID NOs: 885 and 886; SEQ ID NOs: 887 and 888; SEQ ID NOs: 889 and 890; SEQ ID NOs: 891 and 892; SEQ ID NOs: 893 and 894; SEQ ID NOs: 895 and 896; SEQ ID NOs: 897 and 898; SEQ ID NOs: 899 and 900; SEQ ID NOs: 901 and 902; SEQ ID NOs: 903 and 904; SEQ ID NOs: 3599 and 3600; SEQ ID NOs: 3601 and 3602; SEQ ID NOs: 3603 and 3604; SEQ ID NOs: 3605 and 3606; SEQ ID NOs: 3607 and 3608; SEQ ID NOs: 3609 and 3610; SEQ ID NOs: 3611 and 3612; SEQ ID NOs: 3613 and 3614; SEQ ID NOs: 3615 and 3616; SEQ ID NOs: 3617 and 3618; SEQ ID NOs: 3619 and 3620; SEQ ID NOs: 3621 and 3622; SEQ ID NOs: 3623 and 3624; SEQ ID NOs: 3625 and 3626; SEQ ID NOs: 3627 and 3628; SEQ ID NOs: 3629 and 3630; SEQ ID NOs: 3631 and 3632; SEQ ID NOs: 3633 and 3634; SEQ ID NOs: 3635 and 3636; SEQ ID NOs: 3637 and 3638; SEQ ID NOs: 3639 and 3640; SEQ ID NOs: 3641 and 3642; SEQ ID NOs: 3643 and 3644; SEQ ID NOs: 3645 and 3646; SEQ ID NOs: 3647 and 3648; SEQ ID NOs: 3649 and 3650; SEQ ID NOs: 3651 and 3652; SEQ ID NOs: 3653 and 3654; SEQ ID NOs: 3655 and 3656; SEQ ID NOs: 3657 and 3658; SEQ ID NOs: 3659 and 3660; SEQ ID NOs: 3661 and 3662; SEQ ID NOs: 3663 and 3664; SEQ ID NOs: 3665 and 3666; SEQ ID NOs: 3667 and 3668; SEQ ID NOs: 3669 and 3670; SEQ ID NOs: 3671 and 3672; SEQ ID NOs: 3673 and 3674; SEQ ID NOs: 3675 and 3676; SEQ ID NOs: 3677 and 3678; SEQ ID NOs: 3679 and 3680; SEQ ID NOs: 3681 and 3682; SEQ ID NOs: 3683 and 3684; SEQ ID NOs: 3685 and 3686; SEQ ID NOs: 3687 and 3688; SEQ ID NOs: 3689 and 3690; SEQ ID NOs: 3691 and 3692; SEQ ID NOs: 3693 and 3694; SEQ ID NOs: 3695 and 3696; SEQ ID NOs: 3697 and 3698; SEQ ID NOs: 3699 and 3700; SEQ ID NOs: 3701 and 3702; SEQ ID NOs: 3703 and 3704; SEQ ID NOs: 3705 and 3706; SEQ ID NOs: 3707 and 3708; SEQ ID NOs: 3709 and 3710; SEQ ID NOs: 3711 and 3712; SEQ ID NOs: 3713 and 3714; SEQ ID NOs: 3715 and 3716; SEQ ID NOs: 3717 and 3718; SEQ ID NOs: 3719 and 3720; SEQ ID NOs: 3721 and 3722; SEQ ID NOs: 3723 and 3724; SEQ ID NOs: 3725 and 3726; SEQ ID NOs: 3727 and 3728; SEQ ID NOs: 3729 and 3730; SEQ ID NOs: 3731 and 3732; SEQ ID NOs: 3733 and 3734; SEQ ID NOs: 3735 and 3736; SEQ ID NOs: 3737 and 3738; SEQ ID NOs: 3739 and 3740; SEQ ID NOs: 3741 and 3742; SEQ ID NOs: 3743 and 3744; SEQ ID NOs: 3745 and 3746; SEQ ID NOs: 3747 and 3748; SEQ ID NOs: 3749 and 3750; SEQ ID NOs: 3751 and 3752; SEQ ID NOs: 3753 and 3754; SEQ ID NOs: 3755 and 3756; SEQ ID NOs: 3757 and 3758; SEQ ID NOs: 3759 and 3760. As a non-limiting example, these FOXA2-saRNA sequences which are saRNA duplexes may be used to modulate FOXA2 protein levels, modulating glucose sensing or glucose homeostasis and/or treating Meckel's diverticulum or maturity-onset diabetes of the young.

### FOXP3 Gene

As another non-limiting example, provided is a method of modulating the expression of FOXP3 gene comprising administering saRNA of the present invention, wherein the saRNA targets an antisense RNA transcript of FOXP3 gene. These saRNAs are called FOXP3-saRNA. In one embodiment, the expression of FOXP3 gene is increased by at least 20, 30, 40%, or at least 45, 50, 55, 60, 65, 70, 75%, or at least 80% in the presence of the FOXP3-saRNA of the present invention compared to the expression of FOXP3 gene in the absence of the FOXP3-saRNA of the present invention. In a further embodiment, the expression of FOXP3 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FOXP3-saRNA of the present invention compared to the expression of FOXP3 gene in the absence of the FOXP3-saRNA of the present invention. The modulation of the expression of FOXP3 gene may be reflected or determined by the change of FOXP3 mRNA levels.

FOXP3-saRNAs may be single-stranded and comprise 14-30 nucleotides. The sequence of a single-stranded FOXP3-saRNA may have at least 60%, 70%, 80% or 90% identity with a sequence selected from the sequences of the antisense strands in Table 18-2. In one embodiment, the single-stranded saRNA comprises a sequence selected from the sequences of the antisense strands in Table 18-2. In one embodiment, the single-stranded FOXP3-saRNA may have a 3′ tail. The sequence of a single-stranded FOXP3-saRNA with a 3′ tail may have at least 60%, 70%, 80% or 90% identity with a sequence selected from the sequences of the antisense strands in Table 19. In one embodiment, the single-stranded saRNA comprises a sequence selected from the sequences of the antisense strands in Table 19.

FOXP3-saRNAs may be double-stranded. The two strands form a duplex and each strand comprises 14-30 nucleotides. The first strand of a double-stranded FOXP3-saRNA may have at least 60%, 70%, 80% or 90% identity with a sequence selected from the sequences of the antisense strands in Table 18-2. In one embodiment, the first strand of the double-stranded saRNA comprises a sequence selected from the sequences of the antisense strands in Table 18-2. The second strand of a double-stranded FOXP3-saRNA may have at least 60%, 70%, 80% or 90% identity with a sequence selected from the sequences of the sense strands in Table 18-2. In one embodiment, the second strand of the double-stranded saRNA comprises a sequence selected from the sequences of the sense strands in Table 18-2. In one embodiment, the double-stranded FOXP3-saRNA may have a 3′ overhang on each strand. The first strand of a double-stranded FOXP3-saRNA may have at least 60%, 70%, 80% or 90% identity with a sequence selected from the sequences of the antisense strands in Table 19. In one embodiment, the first strand of the double-stranded saRNA comprises a sequence selected from the sequences of the antisense strands in Table 19. The second strand of a double-stranded FOXP3-saRNA may have at least 60%, 70%, 80% or 90% identity with a sequence selected from the sequences of the sense strands in Table 19. In one embodiment, the second strand of the double-stranded saRNA comprises a sequence selected from the sequences of the sense strands in Table 19.

FOXP3-saRNAs may be modified or unmodified.

Table 18-1 describes the target gene, the targeted sequence, the genomic location of the targeted sequence and the relative location of FOXP3-saRNAs with no 3′ overhang. In Table 18-1, the targeted sequence is defined as a region on the template strand of the target gene which is identical to a region on the target antisense RNA transcript to which the antisense strand of an saRNA hybridizes. In Table 18-1, the relative location is the distance from the 5′ end of the targeted sequence to the TSS. A negative number represents a location upstream of the TSS and a positive number represents a location downstream of the TSS.

TABLE 18-1
Sequences of FOXP3-saRNAs (with no 3′ overhang)

| | | | Targeted | | |
|---|---|---|---|---|---|
| | Target | | sequence | Genomic Location of the | Relative |
| saRNA ID | Gene | Targeted sequence | ID | Targeted sequence | Location |
| FOXP3.NM_014009- | FOXP3 | taatagggctcatgagaaa | 907 | chrX: 49121476 plus | −188 |
| Pr-15 | human | | | strand | |
| FOXP3.NM_014009- | FOXP3 | ctacgtatcaattgatgaa | 908 | chrX: 49120882 plus | 406 |
| Pr-19 | human | | | strand | |
| FOXP3.NM_014009- | FOXP3 | tcttgattatgagacttaa | 909 | chrX: 49121356 plus | −68 |
| Pr-21 | human | | | strand | |
| FOXP3.NM_014009- | FOXP3 | agaggtgagaggtatcaat | 910 | chrX: 49121453 plus | −165 |
| Pr-23 | human | | | strand | |
| FOXP3.NM_014009- | FOXP3 | acttgaagagtcggagaat | 911 | chrX: 49121898 plus | −610 |
| Pr-29 | human | | | strand | |
| FOXP3.NM_014009- | FOXP3 | gaacatgtctcacaagtga | 912 | chrX: 49122268 plus | −980 |
| Pr-34 | human | | | strand | |
| FOXP3.NM_014009- | FOXP3 | ggtctcctctaaagcgata | 913 | chrX: 49120326 plus | 962 |
| Pr-40 | human | | | strand | |

TABLE 18-2
Sequences of FOXP3-saRNAs (with no 3′ overhang)

| | | Sense | | Anti-sense |
|---|---|---|---|---|
| saRNA ID | Sense (passenger) | SEQ ID | Antisense (guide) | SEQ ID |
| FOXP3.NM_014009- | UAAUAGGGCUCAUGAG | 914 | UUUCUCAUGAGCCCUA | 915 |
| Pr-15 | AAA | | UUA | |
| FOXP3.NM_014009- | CUACGUAUCAAUUGAU | 916 | UUCAUCAAUUGAUACG | 917 |
| Pr-19 | GAA | | UAG | |
| FOXP3.NM_014009- | UCUUGAUUAUGAGACU | 918 | UUAAGUCUCAUAAUCA | 919 |
| Pr-21 | UAA | | AGA | |
| FOXP3.NM_014009- | AGAGGUGAGAGGUAUC | 920 | AUUGAUACCUCUCACCU | 921 |
| Pr-23 | AAU | | CU | |
| FOXP3.NM_014009- | ACUUGAAGAGUCGGAG | 922 | AUUCUCCGACUCUUCAA | 923 |
| Pr-29 | AAU | | GU | |
| FOXP3.NM_014009- | GAACAUGUCUCACAAG | 924 | UCACUUGUGAGACAUG | 925 |
| Pr-34 | UGA | | UUC | |
| FOXP3.NM_014009- | GGUCUCCUCUAAAGCG | 926 | UAUCGCUUUAGAGGAG | 927 |
| Pr-40 | AUA | | ACC | |

TABLE 19
Sequences of FOXP3-saRNAs (with 3′ overhang)

| | | Sense | | Antisense |
|---|---|---|---|---|
| saRNA ID | Sense (passenger) | SEQ ID | Antisense (guide) | SEQ ID |
| FOXP3.NM_014009- | UAAUAGGGCUCAUG | 928 | UUUCUCAUGAGCCCUAUU | 929 |
| Pr-15′ | AGAAAUU | | AUU | |
| FOXP3.NM_014009- | CUACGUAUCAAUUG | 930 | UUCAUCAAUUGAUACGUA | 931 |
| Pr-19′ | AUGAAUU | | GUU | |
| FOXP3.NM_014009- | UCUUGAUUAUGAGA | 932 | UUAAGUCUCAUAAUCAAG | 933 |
| Pr-21′ | CUUAAUU | | AUU | |
| FOXP3.NM_014009- | AGAGGUGAGAGGUA | 934 | AUUGAUACCUCUCACCUC | 935 |
| Pr-23′ | UCAAUUU | | UUU | |
| FOXP3.NM_014009- | ACUUGAAGAGUCGG | 936 | AUUCUCCGACUCUUCAAG | 937 |
| Pr-29′ | AGAAUUU | | UUU | |
| FOXP3.NM_014009- | GAACAUGUCUCACA | 938 | UCACUUGUGAGACAUGUU | 939 |
| Pr-34′ | AGUGAUU | | CUU | |
| FOXP3.NM_014009- | GGUCUCCUCUAAAG | 940 | UAUCGCUUUAGAGGAGAC | 941 |
| Pr-40′ | CGAUAUU | | CUU | |

FOXP3 gene encodes the forkhead box P3 (FOXP3) protein. FOXP3 protein is a transcription factor that binds to specific regions of DNA and helps control the activity of genes that are involved in regulating the immune system. FOXP3 protein regulates the production and normal function of immune cells called regulatory T cells, which play an important role in preventing autoimmunity. Autoimmunity occurs when the body attacks its own tissues and organs by mistake. FOXP3 protein is found primarily in an immune system gland called the thymus, where regulatory T cells are produced. Mutations in FOXP3 gene cause immune dysregulation, polyendocrinopathy, enteropathy, X-linked (IPEX) syndrome. Mutations in the FOXP3 gene result in reduced numbers or a complete absence of regulatory T cells. Without the proper number of regulatory T cells, the body cannot control immune responses. Normal body tissues and organs are attacked, causing the multiple autoimmune disorders present in people with IPEX syndrome (Barzaghi et al., Front Immunol., vol 3:211 (2012), the contents of which are incorporated herein by reference in their entirety). Mutations of FOXP3 gene are also associated with type 1 diabetes (Petzold, J Diabetes Res., vol. 2013:940710 (2013), the contents of which are incorporated herein by reference in their entirety).

In one embodiment, provided is a method of modulating FOXP3 protein levels comprising administering FOXP3-saRNA of the present invention, wherein the FOXP3-saRNA targets an antisense RNA transcript of FOXP3 gene. In one embodiment, FOXP3 protein level is increased by at least 20, 30, 40%, or at least 45, 50, 55, 60, 65, 70, 75%, or at least 80% in the presence of the FOXP3-saRNA of the present invention compared to FOXP3 protein level in the absence of the FOXP3-saRNA of the present invention. In a further embodiment, FOXP3 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FOXP3-saRNA of the present invention compared to FOXP3 protein level in the absence of the FOXP3-saRNA of the present invention.

In another embodiment, provided is a method of modulating regulatory T cell levels comprising administering FOXP3-saRNA of the present invention, wherein the FOXP3-saRNA targets an antisense RNA transcript of FOXP3 gene. In one embodiment, regulatory T cell level is increased by at least 20, 30, 40%, or at least 45, 50, 55, 60, 65, 70, 75%, or at least 80% in the presence of the FOXP3-saRNA of the present invention compared to regulatory T cell level in the absence of the FOXP3-saRNA of the present invention. In a further embodiment, regulatory T cell level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the FOXP3-saRNA of the present invention compared to regulatory T cell level in the absence of the FOXP3-saRNA of the present invention.

In another embodiment, provided is a method of prevent autoimmunity comprising administering FOXP3-saRNA of the present invention, wherein the FOXP3-saRNA targets an antisense RNA transcript of FOXP3 gene, and wherein the patient's chance of getting autoimmunity is reduced.

In another embodiment, provided is a method of treating IPEX syndrome comprising administering FOXP3-saRNA of the present invention, wherein the FOXP3-saRNA targets an antisense RNA transcript of FOXP3 gene, and wherein the symptoms of IPEX syndrome are reduced.

In another embodiment, provided is a method of treating type 1 diabetes comprising administering FOXP3-saRNA of the present invention, wherein the FOXP3-saRNA targets an antisense RNA transcript of FOXP3 gene, and wherein the symptoms of type 1 diabetes are reduced.

In one embodiment, the FOXP3-saRNA of the present invention is an antisense sequence such as, but not limited to, SEQ ID NO: 915, 917, 919, 921, 923, 925, 927, 929, 931, 933, 935, 937, 939, 941, 3762, 3764, 3766, 3768, 3770, 3772, 3774, 3776, 3778, 3780, 3782, 3784, 3786, 3788, 3790, 3792, 3794, 3796, 3798, 3800, 3802, 3804, 3806, 3808, 3810, 3812, 3814, 3816, 3818, 3820, 3822, 3824, 3826, 3828, 3830, 3832, 3834, 3836, 3838, 3840 and 3842. As a non-limiting example, these FOXP3-saRNA sequences may be used to modulate FOXP3 protein levels, modulate regulatory T cell levels, prevent autoimmunity, treat IPEX syndrome and/or treat type 1 diabetes.

In one embodiment, the FOXP3-saRNA of the present invention is an saRNA duplex. The saRNA duplex may be a pair of sense and antisense sequences such as, but not limited to, SEQ ID NOs: 914 and 915; SEQ ID NO: 916 and 917; SEQ ID NO: 918 and 919; SEQ ID NO: 920 and 921; SEQ ID NO: 922 and 923; SEQ ID NO: 924 and 925; SEQ ID NO: 926 and 927; SEQ ID NO: 928 and 929; SEQ ID NO: 930 and 931; SEQ ID NO: 932 and 933; SEQ ID NO: 934 and 935; SEQ ID NO: 936 and 937; SEQ ID NO: 938 and 939; SEQ ID NO: 940 and 941; SEQ ID NO: 3761 and 3762; SEQ ID NO: 3763 and 3764; SEQ ID NO: 3765 and 3766; SEQ ID NO: 3767 and 3768; SEQ ID NO: 3769 and 3770; SEQ ID NO: 3771 and 3772; SEQ ID NO: 3773 and 3774; SEQ ID NO: 3775 and 3776; SEQ ID NO: 3777 and 3778; SEQ ID NO: 3779 and 3780; SEQ ID NO: 3781 and 3782; SEQ ID NO: 3783 and 3784; SEQ ID NO: 3785 and 3786; SEQ ID NO: 3787 and 3788; SEQ ID NO: 3789 and 3790; SEQ ID NO: 3791 and 3792; SEQ ID NO: 3793 and 3794; SEQ ID NO: 3795 and 3796; SEQ ID NO: 3797 and 3798; SEQ ID NO: 3799 and 3800; SEQ ID NO: 3801 and 3802; SEQ ID NO: 3803 and 3804; SEQ ID NO: 3805 and 3806; SEQ ID NO: 3807 and 3808; SEQ ID NO: 3809 and 3810; SEQ ID NO: 3811 and 3812; SEQ ID NO: 3813 and 3814; SEQ ID NO: 3815 and 3816; SEQ ID NO: 3817 and 3818; SEQ ID NO: 3819 and 3820; SEQ ID NO: 3821 and 3822; SEQ ID NO: 3823 and 3824; SEQ ID NO: 3825 and 3826; SEQ ID NO: 3827 and 3828; SEQ ID NO: 3829 and 3830; SEQ ID NO: 3831 and 3832; SEQ ID NO: 3833 and 3834; SEQ ID NO: 3835 and 3836; SEQ ID NO: 3837 and 3838; SEQ ID NO: 3839 and 3840; SEQ ID NO: 3841 and 3842. As a non-limiting example, these FOXP3-saRNA sequences which are saRNA duplexes may be used to modulate FOXP3 protein levels, modulate regulatory T cell levels, prevent autoimmunity, treat IPEX syndrome and/or treat type 1 diabetes.

### HNF4A Gene

As another non-limiting example, provided is a method of modulating the expression of HNF4A gene comprising administering saRNA of the present invention, wherein the saRNA targets an antisense RNA transcript of HNF4A gene. These saRNAs are called HNF4A-saRNA. In one embodiment, the expression of HNF4A gene is increased by at least 20, 30, 40%, or at least 45, 50, 55, 60, 65, 70, 75%, or at least 80% in the presence of the HNF4A-saRNA of the present invention compared to the expression of HNF4A gene in the absence of the HNF4A-saRNA of the present invention. In a further embodiment, the expression of HNF4A gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the HNF4A-saRNA of the present invention compared to the expression of HNF4A gene in the absence of the HNF4A-saRNA of the present invention. The modulation of the expression of HNF4A gene may be reflected or determined by the change of HNF4A mRNA levels.

HNF4A-saRNAs may be single-stranded and comprise 14-30 nucleotides. The sequence of a single-stranded HNF4A-saRNA may have at least 60%, 70%, 80% or 90% identity with a sequence selected from the sequences of the antisense strands in Table 20-2. In one embodiment, the single-stranded saRNA comprises a sequence selected from the sequences of the antisense strands in Table 20-2. In one embodiment, the single-stranded HNF4A-saRNA may have a 3′ tail. The sequence of a single-stranded HNF4A-saRNA with a 3′ tail may have at least 60%, 70%, 80% or 90% identity with a sequence selected from the sequences of the antisense strands in Table 21. In one embodiment, the single-stranded saRNA comprises a sequence selected from the sequences of the antisense strands in Table 21.

HNF4A-saRNAs may be double-stranded. The two strands form a duplex and each strand comprises 14-30 nucleotides. The first strand of a double-stranded HNF4A-saRNA may have at least 60%, 70%, 80% or 90% identity with a sequence selected from the sequences of the antisense strands in Table 20-2. In one embodiment, the first strand of the double-stranded saRNA comprises a sequence selected from the sequences of the antisense strands in Table 20-2. The second strand of a double-stranded HNF4A-saRNA may have at least 60%, 70%, 80% or 90% identity with a sequence selected from the sequences of the sense strands in Table 20-2. In one embodiment, the second strand of the double-stranded saRNA comprises a sequence selected from the sequences of the sense strands in Table 20-2. In one embodiment, the double-stranded HNF4A-saRNA may have a 3′ overhang on each strand. The first strand of a double-stranded HNF4A-saRNA may have at least 60%, 70%, 80% or 90% identity with a sequence selected from the sequences of the antisense strands in Table 21. In one embodiment, the first strand of the double-stranded saRNA comprises a sequence selected from the sequences of the antisense strands in Table 21. The second strand of a double-stranded HNF4A-saRNA may have at least 60%, 70%, 80% or 90% identity with a sequence selected from the sequences of the sense strands in Table 21. In one embodiment, the second strand of the double-stranded saRNA comprises a sequence selected from the sequences of the sense strands in Table 21.

HNF4A-saRNAs may be modified or unmodified.

Table 20-1 describes the target gene, the targeted sequence, the genomic location of the targeted sequence and the relative location of HNF4A-saRNAs with no 3′ overhang. In Table 20-1, the targeted sequence is defined as a region on the template strand of the target gene which is identical to a region on the target antisense RNA transcript to which the antisense strand of an saRNA hybridizes. In Table 20-1, the relative location is the distance from the 5′ end of the targeted sequence to the TSS. A negative number represents a location upstream of the TSS and a positive number represents a location downstream of the TSS.

TABLE 20-1
Sequences of HNF4A-saRNAs (with no 3′ overhang)

| | Target | | Targeted | Genomic Location of the | Relative |
|---|---|---|---|---|---|
| saRNA ID | Gene | Targeted sequence | sequence ID | Targeted sequence | Location |
| HNF4A.NM_000457- | HNF4A | tcagttatcaattgtacaa | 942 | chr20: 43029671 minus | −205 |
| Pr-3 | human | | | strand | |
| HNF4A.NM_000457- | HNF4A | ggcattctaactgatacta | 943 | chr20: 43029575 minus | −301 |
| Pr-16 | human | | | strand | |
| HNF4A.NM_000457- | HNF4A | gcggttatgtctccaacca | 944 | chr20: 43030554 minus | 678 |
| Pr-19 | human | | | strand | |
| HNF4A.NM_000457- | HNF4A | tgagttggcttcgtagtta | 945 | chr20: 43030652 minus | 776 |
| Pr-23 | human | | | strand | |
| HNF4A.NM_000457- | HNF4A | gagaaatgcggttatgtct | 946 | chr20: 43030561 minus | 685 |
| Pr-26 | human | | | strand | |
| HNF4A.NM_000457- | HNF4A | cgagaaatgcggttatgtc | 947 | chr20: 43030562 minus | 686 |
| Pr-36 | human | | | strand | |
| HNF4A.NM_000457- | HNF4A | aggcattctaactgatact | 948 | chr20: 43029576 minus | −300 |
| Pr-37 | human | | | strand | |
| HNF4A.NM_175914- | HNF4A | agatcgtcattcaaagata | 949 | chr20: 42983671 minus | −750 |
| Pr-1 | human | | | strand | |
| HNF4A.NM_175914- | HNF4A | gcgttgtttccttgaacaa | 950 | chr20: 42985183 minus | 762 |
| Pr-3 | human | | | strand | |
| HNF4A.NM_175914- | HNF4A | cgttgtttccttgaacaat | 951 | chr20: 42985182 minus | 761 |
| Pr-4 | human | | | strand | |
| HNF4A.NM_175914- | HNF4A | tgtgtcattccacgaattt | 952 | chr20: 42985343 minus | 922 |
| Pr-7 | human | | | strand | |
| HNF4A.NM_175914- | HNF4A | ggcgttaccatatttaaat | 953 | chr20: 42983904 minus | −517 |
| Pr-8 | human | | | strand | |
| HNF4A.NM_175914- | HNF4A | aattaaaccttgagatgaa | 954 | chr20: 42983888 minus | −533 |
| Pr-9 | human | | | strand | |
| HNF4A.NM_175914- | HNF4A | gagctttgggcccgtaaga | 955 | chr20: 42984573 minus | 152 |
| Pr-15 | human | | | strand | |
| HNF4A.NM_175914- | HNF4A | ggtggatacgttaaagagt | 956 | chr20: 42984266 minus | −155 |
| Pr-16 | human | | | strand | |
| HNF4A.NM_175914- | HNF4A | gcgttaccatatttaaatt | 957 | chr20: 42983903 minus | −518 |
| Pr-19 | human | | | strand | |
| HNF4A.NM_175914- | HNF4A | ggagaactgtgtttagtga | 958 | chr20: 42983953 minus | −468 |
| Pr-22 | human | | | strand | |
| HNF4A.NM_175914- | HNF4A | caggcgttaccatatttaa | 959 | chr20: 42983906 minus | −515 |
| Pr-25 | human | | | strand | |
| HNF4A.NM_175914- | HNF4A | ccttaatcagatcgtcatt | 960 | chr20: 42983679 minus | −742 |
| Pr-27 | human | | | strand | |
| HNF4A.NM_175914- | HNF4A | gctttgggcccgtaagaaa | 961 | chr20: 42984571 minus | 150 |
| Pr-31 | human | | | strand | |
| HNF4A.NM_175914- | HNF4A | gcccggttatcttattgat | 962 | chr20: 42984305 minus | −116 |
| Pr-32 | human | | | strand | |
| HNF4A.NM_175914- | HNF4A | ggatagcattccactgtaa | 963 | chr20: 42985162 minus | 741 |
| Pr-36 | human | | | strand | |
| HNF4A.NM_175914- | HNF4A | gtggatacgttaaagagta | 964 | chr20: 42984265 minus | −156 |
| Pr-39 | human | | | strand | |

TABLE 20-2
Sequences of HNF4A-saRNAs (with no 3′ overhang)

| | | Sense SEQ | | Antisense |
|---|---|---|---|---|
| saRNA ID | Sense (passenger) | ID | Antisense (guide) | SEQ ID |
| HNF4A.NM_000457- | UCAGUUAUCAAU | 965 | UUGUACAAUU | 966 |
| Pr-3 | UGUACAA | | GAUAACUGA | |
| HNF4A.NM_000457- | GGCAUUCUAACU | 967 | UAGUAUCAGU | 968 |
| Pr-16 | GAUACUA | | UAGAAUGCC | |
| HNF4A.NM_000457- | GCGGUUAUGUCU | 969 | UGGUUGGAGA | 970 |
| Pr-19 | CCAACCA | | CAUAACCGC | |
| HNF4A.NM_000457- | UGAGUUGGCUUC | 971 | UAACUACGAA | 972 |
| Pr-23 | GUAGUUA | | GCCAACUCA | |
| HNF4A.NM_000457- | GAGAAAUGCGG | 973 | AGACAUAACC | 974 |
| Pr-26 | UUAUGUCU | | GCAUUUCUC | |
| HNF4A.NM_000457- | CGAGAAAUGCGG | 975 | GACAUAACCG | 976 |
| Pr-36 | UUAUGUC | | CAUUUCUCG | |
| HNF4A.NM_000457- | AGGCAUUCUAAC | 977 | AGUAUCAGUU | 978 |
| Pr-37 | UGAUACU | | AGAAUGCCU | |
| HNF4A.NM_175914- | AGAUCGUCAUUC | 979 | UAUCUUUGAA | 980 |
| Pr-1 | AAAGAUA | | UGACGAUCU | |
| HNF4A.NM_175914- | GCGUUGUUUCCU | 981 | UUGUUCAAGG | 982 |
| Pr-3 | UGAACAA | | AAACAACGC | |
| HNF4A.NM_175914- | CGUUGUUUCCUU | 983 | AUUGUUCAAG | 984 |
| Pr-4 | GAACAAU | | GAAACAACG | |
| HNF4A.NM_175914- | UGUGUCAUUCCA | 985 | AAAUUCGUGG | 986 |
| Pr-7 | CGAAUUU | | AAUGACACA | |
| HNF4A.NM_175914- | GGCGUUACCAUA | 987 | AUUUAAAUAU | 988 |
| Pr-8 | UUUAAAU | | GGUAACGCC | |
| HNF4A.NM_175914- | AAUUAAACCUUG | 989 | UUCAUCUCAA | 990 |
| Pr-9 | AGAUGAA | | GGUUUAAUU | |
| HNF4A.NM_175914- | GAGCUUUGGGCC | 991 | UCUUACGGGC | 992 |
| Pr-15 | CGUAAGA | | CCAAAGCUC | |
| HNF4A.NM_175914- | GGUGGAUACGU | 993 | ACUCUUUAAC | 994 |
| Pr-16 | UAAAGAGU | | GUAUCCACC | |
| HNF4A.NM_175914- | GCGUUACCAUAU | 995 | AAUUUAAAUA | 996 |
| Pr-19 | UUAAAUU | | UGGUAACGC | |
| HNF4A.NM_175914- | GGAGAACUGUG | 997 | UCACUAAACA | 998 |
| Pr-22 | UUUAGUGA | | CAGUUCUCC | |
| HNF4A.NM_175914- | CAGGCGUUACCA | 999 | UUAAAUAUGG | 1000 |
| Pr-25 | UAUUUAA | | UAACGCCUG | |
| HNF4A.NM_175914- | CCUUAAUCAGAU | 1001 | AAUGACGAUC | 1002 |
| Pr-27 | CGUCAUU | | UGAUUAAGG | |
| HNF4A.NM_175914- | GCUUUGGGCCCG | 1003 | UUUCUUACGG | 1004 |
| Pr-31 | UAAGAAA | | GCCCAAAGC | |
| HNF4A.NM_175914- | GCCCGGUUAUCU | 1005 | AUCAAUAAGA | 1006 |
| Pr-32 | UAUUGAU | | UAACCGGGC | |
| HNF4A.NM_175914- | GGAUAGCAUUCC | 1007 | UUACAGUGGA | 1008 |
| Pr-36 | ACUGUAA | | AUGCUAUCC | |
| HNF4A.NM_175914- | GUGGAUACGUU | 1009 | UACUCUUUAA | 1010 |
| Pr-39 | AAAGAGUA | | CGUAUCCAC | |

TABLE 21
Sequences of HNF4A-saRNAs (with 3′ overhang)

| | | Sense | | Antisense |
|---|---|---|---|---|
| saRNA ID | Sense (passenger) | SEQ ID | Antisense (guide) | SEQ ID |
| HNF4A.NM_000457- | UCAGUUAUCAAUUGU | 1011 | UUGUACAAUUGAUAA | 1012 |
| Pr-3′ | ACAAUU | | CUGAUU | |
| HNF4A.NM_000457- | GGCAUUCUAACUGAU | 1013 | UAGUAUCAGUUAGAA | 1014 |
| Pr-16′ | ACUAUU | | UGCCUU | |
| HNF4A.NM_000457- | GCGGUUAUGUCUCCA | 1015 | UGGUUGGAGACAUAA | 1016 |
| Pr-19′ | ACCAUU | | CCGCUU | |
| HNF4A.NM_000457- | UGAGUUGGCUUCGUA | 1017 | UAACUACGAAGCCAA | 1018 |
| Pr-23′ | GUUAUU | | CUCAUU | |
| HNF4A.NM_000457- | GAGAAAUGCGGUUAU | 1019 | AGACAUAACCGCAUU | 1020 |
| Pr-26′ | GUCUUU | | UCUCUU | |
| HNF4A.NM_000457- | CGAGAAAUGCGGUUA | 1021 | GACAUAACCGCAUUU | 1022 |
| Pr-36′ | UGUCUU | | CUCGUU | |
| HNF4A.NM_000457- | AGGCAUUCUAACUGA | 1023 | AGUAUCAGUUAGAAU | 1024 |
| Pr-37′ | UACUUU | | GCCUUU | |
| HNF4A.NM_175914- | AGAUCGUCAUUCAAA | 1025 | UAUCUUUGAAUGACG | 1026 |
| Pr-1′ | GAUAUU | | AUCUUU | |
| HNF4A.NM_175914- | GCGUUGUUUCCUUGA | 1027 | UUGUUCAAGGAAACA | 1028 |
| Pr-3′ | ACAAUU | | ACGCUU | |
| HNF4A.NM_175914- | CGUUGUUUCCUUGAA | 1029 | AUUGUUCAAGGAAAC | 1030 |
| Pr-4′ | CAAUUU | | AACGUU | |
| HNF4A.NM_175914- | UGUGUCAUUCCACGA | 1031 | AAAUUCGUGGAAUGA | 1032 |
| Pr-7′ | AUUUUU | | CACAUU | |
| HNF4A.NM_175914- | GGCGUUACCAUAUUU | 1033 | AUUUAAAUAUGGUAA | 1034 |
| Pr-8′ | AAAUUU | | CGCCUU | |
| HNF4A.NM_175914- | AAUUAAACCUUGAGA | 1035 | UUCAUCUCAAGGUUU | 1036 |
| Pr-9′ | UGAAUU | | AAUUUU | |
| HNF4A.NM_175914- | GAGCUUUGGGCCCGU | 1037 | UCUUACGGGCCCAAA | 1038 |
| Pr-15′ | AAGAUU | | GCUCUU | |
| HNF4A.NM_175914- | GGUGGAUACGUUAAA | 1039 | ACUCUUUAACGUAUC | 1040 |
| Pr-16′ | GAGUUU | | CACCUU | |
| HNF4A.NM_175914- | GCGUUACCAUAUUUA | 1041 | AAUUUAAAUAUGGUA | 1042 |
| Pr-19′ | AAUUUU | | ACGCUU | |
| HNF4A.NM_175914- | GGAGAACUGUGUUUA | 1043 | UCACUAAACACAGUU | 1044 |
| Pr-22′ | GUGAUU | | CUCCUU | |
| HNF4A.NM_175914- | CAGGCGUUACCAUAU | 1045 | UUAAAUAUGGUAACG | 1046 |
| Pr-25′ | UUAAUU | | CCUGUU | |
| HNF4A.NM_175914- | CCUUAAUCAGAUCGU | 1047 | AAUGACGAUCUGAUU | 1048 |
| Pr-27′ | CAUUUU | | AAGGUU | |
| HNF4A.NM_175914- | GCUUUGGGCCCGUAA | 1049 | UUUCUUACGGGCCCA | 1050 |
| Pr-31′ | GAAAUU | | AAGCUU | |
| HNF4A.NM_175914- | GCCCGGUUAUCUUAU | 1051 | AUCAAUAAGAUAACC | 1052 |
| Pr-32′ | UGAUUU | | GGGCUU | |
| HNF4A.NM_175914- | GGAUAGCAUUCCACU | 1053 | UUACAGUGGAAUGCU | 1054 |
| Pr-36′ | GUAAUU | | AUCCUU | |
| HNF4A.NM_175914- | GUGGAUACGUUAAAG | 1055 | UACUCUUUAACGUAU | 1056 |
| Pr-39′ | AGUAUU | | CCACUU | |

HNF4A gene encodes HNF4A protein which is a member of the nuclear receptor family of transcription factors and is the most abundant DNA-binding protein in the liver, where it regulates genes largely involved in the hepatic gluconeogenic program and lipid metabolism. HNF4A is a liver master regulator. It is involved in non-alcoholic fatty liver disease (NAFLD), liver fibrosis, cirrhosis, hepatocellular carcinoma (HCC), diabetes, inflammation, drug metabolism, encephalopathy, and bleeding disorders. HNF4A haplotypes are associated with high serum lipids and metabolic syndrome. NHF4A polymorphisms are associated with cytochrome P450 activity. Reduced HNF4A expression levels in biopsies correlate with fibrosis and progression to HCC. Low HNF4A correlates with aggressive HCC phenotype and poor prognosis. Mutations in HNF4A promoter and response elements are linked to Mature Onset Diabetes of the Young (MODY). In the pancreas HNF-4a is also a master regulator, controlling an estimated 11% of islet genes. HNF-4a gene mutations are linked to maturity-onset diabetes of the young, type 1 (MODY1), type 2 diabetes, and hyperinsulinaemic hypoglycaemia (Chandra et al., Nature, vol. 495:394 (2013), the contents of which are incorporated herein by reference in their entirety).

In one embodiment, provided is a method of modulating HNF4A protein levels comprising administering HNF4A-saRNA of the present invention, wherein the HNF4A-saRNA targets an antisense RNA transcript of HNF4A gene. In one embodiment, HNF4A protein level is increased by at least 20, 30, 40%, or at least 45, 50, 55, 60, 65, 70, 75%, or at least 80% in the presence of the HNF4A-saRNA of the present invention compared to HNF4A protein level in the absence of the HNF4A-saRNA of the present invention. In a further embodiment, HNF4A protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the HNF4A-saRNA of the present invention compared to HNF4A protein level in the absence of the HNF4A-saRNA of the present invention.

In another embodiment, provided is a method of regulating liver metabolism comprising administering HNF4A-saRNA of the present invention, wherein the HNF4A-saRNA targets an antisense RNA transcript of HNF4A gene. In another embodiment, provided is a method of treating MODY, diabetes, NAFLD, HCC, liver fibrosis, cirrhosis, HCC, inflammation, encephalopathy, bleeding disorders, or hyperinsulinaemic hypoglycemia by administering the HNF4A-saRNA of the present invention, wherein the HNF4A-saRNA targets an antisense RNA transcript of HNF4A gene, and wherein the symptoms of MODY, diabetes, NAFLD, HCC, liver fibrosis, cirrhosis, HCC, inflammation, encephalopathy, bleeding disorders, or hyperinsulinaemic hypoglycemia are reduced.

In one embodiment, the HNF4A-saRNA of the present invention is an antisense sequence such as, but not limited to, SEQ ID NO: 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 3844, 3846, 3848, 3850, 3852, 3854, 3856, 3858, 3860, 3862, 3864, 3866, 3868, 3870, 3872, 3874, 3876, 3878, 3880, 3882, 3884, 3886, 3888, 3890, 3892, 3894, 3896, 3898, 3900, 3902, 3904, 3906, 3908, 3910, 3912, 3914, 3916, 3918, 3920, 3922, 3924, 3926, 3928, 3930, 3932, 3934, 3936, 3938, 3940, 3942, 3944, 3946, 3948, 3950, 3952, 3954, 3956, 3958, 3960, 3962, 3964, 3966, 3968, 3970, 3972, 3974, 3976, 3978, 3980, 3982, 3984, 3986, 3988, 3990, 3992, 3994, 3996, 3998, 4000, 4002, 4004, 4006, 4008, 4010, 4012, 4014, 4016, 4018, 4020, 4022, 4024, 4026, 4028, 4030, 4032, 4034, 4036, 4038 and 4040. As a non-limiting example, these HNF4A-saRNA sequences may be used to modulate HNF4A protein levels, modulating liver metabolism and/or treating MODY, diabetes, NAFLD, HCC, liver fibrosis, cirrhosis, HCC, inflammation, encephalopathy, bleeding disorders, or hyperinsulinaemic hypoglycemia.

In one embodiment, the HNF4A-saRNA of the present invention is an saRNA duplex. The saRNA duplex may be a pair of sense and antisense sequences such as, but not limited to, SEQ ID NOs: 965 and 966; SEQ ID NOs: 967 and 968; SEQ ID NOs: 969 and 970; SEQ ID NOs: 971 and 972; SEQ ID NOs: 973 and 974; SEQ ID NOs: 975 and 976; SEQ ID NOs: 977 and 978; SEQ ID NOs: 979 and 980; SEQ ID NOs: 981 and 982; SEQ ID NOs: 983 and 984; SEQ ID NOs: 985 and 986; SEQ ID NOs: 987 and 988; SEQ ID NOs: 989 and 990; SEQ ID NOs: 991 and 992; SEQ ID NOs: 993 and 994; SEQ ID NOs: 995 and 996; SEQ ID NOs: 997 and 998; SEQ ID NOs: 999 and 1000; SEQ ID NOs: 1001 and 1002; SEQ ID NOs: 1003 and 1004; SEQ ID NOs: 1005 and 1006; SEQ ID NOs: 1007 and 1008; SEQ ID NOs: 1009 and 1010; SEQ ID NOs: 1011 and 1012; SEQ ID NOs: 1013 and 1014; SEQ ID NOs: 1015 and 1016; SEQ ID NOs: 1017 and 1018; SEQ ID NOs: 1019 and 1020; SEQ ID NOs: 1021 and 1022; SEQ ID NOs: 1023 and 1024; SEQ ID NOs: 1025 and 1026; SEQ ID NOs: 1027 and 1028; SEQ ID NOs: 1029 and 1030; SEQ ID NOs: 1031 and 1032; SEQ ID NOs: 1033 and 1034; SEQ ID NOs: 1035 and 1036; SEQ ID NOs: 1037 and 1038; SEQ ID NOs: 1039 and 1040; SEQ ID NOs: 1041 and 1042; SEQ ID NOs: 1043 and 1044; SEQ ID NOs: 1045 and 1046; SEQ ID NOs: 1047 and 1048; SEQ ID NOs: 1049 and 1050; SEQ ID NOs: 1051 and 1052; SEQ ID NOs: 1053 and 1054; SEQ ID NOs: 1055 and 1056; SEQ ID NOs: 3843 and 3844; SEQ ID NOs: 3845 and 3846; SEQ ID NOs: 3847 and 3848; SEQ ID NOs: 3849 and 3850; SEQ ID NOs: 3851 and 3852; SEQ ID NOs: 3853 and 3854; SEQ ID NOs: 3855 and 3856; SEQ ID NOs: 3857 and 3858; SEQ ID NOs: 3859 and 3860; SEQ ID NOs: 3861 and 3862; SEQ ID NOs: 3863 and 3864; SEQ ID NOs: 3865 and 3866; SEQ ID NOs: 3867 and 3868; SEQ ID NOs: 3869 and 3870; SEQ ID NOs: 3871 and 3872; SEQ ID NOs: 3873 and 3874; SEQ ID NOs: 3875 and 3876; SEQ ID NOs: 3877 and 3878; SEQ ID NOs: 3879 and 3880; SEQ ID NOs: 3881 and 3882; SEQ ID NOs: 3883 and 3884; SEQ ID NOs: 3885 and 3886; SEQ ID NOs: 3887 and 3888; SEQ ID NOs: 3889 and 3890; SEQ ID NOs: 3891 and 3892; SEQ ID NOs: 3893 and 3894; SEQ ID NOs: 3895 and 3896; SEQ ID NOs: 3897 and 3898; SEQ ID NOs: 3899 and 3900; SEQ ID NOs: 3901 and 3902; SEQ ID NOs: 3903 and 3904; SEQ ID NOs: 3905 and 3906; SEQ ID NOs: 3907 and 3908; SEQ ID NOs: 3909 and 3910; SEQ ID NOs: 3911 and 3912; SEQ ID NOs: 3913 and 3914; SEQ ID NOs: 3915 and 3916; SEQ ID NOs: 3917 and 3918; SEQ ID NOs: 3919 and 3920; SEQ ID NOs: 3921 and 3922; SEQ ID NOs: 3923 and 3924; SEQ ID NOs: 3925 and 3926; SEQ ID NOs: 3927 and 3928; SEQ ID NOs: 3929 and 3930; SEQ ID NOs: 3931 and 3932; SEQ ID NOs: 3933 and 3934; SEQ ID NOs: 3935 and 3936; SEQ ID NOs: 3937 and 3938; SEQ ID NOs: 3939 and 3940; SEQ ID NOs: 3941 and 3942; SEQ ID NOs: 3943 and 3944; SEQ ID NOs: 3945 and 3946; SEQ ID NOs: 3947 and 3948; SEQ ID NOs: 3949 and 3950; SEQ ID NOs: 3951 and 3952; SEQ ID NOs: 3953 and 3954; SEQ ID NOs: 3955 and 3956; SEQ ID NOs: 3957 and 3958; SEQ ID NOs: 3959 and 3960; SEQ ID NOs: 3961 and 3962; SEQ ID NOs: 3963 and 3964; SEQ ID NOs: 3965 and 3966; SEQ ID NOs: 3967 and 3968; SEQ ID NOs: 3969 and 3970; SEQ ID NOs: 3971 and 3972; SEQ ID NOs: 3973 and 3974; SEQ ID NOs: 3975 and 3976; SEQ ID NOs: 3977 and 3978; SEQ ID NOs: 3979 and 3980; SEQ ID NOs: 3981 and 3982; SEQ ID NOs: 3983 and 3984; SEQ ID NOs: 3985 and 3986; SEQ ID NOs: 3987 and 3988; SEQ ID NOs: 3989 and 3990; SEQ ID NOs: 3991 and 3992; SEQ ID NOs: 3993 and 3994; SEQ ID NOs: 3995 and 3996; SEQ ID NOs: 3997 and 3998; SEQ ID NOs: 3999 and 4000; SEQ ID NOs: 4001 and 4002; SEQ ID NOs: 4003 and 4004; SEQ ID NOs: 4005 and 4006; SEQ ID NOs: 4007 and 4008; SEQ ID NOs: 4009 and 4010; SEQ ID NOs: 4011 and 4012; SEQ ID NOs: 4013 and 4014; SEQ ID NOs: 4015 and 4016; SEQ ID NOs: 4017 and 4018; SEQ ID NOs: 4019 and 4020; SEQ ID NOs: 4021 and 4022; SEQ ID NOs: 4023 and 4024; SEQ ID NOs: 4025 and 4026; SEQ ID NOs: 4027 and 4028; SEQ ID NOs: 4029 and 4030; SEQ ID NOs: 4031 and 4032; SEQ ID NOs: 4033 and 4034; SEQ ID NOs: 4035 and 4036; SEQ ID NOs: 4037 and 4038; SEQ ID NOs: 4039 and 4040. As a non-limiting example, these HNF4A-saRNA sequences which are saRNA duplexes may be used to modulate HNF4A protein levels, modulating liver metabolism and/or treating MODY, diabetes, NAFLD, HCC, liver fibrosis, cirrhosis, HCC, inflammation, encephalopathy, bleeding disorders, or hyperinsulinaemic hypoglycemia.

### IFNG Gene

As another non-limiting example, provided is a method of modulating the expression of IFNG gene comprising administering saRNA of the present invention, wherein the saRNA targets an antisense RNA transcript of IFNG gene. These saRNAs are called IFNG-saRNA. In one embodiment, the expression of IFNG gene is increased by at least 20, 30, 40%, or at least 45, 50, 55, 60, 65, 70, 75%, or at least 80% in the presence of the IFNG-saRNA of the present invention compared to the expression of IFNG gene in the absence of the IFNG-saRNA of the present invention. In a further embodiment, the expression of IFNG gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IFNG-saRNA of the present invention compared to the expression of IFNG gene in the absence of the IFNG-saRNA of the present invention. The modulation of the expression of HNF4A gene may be reflected or determined by the change of IFNG mRNA levels.

IFNG-saRNAs may be single-stranded and comprise 14-30 nucleotides. The sequence of a single-stranded IFNG-saRNA may have at least 60%, 70%, 80% or 90% identity with a sequence selected from the sequences of the antisense strands in Table 22-2. In one embodiment, the single-stranded saRNA comprises a sequence selected from the sequences of the antisense strands in Table 22-2. In one embodiment, the single-stranded IFNG-saRNA may have a 3′ tail. The sequence of a single-stranded IFNG-saRNA with a 3′ tail may have at least 60%, 70%, 80% or 90% identity with a sequence selected from the sequences of the antisense strands in Table 23. In one embodiment, the single-stranded saRNA comprises a sequence selected from the sequences of the antisense strands in Table 23.

IFNG-saRNAs may be double-stranded. The two strands form a duplex and each strand comprises 14-30 nucleotides. The first strand of a double-stranded IFNG-saRNA may have at least 60%, 70%, 80% or 90% identity with a sequence selected from the sequences of the antisense strands in Table 22-2. In one embodiment, the first strand of the double-stranded saRNA comprises a sequence selected from the sequences of the antisense strands in Table 22-2. The second strand of a double-stranded IFNG-saRNA may have at least 60%, 70%, 80% or 90% identity with a sequence selected from the sequences of the sense strands in Table 22-2. In one embodiment, the second strand of the double-stranded saRNA comprises a sequence selected from the sequences of the sense strands in Table 22-2. In one embodiment, the double-stranded IFNG-saRNA may have a 3′ overhang on each strand. The first strand of a double-stranded IFNG-saRNA may have at least 60%, 70%, 80% or 90% identity with a sequence selected from the sequences of the antisense strands in Table 23. In one embodiment, the first strand of the double-stranded saRNA comprises a sequence selected from the sequences of the antisense strands in Table 23. The second strand of a double-stranded IFNG-saRNA may have at least 60%, 70%, 80% or 90% identity with a sequence selected from the sequences of the sense strands in Table 23. In one embodiment, the second strand of the double-stranded saRNA comprises a sequence selected from the sequences of the sense strands in Table 23.

IFNG-saRNAs may be modified or unmodified.

Table 22-1 describes the target gene, the targeted sequence, the genomic location of the targeted sequence and the relative location of IFNG-saRNAs with no 3′ overhang. In Table 22-1, the targeted sequence is defined as a region on the template strand of the target gene which is identical to a region on the target antisense RNA transcript to which the antisense strand of an saRNA hybridizes. In Table 22-1, the relative location is the distance from the 5′ end of the targeted sequence to the TSS. A negative number represents a location upstream of the TSS and a positive number represents a location downstream of the TSS

TABLE 22-1
Sequences of IFNG-saRNAs (with no 3′ overhang)

| | Target | | Targeted | Genomic Location of the | Relative |
|---|---|---|---|---|---|
| saRNA ID | Gene | Targeted sequence | sequence ID | Targeted sequence | Location |
| IFNG.NM_000619- | IFNG | catcgtttccgagagaatt | 1057 | chr12: 68553392 plus | 129 |
| Pr-2 | human | | | strand | |
| IFNG.NM_000619- | IFNG | atcgtttccgagagaatta | 1058 | chr12: 68553393 plus | 128 |
| Pr-3 | human | | | strand | |
| IFNG.NM_000619- | IFNG | attgaagtctcctgaggat | 1059 | chr12: 68553555 plus | −34 |
| Pr-11 | human | | | strand | |
| IFNG.NM_000619- | IFNG | tcgtttccgagagaattaa | 1060 | chr12: 68553394 plus | 127 |
| Pr-16 | human | | | strand | |
| IFNG.NM_000619- | IFNG | ttcatcgtttccgagagaa | 1061 | chr12: 68553390 plus | 131 |
| Pr-35 | human | | | strand | |

TABLE 22-2
Sequences of IFNG-saRNAs (with no 3′ overhang)

| | | | | Antisense SEQ |
|---|---|---|---|---|
| saRNA ID | Sense (passenger) | Sense SEQ ID | Antisense (guide) | ID |
| IFNG.NM_000619- | CAUCGUUUCCGA | 1062 | AAUUCUCUCGGAAA | 1071 |
| Pr-2 | GAGAAUU | | CGAUG | |
| IFNG.NM_000619- | AUCGUUUCCGAG | 1064 | UAAUUCUCUCGGAA | 1073 |
| Pr-3 | AGAAUUA | | ACGAU | |
| IFNG.NM_000619- | AUUGAAGUCUCC | 1066 | AUCCUCAGGAGACU | 1075 |
| Pr- | UGAGGAU | | UCAAU | |
| 11 | | | | |
| IFNG.NM_000619- | UCGUUUCCGAGA | 1068 | UUAAUUCUCUCGGA | 1077 |
| Pr- | GAAUUAA | | AACGA | |
| 16 | | | | |
| IFNG.NM_000619- | UUCAUCGUUUCC | 1070 | UUCUCUCGGAAACG | 1079 |
| Pr- | GAGAGAA | | AUGAA | |
| 35 | | | | |

TABLE 23
Sequences of IFNG-saRNAs (with 3′ overhang)

| | | | | Antisense SEQ |
|---|---|---|---|---|
| saRNA ID | Sense (passenger) | Sense SEQ ID | Antisense (guide) | ID |
| IFNG.NM_000619- | CAUCGUUUCCGA | 1080 | AAUUCUCUCGGAAA | 1081 |
| Pr-2′ | GAGAAUUUU | | CGAUGUU | |
| IFNG.NM_000619- | AUCGUUUCCGAG | 1082 | UAAUUCUCUCGGAA | 1083 |
| Pr-3′ | AGAAUUAUU | | ACGAUUU | |
| IFNG.NM_000619- | AUUGAAGUCUCC | 1084 | AUCCUCAGGAGACU | 1085 |
| Pr-11′ | UGAGGAUUU | | UCAAUUU | |
| IFNG.NM_000619- | UCGUUUCCGAGA | 1086 | UUAAUUCUCUCGGA | 1087 |
| Pr-16′ | GAAUUAAUU | | AACGAUU | |
| IFNG.NM_000619- | UUCAUCGUUUCC | 1088 | UUCUCUCGGAAACG | 1089 |
| Pr-35′ | GAGAGAAUU | | AUGAAUU | |

IFNG gene encodes interferon gamma which is a member of the type II interferon family. Interferon gamma has antiviral, immunoregulatory and anti-tumor activities. It is a potent activator of macrophages, has antiproliferative effects on transformed cells, and can potentiate the antiviral and antitumor effects of the type I interferon. Mutations in IFNG gene cause aplastic anemia (AA), a form of anemia in which the bone marrow does not produce enough numbers of peripheral blood elements. AA is characterized by peripheral pancytopenia and marrow hypoplasia (Dufour et al., British J of Haematology, vol. 126 (5):682 (2004), the contents of which are incorporated herein by reference in their entirety). Mutations in IFNG gene are also involved in susceptibility to congenital HIV, hepatitis C virus, mycobacterium tuberculosis, and tuberous sclerosis.

In one embodiment, provided is a method of modulating interferon gamma protein levels comprising administering IFNG-saRNA of the present invention, wherein the IFNG-saRNA targets an antisense RNA transcript of IFNG gene. In one embodiment, interferon gamma protein level is increased by at least 20, 30, 40%, or at least 45, 50, 55, 60, 65, 70, 75%, or at least 80% in the presence of the IFNG-saRNA of the present invention compared to interferon gamma protein level in the absence of the IFNG-saRNA of the present invention. In a further embodiment, interferon gamma protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IFNG-saRNA of the present invention compared to interferon gamma protein level in the absence of the IFNG-saRNA of the present invention.

In another embodiment, provided is a method of treating aplastic anemia comprising administering IFNG-saRNA of the present invention, wherein the IFNG-saRNA targets an antisense RNA transcript of IFNG gene, and wherein the symptoms of aplastic anemia are reduced.

In another embodiment, provided is a method of reducing a patient's susceptibility to congenital HIV, hepatitis C virus, mycobacterium tuberculosis and tuberous sclerosis, comprising administering IFNG-saRNA of the present invention to a patient in need thereof, wherein the IFNG-saRNA targets an antisense RNA transcript of IFNG gene.

In one embodiment, the IFNG-saRNA of the present invention is an antisense sequence such as, but not limited to, SEQ ID NO: 1071, 1073, 1075, 1077, 1079, 1081, 1083, 1085, 1087, 1089, 4110, 4112, 4114, 4116, 4118, 4120, 4122, 4124, 4126, 4128, 4130, 4132, 4134, 4136, 4138, 4140, 4142, 4144, 4146, 4148, 4150, 4152, 4154, 4156, 4158, 4160, 4162, 4164, 4166, 4168, 4170, 4172, 4174, 4176, 4178, 4180, 4182, 4184, 4186, 4188, 4190, 4192, 4194, 4196, 4198, 4200, 4202, 4204, 4206 and 4208. As a non-limiting example, these IFNG-saRNA sequences may be used to modulate interferon gamma protein levels, treat aplastic anemia and/or reduce a patient's susceptibility to congenital HIV, hepatitis C virus, mycobacterium tuberculosis and tuberous sclerosis.

In one embodiment, the IFNG-saRNA of the present invention is an saRNA duplex. The saRNA duplex may be a pair of sense and antisense sequences such as, but not limited to, SEQ ID NOs: 1062 and 1071; SEQ ID NOs: 1064 and 1073; SEQ ID NOs: 1066 and 1075; SEQ ID NOs: 1068 and 1077; SEQ ID NOs: 1070 and 1079; SEQ ID NOs: 1080 and 1081; SEQ ID NOs: 1082 and 1083; SEQ ID NOs: 1084 and 1085; SEQ ID NOs: 1086 and 1087; SEQ ID NOs: 1088 and 1089; SEQ ID NOs: 4109 and 4110; SEQ ID NOs: 4111 and 4112; SEQ ID NOs: 4113 and 4114; SEQ ID NOs: 4115 and 4116; SEQ ID NOs: 4117 and 4118; SEQ ID NOs: 4119 and 4120; SEQ ID NOs: 4121 and 4122; SEQ ID NOs: 4123 and 4124; SEQ ID NOs: 4125 and 4126; SEQ ID NOs: 4127 and 4128; SEQ ID NOs: 4129 and 4130; SEQ ID NOs: 4131 and 4132; SEQ ID NOs: 4133 and 4134; SEQ ID NOs: 4135 and 4136; SEQ ID NOs: 4137 and 4138; SEQ ID NOs: 4139 and 4140; SEQ ID NOs: 4141 and 4142; SEQ ID NOs: 4143 and 4144; SEQ ID NOs: 4145 and 4146; SEQ ID NOs: 4147 and 4148; SEQ ID NOs: 4149 and 4150; SEQ ID NOs: 4151 and 4152; SEQ ID NOs: 4153 and 4154; SEQ ID NOs: 4155 and 4156; SEQ ID NOs: 4157 and 4158; SEQ ID NOs: 4159 and 4160; SEQ ID NOs: 4161 and 4162; SEQ ID NOs: 4163 and 4164; SEQ ID NOs: 4165 and 4166; SEQ ID NOs: 4167 and 4168; SEQ ID NOs: 4169 and 4170; SEQ ID NOs: 4171 and 4172; SEQ ID NOs: 4173 and 4174; SEQ ID NOs: 4175 and 4176; SEQ ID NOs: 4177 and 4178; SEQ ID NOs: 4179 and 4180; SEQ ID NOs: 4181 and 4182; SEQ ID NOs: 4183 and 4184; SEQ ID NOs: 4185 and 4186; SEQ ID NOs: 4187 and 4188; SEQ ID NOs: 4189 and 4190; SEQ ID NOs: 4191 and 4192; SEQ ID NOs: 4193 and 4194; SEQ ID NOs: 4195 and 4196; SEQ ID NOs: 4197 and 4198; SEQ ID NOs: 4199 and 4200; SEQ ID NOs: 4201 and 4202; SEQ ID NOs: 4203 and 4204; SEQ ID NOs: 4205 and 4206; SEQ ID NOs: 4207 and 4208. As a non-limiting example, these IFNG-saRNA sequences which are saRNA duplexes may be used to modulate interferon gamma protein levels, treat aplastic anemia and/or reduce a patient's susceptibility to congenital HIV, hepatitis C virus, mycobacterium tuberculosis and tuberous sclerosis.

### IL10 Gene

As another non-limiting example, provided is a method of modulating the expression of IL10 gene comprising administering saRNA of the present invention, wherein the saRNA targets an antisense RNA transcript of IL10 gene. These saRNAs are called IL10-saRNA. In one embodiment, the expression of IL10 gene is increased by at least 20, 30, 40%, or at least 45, 50, 55, 60, 65, 70, 75%, or at least 80% in the presence of the IL10-saRNA of the present invention compared to the expression of IL10 gene in the absence of the IL10-saRNA of the present invention. In a further embodiment, the expression of IL10 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL10-saRNA of the present invention compared to the expression of IL10 gene in the absence of the IL10-saRNA of the present invention. The modulation of the expression of IL10 gene may be reflected or determined by the change of IL10 mRNA levels.

IL10-saRNAs may be single-stranded and comprise 14-30 nucleotides. The sequence of a single-stranded IL10-saRNA may have at least 60%, 70%, 80% or 90% identity with a sequence selected from the sequences of the antisense strands in Table 24-2. In one embodiment, the single-stranded saRNA comprises a sequence selected from the sequences of the antisense strands in Table 24-2. In one embodiment, the single-stranded IL10-saRNA may have a 3′ tail. The sequence of a single-stranded IL10-saRNA with a 3′ tail may have at least 60%, 70%, 80% or 90% identity with a sequence selected from the sequences of the antisense strands in Table 25. In one embodiment, the single-stranded saRNA comprises a sequence selected from the sequences of the antisense strands in Table 25.

IL10-saRNAs may be double-stranded. The two strands form a duplex and each strand comprises 14-30 nucleotides. The first strand of a double-stranded IL10-saRNA may have at least 60%, 70%, 80% or 90% identity with a sequence selected from the sequences of the antisense strands in Table 24-2. In one embodiment, the first strand of the double-stranded saRNA comprises a sequence selected from the sequences of the antisense strands in Table 24-2. The second strand of a double-stranded IL10-saRNA may have at least 60%, 70%, 80% or 90% identity with a sequence selected from the sequences of the sense strands in Table 24-2. In one embodiment, the second strand of the double-stranded saRNA comprises a sequence selected from the sequences of the sense strands in Table 24-2. In one embodiment, the double-stranded IL10-saRNA may have a 3′ overhang on each strand. The first strand of a double-stranded IL10-saRNA may have at least 60%, 70%, 80% or 90% identity with a sequence selected from the sequences of the antisense strands in Table 25. In one embodiment, the first strand of the double-stranded saRNA comprises a sequence selected from the sequences of the antisense strands in Table 25. The second strand of a double-stranded IL10-saRNA may have at least 60%, 70%, 80% or 90% identity with a sequence selected from the sequences of the sense strands in Table 25. In one embodiment, the second strand of the double-stranded saRNA comprises a sequence selected from the sequences of the sense strands in Table 25.

IL10-saRNAs may be modified or unmodified.

Table 24-1 describes the target gene, the targeted sequence, the genomic location of the targeted sequence and the relative location of IL10-saRNAs with no 3′ overhang. In Table 24-1, the targeted sequence is defined as a region on the template strand of the target gene which is identical to a region on the target antisense RNA transcript to which the antisense strand of an saRNA hybridizes. In Table 24-1, the relative location is the distance from the 5′ end of the targeted sequence to the TSS. A negative number represents a location upstream of the TSS and a positive number represents a location downstream of the TSS.

TABLE 24-1
Sequences of IL10-saRNAs (with no 3′ overhang)

| | | | | Genomic Location | |
|---|---|---|---|---|---|
| | Target | | Targeted | of the Targeted | Relative |
| saRNA ID | Gene | Targeted sequence | sequence ID | sequence | Location |
| IL10.NM_000572- | IL10 | ggaaagatcccagggatta | 1090 | chr1: 206945148 | 691 |
| Pr-4 | human | | | plus strand | |
| IL10.NM_000572- | IL10 | agaatgaaaccttggatta | 1091 | chr1: 206946715 | −876 |
| Pr-12 | human | | | plus strand | |
| IL10.NM_000572- | IL10 | ggctacctctcttagaata | 1092 | chr1: 206946056 | −217 |
| Pr-15 | human | | | plus strand | |
| IL10.NM_000572- | IL10 | cggagatctcgaagcatgt | 1093 | chr1: 206945646 | 193 |
| Pr-20 | human | | | plus strand | |
| IL10.NM_000572- | IL10 | tcctcagagtcaagttatt | 1094 | chr1: 206944951 | 888 |
| Pr-30 | human | | | plus strand | |
| IL10.NM_000572- | IL10 | ggaggagggttcttatagt | 1095 | chr1: 206945530 | 309 |
| Pr-32 | human | | | plus strand | |
| IL10.NM_000572- | IL10 | gaatcatactcacaaagaa | 1096 | chr 1: 206945602 | 237 |
| Pr-33 | human | | | plus strand | |
| IL10.NM_000572- | IL10 | cagtttagcttggaaagat | 1097 | chr1: 206945137 | 702 |
| Pr-34 | human | | | plus strand | |
| IL10.NM_000572- | IL10 | acaatgggattgagaaata | 1098 | chr1: 206946212 | −373 |
| Pr-37 | human | | | plus strand | |
| IL10.NM_000572- | IL10 | gctggagtctaaagtttaa | 1099 | chr 1: 206946773 | −934 |
| Pr-39 | human | | | plus strand | |
| IL10.NM_000572- | IL10 | agaggattcaacagtgatg | 1100 | chr 1: 206944906 | 933 |
| Pr-40 | human | | | plus strand | |

TABLE 24-2
Sequences of IL10-saRNAs (with no 3′ overhang)

| | | | | Antisense |
|---|---|---|---|---|
| saRNA ID | Sense (passenger) | Sense SEQ ID | Antisense (guide) | SEQ ID |
| IL10.NM_000572- | GGAAAGAUCCCA | 1101 | UAAUCCCUGGGAU | 1102 |
| Pr-4 | GGGAUUA | | CUUUCC | |
| IL10.NM_000572- | AGAAUGAAACCU | 1103 | UAAUCCAAGGUUU | 1104 |
| Pr-12 | UGGAUUA | | CAUUCU | |
| IL10.NM_000572- | GGCUACCUCUCU | 1105 | UAUUCUAAGAGAG | 1106 |
| Pr-15 | UAGAAUA | | GUAGCC | |
| IL10.NM_000572- | CGGAGAUCUCGA | 1107 | ACAUGCUUCGAGA | 1108 |
| Pr-20 | AGCAUGU | | UCUCCG | |
| IL10.NM_000572- | UCCUCAGAGUCA | 1109 | AAUAACUUGACUC | 1110 |
| Pr-30 | AGUUAUU | | UGAGGA | |
| IL10.NM_000572- | GGAGGAGGGUUC | 1111 | ACUAUAAGAACCC | 1112 |
| Pr-32 | UUAUAGU | | UCCUCC | |
| IL10.NM_000572- | GAAUCAUACUCA | 1113 | UUCUUUGUGAGUA | 1114 |
| Pr-33 | CAAAGAA | | UGAUUC | |
| IL10.NM_000572- | CAGUUUAGCUUG | 1115 | AUCUUUCCAAGCU | 1116 |
| Pr-34 | GAAAGAU | | AAACUG | |
| IL10.NM_000572- | ACAAUGGGAUUG | 1117 | UAUUUCUCAAUCC | 1118 |
| Pr-37 | AGAAAUA | | CAUUGU | |
| IL10.NM_000572- | GCUGGAGUCUAA | 1119 | UUAAACUUUAGAC | 1120 |
| Pr-39 | AGUUUAA | | UCCAGC | |
| IL10.NM_000572- | AGAGGAUUCAAC | 1121 | CAUCACUGUUGAA | 1122 |
| Pr-40 | AGUGAUG | | UCCUCU | |

TABLE 25
Sequences of IL10-saRNAs (with 3′ overhang)

| | | | | Antisense |
|---|---|---|---|---|
| saRNA ID | Sense (passenger) | Sense SEQ ID | Antisense (guide) | SEQ ID |
| IL10.NM_000572- | GGAAAGAUCCCAG | 1123 | UAAUCCCUGGGAU | 1124 |
| Pr-4′ | GGAUUAUU | | CUUUCCUU | |
| IL10.NM_000572- | AGAAUGAAACCU | 1125 | UAAUCCAAGGUUU | 1126 |
| Pr-12′ | UGGAUUAUU | | CAUUCUUU | |
| IL10.NM_000572- | GGCUACCUCUCUU | 1127 | UAUUCUAAGAGAG | 1128 |
| Pr-15′ | AGAAUAUU | | GUAGCCUU | |
| IL10.NM_000572- | CGGAGAUCUCGAA | 1129 | ACAUGCUUCGAGA | 1130 |
| Pr-20′ | GCAUGUUU | | UCUCCGUU | |
| IL10.NM_000572- | UCCUCAGAGUCAA | 1131 | AAUAACUUGACUC | 1132 |
| Pr-30′ | GUUAUUUU | | UGAGGAUU | |
| IL10.NM_000572- | GGAGGAGGGUUC | 1133 | ACUAUAAGAACCC | 1134 |
| Pr-32′ | UUAUAGUUU | | UCCUCCUU | |
| IL10.NM_000572- | GAAUCAUACUCAC | 1135 | UUCUUUGUGAGUA | 1136 |
| Pr-33′ | AAAGAAUU | | UGAUUCUU | |
| IL10.NM_000572- | CAGUUUAGCUUG | 1137 | AUCUUUCCAAGCU | 1138 |
| Pr-34′ | GAAAGAUUU | | AAACUGUU | |
| IL10.NM_000572- | ACAAUGGGAUUG | 1139 | UAUUUCUCAAUCC | 1140 |
| Pr-37′ | AGAAAUAUU | | CAUUGUUU | |
| IL10.NM_000572- | GCUGGAGUCUAA | 1141 | UUAAACUUUAGAC | 1142 |
| Pr-39′ | AGUUUAAUU | | UCCAGCUU | |
| IL10.NM_000572- | AGAGGAUUCAAC | 1143 | CAUCACUGUUGAA | 1144 |
| Pr-40′ | AGUGAUGUU | | UCCUCUUU | |

IL10 gene encodes interleukin 10 protein which is a cytokine produced primarily by monocytes and also by lymphocytes. IL10 protein is important in immunoregulation and inflammation. It down-regulates the expression of Th1 cytokines, MHC class II Ags, and costimulatory molecules on macrophages. It also enhances B cell survival, proliferation, and antibody production. It can block NF-kappa B activity, and is involved in the regulation of the JAK-STAT signaling pathway. Mutations in IL10 gene cause increased susceptibility to congenital HIV, graft-versus-host disease, and rheumatoid arthritis (Lard et al., Arthritis & Rheumatism, vol. 48 (7):1841 (2003), the contents of which are incorporated herein by reference in their entirety).

In one embodiment, provided is a method of modulating interleukin 10 protein levels comprising administering IL10-saRNA of the present invention, wherein the IL10-saRNA targets an antisense RNA transcript of IL10 gene. In one embodiment, interferon gamma protein level is increased by at least 20, 30, 40%, or at least 45, 50, 55, 60, 65, 70, 75%, or at least 80% in the presence of the IL10-saRNA of the present invention compared to interleukin 10 protein level in the absence of the IL10-saRNA of the present invention. In a further embodiment, interleukin 10 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL10-saRNA of the present invention compared to interleukin 10 protein level in the absence of the IL10-saRNA of the present invention.

In another embodiment, provided is a method of reducing a patient's susceptibility to congenital HIV or graft-versus-host disease, comprising administering the IL10-saRNA of the present invention to a patient in need thereof, wherein the IL10-saRNA targets an antisense RNA transcript of IL10 gene.

In another embodiment, provided is a method of treating graft-versus-host disease or rheumatoid arthritis comprising administering IL10-saRNA of the present invention, wherein the IL10-saRNA targets an antisense RNA transcript of IL10 gene, and wherein the symptoms of graft-versus-host disease or rheumatoid arthritis are reduced.

In one embodiment, the IL10-saRNA of the present invention is an antisense sequence such as, but not limited to, SEQ ID NO: 1102, 1104, 1106, 1108, 1110, 1112, 1114, 1116, 1118, 1120, 1122, 1124, 1126, 1128, 1130, 1132, 1134, 1136, 1138, 1140, 1142, 1144, 4210, 4212, 4214, 4216, 4218, 4220, 4222, 4224, 4226, 4228, 4230, 4232, 4234, 4236, 4238, 4240, 4242, 4244, 4246, 4248, 4250, 4252, 4254, 4256, 4258, 4260, 4262, 4264, 4266, 4268, 4270, 4272, 4274, 4276, 4278, 4280, 4282, 4284, 4286, 4288, 4290, 4292, 4294, 4296, 4298, 4300, 4302, 4304, 4306 and 4308. As a non-limiting example, these IL10-saRNA sequences may be used to modulate IL10 protein levels, treating graft-versus-host disease or rheumatoid arthritis and/or reduce a patient's susceptibility to congenital HIV or graft-versus-host disease.

In one embodiment, the IL10-saRNA of the present invention is an saRNA duplex. The saRNA duplex may be a pair of sense and antisense sequences such as, but not limited to, SEQ ID NOs: 1101 and 1102; SEQ ID NOs: 1103 and 1104; SEQ ID NOs: 1105 and 1106; SEQ ID NOs: 1107 and 1108; SEQ ID NOs: 1109 and 1110; SEQ ID NOs: 1111 and 1112; SEQ ID NOs: 1113 and 1114; SEQ ID NOs: 1115 and 1116; SEQ ID NOs: 1117 and 1118; SEQ ID NOs: 1119 and 1120; SEQ ID NOs: 1121 and 1122; SEQ ID NOs: 1123 and 1124; SEQ ID NOs: 1125 and 1126; SEQ ID NOs: 1127 and 1128; SEQ ID NOs: 1129 and 1130; SEQ ID NOs: 1131 and 1132; SEQ ID NOs: 1133 and 1134; SEQ ID NOs: 1135 and 1136; SEQ ID NOs: 1137 and 1138; SEQ ID NOs: 1139 and 1140; SEQ ID NOs: 1141 and 1142; SEQ ID NOs: 1143 and 1144; SEQ ID NOs: 4209 and 4210; SEQ ID NOs: 4211 and 4212; SEQ ID NOs: 4213 and 4214; SEQ ID NOs: 4215 and 4216; SEQ ID NOs: 4217 and 4218; SEQ ID NOs: 4219 and 4220; SEQ ID NOs: 4221 and 4222; SEQ ID NOs: 4223 and 4224; SEQ ID NOs: 4225 and 4226; SEQ ID NOs: 4227 and 4228; SEQ ID NOs: 4229 and 4230; SEQ ID NOs: 4231 and 4232; SEQ ID NOs: 4233 and 4234; SEQ ID NOs: 4235 and 4236; SEQ ID NOs: 4237 and 4238; SEQ ID NOs: 4239 and 4240; SEQ ID NOs: 4241 and 4242; SEQ ID NOs: 4243 and 4244; SEQ ID NOs: 4245 and 4246; SEQ ID NOs: 4247 and 4248; SEQ ID NOs: 4249 and 4250; SEQ ID NOs: 4251 and 4252; SEQ ID NOs: 4253 and 4254; SEQ ID NOs: 4255 and 4256; SEQ ID NOs: 4257 and 4258; SEQ ID NOs: 4259 and 4260; SEQ ID NOs: 4261 and 4262; SEQ ID NOs: 4263 and 4264; SEQ ID NOs: 4265 and 4266; SEQ ID NOs: 4267 and 4268; SEQ ID NOs: 4269 and 4270; SEQ ID NOs: 4271 and 4272; SEQ ID NOs: 4273 and 4274; SEQ ID NOs: 4275 and 4276; SEQ ID NOs: 4277 and 4278; SEQ ID NOs: 4279 and 4280; SEQ ID NOs: 4281 and 4282; SEQ ID NOs: 4283 and 4284; SEQ ID NOs: 4285 and 4286; SEQ ID NOs: 4287 and 4288; SEQ ID NOs: 4289 and 4290; SEQ ID NOs: 4291 and 4292; SEQ ID NOs: 4293 and 4294; SEQ ID NOs: 4295 and 4296; SEQ ID NOs: 4297 and 4298; SEQ ID NOs: 4299 and 4300; SEQ ID NOs: 4301 and 4302; SEQ ID NOs: 4303 and 4304; SEQ ID NOs: 4305 and 4306; SEQ ID NOs: 4307 and 4308. As a non-limiting example, these IL10-saRNA sequences which are saRNA duplexes may be used to modulate IL10 protein levels, treating graft-versus-host disease or rheumatoid arthritis and/or reduce a patient's susceptibility to congenital HIV or graft-versus-host disease.

### IL2 Gene

As another non-limiting example, provided is a method of modulating the expression of IL2 gene comprising administering saRNA of the present invention, wherein the saRNA targets an antisense RNA transcript of IL2 gene. These saRNAs are called IL2-saRNA. In one embodiment, the expression of IL2 gene is increased by at least 20, 30, 40%, or at least 45, 50, 55, 60, 65, 70, 75%, or at least 80% in the presence of the IL2-saRNA of the present invention compared to the expression of IL2 gene in the absence of the IL2-saRNA of the present invention. In a further embodiment, the expression of IL2 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL2-saRNA of the present invention compared to the expression of IL2 gene in the absence of the IL2-saRNA of the present invention. The modulation of the expression of IL2 gene may be reflected or determined by the change of IL2 mRNA levels.

IL2-saRNAs may be single-stranded and comprise 14-30 nucleotides. The sequence of a single-stranded IL2-saRNA may have at least 60%, 70%, 80% or 90% identity with a sequence selected from the sequences of the antisense strands in Table 26-2. In one embodiment, the single-stranded saRNA comprises a sequence selected from the sequences of the antisense strands in Table 26-2. In one embodiment, the single-stranded IL2-saRNA may have a 3′ tail. The sequence of a single-stranded IL2-saRNA with a 3′ tail may have at least 60%, 70%, 80% or 90% identity with a sequence selected from the sequences of the antisense strands in Table 27. In one embodiment, the single-stranded saRNA comprises a sequence selected from the sequences of the antisense strands in Table 27.

IL2-saRNAs may be double-stranded. The two strands form a duplex and each strand comprises 14-30 nucleotides. The first strand of a double-stranded IL2-saRNA may have at least 60%, 70%, 80% or 90% identity with a sequence selected from the sequences of the antisense strands in Table 26-2. In one embodiment, the first strand of the double-stranded saRNA comprises a sequence selected from the sequences of the antisense strands in Table 26-2. The second strand of a double-stranded IL2-saRNA may have at least 60%, 70%, 80% or 90% identity with a sequence selected from the sequences of the sense strands in Table 26-2. In one embodiment, the second strand of the double-stranded saRNA comprises a sequence selected from the sequences of the sense strands in Table 26-2. In one embodiment, the double-stranded IL2-saRNA may have a 3′ overhang on each strand. The first strand of a double-stranded IL2-saRNA may have at least 60%, 70%, 80% or 90% identity with a sequence selected from the sequences of the antisense strands in Table 27. In one embodiment, the first strand of the double-stranded saRNA comprises a sequence selected from the sequences of the antisense strands in Table 27. The second strand of a double-stranded IL2-saRNA may have at least 60%, 70%, 80% or 90% identity with a sequence selected from the sequences of the sense strands in Table 27. In one embodiment, the second strand of the double-stranded saRNA comprises a sequence selected from the sequences of the sense strands in Table 27.

IL2-saRNAs may be modified or unmodified.

Table 26-1 describes the target gene, the targeted sequence, the genomic location of the targeted sequence and the relative location of IL2-saRNAs with no 3′ overhang. In Table 26-1, the targeted sequence is defined as a region on the template strand of the target gene which is identical to a region on the target antisense RNA transcript to which the antisense strand of an saRNA hybridizes. In Table 26-1, the relative location is the distance from the 5′ end of the targeted sequence to the TSS. A negative number represents a location upstream of the TSS and a positive number represents a location downstream of the TSS.

TABLE 26-1
Sequences of IL2-saRNAs (with no 3′ overhang)

| | | | Targeted | | |
|---|---|---|---|---|---|
| | Target | | sequence | Genomic Location of the | Relative |
| saRNA ID | Gene | Targeted sequence | ID | Targeted sequence | Location |
| IL2.NM_000586- | IL2 | gaattgtcttatcgataaa | 1145 | chr4: 123377045 plus strand | 605 |
| Pr-1 | human | | | | |
| IL2.NM_000586- | IL2 | aggtgatagctctaattca | 1146 | chr4: 123377871 plus strand | −221 |
| Pr-4 | human | | | | |
| IL2.NM_000586- | IL2 | ggtgatagctctaattcat | 1147 | chr4: 123377872 plus strand | −222 |
| Pr-5 | human | | | | |
| IL2.NM_000586- | IL2 | gaaatgatctccagctaga | 1148 | chr4: 123377386 plus strand | 264 |
| Pr-11 | human | | | | |
| IL2.NM_000586- | IL2 | ttagaattgtcttatcgat | 1149 | chr4: 123377042 plus strand | 608 |
| Pr-15 | human | | | | |

TABLE 26-2
Sequences of IL2-saRNAs (with no 3′ overhang)

| | | Sense SEQ | | Antisense |
|---|---|---|---|---|
| saRNA ID | Sense (passenger) | ID | Antisense (guide) | SEQ ID |
| IL2.NM_000586- | GAAUUGUCUUAUCGAU | 1150 | UUUAUCGAUAAGACAA | 1151 |
| Pr-1 | AAA | | UUC | |
| IL2.NM_000586- | AGGUGAUAGCUCUAAU | 1152 | UGAAUUAGAGCUAUCA | 1153 |
| Pr-4 | UCA | | CCU | |
| IL2.NM_000586- | GGUGAUAGCUCUAAUU | 1154 | AUGAAUUAGAGCUAUC | 1155 |
| Pr-5 | CAU | | ACC | |
| IL2.NM_000586- | GAAAUGAUCUCCAGCU | 1156 | UCUAGCUGGAGAUCAU | 1157 |
| Pr-11 | AGA | | UUC | |
| IL2.NM_000586- | UUAGAAUUGUCUUAUC | 1158 | AUCGAUAAGACAAUUC | 1159 |
| Pr-15 | GAU | | UAA | |

TABLE 27
Sequences of IL2-saRNAs (with 3′ overhang)

| | | Sense SEQ | | Antisense |
|---|---|---|---|---|
| saRNA ID | Sense (passenger) | ID | Antisense (guide) | SEQ ID |
| IL2.NM_000586- | GAAUUGUCUUAUCGA | 1160 | UUUAUCGAUAAGACAA | 1161 |
| Pr-1′ | UAAAUU | | UUCUU | |
| IL2.NM_000586- | AGGUGAUAGCUCUAA | 1162 | UGAAUUAGAGCUAUCA | 1163 |
| Pr-4′ | UUCAUU | | CCUUU | |
| IL2.NM_000586- | GGUGAUAGCUCUAAU | 1164 | AUGAAUUAGAGCUAUC | 1165 |
| Pr-5′ | UCAUUU | | ACCUU | |
| IL2.NM_000586- | GAAAUGAUCUCCAGC | 1166 | UCUAGCUGGAGAUCAU | 1167 |
| Pr-11′ | UAGAUU | | UUCUU | |
| IL2.NM_000586- | UUAGAAUUGUCUUAU | 1168 | AUCGAUAAGACAAUUC | 1169 |
| Pr-15′ | CGAUUU | | UAAUU | |

IL2 gene encodes interleukin 2, also known as T-cell growth factor, a secreted cytokine important for the proliferation of T and B lymphocytes. Interleukin 2 is produced by T-cells in response to antigenic or mitogenic stimulation. It is required for T-cell proliferation and other activities crucial to regulation of the immune response. It stimulates B-cells, monocytes, lymphokine-activated killer cells, natural killer cells, and glioma cells (Holbrook et al., Proc. Natl. Acad. Sci. USA, vol. 81:1634 (1984), the contents of which are incorporated herein by reference in their entirety). Mutations of IL2 gene may cause T-cell acute lymphoblastic leukemia.

In one embodiment, provided is a method of modulating interleukin 2 protein levels comprising administering IL2-saRNA of the present invention, wherein the IL2-saRNA targets an antisense RNA transcript of IL2 gene. In one embodiment, interferon gamma protein level is increased by at least 20, 30, 40%, or at least 45, 50, 55, 60, 65, 70, 75%, or at least 80% in the presence of the IL2-saRNA of the present invention compared to interleukin 2 protein level in the absence of the IL2-saRNA of the present invention. In a further embodiment, interleukin 2 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL2-saRNA of the present invention compared to interleukin 2 protein level in the absence of the IL2-saRNA of the present invention.

In another embodiment, provided is a method of regulating T cell levels comprising administering IL2-saRNA of the present invention, wherein the IL2-saRNA targets an antisense RNA transcript of IL2 gene. In another embodiment, provided is a method of treating T-cell acute lymphoblastic leukemia, comprising administering IL2-saRNA of the present invention, wherein the IL2-saRNA targets an antisense RNA transcript of IL2 gene.

In one embodiment, the IL2-saRNA of the present invention is an antisense sequence such as, but not limited to, SEQ ID NO: 1151, 1153, 1155, 1157, 1159, 1161, 1163, 1165, 1167, 1169, 4510, 4512, 4514, 4516, 4518, 4520, 4522, 4524, 4526, 4528, 4530, 4532, 4534, 4536, 4538, 4540, 4542, 4544, 4546, 4548, 4550, 4552, 4554, 4556, 4558, 4560, 4562, 4564, 4566, 4568, 4570, 4572, 4574, 4576, 4578, 4580, 4582, 4584, 4586, 4588, 4590, 4592, 4594, 4596, 4598, 4600, 4602, 4604, 4606 and 4608. As a non-limiting example, these IL2-saRNA sequences may be used to modulate IL2 protein levels, regulating T cell levels and/or treating T-cell acute lymphoblastic leukemia.

In one embodiment, the IL2-saRNA of the present invention is an saRNA duplex. The saRNA duplex may be a pair of sense and antisense sequences such as, but not limited to, SEQ ID NOs: 1150 and 1151; SEQ ID NOs: 1152 and 1153; SEQ ID NOs: 1154 and 1155; SEQ ID NOs: 1156 and 1157; SEQ ID NOs: 1158 and 1159; SEQ ID NOs: 1160 and 1161; SEQ ID NOs: 1162 and 1163; SEQ ID NOs: 1164 and 1165; SEQ ID NOs: 1166 and 1167; SEQ ID NOs: 1168 and 1169; SEQ ID NOs: 4509 and 4510; SEQ ID NOs: 4511 and 4512; SEQ ID NOs: 4513 and 4514; SEQ ID NOs: 4515 and 4516; SEQ ID NOs: 4517 and 4518; SEQ ID NOs: 4519 and 4520; SEQ ID NOs: 4521 and 4522; SEQ ID NOs: 4523 and 4524; SEQ ID NOs: 4525 and 4526; SEQ ID NOs: 4527 and 4528; SEQ ID NOs: 4529 and 4530; SEQ ID NOs: 4531 and 4532; SEQ ID NOs: 4533 and 4534; SEQ ID NOs: 4535 and 4536; SEQ ID NOs: 4537 and 4538; SEQ ID NOs: 4539 and 4540; SEQ ID NOs: 4541 and 4542; SEQ ID NOs: 4543 and 4544; SEQ ID NOs: 4545 and 4546; SEQ ID NOs: 4547 and 4548; SEQ ID NOs: 4549 and 4550; SEQ ID NOs: 4551 and 4552; SEQ ID NOs: 4553 and 4554; SEQ ID NOs: 4555 and 4556; SEQ ID NOs: 4557 and 4558; SEQ ID NOs: 4559 and 4560; SEQ ID NOs: 4561 and 4562; SEQ ID NOs: 4563 and 4564; SEQ ID NOs: 4565 and 4566; SEQ ID NOs: 4567 and 4568; SEQ ID NOs: 4569 and 4570; SEQ ID NOs: 4571 and 4572; SEQ ID NOs: 4573 and 4574; SEQ ID NOs: 4575 and 4576; SEQ ID NOs: 4577 and 4578; SEQ ID NOs: 4579 and 4580; SEQ ID NOs: 4581 and 4582; SEQ ID NOs: 4583 and 4584; SEQ ID NOs: 4585 and 4586; SEQ ID NOs: 4587 and 4588; SEQ ID NOs: 4589 and 4590; SEQ ID NOs: 4591 and 4592; SEQ ID NOs: 4593 and 4594; SEQ ID NOs: 4595 and 4596; SEQ ID NOs: 4597 and 4598; SEQ ID NOs: 4599 and 4600; SEQ ID NOs: 4601 and 4602; SEQ ID NOs: 4603 and 4604; SEQ ID NOs: 4605 and 4606; SEQ ID NOs: 4607 and 4608. As a non-limiting example, these IL2-saRNA sequences which are saRNA duplexes may be used to modulate IL2 protein levels, regulating T cell levels and/or treating T-cell acute lymphoblastic leukemia.

### IL19 Gene

As another non-limiting example, provided is a method of modulating the expression of IL19 gene comprising administering saRNA of the present invention, wherein the saRNA targets an antisense RNA transcript of IL19 gene. These saRNAs are called IL19-saRNA. In one embodiment, the expression of IL19 gene is increased by at least 20, 30, 40%, or at least 45, 50, 55, 60, 65, 70, 75%, or at least 80% in the presence of the IL19-saRNA of the present invention compared to the expression of IL19 gene in the absence of the IL19-saRNA of the present invention. In a further embodiment, the expression of IL19 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL19-saRNA of the present invention compared to the expression of IL19 gene in the absence of the IL19-saRNA of the present invention. The modulation of the expression of IL19 gene may be reflected or determined by the change of IL19 mRNA levels.

IL19-saRNAs may be single-stranded and comprise 14-30 nucleotides. The sequence of a single-stranded IL19-saRNA may have at least 60%, 70%, 80% or 90% identity with a sequence selected from the sequences of the antisense strands in Table 28-2. In one embodiment, the single-stranded saRNA comprises a sequence selected from the sequences of the antisense strands in Table 28-2. In one embodiment, the single-stranded IL19-saRNA may have a 3′ tail. The sequence of a single-stranded IL19-saRNA with a 3′ tail may have at least 60%, 70%, 80% or 90% identity with a sequence selected from the sequences of the antisense strands in Table 29. In one embodiment, the single-stranded saRNA comprises a sequence selected from the sequences of the antisense strands in Table 29.

IL19-saRNAs may be double-stranded. The two strands form a duplex and each strand comprises 14-30 nucleotides. The first strand of a double-stranded IL19-saRNA may have at least 60%, 70%, 80% or 90% identity with a sequence selected from the sequences of the antisense strands in Table 28-2. In one embodiment, the first strand of the double-stranded saRNA comprises a sequence selected from the sequences of the antisense strands in Table 28-2. The second strand of a double-stranded IL19-saRNA may have at least 60%, 70%, 80% or 90% identity with a sequence selected from the sequences of the sense strands in Table 28-2. In one embodiment, the second strand of the double-stranded saRNA comprises a sequence selected from the sequences of the sense strands in Table 28-2. In one embodiment, the double-stranded IL19-saRNA may have a 3′ overhang on each strand. The first strand of a double-stranded IL19-saRNA may have at least 60%, 70%, 80% or 90% identity with a sequence selected from the sequences of the antisense strands in Table 29. In one embodiment, the first strand of the double-stranded saRNA comprises a sequence selected from the sequences of the antisense strands in Table 29. The second strand of a double-stranded IL19-saRNA may have at least 60%, 70%, 80% or 90% identity with a sequence selected from the sequences of the sense strands in Table 29. In one embodiment, the second strand of the double-stranded saRNA comprises a sequence selected from the sequences of the sense strands in Table 29.

IL19-saRNAs may be modified or unmodified.

Table 28-1 describes the target gene, the targeted sequence, the genomic location of the targeted sequence and the relative location of IL19-saRNAs with no 3′ overhang. In Table 28-1, the targeted sequence is defined as a region on the template strand of the target gene which is identical to a region on the target antisense RNA transcript to which the antisense strand of an saRNA hybridizes. In Table 28-1, the relative location is the distance from the 5′ end of the targeted sequence to the TSS. A negative number represents a location upstream of the TSS and a positive number represents a location downstream of the TSS.

TABLE 28-1
Sequences of IL19-saRNAs (with no 3′ overhang)

| | | | Targeted | | |
|---|---|---|---|---|---|
| | Target | | sequence | Genomic Location of the | Relative |
| saRNA ID | Gene | Targeted sequence | ID | Targeted sequence | Location |
| IL19.NM_013371- | IL19 | tgacgttatctctgagtaa | 1170 | chr1: 207001584 minus | −618 |
| Pr-2 | human | | | strand | |
| IL19.NM_013371- | IL19 | tgtttatctacctgaaatt | 1171 | chr1: 207002591 minus | 389 |
| Pr-8 | human | | | strand | |
| IL19.NM_013371- | IL19 | cgttatctctgagtaaagt | 1172 | chr1: 207001581 minus | −621 |
| Pr-15 | human | | | strand | |
| IL19.NM_013371- | IL19 | tggtggtgagcttagaaat | 1173 | chr1: 207001480 minus | −722 |
| Pr-20 | human | | | strand | |
| IL19.NM_013371- | IL19 | agagtagactcacactaat | 1174 | chr1: 207001385 minus | −817 |
| Pr-21 | human | | | strand | |
| IL19.NM_013371- | IL19 | tggtcaaagtctgttcaaa | 1175 | chr1: 207003187 minus | 985 |
| Pr-30 | human | | | strand | |
| IL19.NM_013371- | IL19 | gtcacatactcaagatgtt | 1176 | chr1: 207001889 minus | −313 |
| Pr-35 | human | | | strand | |
| IL19.NM_153758- | IL19 | agagagtcatcattacaaa | 1177 | chr1: 206972170 minus | −25 |
| Pr-1 | human | | | strand | |
| IL19.NM_153758- | IL19 | gctggtcccttgaattaaa | 1178 | chr1: 206972955 minus | 760 |
| Pr-7 | human | | | strand | |
| IL19.NM_153758- | IL19 | tggctggtcccttgaatta | 1179 | chr1: 206972957 minus | 762 |
| Pr-12 | human | | | strand | |
| IL19.NM_153758- | IL19 | ggctggtcccttgaattaa | 1180 | chr1: 206972956 minus | 761 |
| Pr-14 | human | | | strand | |
| IL19.NM_153758- | IL19 | ggctatagctgattggaaa | 1181 | chr1: 206973057 minus | 862 |
| Pr-24 | human | | | strand | |
| IL19.NM_153758- | IL19 | ggaactttatgacagacaa | 1182 | chr1: 206972475 minus | 280 |
| Pr-30 | human | | | strand | |
| IL19.NM_153758- | IL19 | ggtccctatagcagaaatt | 1183 | chr1: 206972357 minus | 162 |
| Pr-31 | human | | | strand | |
| IL19.NM_153758- | IL19 | gcagtcctgtcagttcata | 1184 | chr1: 206972454 minus | 259 |
| Pr-35 | human | | | strand | |
| IL19.NM_153758- | IL19 | tatgatcgcacctatgaat | 1185 | chr1: 206971546 minus | −649 |
| Pr-39 | human | | | strand | |

TABLE 28-2
Sequences of IL19-saRNAs (with no 3′ overhang)

| | | Sense SEQ | | Antisense SEQ |
|---|---|---|---|---|
| saRNA ID | Sense (passenger) | ID | Antisense (guide) | ID |
| IL19.NM_013371- | UGACGUUAUCUCU | 1186 | UUACUCAGAGAU | 1187 |
| Pr-2 | GAGUAA | | AACGUCA | |
| IL19.NM_013371- | UGUUUAUCUACCU | 1188 | AAUUUCAGGUA | 1189 |
| Pr-8 | GAAAUU | | GAUAAACA | |
| IL19.NM_013371- | CGUUAUCUCUGAG | 1190 | ACUUUACUCAGA | 1191 |
| Pr-15 | UAAAGU | | GAUAACG | |
| IL19.NM_013371- | UGGUGGUGAGCUU | 1192 | AUUUCUAAGCUC | 1193 |
| Pr-20 | AGAAAU | | ACCACCA | |
| IL19.NM_013371- | AGAGUAGACUCAC | 1194 | AUUAGUGUGAG | 1195 |
| Pr-21 | ACUAAU | | UCUACUCU | |
| IL19.NM_013371- | UGGUCAAAGUCUG | 1196 | UUUGAACAGACU | 1197 |
| Pr-30 | UUCAAA | | UUGACCA | |
| IL19.NM_013371- | GUCACAUACUCAAG | 1198 | AACAUCUUGAGU | 1199 |
| Pr-35 | AUGUU | | AUGUGAC | |
| IL19.NM_153758- | AGAGAGUCAUCAU | 1200 | UUUGUAAUGAU | 1201 |
| Pr-1 | UACAAA | | GACUCUCU | |
| IL19.NM_153758- | GCUGGUCCCUUGAA | 1202 | UUUAAUUCAAG | 1203 |
| Pr-7 | UUAAA | | GGACCAGC | |
| IL19.NM_153758- | UGGCUGGUCCCUUG | 1204 | UAAUUCAAGGG | 1205 |
| Pr-12 | AAUUA | | ACCAGCCA | |
| IL19.NM_153758- | GGCUGGUCCCUUGA | 1206 | UUAAUUCAAGG | 1207 |
| Pr-14 | AUUAA | | GACCAGCC | |
| IL19.NM_153758- | GGCUAUAGCUGAU | 1208 | UUUCCAAUCAGC | 1209 |
| Pr-24 | UGGAAA | | UAUAGCC | |
| IL19.NM_153758- | GGAACUUUAUGAC | 1210 | UUGUCUGUCAUA | 1211 |
| Pr-30 | AGACAA | | AAGUUCC | |
| IL19.NM_153758- | GGUCCCUAUAGCAG | 1212 | AAUUUCUGCUAU | 1213 |
| Pr-31 | AAAUU | | AGGGACC | |
| IL19.NM_153758- | GCAGUCCUGUCAGU | 1214 | UAUGAACUGACA | 1215 |
| Pr-35 | UCAUA | | GGACUGC | |
| IL19.NM_153758- | UAUGAUCGCACCUA | 1216 | AUUCAUAGGUGC | 1217 |
| Pr-39 | UGAAU | | GAUCAUA | |

TABLE 29
Sequences of IL19-saRNAs (with 3′ overhang)

| | | Sense SEQ | | Antisense SEQ |
|---|---|---|---|---|
| saRNA ID | Sense (passenger) | ID | Antisense (guide) | ID |
| IL19.NM_013371- | UGACGUUAUCUCU | 1218 | UUACUCAGAGAU | 1219 |
| Pr-2′ | GAGUAAUU | | AACGUCAUU | |
| IL19.NM_013371- | UGUUUAUCUACCU | 1220 | AAUUUCAGGUA | 1221 |
| Pr-8′ | GAAAUUUU | | GAUAAACAUU | |
| IL19.NM_013371- | CGUUAUCUCUGAG | 1222 | ACUUUACUCAGA | 1223 |
| Pr-15′ | UAAAGUUU | | GAUAACGUU | |
| IL19.NM_013371- | UGGUGGUGAGCUU | 1224 | AUUUCUAAGCUC | 1225 |
| Pr-20′ | AGAAAUUU | | ACCACCAUU | |
| IL19.NM_013371- | AGAGUAGACUCAC | 1226 | AUUAGUGUGAG | 1227 |
| Pr-21′ | ACUAAUUU | | UCUACUCUUU | |
| IL19.NM_013371- | UGGUCAAAGUCUG | 1228 | UUUGAACAGACU | 1229 |
| Pr-30′ | UUCAAAUU | | UUGACCAUU | |
| IL19.NM_013371- | GUCACAUACUCAAG | 1230 | AACAUCUUGAGU | 1231 |
| Pr-35′ | AUGUUUU | | AUGUGACUU | |
| IL19.NM_153758- | AGAGAGUCAUCAU | 1232 | UUUGUAAUGAU | 1233 |
| Pr-1′ | UACAAAUU | | GACUCUCUUU | |
| IL19.NM_153758- | GCUGGUCCCUUGAA | 1234 | UUUAAUUCAAG | 1235 |
| Pr-7′ | UUAAAUU | | GGACCAGCUU | |
| IL19.NM_153758- | UGGCUGGUCCCUUG | 1236 | UAAUUCAAGGG | 1237 |
| Pr-12′ | AAUUAUU | | ACCAGCCAUU | |
| IL19.NM_153758- | GGCUGGUCCCUUGA | 1238 | UUAAUUCAAGG | 1239 |
| Pr-14′ | AUUAAUU | | GACCAGCCUU | |
| IL19.NM_153758- | GGCUAUAGCUGAU | 1240 | UUUCCAAUCAGC | 1241 |
| Pr-24′ | UGGAAAUU | | UAUAGCCUU | |
| IL19.NM_153758- | GGAACUUUAUGAC | 1242 | UUGUCUGUCAUA | 1243 |
| Pr-30′ | AGACAAUU | | AAGUUCCUU | |
| IL19.NM_153758- | GGUCCCUAUAGCAG | 1244 | AAUUUCUGCUAU | 1245 |
| Pr-31′ | AAAUUUU | | AGGGACCUU | |
| IL19.NM_153758- | GCAGUCCUGUCAGU | 1246 | UAUGAACUGACA | 1247 |
| Pr-35′ | UCAUAUU | | GGACUGCUU | |
| IL19.NM_153758- | UAUGAUCGCACCUA | 1248 | AUUCAUAGGUGC | 1249 |
| Pr-39′ | UGAAUUU | | GAUCAUAUU | |

IL19 gene encodes interleukin 19 which is a cytokine in IL10 cytokine family. Interleukin 10 binds the IL20 receptor complex and leads to the activation of the activation of the signal transducer and activator of transcription 3 (STAT3).

In one embodiment, provided is a method of modulating interleukin 19 protein levels comprising administering IL19-saRNA of the present invention, wherein the IL19-saRNA targets an antisense RNA transcript of IL19 gene. In one embodiment, interleukin 19 protein level is increased by at least 20, 30, 40%, or at least 45, 50, 55, 60, 65, 70, 75%, or at least 80% in the presence of the IL19-saRNA of the present invention compared to interleukin 19 protein level in the absence of the IL19-saRNA of the present invention. In a further embodiment, interleukin 19 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the IL19-saRNA of the present invention compared to interleukin 19 protein level in the absence of the IL19-saRNA of the present invention.

In one embodiment, the IL19-saRNA of the present invention is an antisense sequence such as, but not limited to, SEQ ID NO: 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 4310, 4312, 4314, 4316, 4318, 4320, 4322, 4324, 4326, 4328, 4330, 4332, 4334, 4336, 4338, 4340, 4342, 4344, 4346, 4348, 4350, 4352, 4354, 4356, 4358, 4360, 4362, 4364, 4366, 4368, 4370, 4372, 4374, 4376, 4378, 4380, 4382, 4384, 4386, 4388, 4390, 4392, 4394, 4396, 4398, 4400, 4402, 4404, 4406, 4408, 4410, 4412, 4414, 4416, 4418, 4420, 4422, 4424, 4426, 4428, 4430, 4432, 4434, 4436, 4438, 4440, 4442, 4444, 4446, 4448, 4450, 4452, 4454, 4456, 4458, 4460, 4462, 4464, 4466, 4468, 4470, 4472, 4474, 4476, 4478, 4480, 4482, 4484, 4486, 4488, 4490, 4492, 4494, 4496, 4498, 4500, 4502, 4504, 4506 and 4508. As a non-limiting example, these IL19-saRNA sequences may be used to modulate IL19 protein levels.

In one embodiment, the IL19-saRNA of the present invention is an saRNA duplex. The saRNA duplex may be a pair of sense and antisense sequences such as, but not limited to, SEQ ID NOs: 1186 and 1187; SEQ ID NOs: 1188 and 1189; SEQ ID NOs: 1190 and 1191; SEQ ID NOs: 1192 and 1193; SEQ ID NOs: 1194 and 1195; SEQ ID NOs: 1196 and 1197; SEQ ID NOs: 1198 and 1199; SEQ ID NOs: 1200 and 1201; SEQ ID NOs: 1202 and 1203; SEQ ID NOs: 1204 and 1205; SEQ ID NOs: 1206 and 1207; SEQ ID NOs: 1208 and 1209; SEQ ID NOs: 1210 and 1211; SEQ ID NOs: 1212 and 1213; SEQ ID NOs: 1214 and 1215; SEQ ID NOs: 1216 and 1217; SEQ ID NOs: 1218 and 1219; SEQ ID NOs: 1220 and 1221; SEQ ID NOs: 1222 and 1223; SEQ ID NOs: 1224 and 1225; SEQ ID NOs: 1226 and 1227; SEQ ID NOs: 1228 and 1229; SEQ ID NOs: 1230 and 1231; SEQ ID NOs: 1232 and 1233; SEQ ID NOs: 1234 and 1235; SEQ ID NOs: 1236 and 1237; SEQ ID NOs: 1238 and 1239; SEQ ID NOs: 1240 and 1241; SEQ ID NOs: 1242 and 1243; SEQ ID NOs: 1244 and 1245; SEQ ID NOs: 1246 and 1247; SEQ ID NOs: 1248 and 1249; SEQ ID NOs: 4309 and 4310; SEQ ID NOs: 4311 and 4312; SEQ ID NOs: 4313 and 4314; SEQ ID NOs: 4315 and 4316; SEQ ID NOs: 4317 and 4318; SEQ ID NOs: 4319 and 4320; SEQ ID NOs: 4321 and 4322; SEQ ID NOs: 4323 and 4324; SEQ ID NOs: 4325 and 4326; SEQ ID NOs: 4327 and 4328; SEQ ID NOs: 4329 and 4330; SEQ ID NOs: 4331 and 4332; SEQ ID NOs: 4333 and 4334; SEQ ID NOs: 4335 and 4336; SEQ ID NOs: 4337 and 4338; SEQ ID NOs: 4339 and 4340; SEQ ID NOs: 4341 and 4342; SEQ ID NOs: 4343 and 4344; SEQ ID NOs: 4345 and 4346; SEQ ID NOs: 4347 and 4348; SEQ ID NOs: 4349 and 4350; SEQ ID NOs: 4351 and 4352; SEQ ID NOs: 4353 and 4354; SEQ ID NOs: 4355 and 4356; SEQ ID NOs: 4357 and 4358; SEQ ID NOs: 4359 and 4360; SEQ ID NOs: 4361 and 4362; SEQ ID NOs: 4363 and 4364; SEQ ID NOs: 4365 and 4366; SEQ ID NOs: 4367 and 4368; SEQ ID NOs: 4369 and 4370; SEQ ID NOs: 4371 and 4372; SEQ ID NOs: 4373 and 4374; SEQ ID NOs: 4375 and 4376; SEQ ID NOs: 4377 and 4378; SEQ ID NOs: 4379 and 4380; SEQ ID NOs: 4381 and 4382; SEQ ID NOs: 4383 and 4384; SEQ ID NOs: 4385 and 4386; SEQ ID NOs: 4387 and 4388; SEQ ID NOs: 4389 and 4390; SEQ ID NOs: 4391 and 4392; SEQ ID NOs: 4393 and 4394; SEQ ID NOs: 4395 and 4396; SEQ ID NOs: 4397 and 4398; SEQ ID NOs: 4399 and 4400; SEQ ID NOs: 4401 and 4402; SEQ ID NOs: 4403 and 4404; SEQ ID NOs: 4405 and 4406; SEQ ID NOs: 4407 and 4408; SEQ ID NOs: 4409 and 4410; SEQ ID NOs: 4411 and 4412; SEQ ID NOs: 4413 and 4414; SEQ ID NOs: 4415 and 4416; SEQ ID NOs: 4417 and 4418; SEQ ID NOs: 4419 and 4420; SEQ ID NOs: 4421 and 4422; SEQ ID NOs: 4423 and 4424; SEQ ID NOs: 4425 and 4426; SEQ ID NOs: 4427 and 4428; SEQ ID NOs: 4429 and 4430; SEQ ID NOs: 4431 and 4432; SEQ ID NOs: 4433 and 4434; SEQ ID NOs: 4435 and 4436; SEQ ID NOs: 4437 and 4438; SEQ ID NOs: 4439 and 4440; SEQ ID NOs: 4441 and 4442; SEQ ID NOs: 4443 and 4444; SEQ ID NOs: 4445 and 4446; SEQ ID NOs: 4447 and 4448; SEQ ID NOs: 4449 and 4450; SEQ ID NOs: 4451 and 4452; SEQ ID NOs: 4453 and 4454; SEQ ID NOs: 4455 and 4456; SEQ ID NOs: 4457 and 4458; SEQ ID NOs: 4459 and 4460; SEQ ID NOs: 4461 and 4462; SEQ ID NOs: 4463 and 4464; SEQ ID NOs: 4465 and 4466; SEQ ID NOs: 4467 and 4468; SEQ ID NOs: 4469 and 4470; SEQ ID NOs: 4471 and 4472; SEQ ID NOs: 4473 and 4474; SEQ ID NOs: 4475 and 4476; SEQ ID NOs: 4477 and 4478; SEQ ID NOs: 4479 and 4480; SEQ ID NOs: 4481 and 4482; SEQ ID NOs: 4483 and 4484; SEQ ID NOs: 4485 and 4486; SEQ ID NOs: 4487 and 4488; SEQ ID NOs: 4489 and 4490; SEQ ID NOs: 4491 and 4492; SEQ ID NOs: 4493 and 4494; SEQ ID NOs: 4495 and 4496; SEQ ID NOs: 4497 and 4498; SEQ ID NOs: 4499 and 4500; SEQ ID NOs: 4501 and 4502; SEQ ID NOs: 4503 and 4504; SEQ ID NOs: 4505 and 4506; SEQ ID NOs: 4507 and 4508. As a non-limiting example, these IL19-saRNA sequences which are saRNA duplexes may be used to modulate IL19 protein levels.

### LMX1A Gene

As another non-limiting example, provided is a method of modulating the expression of LMX1A gene comprising administering saRNA of the present invention, wherein the saRNA targets an antisense RNA transcript of LMX1A gene. These saRNAs are called LMX1A-saRNA. In one embodiment, the expression of LMX1A gene is increased by at least 20, 30, 40%, or at least 45, 50, 55, 60, 65, 70, 75%, or at least 80% in the presence of the LMX1A-saRNA of the present invention compared to the expression of LMX1A gene in the absence of the LMX1A-saRNA of the present invention. In a further embodiment, the expression of LMX1A gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the LMX1A-saRNA of the present invention compared to the expression of LMX1A gene in the absence of the LMX1A-saRNA of the present invention. The modulation of the expression of LMX1A gene may be reflected or determined by the change of LMX1A mRNA levels.

LMX1A-saRNAs may be single-stranded and comprise 14-30 nucleotides. The sequence of a single-stranded LMX1A-saRNA may have at least 60%, 70%, 80% or 90% identity with a sequence selected from the sequences of the antisense strands in Table 30-2. In one embodiment, the single-stranded saRNA comprises a sequence selected from the sequences of the antisense strands in Table 30-2. In one embodiment, the single-stranded LMX1A-saRNA may have a 3′ tail. The sequence of a single-stranded LMX1A-saRNA with a 3′ tail may have at least 60%, 70%, 80% or 90% identity with a sequence selected from the sequences of the antisense strands in Table 31. In one embodiment, the single-stranded saRNA comprises a sequence selected from the sequences of the antisense strands in Table 31.

LMX1A-saRNAs may be double-stranded. The two strands form a duplex and each strand comprises 14-30 nucleotides. The first strand of a double-stranded LMX1A-saRNA may have at least 60%, 70%, 80% or 90% identity with a sequence selected from the sequences of the antisense strands in Table 30-2. In one embodiment, the first strand of the double-stranded saRNA comprises a sequence selected from the sequences of the antisense strands in Table 30-2. The second strand of a double-stranded LMX1A-saRNA may have at least 60%, 70%, 80% or 90% identity with a sequence selected from the sequences of the sense strands in Table 30-2. In one embodiment, the second strand of the double-stranded saRNA comprises a sequence selected from the sequences of the sense strands in Table 30-2. In one embodiment, the double-stranded LMX1A-saRNA may have a 3′ overhang on each strand. The first strand of a double-stranded LMX1A-saRNA may have at least 60%, 70%, 80% or 90% identity with a sequence selected from the sequences of the antisense strands in Table 31. In one embodiment, the first strand of the double-stranded saRNA comprises a sequence selected from the sequences of the antisense strands in Table 31. The second strand of a double-stranded LMX1A-saRNA may have at least 60%, 70%, 80% or 90% identity with a sequence selected from the sequences of the sense strands in Table 31. In one embodiment, the second strand of the double-stranded saRNA comprises a sequence selected from the sequences of the sense strands in Table 31.

LMX1A-saRNA may be modified or unmodified.

Table 30-1 describes the target gene, the targeted sequence, the genomic location of the targeted sequence and the relative location of LMX1A-saRNAs with no 3′ overhang. In Table 30-1, the targeted sequence is defined as a region on the template strand of the target gene which is identical to a region on the target antisense RNA transcript to which the antisense strand of an saRNA hybridizes. In Table 30-1, the relative location is the distance from the 5′ end of the targeted sequence to the TSS. A negative number represents a location upstream of the TSS and a positive number represents a location downstream of the TSS.

TABLE 30-1
Sequences of LMX1A-saRNAs (with no 3′ overhang)

| | | | | Genomic Location | |
|---|---|---|---|---|---|
| | Target | | Targeted | of the Targeted | Relative |
| saRNA ID | Gene | Targeted sequence | sequence ID | sequence | Location |
| LMX1A.NM_001174069- | LMX1A | ggatttatttgggtaggga | 1250 | chr1: 165324538 | 940 |
| Pr-3 | human | | | plus strand | |
| LMX1A.NM_001174069- | LMX1A | gggtatattgggtatataa | 1251 | chr1: 165325193 | 285 |
| Pr-9 | human | | | plus strand | |
| LMX1A.NM_001174069- | LMX1A | cctttaaggtcgaaccata | 1252 | chr1: 165326422 | −944 |
| Pr-13 | human | | | plus strand | |
| LMX1A.NM_001174069- | LMX1A | ctgtttagcgccagtagat | 1253 | chr1: 165326336 | −858 |
| Pr-17 | human | | | plus strand | |
| LMX1A.NM_001174069- | LMX1A | gctgtttagcgccagtaga | 1254 | chr1: 165326335 | −857 |
| Pr-19 | human | | | plus strand | |
| LMX1A.NM_001174069- | LMX1A | agagatcagtcacagcgaa | 1255 | chr1: 165324974 | 504 |
| Pr-24 | human | | | plus strand | |
| LMX1A.NM_001174069- | LMX1A | ggctcatgccagtttccaa | 1256 | chr1: 165326400 | −922 |
| Pr-26 | human | | | plus strand | |
| LMX1A.NM_001174069- | LMX1A | tgttggatttatttgggta | 1257 | chr1: 165324534 | 944 |
| Pr-35 | human | | | plus strand | |
| LMX1A.NM_001174069- | LMX1A | Cggcttgttggatttattt | 1258 | chr1: 165324529 | 949 |
| Pr-40 | human | | | plus strand | |
| LMX1A.NM_177398- | LMX1A | gaatcatgctcttgggaga | 1259 | chr1: 165326484 | −532 |
| Pr-2 | human | | | plus strand | |
| LMX1A.NM_177398- | LMX1A | agttcaattcagtgtgatt | 1260 | chr1: 165326930 | −978 |
| Pr-6 | human | | | plus strand | |
| LMX1A.NM_177398- | LMX1A | ggccttatccgtctgtata | 1261 | chr1: 165326676 | −724 |
| Pr-9 | human | | | plus strand | |
| LMX1A.NM_177398- | LMX1A | agacaattgttgtcccaat | 1262 | chr1: 165326839 | −887 |
| Pr-11 | human | | | plus strand | |
| LMX1A.NM_177398- | LMX1A | gggtatattgggtatataa | 1263 | chr1: 165325193 | 759 |
| Pr-19 | human | | | plus strand | |
| LMX1A.NM_177398- | LMX1A | cctttaaggtcgaaccata | 1264 | chr1: 165326422 | −470 |
| Pr-26 | human | | | plus strand | |
| LMX1A.NM_177398- | LMX1A | ctgtttagcgccagtagat | 1265 | chr1: 165326336 | −384 |
| Pr-30 | human | | | plus strand | |
| LMX1A.NM_177398- | LMX1A | gggaattcatggaatccga | 1266 | chr1: 165326880 | −928 |
| Pr-32 | human | | | plus strand | |
| LMX1A.NM_177398- | LMX1A | gcctaagaatcatgctctt | 1267 | chr1: 165326478 | −526 |
| Pr-33 | human | | | plus strand | |
| LMX1A.NM_177398- | LMX1A | Gctgtttagcgccagtaga | 1268 | chr1: 165326335 | −383 |
| Pr-35 | human | | | plus strand | |

TABLE 30-2
Sequences of LMX1A-saRNAs (with no 3′ overhang)

| | | Sense SEQ | | Antisense |
|---|---|---|---|---|
| saRNA ID | Sense (passenger) | ID | Antisense (guide) | SEQ ID |
| LMX1A.NM_001174069- | GGAUUUAUUUGGG | 1269 | UCCCUACCCAAAUAA | 1270 |
| Pr-3 | UAGGGA | | AUCC | |
| LMX1A.NM_001174069- | GGGUAUAUUGGGU | 1271 | UUAUAUACCCAAUAU | 1272 |
| Pr-9 | AUAUAA | | ACCC | |
| LMX1A.NM_001174069- | CCUUUAAGGUCGA | 1273 | UAUGGUUCGACCUUA | 1274 |
| Pr-13 | ACCAUA | | AAGG | |
| LMX1A.NM_001174069- | CUGUUUAGCGCCAG | 1275 | AUCUACUGGCGCUAA | 1276 |
| Pr-17 | UAGAU | | ACAG | |
| LMX1A.NM_001174069- | GCUGUUUAGCGCCA | 1277 | UCUACUGGCGCUAAA | 1278 |
| Pr-19 | GUAGA | | CAGC | |
| LMX1A.NM_001174069- | AGAGAUCAGUCAC | 1279 | UUCGCUGUGACUGAU | 1280 |
| Pr-24 | AGCGAA | | CUCU | |
| LMX1A.NM_001174069- | GGCUCAUGCCAGUU | 1281 | UUGGAAACUGGCAUG | 1282 |
| Pr-26 | UCCAA | | AGCC | |
| LMX1A.NM_001174069- | UGUUGGAUUUAUU | 1283 | UACCCAAAUAAAUCC | 1284 |
| Pr-35 | UGGGUA | | AACA | |
| LMX1A.NM_001174069- | CGGCUUGUUGGAU | 1285 | AAAUAAAUCCAACAA | 1286 |
| Pr-40 | UUAUUU | | GCCG | |
| LMX1A.NM_177398- | GAAUCAUGCUCUU | 1287 | UCUCCCAAGAGCAUG | 1288 |
| Pr-2 | GGGAGA | | AUUC | |
| LMX1A.NM_177398- | AGUUCAAUUCAGU | 1289 | AAUCACACUGAAUUG | 1290 |
| Pr-6 | GUGAUU | | AACU | |
| LMX1A.NM_177398- | GGCCUUAUCCGUCU | 1291 | UAUACAGACGGAUAA | 1292 |
| Pr-9 | GUAUA | | GGCC | |
| LMX1A.NM_177398- | AGACAAUUGUUGU | 1293 | AUUGGGACAACAAUU | 1294 |
| Pr-11 | CCCAAU | | GUCU | |
| LMX1A.NM_177398- | GGGUAUAUUGGGU | 1295 | UUAUAUACCCAAUAU | 1296 |
| Pr-19 | AUAUAA | | ACCC | |
| LMX1A.NM_177398- | CCUUUAAGGUCGA | 1297 | UAUGGUUCGACCUUA | 1298 |
| Pr-26 | ACCAUA | | AAGG | |
| LMX1A.NM_177398- | CUGUUUAGCGCCAG | 1299 | AUCUACUGGCGCUAA | 1300 |
| Pr-30 | UAGAU | | ACAG | |
| LMX1A.NM_177398- | GGGAAUUCAUGGA | 1301 | UCGGAUUCCAUGAAU | 1302 |
| Pr-32 | AUCCGA | | UCCC | |
| LMX1A.NM_177398- | GCCUAAGAAUCAU | 1303 | AAGAGCAUGAUUCUU | 1304 |
| Pr-33 | GCUCUU | | AGGC | |
| LMX1A.NM_177398- | GCUGUUUAGCGCCA | 1305 | UCUACUGGCGCUAAA | 1306 |
| Pr-35 | GUAGA | | CAGC | |

TABLE 31
Sequences of LMX1A-saRNAs (with 3′ overhang)

| | | Sense SEQ | | Antisense |
|---|---|---|---|---|
| saRNA ID | Sense (passenger) | ID | Antisense (guide) | SEQ ID |
| LMX1A.NM_001174069- | GGAUUUAUUUGGGU | 1307 | UCCCUACCCAAAUAA | 1308 |
| Pr-3′ | AGGGAUU | | AUCCUU | |
| LMX1A.NM_001174069- | GGGUAUAUUGGGUA | 1309 | UUAUAUACCCAAUAU | 1310 |
| Pr-9′ | UAUAAUU | | ACCCUU | |
| LMX1A.NM_001174069- | CCUUUAAGGUCGAAC | 1311 | UAUGGUUCGACCUUA | 1312 |
| Pr-13′ | CAUAUU | | AAGGUU | |
| LMX1A.NM_001174069- | CUGUUUAGCGCCAGU | 1313 | AUCUACUGGCGCUAA | 1314 |
| Pr-17′ | AGAUUU | | ACAGUU | |
| LMX1A.NM_001174069- | GCUGUUUAGCGCCAG | 1315 | UCUACUGGCGCUAAA | 1316 |
| Pr-19′ | UAGAUU | | CAGCUU | |
| LMX1A.NM_001174069- | AGAGAUCAGUCACA | 1317 | UUCGCUGUGACUGAU | 1318 |
| Pr-24′ | GCGAAUU | | CUCUUU | |
| LMX1A.NM_001174069- | GGCUCAUGCCAGUUU | 1319 | UUGGAAACUGGCAUG | 1320 |
| Pr-26′ | CCAAUU | | AGCCUU | |
| LMX1A.NM_001174069- | UGUUGGAUUUAUUU | 1321 | UACCCAAAUAAAUCC | 1322 |
| Pr-35′ | GGGUAUU | | AACAUU | |
| LMX1A.NM_001174069- | CGGCUUGUUGGAUU | 1323 | AAAUAAAUCCAACAA | 1324 |
| Pr-40′ | UAUUUUU | | GCCGUU | |
| LMX1A.NM_177398- | GAAUCAUGCUCUUG | 1325 | UCUCCCAAGAGCAUG | 1326 |
| Pr-2′ | GGAGAUU | | AUUCUU | |
| LMX1A.NM_177398- | AGUUCAAUUCAGUG | 1327 | AAUCACACUGAAUUG | 1328 |
| Pr-6′ | UGAUUUU | | AACUUU | |
| LMX1A.NM_177398- | GGCCUUAUCCGUCUG | 1329 | UAUACAGACGGAUAA | 1330 |
| Pr-9′ | UAUAUU | | GGCCUU | |
| LMX1A.NM_177398- | AGACAAUUGUUGUC | 1331 | AUUGGGACAACAAUU | 1332 |
| Pr-11′ | CCAAUUU | | GUCUUU | |
| LMX1A.NM_177398- | GGGUAUAUUGGGUA | 1333 | UUAUAUACCCAAUAU | 1334 |
| Pr-19′ | UAUAAUU | | ACCCUU | |
| LMX1A.NM_177398- | CCUUUAAGGUCGAAC | 1335 | UAUGGUUCGACCUUA | 1336 |
| Pr-26′ | CAUAUU | | AAGGUU | |
| LMX1A.NM_177398- | CUGUUUAGCGCCAGU | 1337 | AUCUACUGGCGCUAA | 1338 |
| Pr-30′ | AGAUUU | | ACAGUU | |
| LMX1A.NM_177398- | GGGAAUUCAUGGAA | 1339 | UCGGAUUCCAUGAAU | 1340 |
| Pr-32′ | UCCGAUU | | UCCCUU | |
| LMX1A.NM_177398- | GCCUAAGAAUCAUGC | 1341 | AAGAGCAUGAUUCUU | 1342 |
| Pr-33′ | UCUUUU | | AGGCUU | |
| LMX1A.NM_177398- | GCUGUUUAGCGCCAG | 1343 | UCUACUGGCGCUAAA | 1344 |
| Pr-35′ | UAGAUU | | CAGCUU | |

LMX1A gene encodes LIM homeobox transcription factor 1 alpha (LMX1A) protein, which is required for the development of midbrain dopaminergic neurons, roof plate formation, and cortical hem development (Nefzger et al, Stem Cells, vol. 30(7):1349 (2012), the contents of which are incorporated herein by reference in their entirety). Mutations of LMX1A are associated with monogenic diabetes (Edghill et al., J of Pancreas, vol. 11 (1): 14 (2012), the contents of which are incorporated herein by reference in their entirety).

In one embodiment, provided is a method of modulating LMX1A protein levels comprising administering LMX1A-saRNA of the present invention, wherein the LMX1A-saRNA targets an antisense RNA transcript of LMX1A gene. In one embodiment, LMX1A protein level is increased by at least 20, 30, 40%, or at least 45, 50, 55, 60, 65, 70, 75%, or at least 80% in the presence of the LMX1A-saRNA of the present invention compared to LMX1A protein level in the absence of the LMX1A-saRNA of the present invention. In a further embodiment, LMX1A protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the LMX1A-saRNA of the present invention compared to LMX1A protein level in the absence of the LMX1A-saRNA of the present invention.

In another embodiment, provided is a method of treating monogenic diabetes comprising administering LMX1A-saRNA of the present invention, wherein the LMX1A-saRNA targets an antisense RNA transcript of LMX1A gene.

In one embodiment, the LMX1A-saRNA of the present invention is an antisense sequence such as, but not limited to, SEQ ID NO: 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 4710, 4712, 4714, 4716, 4718, 4720, 4722, 4724, 4726, 4728, 4730, 4732, 4734, 4736, 4738, 4740, 4742, 4744, 4746, 4748, 4750, 4752, 4754, 4756, 4758, 4760, 4762, 4764, 4766, 4768, 4770, 4772, 4774, 4776, 4778, 4780, 4782, 4784, 4786, 4788, 4790, 4792, 4794, 4796, 4798, 4800, 4802, 4804, 4806, 4808, 4810, 4812, 4814, 4816, 4818, 4820, 4822, 4824, 4826, 4828, 4830, 4832, 4834, 4836, 4838, 4840, 4842, 4844, 4846, 4848, 4850, 4852, 4854, 4856, 4858, 4860, 4862, 4864, 4866, 4868, 4870, 4872, 4874, 4876, 4878, 4880, 4882, 4884, 4886, 4888, 4890, 4892, 4894, 4896, 4898, 4900, 4902, 4904, 4906 and 4908. As a non-limiting example, these LMX1A-saRNA sequences may be used to modulate LMX1A protein levels and/or treat monogenic diabetes.

In one embodiment, the LMX1A-saRNA of the present invention is an saRNA duplex. The saRNA duplex may be a pair of sense and antisense sequences such as, but not limited to, SEQ ID NOs: 1269 and 1270; SEQ ID NOs: 1271 and 1272; SEQ ID NOs: 1273 and 1274; SEQ ID NOs: 1275 and 1276; SEQ ID NOs: 1277 and 1278; SEQ ID NOs: 1279 and 1280; SEQ ID NOs: 1281 and 1282; SEQ ID NOs: 1283 and 1284; SEQ ID NOs: 1285 and 1286; SEQ ID NOs: 1287 and 1288; SEQ ID NOs: 1289 and 1290; SEQ ID NOs: 1291 and 1292; SEQ ID NOs: 1293 and 1294; SEQ ID NOs: 1295 and 1296; SEQ ID NOs: 1297 and 1298; SEQ ID NOs: 1299 and 1300; SEQ ID NOs: 1301 and 1302; SEQ ID NOs: 1303 and 1304; SEQ ID NOs: 1305 and 1306; SEQ ID NOs: 1307 and 1308; SEQ ID NOs: 1309 and 1310; SEQ ID NOs: 1311 and 1312; SEQ ID NOs: 1313 and 1314; SEQ ID NOs: 1315 and 1316; SEQ ID NOs: 1317 and 1318; SEQ ID NOs: 1319 and 1320; SEQ ID NOs: 1321 and 1322; SEQ ID NOs: 1323 and 1324; SEQ ID NOs: 1325 and 1326; SEQ ID NOs: 1327 and 1328; SEQ ID NOs: 1329 and 1330; SEQ ID NOs: 1331 and 1332; SEQ ID NOs: 1333 and 1334; SEQ ID NOs: 1335 and 1336; SEQ ID NOs: 1337 and 1338; SEQ ID NOs: 1339 and 1340; SEQ ID NOs: 1341 and 1342; SEQ ID NOs: 1343 and 1344; SEQ ID NOs: 4709 and 4710; SEQ ID NOs: 4711 and 4712; SEQ ID NOs: 4713 and 4714; SEQ ID NOs: 4715 and 4716; SEQ ID NOs: 4717 and 4718; SEQ ID NOs: 4719 and 4720; SEQ ID NOs: 4721 and 4722; SEQ ID NOs: 4723 and 4724; SEQ ID NOs: 4725 and 4726; SEQ ID NOs: 4727 and 4728; SEQ ID NOs: 4729 and 4730; SEQ ID NOs: 4731 and 4732; SEQ ID NOs: 4733 and 4734; SEQ ID NOs: 4735 and 4736; SEQ ID NOs: 4737 and 4738; SEQ ID NOs: 4739 and 4740; SEQ ID NOs: 4741 and 4742; SEQ ID NOs: 4743 and 4744; SEQ ID NOs: 4745 and 4746; SEQ ID NOs: 4747 and 4748; SEQ ID NOs: 4749 and 4750; SEQ ID NOs: 4751 and 4752; SEQ ID NOs: 4753 and 4754; SEQ ID NOs: 4755 and 4756; SEQ ID NOs: 4757 and 4758; SEQ ID NOs: 4759 and 4760; SEQ ID NOs: 4761 and 4762; SEQ ID NOs: 4763 and 4764; SEQ ID NOs: 4765 and 4766; SEQ ID NOs: 4767 and 4768; SEQ ID NOs: 4769 and 4770; SEQ ID NOs: 4771 and 4772; SEQ ID NOs: 4773 and 4774; SEQ ID NOs: 4775 and 4776; SEQ ID NOs: 4777 and 4778; SEQ ID NOs: 4779 and 4780; SEQ ID NOs: 4781 and 4782; SEQ ID NOs: 4783 and 4784; SEQ ID NOs: 4785 and 4786; SEQ ID NOs: 4787 and 4788; SEQ ID NOs: 4789 and 4790; SEQ ID NOs: 4791 and 4792; SEQ ID NOs: 4793 and 4794; SEQ ID NOs: 4795 and 4796; SEQ ID NOs: 4797 and 4798; SEQ ID NOs: 4799 and 4800; SEQ ID NOs: 4801 and 4802; SEQ ID NOs: 4803 and 4804; SEQ ID NOs: 4805 and 4806; SEQ ID NOs: 4807 and 4808; SEQ ID NOs: 4809 and 4810; SEQ ID NOs: 4811 and 4812; SEQ ID NOs: 4813 and 4814; SEQ ID NOs: 4815 and 4816; SEQ ID NOs: 4817 and 4818; SEQ ID NOs: 4819 and 4820; SEQ ID NOs: 4821 and 4822; SEQ ID NOs: 4823 and 4824; SEQ ID NOs: 4825 and 4826; SEQ ID NOs: 4827 and 4828; SEQ ID NOs: 4829 and 4830; SEQ ID NOs: 4831 and 4832; SEQ ID NOs: 4833 and 4834; SEQ ID NOs: 4835 and 4836; SEQ ID NOs: 4837 and 4838; SEQ ID NOs: 4839 and 4840; SEQ ID NOs: 4841 and 4842; SEQ ID NOs: 4843 and 4844; SEQ ID NOs: 4845 and 4846; SEQ ID NOs: 4847 and 4848; SEQ ID NOs: 4849 and 4850; SEQ ID NOs: 4851 and 4852; SEQ ID NOs: 4853 and 4854; SEQ ID NOs: 4855 and 4856; SEQ ID NOs: 4857 and 4858; SEQ ID NOs: 4859 and 4860; SEQ ID NOs: 4861 and 4862; SEQ ID NOs: 4863 and 4864; SEQ ID NOs: 4865 and 4866; SEQ ID NOs: 4867 and 4868; SEQ ID NOs: 4869 and 4870; SEQ ID NOs: 4871 and 4872; SEQ ID NOs: 4873 and 4874; SEQ ID NOs: 4875 and 4876; SEQ ID NOs: 4877 and 4878; SEQ ID NOs: 4879 and 4880; SEQ ID NOs: 4881 and 4882; SEQ ID NOs: 4883 and 4884; SEQ ID NOs: 4885 and 4886; SEQ ID NOs: 4887 and 4888; SEQ ID NOs: 4889 and 4890; SEQ ID NOs: 4891 and 4892; SEQ ID NOs: 4893 and 4894; SEQ ID NOs: 4895 and 4896; SEQ ID NOs: 4897 and 4898; SEQ ID NOs: 4899 and 4900; SEQ ID NOs: 4901 and 4902; SEQ ID NOs: 4903 and 4904; SEQ ID NOs: 4905 and 4906; SEQ ID NOs: 4907 and 4908. As a non-limiting example, these LMX1A-saRNA sequences which are saRNA duplexes may be used to modulate LMX1A protein levels and/or treat monogenic diabetes.

### METRNL Gene

As another non-limiting example, provided is a method of modulating the expression of METRNL gene comprising administering saRNA of the present invention, wherein the saRNA targets an antisense RNA transcript of METRNL gene. These saRNAs are called METRNL-saRNA. In one embodiment, the expression of METRNL gene is increased by at least 20, 30, 40%, or at least 45, 50, 55, 60, 65, 70, 75%, or at least 80% in the presence of the METRNL-saRNA of the present invention compared to the expression of METRNL gene in the absence of the METRNL-saRNA of the present invention. In a further embodiment, the expression of METRNL gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the METRNL-saRNA of the present invention compared to the expression of METRNL gene in the absence of the METRNL-saRNA of the present invention. The modulation of the expression of METRNL gene may be reflected or determined by the change of METRNL mRNA levels.

METRNL-saRNAs may be single-stranded and comprise 14-30 nucleotides. The sequence of a single-stranded METRNL-saRNA may have at least 60%, 70%, 80% or 90% identity with a sequence selected from the sequences of the antisense strands in Table 32-2. In one embodiment, the single-stranded saRNA comprises a sequence selected from the sequences of the antisense strands in Table 32-2. In one embodiment, the single-stranded METRNL-saRNA may have a 3′ tail. The sequence of a single-stranded METRNL-saRNA with a 3′ tail may have at least 60%, 70%, 80% or 90% identity with a sequence selected from the sequences of the antisense strands in Table 33. In one embodiment, the single-stranded saRNA comprises a sequence selected from the sequences of the antisense strands in Table 33.

METRNL-saRNAs may be double-stranded. The two strands form a duplex and each strand comprises 14-30 nucleotides. The first strand of a double-stranded METRNL-saRNA may have at least 60%, 70%, 80% or 90% identity with a sequence selected from the sequences of the antisense strands in Table 32-2. In one embodiment, the first strand of the double-stranded saRNA comprises a sequence selected from the sequences of the antisense strands in Table 32-2. The second strand of a double-stranded METRNL-saRNA may have at least 60%, 70%, 80% or 90% identity with a sequence selected from the sequences of the sense strands in Table 32-2. In one embodiment, the second strand of the double-stranded saRNA comprises a sequence selected from the sequences of the sense strands in Table 32-2. In one embodiment, the double-stranded METRNL-saRNA may have a 3′ overhang on each strand. The first strand of a double-stranded METRNL-saRNA may have at least 60%, 70%, 80% or 90% identity with a sequence selected from the sequences of the antisense strands in Table 33. In one embodiment, the first strand of the double-stranded saRNA comprises a sequence selected from the sequences of the antisense strands in Table 33. The second strand of a double-stranded METRNL-saRNA may have at least 60%, 70%, 80% or 90% identity with a sequence selected from the sequences of the sense strands in Table 33. In one embodiment, the second strand of the double-stranded saRNA comprises a sequence selected from the sequences of the sense strands in Table 33.

METRNL-saRNAs may be modified or unmodified.

Table 32-1 describes the target gene, the targeted sequence, the genomic location of the targeted sequence and the relative location of METRNL-saRNAs with no 3′ overhang. In Table 32-1, the targeted sequence is defined as a region on the template strand of the target gene which is identical to a region on the target antisense RNA transcript to which the antisense strand of an saRNA hybridizes. In Table 32-1, the relative location is the distance from the 5′ end of the targeted sequence to the TSS. A negative number represents a location upstream of the TSS and a positive number represents a location downstream of the TSS.

TABLE 32-1
Sequences of METRNL-saRNAs (with no 3′ overhang)

| | | | Targeted | | |
|---|---|---|---|---|---|
| | Target | | sequence | Genomic Location of | Relative |
| saRNA ID | Gene | Targeted sequence | ID | the Targeted sequence | Location |
| METRNL.NM_001004431- | METRNL | cgagtgtgttcctcggagt | 1345 | chr17: 81036603 minus | −944 |
| Pr-2 | human | | | strand | |
| METRNL.NM_001004431- | METRNL | caccgagtcagcagcgaat | 1346 | chr17: 81037324 minus | −223 |
| Pr-12 | human | | | strand | |
| METRNL.NM_001004431- | METRNL | gcgtcctgctcgagtgtgt | 1347 | chr17: 81036613 minus | −934 |
| Pr-18 | human | | | strand | |
| METRNL.NM_001004431- | METRNL | cgtcctgctcgagtgtgtt | 1348 | chr17: 81036612 minus | −935 |
| Pr-19 | human | | | strand | |
| METRNL.NM_001004431- | METRNL | ccgcactctgggtatcagt | 1349 | chr17: 81036728 minus | −819 |
| Pr-20 | human | | | strand | |
| METRNL.NM_001004431- | METRNL | gagttggtgcctcgggagt | 1350 | chr17: 81036942 minus | −605 |
| Pr-22 | human | | | strand | |
| METRNL.NM_001004431- | METRNL | agttggtgcctcgggagtt | 1351 | chr17: 81036941 minus | −606 |
| Pr-23 | human | | | strand | |
| METRNL.NM_001004431- | METRNL | ctctgcagatttggccaaa | 1352 | chr17: 81036675 minus | −872 |
| Pr-24 | human | | | strand | |
| METRNL.NM_001004431- | METRNL | ccgccgagcctctgaagtt | 1353 | chr17: 81037478 minus | −69 |
| Pr-25 | human | | | strand | |
| METRNL.NM_001004431- | METRNL | ctgaagttggcgagtccga | 1354 | chr17: 81037467 minus | −80 |
| Pr-31 | human | | | strand | |
| METRNL.NM_001004431- | METRNL | gagcgtcctgctcgagtgt | 1355 | chr17: 81036615 minus | −932 |
| Pr-33 | human | | | strand | |
| METRNL.NM_001004431- | METRNL | cgcgttccgcagagttggt | 1356 | chr17: 81036953 minus | −594 |
| Pr-35 | human | | | strand | |
| METRNL.NM_001004431- | METRNL | cagagttggtgcctcggga | 1357 | chr17: 81036944 minus | −603 |
| Pr-36 | human | | | strand | |
| METRNL.NM_001004431- | METRNL | tcgagtgtgttcctcggag | 1358 | chr17: 81036604 minus | −943 |
| Pr-38 | human | | | strand | |
| METRNL.NM_001004431- | METRNL | gtcctgctcgagtgtgttc | 1359 | chr17: 81036611 minus | −936 |
| Pr-40 | human | | | strand | |

TABLE 32-2
Sequences of METRNL-saRNAs (with no 3′ overhang)

| | | | | Antisense |
|---|---|---|---|---|
| saRNA ID | Sense (passenger) | Sense SEQ ID | Antisense (guide) | SEQ ID |
| METRNL.NM_001004431- | CGAGUGUGUUCCUC | 1361 | ACUCCGAGGAACAC | 1362 |
| Pr-2 | GGAGU | | ACUCG | |
| METRNL.NM_001004431- | CACCGAGUCAGCAG | 1363 | AUUCGCUGCUGACU | 1364 |
| Pr-12 | CGAAU | | CGGUG | |
| METRNL.NM_001004431- | GCGUCCUGCUCGAG | 1365 | ACACACUCGAGCAG | 1366 |
| Pr-18 | UGUGU | | GACGC | |
| METRNL.NM_001004431- | CGUCCUGCUCGAGU | 1367 | AACACACUCGAGCA | 1368 |
| Pr-19 | GUGUU | | GGACG | |
| METRNL.NM_001004431- | CCGCACUCUGGGUA | 1369 | ACUGAUACCCAGAG | 1370 |
| Pr-20 | UCAGU | | UGCGG | |
| METRNL.NM_001004431- | GAGUUGGUGCCUCG | 1371 | ACUCCCGAGGCACC | 1372 |
| Pr-22 | GGAGU | | AACUC | |
| METRNL.NM_001004431- | AGUUGGUGCCUCGG | 1373 | AACUCCCGAGGCAC | 1374 |
| Pr-23 | GAGUU | | CAACU | |
| METRNL.NM_001004431- | CUCUGCAGAUUUGG | 1375 | UUUGGCCAAAUCUG | 1376 |
| Pr-24 | CCAAA | | CAGAG | |
| METRNL.NM_001004431- | CCGCCGAGCCUCUG | 1377 | AACUUCAGAGGCUC | 1378 |
| Pr-25 | AAGUU | | GGCGG | |
| METRNL.NM_001004431- | CUGAAGUUGGCGAG | 1379 | UCGGACUCGCCAAC | 1380 |
| Pr-31 | UCCGA | | UUCAG | |
| METRNL.NM_001004431- | GAGCGUCCUGCUCG | 1381 | ACACUCGAGCAGGA | 1382 |
| Pr-33 | AGUGU | | CGCUC | |
| METRNL.NM_001004431- | CGCGUUCCGCAGAG | 1383 | ACCAACUCUGCGGA | 1384 |
| Pr-35 | UUGGU | | ACGCG | |
| METRNL.NM_001004431- | CAGAGUUGGUGCCU | 1385 | UCCCGAGGCACCAA | 1386 |
| Pr-36 | CGGGA | | CUCUG | |
| METRNL.NM_001004431- | UCGAGUGUGUUCCU | 1387 | CUCCGAGGAACACA | 1388 |
| Pr-38 | CGGAG | | CUCGA | |
| METRNL.NM_001004431- | GUCCUGCUCGAGUG | 1389 | GAACACACUCGAGC | 1390 |
| Pr-40 | UGUUC | | AGGAC | |

TABLE 33
Sequences of METRNL-saRNAs (with 3′ overhang)

| | | Sense SEQ | | Antisense |
|---|---|---|---|---|
| saRNA ID | Sense (passenger) | ID | Antisense (guide) | SEQ ID |
| METRNL.NM_001004431- | CGAGUGUGUUCCUCG | 1393 | ACUCCGAGGAACACACU | 1394 |
| Pr-2′ | GAGUUU | | CGUU | |
| METRNL.NM_001004431- | CACCGAGUCAGCAGC | 1395 | AUUCGCUGCUGACUCGG | 1396 |
| Pr-12′ | GAAUUU | | UGUU | |
| METRNL.NM_001004431- | GCGUCCUGCUCGAGU | 1397 | ACACACUCGAGCAGGAC | 1398 |
| Pr-18′ | GUGUUU | | GCUU | |
| METRNL.NM_001004431- | CGUCCUGCUCGAGUG | 1399 | AACACACUCGAGCAGGA | 1400 |
| Pr-19′ | UGUUUU | | CGUU | |
| METRNL.NM_001004431- | CCGCACUCUGGGUAU | 1401 | ACUGAUACCCAGAGUGC | 1402 |
| Pr-20′ | CAGUUU | | GGUU | |
| METRNL.NM_001004431- | GAGUUGGUGCCUCG | 1403 | ACUCCCGAGGCACCAAC | 1404 |
| Pr-22′ | GGAGUUU | | UCUU | |
| METRNL.NM_001004431- | AGUUGGUGCCUCGG | 1405 | AACUCCCGAGGCACCAA | 1406 |
| Pr-23′ | GAGUUUU | | CUUU | |
| METRNL.NM_001004431- | CUCUGCAGAUUUGGC | 1407 | UUUGGCCAAAUCUGCAG | 1408 |
| Pr-24′ | CAAAUU | | AGUU | |
| METRNL.NM_001004431- | CCGCCGAGCCUCUGA | 1409 | AACUUCAGAGGCUCGGC | 1410 |
| Pr-25′ | AGUUUU | | GGUU | |
| METRNL.NM_001004431- | CUGAAGUUGGCGAG | 1411 | UCGGACUCGCCAACUUC | 1412 |
| Pr-31′ | UCCGAUU | | AGUU | |
| METRNL.NM_001004431- | GAGCGUCCUGCUCGA | 1413 | ACACUCGAGCAGGACGC | 1414 |
| Pr-33′ | GUGUUU | | UCUU | |
| METRNL.NM_001004431- | CGCGUUCCGCAGAGU | 1415 | ACCAACUCUGCGGAACG | 1416 |
| Pr-35′ | UGGUUU | | CGUU | |
| METRNL.NM_001004431- | CAGAGUUGGUGCCUC | 1417 | UCCCGAGGCACCAACUC | 1418 |
| Pr-36′ | GGGAUU | | UGUU | |
| METRNL.NM_001004431- | UCGAGUGUGUUCCUC | 1419 | CUCCGAGGAACACACUC | 1420 |
| Pr-38′ | GGAGUU | | GAUU | |
| METRNL.NM_001004431- | GUCCUGCUCGAGUGU | 1421 | GAACACACUCGAGCAGG | 1422 |
| Pr-40′ | GUUCUU | | ACUU | |

METRNL gene is an important gene for neurogenesis. It is a latent process gene that induces expression during the latent process and is required for subsequent neurite extension (Watanabe et al., Journal of Cell Science, vol. 125:2198 (2012), the contents of which are incorporated herein by reference in their entirety). In one embodiment, provided is a method of regulating neurogenesis comprising administering METRNL-saRNA of the present invention, wherein the METRNL-saRNA targets an antisense RNA transcript of METRNL gene. In another embodiment, provided is a method of regulating latent process comprising administering METRNL-saRNA of the present invention, wherein the METRNL-saRNA targets an antisense RNA transcript of METRNL gene

In one embodiment, the METRNL-saRNA of the present invention is an antisense sequence such as, but not limited to, SEQ ID NO: 1362, 1364, 1366, 1368, 1370, 1372, 1374, 1376, 1378, 1380, 1382, 1384, 1386, 1388, 1390, 1394, 1396, 1398, 1400, 1402, 1404, 1406, 1408, 1410, 1412, 1414, 1416, 1418, 1420, 1422, 5076, 5078, 5080, 5082, 5084, 5086, 5088, 5090, 5092, 5094, 5096, 5098, 5100, 5102, 5104, 5106, 5108, 5110, 5112 and 5114. As a non-limiting example, these METRNL-saRNA sequences may be used to modulate METRNL protein levels, regulating neurogenesis and/or regulating latent process.

In one embodiment, the METRNL-saRNA of the present invention is an saRNA duplex. The saRNA duplex may be a pair of sense and antisense sequences such as, but not limited to, SEQ ID NOs: 1361 and 1362; SEQ ID NOs: 1363 and 1364; SEQ ID NOs: 1365 and 1366; SEQ ID NOs: 1367 and 1368; SEQ ID NOs: 1369 and 1370; SEQ ID NOs: 1371 and 1372; SEQ ID NOs: 1373 and 1374; SEQ ID NOs: 1375 and 1376; SEQ ID NOs: 1377 and 1378; SEQ ID NOs: 1379 and 1380; SEQ ID NOs: 1381 and 1382; SEQ ID NOs: 1383 and 1384; SEQ ID NOs: 1385 and 1386; SEQ ID NOs: 1387 and 1388; SEQ ID NOs: 1389 and 1390; SEQ ID NOs: 1393 and 1394; SEQ ID NOs: 1395 and 1396; SEQ ID NOs: 1397 and 1398; SEQ ID NOs: 1399 and 1400; SEQ ID NOs: 1401 and 1402; SEQ ID NOs: 1403 and 1404; SEQ ID NOs: 1405 and 1406; SEQ ID NOs: 1407 and 1408; SEQ ID NOs: 1409 and 1410; SEQ ID NOs: 1411 and 1412; SEQ ID NOs: 1413 and 1414; SEQ ID NOs: 1415 and 1416; SEQ ID NOs: 1417 and 1418; SEQ ID NOs: 1419 and 1420; SEQ ID NOs: 1421 and 1422; SEQ ID NOs: 5075 and 5076; SEQ ID NOs: 5077 and 5078; SEQ ID NOs: 5079 and 5080; SEQ ID NOs: 5081 and 5082; SEQ ID NOs: 5083 and 5084; SEQ ID NOs: 5085 and 5086; SEQ ID NOs: 5087 and 5088; SEQ ID NOs: 5089 and 5090; SEQ ID NOs: 5091 and 5092; SEQ ID NOs: 5093 and 5094; SEQ ID NOs: 5095 and 5096; SEQ ID NOs: 5097 and 5098; SEQ ID NOs: 5099 and 5100; SEQ ID NOs: 5101 and 5102; SEQ ID NOs: 5103 and 5104; SEQ ID NOs: 5105 and 5106; SEQ ID NOs: 5107 and 5108; SEQ ID NOs: 5109 and 5110; SEQ ID NOs: 5111 and 5112; SEQ ID NOs: 5113 and 5114. As a non-limiting example, these METRNL-saRNA sequences which are saRNA duplexes may be used to modulate METRNL protein levels, regulating neurogenesis and/or regulating latent process.

### NR4A2 Gene

As another non-limiting example, provided is a method of modulating the expression of NR4A2 gene comprising administering saRNA of the present invention, wherein the saRNA targets an antisense RNA transcript of NR4A2 gene. These saRNAs are called NR4A2-saRNA. In one embodiment, the expression of NR4A2 gene is increased by at least 20, 30, 40%, or at least 45, 50, 55, 60, 65, 70, 75%, or at least 80% in the presence of the NR4A2-saRNA of the present invention compared to the expression of NR4A2 gene in the absence of the NR4A2-saRNA of the present invention. In a further embodiment, the expression of NR4A2 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the NR4A2-saRNA of the present invention compared to the expression of NR4A2 gene in the absence of the NR4A2-saRNA of the present invention. The modulation of the expression of NR4A2 gene may be reflected or determined by the change of NR4A2 mRNA levels.

NR4A2-saRNAs may be single-stranded and comprise 14-30 nucleotides. The sequence of a single-stranded NR4A2-saRNA may have at least 60%, 70%, 80% or 90% identity with a sequence selected from the sequences of the antisense strands in Table 34-2. In one embodiment, the single-stranded saRNA comprises a sequence selected from the sequences of the antisense strands in Table 34-2. In one embodiment, the single-stranded NR4A2-saRNA may have a 3′ tail. The sequence of a single-stranded NR4A2-saRNA with a 3′ tail may have at least 60%, 70%, 80% or 90% identity with a sequence selected from the sequences of the antisense strands in Table 35. In one embodiment, the single-stranded saRNA comprises a sequence selected from the sequences of the antisense strands in Table 35.

NR4A2-saRNAs may be double-stranded. The two strands form a duplex and each strand comprises 14-30 nucleotides. The first strand of a double-stranded NR4A2-saRNA may have at least 60%, 70%, 80% or 90% identity with a sequence selected from the sequences of the antisense strands in Table 34-2. In one embodiment, the first strand of the double-stranded saRNA comprises a sequence selected from the sequences of the antisense strands in Table 34-2. The second strand of a double-stranded NR4A2-saRNA may have at least 60%, 70%, 80% or 90% identity with a sequence selected from the sequences of the sense strands in Table 34-2. In one embodiment, the second strand of the double-stranded saRNA comprises a sequence selected from the sequences of the sense strands in Table 34-2. In one embodiment, the double-stranded NR4A2-saRNA may have a 3′ overhang on each strand. The first strand of a double-stranded NR4A2-saRNA may have at least 60%, 70%, 80% or 90% identity with a sequence selected from the sequences of the antisense strands in Table 35. In one embodiment, the first strand of the double-stranded saRNA comprises a sequence selected from the sequences of the antisense strands in Table 35. The second strand of a double-stranded NR4A2-saRNA may have at least 60%, 70%, 80% or 90% identity with a sequence selected from the sequences of the sense strands in Table 35. In one embodiment, the second strand of the double-stranded saRNA comprises a sequence selected from the sequences of the sense strands in Table 35.

NR4A2-saRNAs may be modified or unmodified.

Table 34-1 describes the target gene, the targeted sequence, the genomic location of the targeted sequence and the relative location of NR4A2-saRNAs with no 3′ overhang. In Table 34-1, the targeted sequence is defined as a region on the template strand of the target gene which is identical to a region on the target antisense RNA transcript to which the antisense strand of an saRNA hybridizes. In Table 34-1, the relative location is the distance from the 5′ end of the targeted sequence to the TSS. A negative number represents a location upstream of the TSS and a positive number represents a location downstream of the TSS.

TABLE 34-1
Sequences of NR4A2-saRNAs (with no 3′ overhang)

| | | | | Genomic Location of | |
|---|---|---|---|---|---|
| | Target | | Targeted | the Targeted | Relative |
| saRNA ID | Gene | Targeted sequence | sequence ID | sequence | Location |
| NR4A2.NM_006186- | NR4A2 | agccggagttcgaacaaat | 1425 | chr2: 157188803 plus | 484 |
| Pr-5 | human | | | strand | |
| NR4A2.NM_006186- | NR4A2 | ggcatatattcattctaat | 1426 | chr2: 157189831 plus | −544 |
| Pr-6 | human | | | strand | |
| NR4A2.NM_006186- | NR4A2 | tcgatcttcgtttgaaaca | 1427 | chr2: 157190238 plus | −951 |
| Pr-7 | human | | | strand | |
| NR4A2.NM_006186- | NR4A2 | gtacagatgtcccatgaca | 1428 | chr2: 157189715 plus | −428 |
| Pr-10 | human | | | strand | |
| NR4A2.NM_006186- | NR4A2 | ggaataaatcgatcttcgt | 1429 | chr2: 157190230 plus | −943 |
| Pr-18 | human | | | strand | |
| NR4A2.NM_006186- | NR4A2 | atattctcgctctgataca | 1430 | chr2: 157188407 plus | 880 |
| Pr-22 | human | | | strand | |
| NR4A2.NM_006186- | NR4A2 | aaatcgatcttcgtttgaa | 1431 | chr2: 157190235 plus | −948 |
| Pr-29 | human | | | strand | |
| NR4A2.NM_006186- | NR4A2 | tggtatatttccgacctga | 1432 | chr2: 157189346 plus | −59 |
| Pr-31 | human | | | strand | |
| NR4A2.NM_006186- | NR4A2 | ctctaatactttcgatact | 1433 | chr2: 157190169 plus | −882 |
| Pr-32 | human | | | strand | |
| NR4A2.NM_006186- | NR4A2 | gatcttcgtttgaaacaca | 1434 | chr2: 157190240 plus | −953 |
| Pr-35 | human | | | strand | |
| NR4A2.NM_006186- | NR4A2 | ttcatattctcgctctgat | 1435 | chr2: 157188404 plus | 883 |
| Pr-38 | human | | | strand | |

TABLE 34-2
Sequences of NR4A2-saRNAs (with no 3′ overhang)

| | | | | Antisense |
|---|---|---|---|---|
| saRNA ID | Sense (passenger) | Sense SEQ ID | Antisense (guide) | SEQ ID |
| NR4A2.NM_006186- | AGCCGGAGUUCGAA | 1436 | AUUUGUUCGAACUCC | 1437 |
| Pr-5 | CAAAU | | GGCU | |
| NR4A2.NM_006186- | GGCAUAUAUUCAUU | 1438 | AUUAGAAUGAAUAU | 1439 |
| Pr-6 | CUAAU | | AUGCC | |
| NR4A2.NM_006186- | UCGAUCUUCGUUUG | 1440 | UGUUUCAAACGAAGA | 1441 |
| Pr-7 | AAACA | | UCGA | |
| NR4A2.NM_006186- | GUACAGAUGUCCCA | 1442 | UGUCAUGGGACAUCU | 1443 |
| Pr-10 | UGACA | | GUAC | |
| NR4A2.NM_006186- | GGAAUAAAUCGAUC | 1444 | ACGAAGAUCGAUUUA | 1445 |
| Pr-18 | UUCGU | | UUCC | |
| NR4A2.NM_006186- | AUAUUCUCGCUCUG | 1446 | UGUAUCAGAGCGAGA | 1447 |
| Pr-22 | AUACA | | AUAU | |
| NR4A2.NM_006186- | AAAUCGAUCUUCGU | 1448 | UUCAAACGAAGAUCG | 1449 |
| Pr-29 | UUGAA | | AUUU | |
| NR4A2.NM_006186- | UGGUAUAUUUCCGA | 1450 | UCAGGUCGGAAAUAU | 1451 |
| Pr-31 | CCUGA | | ACCA | |
| NR4A2.NM_006186- | CUCUAAUACUUUCG | 1452 | AGUAUCGAAAGUAU | 1453 |
| Pr-32 | AUACU | | UAGAG | |
| NR4A2.NM_006186- | GAUCUUCGUUUGAA | 1454 | UGUGUUUCAAACGAA | 1455 |
| Pr-35 | ACACA | | GAUC | |
| NR4A2.NM_006186- | UUCAUAUUCUCGCU | 1456 | AUCAGAGCGAGAAUA | 1457 |
| Pr-38 | CUGAU | | UGAA | |

TABLE 35
Sequences of NR4A2-saRNAs (with 3′ overhang)

| | | | | Antisense |
|---|---|---|---|---|
| saRNA ID | Sense (passenger) | Sense SEQ ID | Antisense (guide) | SEQ ID |
| NR4A2.NM_006186- | AGCCGGAGUUCGAA | 1458 | AUUUGUUCGAACUCC | 1459 |
| Pr-5 | CAAAUUU | | GGCUUU | |
| NR4A2.NM_006186- | GGCAUAUAUUCAUU | 1460 | AUUAGAAUGAAUAU | 1461 |
| Pr-6 | CUAAUUU | | AUGCCUU | |
| NR4A2.NM_006186- | UCGAUCUUCGUUUG | 1462 | UGUUUCAAACGAAGA | 1463 |
| Pr-7 | AAACAUU | | UCGAUU | |
| NR4A2.NM_006186- | GUACAGAUGUCCCA | 1464 | UGUCAUGGGACAUCU | 1465 |
| Pr-10 | UGACAUU | | GUACUU | |
| NR4A2.NM_006186- | GGAAUAAAUCGAUC | 1466 | ACGAAGAUCGAUUUA | 1467 |
| Pr-18 | UUCGUUU | | UUCCUU | |
| NR4A2.NM_006186- | AUAUUCUCGCUCUG | 1468 | UGUAUCAGAGCGAGA | 1469 |
| Pr-22 | AUACAUU | | AUAUUU | |
| NR4A2.NM_006186- | AAAUCGAUCUUCGU | 1470 | UUCAAACGAAGAUCG | 1471 |
| Pr-29 | UUGAAUU | | AUUUUU | |
| NR4A2.NM_006186- | UGGUAUAUUUCCGA | 1472 | UCAGGUCGGAAAUAU | 1473 |
| Pr-31 | CCUGAUU | | ACCAUU | |
| NR4A2.NM_006186- | CUCUAAUACUUUCG | 1474 | AGUAUCGAAAGUAUU | 1475 |
| Pr-32 | AUACUUU | | AGAGUU | |
| NR4A2.NM_006186- | GAUCUUCGUUUGAA | 1476 | UGUGUUUCAAACGAA | 1477 |
| Pr-35 | ACACAUU | | GAUCUU | |
| NR4A2.NM_006186- | UUCAUAUUCUCGCU | 1478 | AUCAGAGCGAGAAUA | 1479 |
| Pr-38 | CUGAUUU | | UGAAUU | |

NR4A2 gene, also called NURR1 gene, encodes nuclear receptor subfamily 4, group A, member 2 (NR4A2) protein, a member of the nuclear receptor family found in the brain and the adrenal gland. In the brain, NR4A2 protein plays a key role in prompting certain nerve cells to differentiate and produce a chemical messenger called dopamine. Dopamine transmits messages that help the brain control physical movement and emotional behavior. NR4A2 protein is associated with stress and addiction (Campos-Melo et al., Front. Mol. Neurosci., vol. 6:44 (2013), the contents of which are incorporated herein by reference in their entirety).

In one embodiment, provided is a method of modulating NR4A2 protein levels comprising administering NR4A2-saRNA of the present invention, wherein the NR4A2-saRNA targets an antisense RNA transcript of NR4A2 gene. In one embodiment, NR4A2 protein level is increased by at least 20, 30, 40%, or at least 45, 50, 55, 60, 65, 70, 75%, or at least 80% in the presence of the NR4A2-saRNA of the present invention compared to NR4A2 protein level in the absence of the NR4A2-saRNA of the present invention. In a further embodiment, NR4A2 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the NR4A2-saRNA of the present invention compared to NR4A2 protein level in the absence of the NR4A2-saRNA of the present invention.

In another embodiment, provided is a method of regulating dopamine comprising administering NR4A2-saRNA of the present invention, wherein the NR4A2-saRNA targets an antisense RNA transcript of NR4A2 gene.

In another embodiment, provided is a method of reducing stress and addiction comprising administering NR4A2-saRNA of the present invention, wherein the NR4A2-saRNA targets an antisense RNA transcript of NR4A2 gene.

In one embodiment, the NR4A2-saRNA of the present invention is an antisense sequence such as, but not limited to, SEQ ID NO: 1437, 1439, 1441, 1443, 1445, 1447, 1449, 1451, 1453, 1455, 1457, 1459, 1461, 1463, 1465, 1467, 1469, 1471, 1473, 1475, 1477, 1479, 5116, 5118, 5120, 5122, 5124, 5126, 5128, 5130, 5132, 5134, 5136, 5138, 5140, 5142, 5144, 5146, 5148, 5150, 5152, 5154, 5156, 5158, 5160, 5162, 5164, 5166, 5168, 5170, 5172, 5174, 5176, 5178, 5180, 5182, 5184, 5186, 5188, 5190, 5192, 5194, 5196, 5198, 5200, 5202, 5204, 5206, 5208, 5210, 5212 and 5214. As a non-limiting example, these NR4A2-saRNA sequences may be used to modulate NR4A2 protein levels, regulating dopamine and/or reducing stress and addiction.

In one embodiment, the NR4A2-saRNA of the present invention is an saRNA duplex. The saRNA duplex may be a pair of sense and antisense sequences such as, but not limited to, SEQ ID NOs: 1436 and 1437; SEQ ID NOs: 1438 and 1439; SEQ ID NOs: 1440 and 1441; SEQ ID NOs: 1442 and 1443; SEQ ID NOs: 1444 and 1445; SEQ ID NOs: 1446 and 1447; SEQ ID NOs: 1448 and 1449; SEQ ID NOs: 1450 and 1451; SEQ ID NOs: 1452 and 1453; SEQ ID NOs: 1454 and 1455; SEQ ID NOs: 1456 and 1457; SEQ ID NOs: 1458 and 1459; SEQ ID NOs: 1460 and 1461; SEQ ID NOs: 1462 and 1463; SEQ ID NOs: 1464 and 1465; SEQ ID NOs: 1466 and 1467; SEQ ID NOs: 1468 and 1469; SEQ ID NOs: 1470 and 1471; SEQ ID NOs: 1472 and 1473; SEQ ID NOs: 1474 and 1475; SEQ ID NOs: 1476 and 1477; SEQ ID NOs: 1478 and 1479; SEQ ID NOs: 5115 and 5116; SEQ ID NOs: 5117 and 5118; SEQ ID NOs: 5119 and 5120; SEQ ID NOs: 5121 and 5122; SEQ ID NOs: 5123 and 5124; SEQ ID NOs: 5125 and 5126; SEQ ID NOs: 5127 and 5128; SEQ ID NOs: 5129 and 5130; SEQ ID NOs: 5131 and 5132; SEQ ID NOs: 5133 and 5134; SEQ ID NOs: 5135 and 5136; SEQ ID NOs: 5137 and 5138; SEQ ID NOs: 5139 and 5140; SEQ ID NOs: 5141 and 5142; SEQ ID NOs: 5143 and 5144; SEQ ID NOs: 5145 and 5146; SEQ ID NOs: 5147 and 5148; SEQ ID NOs: 5149 and 5150; SEQ ID NOs: 5151 and 5152; SEQ ID NOs: 5153 and 5154; SEQ ID NOs: 5155 and 5156; SEQ ID NOs: 5157 and 5158; SEQ ID NOs: 5159 and 5160; SEQ ID NOs: 5161 and 5162; SEQ ID NOs: 5163 and 5164; SEQ ID NOs: 5165 and 5166; SEQ ID NOs: 5167 and 5168; SEQ ID NOs: 5169 and 5170; SEQ ID NOs: 5171 and 5172; SEQ ID NOs: 5173 and 5174; SEQ ID NOs: 5175 and 5176; SEQ ID NOs: 5177 and 5178; SEQ ID NOs: 5179 and 5180; SEQ ID NOs: 5181 and 5182; SEQ ID NOs: 5183 and 5184; SEQ ID NOs: 5185 and 5186; SEQ ID NOs: 5187 and 5188; SEQ ID NOs: 5189 and 5190; SEQ ID NOs: 5191 and 5192; SEQ ID NOs: 5193 and 5194; SEQ ID NOs: 5195 and 5196; SEQ ID NOs: 5197 and 5198; SEQ ID NOs: 5199 and 5200; SEQ ID NOs: 5201 and 5202; SEQ ID NOs: 5203 and 5204; SEQ ID NOs: 5205 and 5206; SEQ ID NOs: 5207 and 5208; SEQ ID NOs: 5209 and 5210; SEQ ID NOs: 5211 and 5212; SEQ ID NOs: 5213 and 5214. As a non-limiting example, these NR4A2-saRNA sequences which are saRNA duplexes may be used to modulate NR4A2 protein levels, regulating dopamine and/or reducing stress and addiction.

### SIRT1 Gene

As another non-limiting example, provided is a method of modulating the expression of SIRT1 gene comprising administering saRNA of the present invention, wherein the saRNA targets an antisense RNA transcript of SIRT1 gene. These saRNAs are called SIRT1-saRNA. In one embodiment, the expression of SIRT1 gene is increased by at least 20, 30, 40%, or at least 45, 50, 55, 60, 65, 70, 75%, or at least 80% in the presence of the SIRT1-saRNA of the present invention compared to the expression of SIRT1 gene in the absence of the SIRT1-saRNA of the present invention. In a further embodiment, the expression of SIRT1 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the SIRT1-saRNA of the present invention compared to the expression of SIRT1 gene in the absence of the SIRT1-saRNA of the present invention. The modulation of the expression of SIRT1 gene may be reflected or determined by the change of SIRT1 mRNA levels.

SIRT1-saRNAs may be single-stranded and comprise 14-30 nucleotides. The sequence of a single-stranded SIRT1-saRNA may have at least 60%, 70%, 80% or 90% identity with a sequence selected from the sequences of the antisense strands in Table 36-2. In one embodiment, the single-stranded saRNA comprises a sequence selected from the sequences of the antisense strands in Table 36-2. In one embodiment, the single-stranded SIRT1-saRNA may have a 3′ tail. The sequence of a single-stranded SIRT1-saRNA with a 3′ tail may have at least 60%, 70%, 80% or 90% identity with a sequence selected from the sequences of the antisense strands in Table 37. In one embodiment, the single-stranded saRNA comprises a sequence selected from the sequences of the antisense strands in Table 37.

SIRT1-saRNAs may be double-stranded. The two strands form a duplex and each strand comprises 14-30 nucleotides. The first strand of a double-stranded SIRT1-saRNA may have at least 60%, 70%, 80% or 90% identity with a sequence selected from the sequences of the antisense strands in Table 36-2. In one embodiment, the first strand of the double-stranded saRNA comprises a sequence selected from the sequences of the antisense strands in Table 36-2. The second strand of a double-stranded SIRT1-saRNA may have at least 60%, 70%, 80% or 90% identity with a sequence selected from the sequences of the sense strands in Table 36-2. In one embodiment, the second strand of the double-stranded saRNA comprises a sequence selected from the sequences of the sense strands in Table 36-2. In one embodiment, the double-stranded SIRT1-saRNA may have a 3′ overhang on each strand. The first strand of a double-stranded SIRT1-saRNA may have at least 60%, 70%, 80% or 90% identity with a sequence selected from the sequences of the antisense strands in Table 37. In one embodiment, the first strand of the double-stranded saRNA comprises a sequence selected from the sequences of the antisense strands in Table 37. The second strand of a double-stranded SIRT1-saRNA may have at least 60%, 70%, 80% or 90% identity with a sequence selected from the sequences of the sense strands in Table 37. In one embodiment, the second strand of the double-stranded saRNA comprises a sequence selected from the sequences of the sense strands in Table 37.

SIRT1-saRNAs may be modified or unmodified.

Table 36-1 describes the target gene, the targeted sequence, the genomic location of the targeted sequence and the relative location of SIRT1-saRNAs with no 3′ overhang. In Table 36-1, the targeted sequence is defined as a region on the template strand of the target gene which is identical to a region on the target antisense RNA transcript to which the antisense strand of an saRNA hybridizes. In Table 36-1, the relative location is the distance from the 5′ end of the targeted sequence to the TSS. A negative number represents a location upstream of the TSS and a positive number represents a location downstream of the TSS.

TABLE 36-1
Sequences of SIRT1-saRNAs (with no 3′ overhang)

| | | | | Genomic Location | |
|---|---|---|---|---|---|
| | | Targeted | Targeted | of the Targeted | Relative |
| saRNA ID | Target Gene | sequence | sequence ID | sequence | Location |
| SIRT1.NM_001142498- | SIRT1 human | gacttagctccctg | 1480 | chr10: 69643986 | −933 |
| Pr-1 | | aaata | | minus strand | |
| SIRT1.NM_001142498- | SIRT1 human | agacttagctccct | 1481 | chr10: 69643987 | −932 |
| Pr-2 | | gaaat | | minus strand | |
| SIRT1.NM_001142498- | SIRT1 human | gcaagctgtgacta | 1482 | chr10: 69645435 | 516 |
| Pr-13 | | tagaa | | minus strand | |
| SIRT1.NM_001142498- | SIRT1 human | gtgttaagtttgtgt | 1483 | chr10: 69645628 | 709 |
| Pr-18 | | ctca | | minus strand | |
| SIRT1.NM_001142498- | SIRT1 human | gccttagccttgtct | 1484 | chr10: 69644041 | −878 |
| Pr-19 | | atat | | minus strand | |
| SIRT1.NM_001142498- | SIRT1 human | tgctagaagtgtta | 1485 | chr10: 69645636 | 717 |
| Pr-23 | | agttt | | minus strand | |
| SIRT1.NM_001142498- | SIRT1 human | cagccacgttcttta | 1486 | chr10: 69645456 | 537 |
| Pr-24 | | aatt | | minus strand | |
| SIRT1.NM_001142498- | SIRT1 human | ctgaaatacgttgg | 1487 | chr10: 69643975 | −944 |
| Pr-25 | | atagt | | minus strand | |
| SIRT1.NM_001142498- | SIRT1 human | aagtttgtgtctcaa | 1488 | chr10: 69645623 | 704 |
| Pr-26 | | tact | | minus strand | |
| SIRT1.NM_001142498- | SIRT1 human | gttggatagtcgttc | 1489 | chr10: 69643966 | −953 |
| Pr-34 | | tgtt | | minus strand | |
| SIRT1.NM_001142498- | SIRT1 human | agagcaagattag | 1490 | chr10: 69645556 | 637 |
| Pr-36 | | aataaa | | minus strand | |
| SIRT1.NM_001142498- | SIRT1 human | tgcattacagaatg | 1491 | chr10: 69645486 | 567 |
| Pr-37 | | gtaat | | minus strand | |
| SIRT1.NM_012238- | SIRT1 human | gacttagctccctg | 1492 | chr10: 69643986 | −421 |
| Pr-1 | | aaata | | minus strand | |
| SIRT1.NM_012238- | SIRT1 human | agacttagctccct | 1493 | chr10: 69643987 | −420 |
| Pr-2 | | gaaat | | minus strand | |
| SIRT1.NM_012238- | SIRT1 human | gcttcaaacgtgtg | 1494 | chr10: 69643695 | −712 |
| Pr-6 | | tgaat | | minus strand | |
| SIRT1.NM_012238- | SIRT1 human | gccttagccttgtct | 1495 | chr10: 69644041 | −366 |
| Pr-12 | | atat | | minus strand | |
| SIRT1.NM_012238- | SIRT1 human | agccgtdctaaact | 1496 | chr10: 69643532 | −875 |
| Pr-17 | | taat | | minus strand | |
| SIRT1.NM_012238- | SIRT1 human | ctgaaatacgttgg | 1497 | chr10: 69643975 | −432 |
| Pr-19 | | atagt | | minus strand | |
| SIRT1.NM_012238- | SIRT1 human | gttggatagtcgttc | 1498 | chr10: 69643966 | −441 |
| Pr-30 | | tgtt | | minus strand | |
| SIRT1.NM_012238- | SIRT1 human | tgccttagccttgtc | 1499 | chr10: 69644042 | −365 |
| Pr-38 | | tata | | minus strand | |
| SIRT1.NM_012238- | SIRT1 human | ctagccgtttctaaa | 1500 | chr10: 69643534 | −873 |
| Pr-39 | | ctta | | minus strand | |

TABLE 36-2
Sequences of SIRT1-saRNAs (with no 3′ overhang)

| | | Sense SEQ | | Anti-sense |
|---|---|---|---|---|
| saRNA ID | Sense (passenger) | ID | Antisense (guide) | SEQ ID |
| SIRT1.NM_001142498- | GACUUAGCUCCCUGAA | 1501 | UAUUUCAGGGAGC | 1502 |
| Pr-1 | AUA | | UAAGUC | |
| SIRT1.NM_001142498- | AGACUUAGCUCCCUGA | 1503 | AUUUCAGGGAGCU | 1504 |
| Pr-2 | AAU | | AAGUCU | |
| SIRT1.NM_001142498- | GCAAGCUGUGACUAUA | 1505 | UUCUAUAGUCACA | 1506 |
| Pr-13 | GAA | | GCUUGC | |
| SIRT1.NM_001142498- | GUGUUAAGUUUGUGUC | 1507 | UGAGACACAAACU | 1508 |
| Pr-18 | UCA | | UAACAC | |
| SIRT1.NM_001142498- | GCCUUAGCCUUGUCUA | 1509 | AUAUAGACAAGGC | 1510 |
| Pr-19 | UAU | | UAAGGC | |
| SIRT1.NM_001142498- | UGCUAGAAGUGUUAAG | 1511 | AAACUUAACACUU | 1512 |
| Pr-23 | UUU | | CUAGCA | |
| SIRT1.NM_001142498- | CAGCCACGUUCUUUAA | 1513 | AAUUUAAAGAACG | 1514 |
| Pr-24 | AUU | | UGGCUG | |
| SIRT1.NM_001142498- | CUGAAAUACGUUGGAU | 1515 | ACUAUCCAACGUA | 1516 |
| Pr-25 | AGU | | UUUCAG | |
| SIRT1.NM_001142498- | AAGUUUGUGUCUCAAU | 1517 | AGUAUUGAGACAC | 1518 |
| Pr-26 | ACU | | AAACUU | |
| SIRT1.NM_001142498- | GUUGGAUAGUCGUUCU | 1519 | AACAGAACGACUA | 1520 |
| Pr-34 | GUU | | UCCAAC | |
| SIRT1.NM_001142498- | AGAGCAAGAUUAGAAU | 1521 | UUUAUUCUAAUCU | 1522 |
| Pr-36 | AAA | | UGCUCU | |
| SIRT1.NM_001142498- | UGCAUUACAGAAUGGU | 1523 | AUUACCAUUCUGU | 1524 |
| Pr-37 | AAU | | AAUGCA | |
| SIRT1.NM_012238- | GACUUAGCUCCCUGAA | 1525 | UAUUUCAGGGAGC | 1526 |
| Pr-1 | AUA | | UAAGUC | |
| SIRT1.NM_012238- | AGACUUAGCUCCCUGA | 1527 | AUUUCAGGGAGCU | 1528 |
| Pr-2 | AAU | | AAGUCU | |
| SIRT1.NM_012238- | GCUUCAAACGUGUGUG | 1529 | AUUCACACACGUU | 1530 |
| Pr-6 | AAU | | UGAAGC | |
| SIRT1.NM_012238- | GCCUUAGCCUUGUCUA | 1531 | AUAUAGACAAGGC | 1532 |
| Pr-12 | UAU | | UAAGGC | |
| SIRT1.NM_012238- | AGCCGUUUCUAAACUU | 1533 | AUUAAGUUUAGAA | 1534 |
| Pr-17 | AAU | | ACGGCU | |
| SIRT1.NM_012238- | CUGAAAUACGUUGGAU | 1535 | ACUAUCCAACGUA | 1536 |
| Pr-19 | AGU | | UUUCAG | |
| SIRT1.NM_012238- | GUUGGAUAGUCGUUCU | 1537 | AACAGAACGACUA | 1538 |
| Pr-30 | GUU | | UCCAAC | |
| SIRT1.NM_012238- | UGCCUUAGCCUUGUCU | 1539 | UAUAGACAAGGCU | 1540 |
| Pr-38 | AUA | | AAGGCA | |
| SIRT1.NM_012238- | CUAGCCGUUUCUAAAC | 1541 | UAAGUUUAGAAAC | 1542 |
| Pr-39 | UUA | | GGCUAG | |

TABLE 37
Sequences of SIRT1-saRNAs (with 3′ overhang)

| | | Sense SEQ | | Anti-sense |
|---|---|---|---|---|
| saRNA ID | Sense (passenger) | ID | Antisense (guide) | SEQ ID |
| SIRT1.NM_001142498- | GACUUAGCUCCCUGAA | 1543 | UAUUUCAGGGAGC | 1544 |
| Pr-1′ | AUAUU | | UAAGUCUU | |
| SIRT1.NM_001142498- | AGACUUAGCUCCCUGA | 1545 | AUUUCAGGGAGCU | 1546 |
| Pr-2′ | AAUUU | | AAGUCUUU | |
| SIRT1.NM_001142498- | GCAAGCUGUGACUAUA | 1547 | UUCUAUAGUCACA | 1548 |
| Pr-13′ | GAAUU | | GCUUGCUU | |
| SIRT1.NM_001142498- | GUGUUAAGUUUGUGUC | 1549 | UGAGACACAAACU | 1550 |
| Pr-18′ | UCAUU | | UAACACUU | |
| SIRT1.NM_001142498- | GCCUUAGCCUUGUCUA | 1551 | AUAUAGACAAGGC | 1552 |
| Pr-19′ | UAUUU | | UAAGGCUU | |
| SIRT1.NM_001142498- | UGCUAGAAGUGUUAAG | 1553 | AAACUUAACACUU | 1554 |
| Pr-23′ | UUUUU | | CUAGCAUU | |
| SIRT1.NM_001142498- | CAGCCACGUUCUUUAA | 1555 | AAUUUAAAGAACG | 1556 |
| Pr-24′ | AUUUU | | UGGCUGUU | |
| SIRT1.NM_001142498- | CUGAAAUACGUUGGAU | 1557 | ACUAUCCAACGUA | 1558 |
| Pr-25′ | AGUUU | | UUUCAGUU | |
| SIRT1.NM_001142498- | AAGUUUGUGUCUCAAU | 1559 | AGUAUUGAGACAC | 1560 |
| Pr-26′ | ACUUU | | AAACUUUU | |
| SIRT1.NM_001142498- | GUUGGAUAGUCGUUCU | 1561 | AACAGAACGACUA | 1562 |
| Pr-34′ | GUUUU | | UCCAACUU | |
| SIRT1.NM_001142498- | AGAGCAAGAUUAGAAU | 1563 | UUUAUUCUAAUCU | 1564 |
| Pr-36′ | AAAUU | | UGCUCUUU | |
| SIRT1.NM_001142498- | UGCAUUACAGAAUGGU | 1565 | AUUACCAUUCUGU | 1566 |
| Pr-37′ | AAUUU | | AAUGCAUU | |
| SIRT1.NM_012238- | GACUUAGCUCCCUGAA | 1567 | UAUUUCAGGGAGC | 1568 |
| Pr-1′ | AUAUU | | UAAGUCUU | |
| SIRT1.NM_012238- | AGACUUAGCUCCCUGA | 1569 | AUUUCAGGGAGCU | 1570 |
| Pr-2′ | AAUUU | | AAGUCUUU | |
| SIRT1.NM_012238- | GCUUCAAACGUGUGUG | 1571 | AUUCACACACGUU | 1572 |
| Pr-6′ | AAUUU | | UGAAGCUU | |
| SIRT1.NM_012238- | GCCUUAGCCUUGUCUA | 1573 | AUAUAGACAAGGC | 1574 |
| Pr-12′ | UAUUU | | UAAGGCUU | |
| SIRT1.NM_012238- | AGCCGUUUCUAAACUU | 1575 | AUUAAGUUUAGAA | 1576 |
| Pr-17′ | AAUUU | | ACGGCUUU | |
| SIRT1.NM_012238- | CUGAAAUACGUUGGAU | 1577 | ACUAUCCAACGUA | 1578 |
| Pr-19′ | AGUUU | | UUUCAGUU | |
| SIRT1.NM_012238- | GUUGGAUAGUCGUUCU | 1579 | AACAGAACGACUA | 1580 |
| Pr-30′ | GUUUU | | UCCAACUU | |
| SIRT1.NM_012238- | UGCCUUAGCCUUGUCU | 1581 | UAUAGACAAGGCU | 1582 |
| Pr-38′ | AUAUU | | AAGGCAUU | |
| SIRT1.NM_012238- | CUAGCCGUUUCUAAAC | 1583 | UAAGUUUAGAAAC | 1584 |
| Pr-39′ | UUAUU | | GGCUAGUU | |

SIRT1 gene encodes sirtuin 1 (SIRT1) protein, also known as NAD-dependent Deacetylase sirtuin-1, which is an enzyme that deacetylates proteins that contribute to cellular regulation. SIRT1 protein is associated with insulin sensitivity and type 2 diabetes (Sun et al., Cell Metab., vol. 6 (4):307 (2007), the contents of which are incorporated herein by reference in their entirety).

In one embodiment, provided is a method of modulating SIRT1 protein levels comprising administering SIRT1-saRNA of the present invention, wherein the SIRT1-saRNA targets an antisense RNA transcript of SIRT1 gene. In one embodiment, SIRT1 protein level is increased by at least 20, 30, 40%, or at least 45, 50, 55, 60, 65, 70, 75%, or at least 80% in the presence of the SIRT1-saRNA of the present invention compared to SIRT1 protein level in the absence of the SIRT1-saRNA of the present invention. In a further embodiment, SIRT1 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the SIRT1-saRNA of the present invention compared to SIRT1 protein level in the absence of the SIRT1-saRNA of the present invention.

In another embodiment, provided is a method of increasing insulin sensitivity and treating type 2 diabetes, comprising administering SIRT1-saRNA of the present invention, wherein the SIRT1-saRNA targets an antisense RNA transcript of SIRT1 gene.

In one embodiment, the SIRT1-saRNA of the present invention is an antisense sequence such as, but not limited to, SEQ ID NO: 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 5520, 5522, 5524, 5526, 5528, 5530, 5532, 5534, 5536, 5538, 5540, 5542, 5544, 5546, 5548, 5550, 5552, 5554, 5556, 5558, 5560, 5562, 5564, 5566, 5568, 5570, 5572, 5574, 5576, 5578, 5580, 5582, 5584, 5586, 5588, 5590, 5592, 5594, 5596, 5598, 5600, 5602, 5604, 5606, 5608, 5610, 5612, 5614, 5616, 5618, 5620, 5622, 5624, 5626, 5628, 5630, 5632, 5634, 5636, 5638, 5640, 5642, 5644, 5646, 5648, 5650, 5652, 5654, 5656, 5658, 5660, 5662, 5664, 5666, 5668, 5670, 5672, 5674, 5676, 5678, 5680, 5682, 5684, 5686, 5688, 5690, 5692, 5694, 5696, 5698, 5700, 5702, 5704, 5706, 5708, 5710, 5712, 5714, 5716 and 5718. As a non-limiting example, these SIRT1-saRNA sequences may be used to modulate SIRT1 protein levels, increase insulin sensitivity and treat type 2 diabetes.

In one embodiment, the SIRT1-saRNA of the present invention is an saRNA duplex. The saRNA duplex may be a pair of sense and antisense sequences such as, but not limited to, SEQ ID NOs: 1501 and 1502; SEQ ID NOs: 1503 and 1504; SEQ ID NOs: 1505 and 1506; SEQ ID NOs: 1507 and 1508; SEQ ID NOs: 1509 and 1510; SEQ ID NOs: 1511 and 1512; SEQ ID NOs: 1513 and 1514; SEQ ID NOs: 1515 and 1516; SEQ ID NOs: 1517 and 1518; SEQ ID NOs: 1519 and 1520; SEQ ID NOs: 1521 and 1522; SEQ ID NOs: 1523 and 1524; SEQ ID NOs: 1525 and 1526; SEQ ID NOs: 1527 and 1528; SEQ ID NOs: 1529 and 1530; SEQ ID NOs: 1531 and 1532; SEQ ID NOs: 1533 and 1534; SEQ ID NOs: 1535 and 1536; SEQ ID NOs: 1537 and 1538; SEQ ID NOs: 1539 and 1540; SEQ ID NOs: 1541 and 1542; SEQ ID NOs: 1543 and 1544; SEQ ID NOs: 1545 and 1546; SEQ ID NOs: 1547 and 1548; SEQ ID NOs: 1549 and 1550; SEQ ID NOs: 1551 and 1552; SEQ ID NOs: 1553 and 1554; SEQ ID NOs: 1555 and 1556; SEQ ID NOs: 1557 and 1558; SEQ ID NOs: 1559 and 1560; SEQ ID NOs: 1561 and 1562; SEQ ID NOs: 1563 and 1564; SEQ ID NOs: 1565 and 1566; SEQ ID NOs: 1567 and 1568; SEQ ID NOs: 1569 and 1570; SEQ ID NOs: 1571 and 1572; SEQ ID NOs: 1573 and 1574; SEQ ID NOs: 1575 and 1576; SEQ ID NOs: 1577 and 1578; SEQ ID NOs: 1579 and 1580; SEQ ID NOs: 1581 and 1582; SEQ ID NOs: 1583 and 1584; SEQ ID NOs: 5519 and 5520; SEQ ID NOs: 5521 and 5522; SEQ ID NOs: 5523 and 5524; SEQ ID NOs: 5525 and 5526; SEQ ID NOs: 5527 and 5528; SEQ ID NOs: 5529 and 5530; SEQ ID NOs: 5531 and 5532; SEQ ID NOs: 5533 and 5534; SEQ ID NOs: 5535 and 5536; SEQ ID NOs: 5537 and 5538; SEQ ID NOs: 5539 and 5540; SEQ ID NOs: 5541 and 5542; SEQ ID NOs: 5543 and 5544; SEQ ID NOs: 5545 and 5546; SEQ ID NOs: 5547 and 5548; SEQ ID NOs: 5549 and 5550; SEQ ID NOs: 5551 and 5552; SEQ ID NOs: 5553 and 5554; SEQ ID NOs: 5555 and 5556; SEQ ID NOs: 5557 and 5558; SEQ ID NOs: 5559 and 5560; SEQ ID NOs: 5561 and 5562; SEQ ID NOs: 5563 and 5564; SEQ ID NOs: 5565 and 5566; SEQ ID NOs: 5567 and 5568; SEQ ID NOs: 5569 and 5570; SEQ ID NOs: 5571 and 5572; SEQ ID NOs: 5573 and 5574; SEQ ID NOs: 5575 and 5576; SEQ ID NOs: 5577 and 5578; SEQ ID NOs: 5579 and 5580; SEQ ID NOs: 5581 and 5582; SEQ ID NOs: 5583 and 5584; SEQ ID NOs: 5585 and 5586; SEQ ID NOs: 5587 and 5588; SEQ ID NOs: 5589 and 5590; SEQ ID NOs: 5591 and 5592; SEQ ID NOs: 5593 and 5594; SEQ ID NOs: 5595 and 5596; SEQ ID NOs: 5597 and 5598; SEQ ID NOs: 5599 and 5600; SEQ ID NOs: 5601 and 5602; SEQ ID NOs: 5603 and 5604; SEQ ID NOs: 5605 and 5606; SEQ ID NOs: 5607 and 5608; SEQ ID NOs: 5609 and 5610; SEQ ID NOs: 5611 and 5612; SEQ ID NOs: 5613 and 5614; SEQ ID NOs: 5615 and 5616; SEQ ID NOs: 5617 and 5618; SEQ ID NOs: 5619 and 5620; SEQ ID NOs: 5621 and 5622; SEQ ID NOs: 5623 and 5624; SEQ ID NOs: 5625 and 5626; SEQ ID NOs: 5627 and 5628; SEQ ID NOs: 5629 and 5630; SEQ ID NOs: 5631 and 5632; SEQ ID NOs: 5633 and 5634; SEQ ID NOs: 5635 and 5636; SEQ ID NOs: 5637 and 5638; SEQ ID NOs: 5639 and 5640; SEQ ID NOs: 5641 and 5642; SEQ ID NOs: 5643 and 5644; SEQ ID NOs: 5645 and 5646; SEQ ID NOs: 5647 and 5648; SEQ ID NOs: 5649 and 5650; SEQ ID NOs: 5651 and 5652; SEQ ID NOs: 5653 and 5654; SEQ ID NOs: 5655 and 5656; SEQ ID NOs: 5657 and 5658; SEQ ID NOs: 5659 and 5660; SEQ ID NOs: 5661 and 5662; SEQ ID NOs: 5663 and 5664; SEQ ID NOs: 5665 and 5666; SEQ ID NOs: 5667 and 5668; SEQ ID NOs: 5669 and 5670; SEQ ID NOs: 5671 and 5672; SEQ ID NOs: 5673 and 5674; SEQ ID NOs: 5675 and 5676; SEQ ID NOs: 5677 and 5678; SEQ ID NOs: 5679 and 5680; SEQ ID NOs: 5681 and 5682; SEQ ID NOs: 5683 and 5684; SEQ ID NOs: 5685 and 5686; SEQ ID NOs: 5687 and 5688; SEQ ID NOs: 5689 and 5690; SEQ ID NOs: 5691 and 5692; SEQ ID NOs: 5693 and 5694; SEQ ID NOs: 5695 and 5696; SEQ ID NOs: 5697 and 5698; SEQ ID NOs: 5699 and 5700; SEQ ID NOs: 5701 and 5702; SEQ ID NOs: 5703 and 5704; SEQ ID NOs: 5705 and 5706; SEQ ID NOs: 5707 and 5708; SEQ ID NOs: 5709 and 5710; SEQ ID NOs: 5711 and 5712; SEQ ID NOs: 5713 and 5714; SEQ ID NOs: 5715 and 5716; SEQ ID NOs: 5717 and 5718. As a non-limiting example, these SIRT1-saRNA sequences which are saRNA duplexes may be used to modulate SIRT1 protein levels, increase insulin sensitivity and treat type 2 diabetes.

### TH Gene

As another non-limiting example, provided is a method of modulating the expression of TH gene comprising administering saRNA of the present invention, wherein the saRNA targets an antisense RNA transcript of TH gene. These saRNAs are called TH-saRNA. In one embodiment, the expression of TH gene is increased by at least 20, 30, 40%, or at least 45, 50, 55, 60, 65, 70, 75%, or at least 80% in the presence of the TH-saRNA of the present invention compared to the expression of TH gene in the absence of the TH-saRNA of the present invention. In a further embodiment, the expression of TH gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the TH-saRNA of the present invention compared to the expression of TH gene in the absence of the TH-saRNA of the present invention. The modulation of the expression of TH gene may be reflected or determined by the change of TH mRNA levels.

TH-saRNAs may be single-stranded and comprise 14-30 nucleotides. The sequence of a single-stranded TH-saRNA may have at least 60%, 70%, 80% or 90% identity with a sequence selected from the sequences of the antisense strands in Table 38-2. In one embodiment, the single-stranded saRNA comprises a sequence selected from the sequences of the antisense strands in Table 38-2. In one embodiment, the single-stranded TH-saRNA may have a 3′ tail. The sequence of a single-stranded TH-saRNA with a 3′ tail may have at least 60%, 70%, 80% or 90% identity with a sequence selected from the sequences of the antisense strands in Table 39. In one embodiment, the single-stranded saRNA comprises a sequence selected from the sequences of the antisense strands in Table 39.

TH-saRNAs may be double-stranded. The two strands form a duplex and each strand comprises 14-30 nucleotides. The first strand of a double-stranded TH-saRNA may have at least 60%, 70%, 80% or 90% identity with a sequence selected from the sequences of the antisense strands in Table 38-2. In one embodiment, the first strand of the double-stranded saRNA comprises a sequence selected from the sequences of the antisense strands in Table 38-2. The second strand of a double-stranded TH-saRNA may have at least 60%, 70%, 80% or 90% identity with a sequence selected from the sequences of the sense strands in Table 38-2. In one embodiment, the second strand of the double-stranded saRNA comprises a sequence selected from the sequences of the sense strands in Table 38-2. In one embodiment, the double-stranded TH-saRNA may have a 3′ overhang on each strand. The first strand of a double-stranded TH-saRNA may have at least 60%, 70%, 80% or 90% identity with a sequence selected from the sequences of the antisense strands in Table 39. In one embodiment, the first strand of the double-stranded saRNA comprises a sequence selected from the sequences of the antisense strands in Table 39. The second strand of a double-stranded TH-saRNA may have at least 60%, 70%, 80% or 90% identity with a sequence selected from the sequences of the sense strands in Table 39. In one embodiment, the second strand of the double-stranded saRNA comprises a sequence selected from the sequences of the sense strands in Table 39.

TH-saRNAs may be modified or unmodified.

Table 38-1 describes the target gene, the targeted sequence, the genomic location of the targeted sequence and the relative location of TH-saRNAs with no 3′ overhang. In Table 38-1, the targeted sequence is defined as a region on the template strand of the target gene which is identical to a region on the target antisense RNA transcript to which the antisense strand of an saRNA hybridizes. In Table 38-1, the relative location is the distance from the 5′ end of the targeted sequence to the TSS. A negative number represents a location upstream of the TSS and a positive number represents a location downstream of the TSS.

TABLE 38-1
Sequences of TH-saRNAs (with no 3′ overhang)

| | Target | Targeted | Targeted | Genomic Location of | Relative |
|---|---|---|---|---|---|
| saRNA ID | Gene | sequence | sequence ID | the Targeted sequence | Location |
| TH.NM_000360- | TH | cattccgatcgttaa | 1605 | chr11: 2192622 plus | 413 |
| Pr-9 | human | gatt | | strand | |
| TH.NM_000360- | TH | ggatggatttgtttcc | 1606 | chr11: 2192605 plus | 430 |
| Pr-14 | human | aca | | strand | |
| TH.NM_000360- | TH | acattccgatcgtta | 1607 | chr11: 2192621 plus | 414 |
| Pr-15 | human | agat | | strand | |
| TH.NM_000360- | TH | tcgttaagattcaag | 1608 | chr11: 2192630 plus | 405 |
| Pr-17 | human | atga | | strand | |
| TH.NM_000360- | TH | cgatcgttaagattc | 1609 | chr11: 2192627 plus | 408 |
| Pr-24 | human | aaga | | strand | |
| TH.NM_000360- | TH | ctgtgtccctgagaa | 1610 | chr11: 2192075 plus | 960 |
| Pr-30 | human | ggta | | strand | |
| TH.NM_000360- | TH | ttccgatcgttaagat | 1611 | chr11: 2192624 plus | 411 |
| Pr-38 | human | tca | | strand | |

TABLE 38-2
Sequences of TH-saRNAs (with no 3′ overhang)

| | | Sense SEQ | | Anti-sense |
|---|---|---|---|---|
| saRNA ID | Sense (passenger) | ID | Antisense (guide) | SEQ ID |
| TH.NM_000360-Pr-9 | CAUUCCGAUCGU | 1612 | AAUCUUAACGAUC | 1613 |
| | UAAGAUU | | GGAAUG | |
| TH.NM_000360-Pr- | GGAUGGAUUUG | 1614 | UGUGGAAACAAAU | 1615 |
| 14 | UUUCCACA | | CCAUCC | |
| TH.NM_000360-Pr- | ACAUUCCGAUCG | 1616 | AUCUUAACGAUCG | 1617 |
| 15 | UUAAGAU | | GAAUGU | |
| TH.NM_000360-Pr- | UCGUUAAGAUUC | 1618 | UCAUCUUGAAUCU | 1619 |
| 17 | AAGAUGA | | UAACGA | |
| TH.NM_000360-Pr- | CGAUCGUUAAGA | 1620 | UCUUGAAUCUUAA | 1621 |
| 24 | UUCAAGA | | CGAUCG | |
| TH.NM_000360-Pr- | CUGUGUCCCUGA | 1622 | UACCUUCUCAGGGA | 1623 |
| 30 | GAAGGUA | | CACAG | |
| TH.NM_000360-Pr- | UUCCGAUCGUUA | 1624 | UGAAUCUUAACGA | 1625 |
| 38 | AGAUUCA | | UCGGAA | |

TABLE 39
Sequences of TH-saRNAs (with 3′ overhang)

| | | Sense | | Antisense |
|---|---|---|---|---|
| saRNA ID | Sense (passenger) | SEQ ID | Antisense (guide) | SEQ ID |
| TH.NM_000360-Pr- | CAUUCCGAUCGUUA | 1626 | AAUCUUAACGAUC | 1627 |
| 9′ | AGAUUUU | | GGAAUGUU | |
| TH.NM_000360-Pr- | GGAUGGAUUUGUU | 1628 | UGUGGAAACAAAU | 1629 |
| 14′ | UCCACAUU | | CCAUCCUU | |
| TH.NM_000360-Pr- | ACAUUCCGAUCGUU | 1630 | AUCUUAACGAUCG | 1631 |
| 15′ | AAGAUUU | | GAAUGUUU | |
| TH.NM_000360-Pr- | UCGUUAAGAUUCA | 1632 | UCAUCUUGAAUCU | 1633 |
| 17′ | AGAUGAUU | | UAACGAUU | |
| TH.NM_000360-Pr- | CGAUCGUUAAGAU | 1634 | UCUUGAAUCUUAA | 1635 |
| 24′ | UCAAGAUU | | CGAUCGUU | |
| TH.NM_000360-Pr- | CUGUGUCCCUGAGA | 1636 | UACCUUCUCAGGGA | 1637 |
| 30′ | AGGUAUU | | CACAGUU | |
| TH.NM_000360-Pr- | UUCCGAUCGUUAA | 1638 | UGAAUCUUAACGA | 1639 |
| 38′ | GAUUCAUU | | UCGGAAUU | |

TH gene encodes the enzyme tyrosine hydroxylase, which is important for normal functioning of the nervous system. Tyrosine hydroxylase takes part in the first step of the pathway that produces a group of hormones called catecholamines. This enzyme assists the conversion of the amino acid tyrosine to a catecholamine called dopamine. Dopamine is a neurotransmitter because it transmits signals between nerve cells in the brain to help control physical movement and emotional behavior. Other catecholamines such as norepinephrine and epinephrine are produced from dopamine. Norepinephrine and epinephrine are involved in the autonomic nervous system, which controls involuntary body processes such as the regulation of blood pressure and body temperature. A deficiency of tyrosine hydroxylase enzyme results in less dopamine, norepinephrine, and epinephrine. These catecholamines are necessary for normal nervous system function, and changes in their levels contribute to the abnormal movements, nervous system dysfunction, and other neurological problems seen in people with TH deficiency. Mutations of TH gene cause dopa-responsive dystonia (Cai et al., PLoS One, vol. 8(6):e65215 (2013), the contents of which are incorporated herein by reference in their entirety). This condition is characterized by a pattern of involuntary muscle contractions (dystonia), tremors, and other uncontrolled movements. A reduction in normal tyrosine hydroxylase enzyme leads to a decrease in the production of dopamine, which causes the movement problems characteristic of dopa-responsive dystonia. Sudden infant death syndrome (SIDS) has also been associated with the expression levels of TH (Holgert et al., Proc Natl Acad Sci. USA, vol. 92 (16):7575 (1995), the contents of which are incorporated herein by reference in their entirety).

In one embodiment, provided is a method of modulating tyrosine hydroxylase protein levels comprising administering TH-saRNA of the present invention, wherein the TH-saRNA targets an antisense RNA transcript of TH gene. In another embodiment, provided is a method of treating tyrosine hydroxylase deficiency comprising administering TH-saRNA of the present invention, wherein the TH-saRNA targets an antisense RNA transcript of TH gene. In one embodiment, tyrosine hydroxylase protein level is increased by at least 20, 30, 40%, or at least 45, 50, 55, 60, 65, 70, 75%, or at least 80% in the presence of the TH-saRNA of the present invention compared to tyrosine hydroxylase protein level in the absence of the TH-saRNA of the present invention. In a further embodiment, tyrosine hydroxylase protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the TH-saRNA of the present invention compared to tyrosine hydroxylase protein level in the absence of the TH-saRNA of the present invention.

In another embodiment, provided is a method of regulating neurotransmitter levels comprising administering TH-saRNA of the present invention, wherein the TH-saRNA targets an antisense RNA transcript of TH gene. In one embodiment, the neurotransmitter level is increased by at least 20, 30, 40%, or at least 45, 50, 55, 60, 65, 70, 75%, or at least 80% in the presence of the TH-saRNA of the present invention compared to neurotransmitter level in the absence of the TH-saRNA of the present invention. In a further embodiment, tyrosine hydroxylase protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the TH-saRNA of the present invention compared to tyrosine hydroxylase protein level in the absence of the TH-saRNA of the present invention. The neurotransmitter may be a catecholamine. Non-limited examples of catecholamine include dopamine, norepinephrine, or epinephrine.

In another embodiment, provided is a method of treating dopa-responsive dystonia comprising administering TH-saRNA of the present invention, wherein the TH-saRNA targets an antisense RNA transcript of TH gene, and wherein the symptoms of dopa-responsive dystonia are reduced.

In one embodiment, the TH-saRNA of the present invention is an antisense sequence such as, but not limited to, SEQ ID NO: 1613, 1615, 1617, 1619, 1621, 1623, 1625, 1627, 1629, 1631, 1633, 1635, 1637, 1639, 5720, 5722, 5724, 5726, 5728, 5730, 5732, 5734, 5736, 5738, 5740, 5742, 5744, 5746, 5748, 5750, 5752, 5754, 5756, 5758, 5760, 5762, 5764, 5766, 5768, 5770, 5772, 5774, 5776, 5778, 5780, 5782, 5784, 5786, 5788 and 5790. As a non-limiting example, these TH-saRNA sequences may be used to modulate TH protein levels, increase insulin sensitivity and treat type 2 diabetes.

In one embodiment, the TH-saRNA of the present invention is an saRNA duplex. The saRNA duplex may be a pair of sense and antisense sequences such as, but not limited to, SEQ ID NOs: 1612 and 1613; SEQ ID NOs: 1614 and 1615; SEQ ID NOs: 1616 and 1617; SEQ ID NOs: 1618 and 1619; SEQ ID NOs: 1620 and 1621; SEQ ID NOs: 1622 and 1623; SEQ ID NOs: 1624 and 1625; SEQ ID NOs: 1626 and 1627; SEQ ID NOs: 1628 and 1629; SEQ ID NOs: 1630 and 1631; SEQ ID NOs: 1632 and 1633; SEQ ID NOs: 1634 and 1635; SEQ ID NOs: 1636 and 1637; SEQ ID NOs: 1638 and 1639; SEQ ID NOs: 5719 and 5720; SEQ ID NOs: 5721 and 5722; SEQ ID NOs: 5723 and 5724; SEQ ID NOs: 5725 and 5726; SEQ ID NOs: 5727 and 5728; SEQ ID NOs: 5729 and 5730; SEQ ID NOs: 5731 and 5732; SEQ ID NOs: 5733 and 5734; SEQ ID NOs: 5735 and 5736; SEQ ID NOs: 5737 and 5738; SEQ ID NOs: 5739 and 5740; SEQ ID NOs: 5741 and 5742; SEQ ID NOs: 5743 and 5744; SEQ ID NOs: 5745 and 5746; SEQ ID NOs: 5747 and 5748; SEQ ID NOs: 5749 and 5750; SEQ ID NOs: 5751 and 5752; SEQ ID NOs: 5753 and 5754; SEQ ID NOs: 5755 and 5756; SEQ ID NOs: 5757 and 5758; SEQ ID NOs: 5759 and 5760; SEQ ID NOs: 5761 and 5762; SEQ ID NOs: 5763 and 5764; SEQ ID NOs: 5765 and 5766; SEQ ID NOs: 5767 and 5768; SEQ ID NOs: 5769 and 5770; SEQ ID NOs: 5771 and 5772; SEQ ID NOs: 5773 and 5774; SEQ ID NOs: 5775 and 5776; SEQ ID NOs: 5777 and 5778; SEQ ID NOs: 5779 and 5780; SEQ ID NOs: 5781 and 5782; SEQ ID NOs: 5783 and 5784; SEQ ID NOs: 5785 and 5786; SEQ ID NOs: 5787 and 5788; SEQ ID NOs: 5789 and 5790. As a non-limiting example, these TH-saRNA sequences which are saRNA duplexes may be used to modulate TH protein levels, increase insulin sensitivity and treat type 2 diabetes.

### EPO Gene

As another non-limiting example, provided is a method of modulating the expression of EPO gene comprising administering saRNA of the present invention, wherein the saRNA targets an antisense RNA transcript of EPO gene. These saRNAs are called EPO-saRNA. In one embodiment, the expression of EPO gene is increased by at least 20, 30, 40%, or at least 45, 50, 55, 60, 65, 70, 75%, or at least 80% in the presence of the EPO-saRNA of the present invention compared to the expression of EPO gene in the absence of the EPO-saRNA of the present invention. In a further embodiment, the expression of EPO gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the EPO-saRNA of the present invention compared to the expression of EPO gene in the absence of the EPO-saRNA of the present invention. The modulation of the expression of EPO gene may be reflected or determined by the change of EPO mRNA levels.

EPO-saRNAs may be single-stranded and comprise 14-30 nucleotides. The sequence of a single-stranded EPO-saRNA may have at least 60%, 70%, 80% or 90% identity with a sequence selected from the sequences of the antisense strands in Table 40-2. In one embodiment, the single-stranded saRNA comprises a sequence selected from the sequences of the antisense strands in Table 40-2. In one embodiment, the single-stranded EPO-saRNA may have a 3′ tail. The sequence of a single-stranded EPO-saRNA with a 3′ tail may have at least 60%, 70%, 80% or 90% identity with a sequence selected from the sequences of the antisense strands in Table 41. In one embodiment, the single-stranded saRNA comprises a sequence selected from the sequences of the antisense strands in Table 41.

EPO-saRNAs may be double-stranded. The two strands form a duplex and each strand comprises 14-30 nucleotides. The first strand of a double-stranded EPO-saRNA may have at least 60%, 70%, 80% or 90% identity with a sequence selected from the sequences of the antisense strands in Table 40-2. In one embodiment, the first strand of the double-stranded saRNA comprises a sequence selected from the sequences of the antisense strands in Table 40-2. The second strand of a double-stranded EPO-saRNA may have at least 60%, 70%, 80% or 90% identity with a sequence selected from the sequences of the sense strands in Table 40-2. In one embodiment, the second strand of the double-stranded saRNA comprises a sequence selected from the sequences of the sense strands in Table 40-2. In one embodiment, the double-stranded EPO-saRNA may have a 3′ overhang on each strand. The first strand of a double-stranded EPO-saRNA may have at least 60%, 70%, 80% or 90% identity with a sequence selected from the sequences of the antisense strands in Table 41. In one embodiment, the first strand of the double-stranded saRNA comprises a sequence selected from the sequences of the antisense strands in Table 41. The second strand of a double-stranded EPO-saRNA may have at least 60%, 70%, 80% or 90% identity with a sequence selected from the sequences of the sense strands in Table 41. In one embodiment, the second strand of the double-stranded saRNA comprises a sequence selected from the sequences of the sense strands in Table 41.

EPO-saRNAs may be modified or unmodified.

Table 40-1 describes the target gene, the targeted sequence, the genomic location of the targeted sequence and the relative location of EPO-saRNAs with no 3′ overhang. In Table 40-1, the targeted sequence is defined as a region on the template strand of the target gene which is identical to a region on the target antisense RNA transcript to which the antisense strand of an saRNA hybridizes. In Table 40-1, the relative location is the distance from the 5′ end of the targeted sequence to the TSS. A negative number represents a location upstream of the TSS and a positive number represents a location downstream of the TSS.

TABLE 40-1
Sequences of EPO-saRNAs (with no 3′ overhang)

| | | | Targeted | Genomic Location of | |
|---|---|---|---|---|---|
| | | Targeted | sequence | the Targeted | Relative |
| saRNA ID | Target Gene | sequence | ID | sequence | Location |
| EPO.NM_000799- | EPO human | ggacatgttctagaaa | 1640 | chr7: 100317919 | −484 |
| Pr-2 | | caa | | minus strand | |
| EPO.NM_000799- | EPO human | gcgtgagttctgtgga | 1641 | chr7: 100319346 | 943 |
| Pr-6 | | atg | | minus strand | |
| EPO.NM_000799- | EPO human | gagcgtgctatctgg | 1642 | chr7: 100318092 | −311 |
| Pr-9 | | gaaa | | minus strand | |
| EPO.NM_000799- | EPO human | cgtgagttctgtggaa | 1643 | chr7: 100319345 | 942 |
| Pr-14 | | tgt | | minus strand | |
| EPO.NM_000799- | EPO human | tgaccatgttgaggaa | 1644 | chr7: 100317897 | −506 |
| Pr-25 | | ttt | | minus strand | |
| EPO.NM_000799- | EPO human | gggtctcaccgtgata | 1645 | chr7: 100319316 | 913 |
| Pr-27 | | ttc | | minus strand | |
| EPO.NM_000799- | EPO human | ggctgttatctgcatgt | 1646 | chr7: 100318327 | −76 |
| Pr-33 | | gt | | minus strand | |
| EPO.NM_000799- | EPO human | ttgctgagttccgcaa | 1647 | chr7: 100318005 | −398 |
| Pr-39 | | agt | | minus strand | |

TABLE 40-2
Sequences of EPO-saRNAs (with no 3′ overhang)

| | | | | Anti- |
|---|---|---|---|---|
| | | Sense SEQ | | sense |
| saRNA ID | Sense (passenger) | ID | Antisense (guide) | SEQ ID |
| EPO.NM_000799-Pr-2 | GGACAUGUUCUAG | 1648 | UUGUUUCUAGAAC | 1649 |
| | AAACAA | | AUGUCC | |
| EPO.NM_000799-Pr-6 | GCGUGAGUUCUGU | 1650 | CAUUCCACAGAAC | 1651 |
| | GGAAUG | | UCACGC | |
| EPO.NM_000799-Pr-9 | GAGCGUGCUAUCU | 1652 | UUUCCCAGAUAGC | 1653 |
| | GGGAAA | | ACGCUC | |
| EPO.NM_000799-Pr-14 | CGUGAGUUCUGUG | 1654 | ACAUUCCACAGAA | 1655 |
| | GAAUGU | | CUCACG | |
| EPO.NM_000799-Pr-25 | UGACCAUGUUGAG | 1656 | AAAUUCCUCAACA | 1657 |
| | GAAUUU | | UGGUCA | |
| EPO.NM_000799-Pr-27 | GGGUCUCACCGUG | 1658 | GAAUAUCACGGUG | 1659 |
| | AUAUUC | | AGACCC | |
| EPO.NM_000799-Pr-33 | GGCUGUUAUCUGC | 1660 | ACACAUGCAGAUA | 1661 |
| | AUGUGU | | ACAGCC | |
| EPO.NM_000799-Pr-39 | UUGCUGAGUUCCG | 1662 | ACUUUGCGGAACU | 1663 |
| | CAAAGU | | CAGCAA | |

TABLE 41
Sequences of EPO-saRNAs (with 3′ overhang)

| | | Sense | | Antisense |
|---|---|---|---|---|
| saRNA ID | Sense (passenger) | SEQ ID | Antisense (guide) | SEQ ID |
| EPO.NM_000799- | GGACAUGUUCUAGAAACA | 1664 | UUGUUUCUAGAACA | 1665 |
| Pr-2′ | AUU | | UGUCCUU | |
| EPO.NM_000799- | GCGUGAGUUCUGUGGAA | 1666 | CAUUCCACAGAACU | 1667 |
| Pr-6′ | UGUU | | CACGCUU | |
| EPO.NM_000799- | GAGCGUGCUAUCUGGGAA | 1668 | UUUCCCAGAUAGCA | 1669 |
| Pr-9′ | AUU | | CGCUCUU | |
| EPO.NM_000799- | CGUGAGUUCUGUGGAAU | 1670 | ACAUUCCACAGAAC | 1671 |
| Pr-14′ | GUUU | | UCACGUU | |
| EPO.NM_000799- | UGACCAUGUUGAGGAAU | 1672 | AAAUUCCUCAACAU | 1673 |
| Pr-25′ | UUUU | | GGUCAUU | |
| EPO.NM_000799- | GGGUCUCACCGUGAUAUU | 1674 | GAAUAUCACGGUGA | 1675 |
| Pr-27′ | CUU | | GACCCUU | |
| EPO.NM_000799- | GGCUGUUAUCUGCAUGUG | 1676 | ACACAUGCAGAUAA | 1677 |
| Pr-33′ | UUU | | CAGCCUU | |
| EPO.NM_000799- | UUGCUGAGUUCCGCAAAG | 1678 | ACUUUGCGGAACUC | 1679 |
| Pr-39′ | UUU | | AGCAAUU | |

EPO gene encodes erythropoietin protein, a secreted, glycosylated cytokine composed of four alpha helical bundles. Erythropoietin protein is found in the plasma and regulates red cell production by promoting erythroid differentiation and initiating hemoglobin synthesis. It is the principal hormone involved in the maintenance of a physiological level of circulating erythrocyte mass. It also has neuroprotective activity against a variety of potential brain injuries and antiapoptotic functions in several tissue types. Mutations of EPO gene are associated with microvascular complications of diabetes 2 (MVCD2) including diabetic retinopathy, diabetic nephropathy leading to end-stage renal disease, and diabetic neuropathy (Tong et al., PNAS, vol. 105:6998 (2008), the contents of which are incorporated herein by reference in their entirety).

In one embodiment, provided is a method of modulating erythropoietin protein levels comprising administering EPO-saRNA of the present invention, wherein the EPO-saRNA targets an antisense RNA transcript of EPO gene. In one embodiment, erythropoietin protein level is increased by at least 20, 30, 40%, or at least 45, 50, 55, 60, 65, 70, 75%, or at least 80% in the presence of the EPO-saRNA of the present invention compared to erythropoietin protein level in the absence of the EPO-saRNA of the present invention. In a further embodiment, erythropoietin protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the EPO-saRNA of the present invention compared to erythropoietin protein level in the absence of the EPO-saRNA of the present invention.

In another embodiment, provided is a method of preventing or treating MVCD2 comprising administering EPO-saRNA of the present invention, wherein the EPO-saRNA targets an antisense RNA transcript of EPO gene, wherein the patient's susceptibility of MVCD2 is reduced or the symptoms of MVCD2 are reduced.

In one embodiment, the EPO-saRNA of the present invention is an antisense sequence such as, but not limited to, SEQ ID NO: 1649, 1651, 1653, 1655, 1657, 1659, 1661, 1663, 1665, 1667, 1669, 1671, 1673, 1675, 1677, 1679, 3140, 3142, 3144, 3146, 3148, 3150, 3152, 3154, 3156, 3158, 3160, 3162, 3164, 3166, 3168, 3170, 3172, 3174, 3176, 3178, 3180, 3182, 3184, 3186, 3188, 3190, 3192, 3194, 3196, 3198, 3200, 3202, 3204, 3206, 3208, 3210, 3212, 3214 and 3216. As a non-limiting example, these EPO-saRNA sequences may be used to modulate EPO protein levels, preventing or treating MVCD2.

In one embodiment, the EPO-saRNA of the present invention is an saRNA duplex. The saRNA duplex may be a pair of sense and antisense sequences such as, but not limited to, SEQ ID NOs: 1648 and 1649; SEQ ID NOs: 1650 and 1651; SEQ ID NOs: 1652 and 1653; SEQ ID NOs: 1654 and 1655; SEQ ID NOs: 1656 and 1657; SEQ ID NOs: 1658 and 1659; SEQ ID NOs: 1660 and 1661; SEQ ID NOs: 1662 and 1663; SEQ ID NOs: 1664 and 1665; SEQ ID NOs: 1666 and 1667; SEQ ID NOs: 1668 and 1669; SEQ ID NOs: 1670 and 1671; SEQ ID NOs: 1672 and 1673; SEQ ID NOs: 1674 and 1675; SEQ ID NOs: 1676 and 1677; SEQ ID NOs: 1678 and 1679; SEQ ID NOs: 3139 and 3140; SEQ ID NOs: 3141 and 3142; SEQ ID NOs: 3143 and 3144; SEQ ID NOs: 3145 and 3146; SEQ ID NOs: 3147 and 3148; SEQ ID NOs: 3149 and 3150; SEQ ID NOs: 3151 and 3152; SEQ ID NOs: 3153 and 3154; SEQ ID NOs: 3155 and 3156; SEQ ID NOs: 3157 and 3158; SEQ ID NOs: 3159 and 3160; SEQ ID NOs: 3161 and 3162; SEQ ID NOs: 3163 and 3164; SEQ ID NOs: 3165 and 3166; SEQ ID NOs: 3167 and 3168; SEQ ID NOs: 3169 and 3170; SEQ ID NOs: 3171 and 3172; SEQ ID NOs: 3173 and 3174; SEQ ID NOs: 3175 and 3176; SEQ ID NOs: 3177 and 3178; SEQ ID NOs: 3179 and 3180; SEQ ID NOs: 3181 and 3182; SEQ ID NOs: 3183 and 3184; SEQ ID NOs: 3185 and 3186; SEQ ID NOs: 3187 and 3188; SEQ ID NOs: 3189 and 3190; SEQ ID NOs: 3191 and 3192; SEQ ID NOs: 3193 and 3194; SEQ ID NOs: 3195 and 3196; SEQ ID NOs: 3197 and 3198; SEQ ID NOs: 3199 and 3200; SEQ ID NOs: 3201 and 3202; SEQ ID NOs: 3203 and 3204; SEQ ID NOs: 3205 and 3206; SEQ ID NOs: 3207 and 3208; SEQ ID NOs: 3209 and 3210; SEQ ID NOs: 3211 and 3212; SEQ ID NOs: 3213 and 3214; SEQ ID NOs: 3215 and 3216. As a non-limiting example, these EPO-saRNA sequences which are saRNA duplexes may be used to modulate EPO protein levels, preventing or treating MVCD2.

### CDKN1B Gene

As another non-limiting example, provided is a method of modulating the expression of CDKN1B gene comprising administering saRNA of the present invention, wherein the saRNA targets an antisense RNA transcript of CDKN1B gene. These saRNAs are called CDKN1B-saRNA. In one embodiment, the expression of CDKN1B gene is increased by at least 20, 30, 40%, or at least 45, 50, 55, 60, 65, 70, 75%, or at least 80% in the presence of the CDKN1B-saRNA of the present invention compared to the expression of CDKN1B gene in the absence of the CDKN1B-saRNA of the present invention. In a further embodiment, the expression of CDKN1B gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the CDKN1B-saRNA of the present invention compared to the expression of CDKN1B gene in the absence of the CDKN1B-saRNA of the present invention. The modulation of the expression of CDKN1B gene may be reflected or determined by the change of CDKN1B mRNA levels.

CDKN1B-saRNAs may be single-stranded and comprise 14-30 nucleotides. The sequence of a single-stranded CDKN1B-saRNA may have at least 60%, 70%, 80% or 90% identity with a sequence selected from the sequences of the antisense strands in Table 42-2. In one embodiment, the single-stranded saRNA comprises a sequence selected from the sequences of the antisense strands in Table 42-2. In one embodiment, the single-stranded CDKN1B-saRNA may have a 3′ tail. The sequence of a single-stranded CDKN1B-saRNA with a 3′ tail may have at least 60%, 70%, 80% or 90% identity with a sequence selected from the sequences of the antisense strands in Table 43. In one embodiment, the single-stranded saRNA comprises a sequence selected from the sequences of the antisense strands in Table 43.

CDKN1B-saRNAs may be double-stranded. The two strands form a duplex and each strand comprises 14-30 nucleotides. The first strand of a double-stranded CDKN1B-saRNA may have at least 60%, 70%, 80% or 90% identity with a sequence selected from the sequences of the antisense strands in Table 42-2. In one embodiment, the first strand of the double-stranded saRNA comprises a sequence selected from the sequences of the antisense strands in Table 42-2. The second strand of a double-stranded CDKN1B-saRNA may have at least 60%, 70%, 80% or 90% identity with a sequence selected from the sequences of the sense strands in Table 42-2. In one embodiment, the second strand of the double-stranded saRNA comprises a sequence selected from the sequences of the sense strands in Table 42-2. In one embodiment, the double-stranded CDKN1B-saRNA may have a 3′ overhang on each strand. The first strand of a double-stranded CDKN1B-saRNA may have at least 60%, 70%, 80% or 90% identity with a sequence selected from the sequences of the antisense strands in Table 43. In one embodiment, the first strand of the double-stranded saRNA comprises a sequence selected from the sequences of the antisense strands in Table 43. The second strand of a double-stranded CDKN1B-saRNA may have at least 60%, 70%, 80% or 90% identity with a sequence selected from the sequences of the sense strands in Table 43. In one embodiment, the second strand of the double-stranded saRNA comprises a sequence selected from the sequences of the sense strands in Table 43.

CDKN1B-saRNAs may be modified or unmodified.

Table 42-1 describes the target gene, the targeted sequence, the genomic location of the targeted sequence and the relative location of CDKN1B-saRNAs with no 3′ overhang. In Table 42-1, the targeted sequence is defined as a region on the template strand of the target gene which is identical to a region on the target antisense RNA transcript to which the antisense strand of an saRNA hybridizes. In Table 42-1, the relative location is the distance from the 5′ end of the targeted sequence to the TSS. A negative number represents a location upstream of the TSS and a positive number represents a location downstream of the TSS.

TABLE 42-1
Sequences of CDKN1B-saRNAs (with no 3′ overhang)

| | Target | Targeted | Targeted | Genomic Location of the | Relative |
|---|---|---|---|---|---|
| saRNA ID | Gene | sequence | sequence ID | Targeted sequence | Location |
| CDKN1B.NM_004064- | CDKN1B | atgcgtgtcctcagagt | 1680 | chr12: 12871141 minus | 957 |
| Pr-1 | human | ta | | strand | |
| CDKN1B.NM_004064- | CDKN1B | ggctggcctcggagaa | 1681 | chr12: 12870060 minus | −124 |
| Pr-2 | human | att | | strand | |
| CDKN1B.NM_004064- | CDKN1B | gctagtgaatgctttca | 1682 | chr12: 12869231 minus | −953 |
| Pr-3 | human | ga | | strand | |
| CDKN1B.NM_004064- | CDKN1B | cggcgagtctatttaaa | 1683 | chr12: 12870269 minus | 85 |
| Pr-4 | human | ca | | strand | |
| CDKN1B.NM_004064- | CDKN1B | gaaaggaactcaagat | 1684 | chr12: 12869557 minus | −627 |
| Pr-5 | human | taa | | strand | |
| CDKN1B.NM_004064- | CDKN1B | cgagtctccgctgatca | 1685 | chr12: 12870554 minus | 370 |
| Pr-13 | human | aa | | strand | |
| CDKN1B.NM_004064- | CDKN1B | gctggcctcggagaaa | 1686 | chr12: 12870059 minus | −125 |
| Pr-14 | human | tta | | strand | |
| CDKN1B.NM_004064- | CDKN1B | gcgagtctatttaaaca | 1687 | chr12: 12870267 minus | 83 |
| Pr-19 | human | ga | | strand | |
| CDKN1B.NM_004064- | CDKN1B | acaatattcagatgcta | 1688 | chr12: 12869244 minus | −940 |
| Pr-21 | human | gt | | strand | |
| CDKN1B.NM_004064- | CDKN1B | ccgagtctccgctgatc | 1689 | chr12: 12870555 minus | 371 |
| Pr-22 | human | aa | | strand | |
| CDKN1B.NM_004064- | CDKN1B | tggcctcggagaaatta | 1690 | chr12: 12870057 minus | −127 |
| Pr-24 | human | aa | | strand | |

TABLE 42-2
Sequences of CDKN1B-saRNAs (with no 3′ overhang)

| | | Sense SEQ | | Anti-sense SEQ |
|---|---|---|---|---|
| saRNA ID | Sense (passenger) | ID | Antisense (guide) | ID |
| CDKN1B.NM_004064- | AUGCGUGUCCUCAG | 1691 | UAACUCUGAGGACAC | 1692 |
| Pr-1 | AGUUA | | GCAU | |
| CDKN1B.NM_004064- | GGCUGGCCUCGGAG | 1693 | AAUUUCUCCGAGGCC | 1694 |
| Pr-2 | AAAUU | | AGCC | |
| CDKN1B.NM_004064- | GCUAGUGAAUGCUU | 1695 | UCUGAAAGCAUUCAC | 1696 |
| Pr-3 | UCAGA | | UAGC | |
| CDKN1B.NM_004064- | CGGCGAGUCUAUUU | 1697 | UGUUUAAAUAGACUC | 1698 |
| Pr-4 | AAACA | | GCCG | |
| CDKN1B.NM_004064- | GAAAGGAACUCAAG | 1699 | UUAAUCUUGAGUUCC | 1700 |
| Pr-5 | AUUAA | | UUUC | |
| CDKN1B.NM_004064- | CGAGUCUCCGCUGA | 1701 | UUUGAUCAGCGGAGA | 1702 |
| Pr- | UCAAA | | CUCG | |
| 13 | | | | |
| CDKN1B.NM_004064- | GCUGGCCUCGGAGA | 1703 | UAAUUUCUCCGAGGC | 1704 |
| Pr- | AAUUA | | CAGC | |
| 14 | | | | |
| CDKN1B.NM_004064- | GCGAGUCUAUUUAA | 1705 | UCUGUUUAAAUAGAC | 1706 |
| Pr- | ACAGA | | UCGC | |
| 19 | | | | |
| CDKN1B.NM_004064- | ACAAUAUUCAGAUG | 1707 | ACUAGCAUCUGAAUA | 1708 |
| Pr- | CUAGU | | UUGU | |
| 21 | | | | |
| CDKN1B.NM_004064- | CCGAGUCUCCGCUG | 1709 | UUGAUCAGCGGAGAC | 1710 |
| Pr- | AUCAA | | UCGG | |
| 22 | | | | |
| CDKN1B.NM_004064- | UGGCCUCGGAGAAA | 1711 | UUUAAUUUCUCCGAG | 1712 |
| Pr- | UUAAA | | GCCA | |
| 24 | | | | |

TABLE 43
Sequences of CDKN1B-saRNAs (with 3′ overhang)

| | | Sense | | Antisense |
|---|---|---|---|---|
| saRNA ID | Sense (passenger) | SEQ ID | Antisense (guide) | SEQ ID |
| CDKN1B.NM_004064- | AUGCGUGUCCUCAGAGU | 1713 | UAACUCUGAGGACACG | 1714 |
| Pr-1′ | UAUU | | CAUUU | |
| CDKN1B.NM_004064- | GGCUGGCCUCGGAGAAA | 1715 | AAUUUCUCCGAGGCCA | 1716 |
| Pr-2′ | UUUU | | GCCUU | |
| CDKN1B.NM_004064- | GCUAGUGAAUGCUUUC | 1717 | UCUGAAAGCAUUCACU | 1718 |
| Pr-3′ | AGAUU | | AGCUU | |
| CDKN1B.NM_004064- | CGGCGAGUCUAUUUAA | 1719 | UGUUUAAAUAGACUC | 1720 |
| Pr-4′ | ACAUU | | GCCGUU | |
| CDKN1B.NM_004064- | GAAAGGAACUCAAGAU | 1721 | UUAAUCUUGAGUUCCU | 1722 |
| Pr-5′ | UAAUU | | UUCUU | |
| CDKN1B.NM_004064- | CGAGUCUCCGCUGAUCA | 1723 | UUUGAUCAGCGGAGAC | 1724 |
| Pr-13′ | AAUU | | UCGUU | |
| CDKN1B.NM_004064- | GCUGGCCUCGGAGAAAU | 1725 | UAAUUUCUCCGAGGCC | 1726 |
| Pr-14′ | UAUU | | AGCUU | |
| CDKN1B.NM_004064- | GCGAGUCUAUUUAAAC | 1727 | UCUGUUUAAAUAGAC | 1728 |
| Pr-19′ | AGAUU | | UCGCUU | |
| CDKN1B.NM_004064- | ACAAUAUUCAGAUGCU | 1729 | ACUAGCAUCUGAAUAU | 1730 |
| Pr-21′ | AGUUU | | UGUUU | |
| CDKN1B.NM_004064- | CCGAGUCUCCGCUGAUC | 1731 | UUGAUCAGCGGAGACU | 1732 |
| Pr-22′ | AAUU | | CGGUU | |
| CDKN1B.NM_004064- | UGGCCUCGGAGAAAUU | 1733 | UUUAAUUUCUCCGAGG | 1734 |
| Pr-24′ | AAAUU | | CCAUU | |

CDKN1B gene encodes p27 (Kip1) protein, which plays an important role in controlling cell growth and division. p27 protein prevents cells from dividing too quickly or at the wrong time by blocking cell cycle progression. It is also involved in controlling cell differentiation. p27 protein may be used as a tumor suppressor. Mutations of CDKN1B cause multiple endocrine neoplasia type 4 (MEN4) and tumors in single endocrine glands. Lack of p27 protein causes cells to divide abnormally and form a tumor (Occhi et al., Eur J Endocrinol, 163(3):369 (2010), the contents of which are incorporated herein by reference in their entirety). These tumors may be cancerous or non-cancerous. The most common endocrine glands affected in multiple endocrine neoplasia type 4 are the parathyroid glands and the pituitary gland, although additional endocrine glands and other organs can also be involved. Tumors in single endocrine glands that are related to CDKN1B include tumors in pituitary gland or the parathyroid glands.

In one embodiment, provided is a method of modulating p27 protein levels comprising administering CDKN1B-saRNA of the present invention, wherein the CDKN1B-saRNA targets an antisense RNA transcript of CDKN1B gene. In one embodiment, p27 protein level is increased by at least 20, 30, 40%, or at least 45, 50, 55, 60, 65, 70, 75%, or at least 80% in the presence of the CDKN1B-saRNA of the present invention compared to p27 protein level in the absence of the CDKN1B-saRNA of the present invention. In a further embodiment, p27 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the CDKN1B-saRNA of the present invention compared to p27 protein level in the absence of the CDKN1B-saRNA of the present invention.

In another embodiment, provided is a method of treating multiple endocrine neoplasia type 4 comprising administering CDKN1B-saRNA of the present invention, wherein the CDKN1B-saRNA targets an antisense RNA transcript of CDKN1B gene. The multiple endocrine neoplasia type 4 may be in parathyroid glands and the pituitary gland. In another embodiment, provided is a method of treating tumors in a single endocrine gland comprising administering CDKN1B-saRNA of the present invention, wherein the CDKN1B-saRNA targets an antisense RNA transcript of CDKN1B gene. The single endocrine gland may be pituitary gland or the parathyroid glands.

In one embodiment, the CDKN1B-saRNA of the present invention is an antisense sequence such as, but not limited to, SEQ ID NO: 1692, 1694, 1696, 1698, 1700, 1702, 1704, 1706, 1708, 1710, 1712, 1714, 1716, 1718, 1720, 1722, 1724, 1726, 1728, 1730, 1732, 1734, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358 and 2360. As a non-limiting example, these CDKN1B-saRNA sequences may be used to modulate CDKN1B protein levels, p27 protein levels, and/or treating multiple endocrine neoplasia type 4 and tumors in a single endocrine gland.

In one embodiment, the CDKN1B-saRNA of the present invention is an saRNA duplex. The saRNA duplex may be a pair of sense and antisense sequences such as, but not limited to, SEQ ID NOs: 1691 and 1692; SEQ ID NOs: 1693 and 1694; SEQ ID NOs: 1695 and 1696; SEQ ID NOs: 1697 and 1698; SEQ ID NOs: 1699 and 1700; SEQ ID NOs: 1701 and 1702; SEQ ID NOs: 1703 and 1704; SEQ ID NOs: 1705 and 1706; SEQ ID NOs: 1707 and 1708; SEQ ID NOs: 1709 and 1710; SEQ ID NOs: 1711 and 1712; SEQ ID NOs: 1713 and 1714; SEQ ID NOs: 1715 and 1716; SEQ ID NOs: 1717 and 1718; SEQ ID NOs: 1719 and 1720; SEQ ID NOs: 1721 and 1722; SEQ ID NOs: 1723 and 1724; SEQ ID NOs: 1725 and 1726; SEQ ID NOs: 1727 and 1728; SEQ ID NOs: 1729 and 1730; SEQ ID NOs: 1731 and 1732; SEQ ID NOs: 1733 and 1734; SEQ ID NOs: 2261 and 2262; SEQ ID NOs: 2263 and 2264; SEQ ID NOs: 2265 and 2266; SEQ ID NOs: 2267 and 2268; SEQ ID NOs: 2269 and 2270; SEQ ID NOs: 2271 and 2272; SEQ ID NOs: 2273 and 2274; SEQ ID NOs: 2275 and 2276; SEQ ID NOs: 2277 and 2278; SEQ ID NOs: 2279 and 2280; SEQ ID NOs: 2281 and 2282; SEQ ID NOs: 2283 and 2284; SEQ ID NOs: 2285 and 2286; SEQ ID NOs: 2287 and 2288; SEQ ID NOs: 2289 and 2290; SEQ ID NOs: 2291 and 2292; SEQ ID NOs: 2293 and 2294; SEQ ID NOs: 2295 and 2296; SEQ ID NOs: 2297 and 2298; SEQ ID NOs: 2299 and 2300; SEQ ID NOs: 2301 and 2302; SEQ ID NOs: 2303 and 2304; SEQ ID NOs: 2305 and 2306; SEQ ID NOs: 2307 and 2308; SEQ ID NOs: 2309 and 2310; SEQ ID NOs: 2311 and 2312; SEQ ID NOs: 2313 and 2314; SEQ ID NOs: 2315 and 2316; SEQ ID NOs: 2317 and 2318; SEQ ID NOs: 2319 and 2320; SEQ ID NOs: 2321 and 2322; SEQ ID NOs: 2323 and 2324; SEQ ID NOs: 2325 and 2326; SEQ ID NOs: 2327 and 2328; SEQ ID NOs: 2329 and 2330; SEQ ID NOs: 2331 and 2332; SEQ ID NOs: 2333 and 2334; SEQ ID NOs: 2335 and 2336; SEQ ID NOs: 2337 and 2338; SEQ ID NOs: 2339 and 2340; SEQ ID NOs: 2341 and 2342; SEQ ID NOs: 2343 and 2344; SEQ ID NOs: 2345 and 2346; SEQ ID NOs: 2347 and 2348; SEQ ID NOs: 2349 and 2350; SEQ ID NOs: 2351 and 2352; SEQ ID NOs: 2353 and 2354; SEQ ID NOs: 2355 and 2356; SEQ ID NOs: 2357 and 2358; SEQ ID NOs: 2359 and 2360. As a non-limiting example, these CDKN1B-saRNA sequences which are saRNA duplexes may be used to modulate CDKN1B protein levels, p27 protein levels, and/or treating multiple endocrine neoplasia type 4 and tumors in a single endocrine gland.

### MDM2 Gene

As another non-limiting example, provided is a method of modulating the expression of MDM2 gene comprising administering saRNA of the present invention, wherein the saRNA targets an antisense RNA transcript of MDM2 gene. These saRNAs are called MDM2-saRNA. In one embodiment, the expression of MDM2 gene is increased by at least 20, 30, 40%, or at least 45, 50, 55, 60, 65, 70, 75%, or at least 80% in the presence of the MDM2-saRNA of the present invention compared to the expression of MDM2 gene in the absence of the MDM2-saRNA of the present invention. In a further embodiment, the expression of MDM2 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the MDM2-saRNA of the present invention compared to the expression of MDM2 gene in the absence of the MDM2-saRNA of the present invention. The modulation of the expression of MDM2 gene may be reflected or determined by the change of MDM2 mRNA levels.

MDM2-saRNAs may be single-stranded and comprise 14-30 nucleotides. The sequence of a single-stranded MDM2-saRNA may have at least 60%, 70%, 80% or 90% identity with a sequence selected from the sequences of the antisense strands in Table 44-2. In one embodiment, the single-stranded saRNA comprises a sequence selected from the sequences of the antisense strands in Table 44-2. In one embodiment, the single-stranded MDM2-saRNA may have a 3′ tail. The sequence of a single-stranded MDM2-saRNA with a 3′ tail may have at least 60%, 70%, 80% or 90% identity with a sequence selected from the sequences of the antisense strands in Table 45. In one embodiment, the single-stranded saRNA comprises a sequence selected from the sequences of the antisense strands in Table 45.

MDM2-saRNAs may be double-stranded. The two strands form a duplex and each strand comprises 14-30 nucleotides. The first strand of a double-stranded MDM2-saRNA may have at least 60%, 70%, 80% or 90% identity with a sequence selected from the sequences of the antisense strands in Table 44-2. In one embodiment, the first strand of the double-stranded saRNA comprises a sequence selected from the sequences of the antisense strands in Table 44-2. The second strand of a double-stranded MDM2-saRNA may have at least 60%, 70%, 80% or 90% identity with a sequence selected from the sequences of the sense strands in Table 44-2. In one embodiment, the second strand of the double-stranded saRNA comprises a sequence selected from the sequences of the sense strands in Table 44-2. In one embodiment, the double-stranded MDM2-saRNA may have a 3′ overhang on each strand. The first strand of a double-stranded MDM2-saRNA may have at least 60%, 70%, 80% or 90% identity with a sequence selected from the sequences of the antisense strands in Table 45. In one embodiment, the first strand of the double-stranded saRNA comprises a sequence selected from the sequences of the antisense strands in Table 45. The second strand of a double-stranded MDM2-saRNA may have at least 60%, 70%, 80% or 90% identity with a sequence selected from the sequences of the sense strands in Table 45. In one embodiment, the second strand of the double-stranded saRNA comprises a sequence selected from the sequences of the sense strands in Table 45.

MDM2-saRNAs may be modified or unmodified.

Table 44-1 describes the target gene, the targeted sequence, the genomic location of the targeted sequence and the relative location of MDM2-saRNAs with no 3′ overhang. In Table 44-1, the targeted sequence is defined as a region on the template strand of the target gene which is identical to a region on the target antisense RNA transcript to which the antisense strand of an saRNA hybridizes. In Table 44-1, the relative location is the distance from the 5′ end of the targeted sequence to the TSS. A negative number represents a location upstream of the TSS and a positive number represents a location downstream of the TSS.

TABLE 44-1
Sequences of MDM2-saRNAs (with no 3′ overhang)

| | | Targeted | Targeted | Genomic Location of | Relative |
|---|---|---|---|---|---|
| saRNA ID | Target Gene | sequence | sequence ID | the Targeted sequence | Location |
| MDM2.NM_001145337- | MDM2 human | aattgaggtatacgaa | 1735 | chr12: 69203132 minus | 355 |
| Pr-10 | | att | | strand | |
| MDM2.NM_001145337- | MDM2 human | gtttcatcttgttagtaca | 1736 | chr12: 69203563 minus | 786 |
| Pr-14 | | | | strand | |
| MDM2.NM_001145337- | MDM2 human | agcggcactatctgta | 1737 | chr12: 69203736 minus | 959 |
| Pr-18 | | ata | | strand | |
| MDM2.NM_001145337- | MDM2 human | cgaaatttagtttatca | 1738 | chr12: 69203120 minus | 343 |
| Pr-20 | | ct | | strand | |
| MDM2.NM_001145337- | MDM2 human | cggcactatctgtaat | 1739 | chr12: 69203734 minus | 957 |
| Pr-22 | | act | | strand | |
| MDM2.NM_001145337- | MDM2 human | ggagacaagtcagga | 1740 | chr12: 69202743 minus | −34 |
| Pr-33 | | ctta | | strand | |
| MDM2.NM_001145337- | MDM2 human | gtatactcatgctgtgg | 1741 | chr12: 69203655 minus | 878 |
| Pr-35 | | aa | | strand | |
| MDM2.NM_001145337- | MDM2 human | aactggaaatcatgaa | 1742 | chr12: 69202941 minus | 164 |
| Pr-40 | | aca | | strand | |
| MDM2.NM_002392- | MDM2 human | tggagttgtacccaaa | 1743 | chr12: 69201539 minus | −393 |
| Pr-1 | | tga | | strand | |
| MDM2.NM_002392- | MDM2 human | ttgatgcggtctcataa | 1744 | chr12: 69200954 minus | −978 |
| Pr-6 | | at | | strand | |
| MDM2.NM_002392- | MDM2 human | gttgtacccaaatgag | 1745 | chr12: 69201535 minus | −397 |
| Pr-12 | | tta | | strand | |
| MDM2.NM_002392- | MDM2 human | aaagtttgggtttgtta | 1746 | chr12: 69201065 minus | −867 |
| Pr-22 | | at | | strand | |
| MDM2.NM_002392- | MDM2 human | ggagacaagtcagga | 1747 | chr12: 69202743 minus | 811 |
| Pr-23 | | ctta | | strand | |
| MDM2.NM_002392- | MDM2 human | ggtggaaactgcgac | 1748 | chr12: 69201638 minus | −294 |
| Pr-24 | | aaat | | strand | |
| MDM2.NM_002392- | MDM2 human | gtttgggtttgttaatcta | 1749 | chr12: 69201062 minus | −870 |
| Pr-26 | | | | strand | |
| MDM2.NM_002392- | MDM2 human | gctccatctttccgaca | 1750 | chr12: 69202014 minus | 82 |
| Pr-33 | | ca | | strand | |
| MDM2.NM_002392- | MDM2 human | aaggtttggcgctgtg | 1751 | chr12: 69201712 minus | −220 |
| Pr-34 | | aca | | strand | |

TABLE 44-2
Sequences of MDM2-saRNAs (with no 3′ overhang)

| | | Sense SEQ | | Anti-sense |
|---|---|---|---|---|
| saRNA ID | Sense (passenger) | ID | Antisense (guide) | SEQ ID |
| MDM2.NM_001145337- | AAUUGAGGUAUA | 1752 | AAUUUCGUAUAC | 1753 |
| Pr-10 | CGAAAUU | | CUCAAUU | |
| MDM2.NM_001145337- | GUUUCAUCUUGU | 1754 | UGUACUAACAAG | 1755 |
| Pr-14 | UAGUACA | | AUGAAAC | |
| MDM2.NM_001145337- | AGCGGCACUAUC | 1756 | UAUUACAGAUAG | 1757 |
| Pr-18 | UGUAAUA | | UGCCGCU | |
| MDM2.NM_001145337- | CGAAAUUUAGUU | 1758 | AGUGAUAAACUA | 1759 |
| Pr-20 | UAUCACU | | AAUUUCG | |
| MDM2.NM_001145337- | CGGCACUAUCUG | 1760 | AGUAUUACAGAU | 1761 |
| Pr-22 | UAAUACU | | AGUGCCG | |
| MDM2.NM_001145337- | GGAGACAAGUCA | 1762 | UAAGUCCUGACU | 1763 |
| Pr-33 | GGACUUA | | UGUCUCC | |
| MDM2.NM_001145337- | GUAUACUCAUGC | 1764 | UUCCACAGCAUG | 1765 |
| Pr-35 | UGUGGAA | | AGUAUAC | |
| MDM2.NM_001145337- | AACUGGAAAUCA | 1766 | UGUUUCAUGAUU | 1767 |
| Pr-40 | UGAAACA | | UCCAGUU | |
| MDM2.NM_002392- | UGGAGUUGUACC | 1768 | UCAUUUGGGUAC | 1769 |
| Pr-1 | CAAAUGA | | AACUCCA | |
| MDM2.NM_002392- | UUGAUGCGGUCU | 1770 | AUUUAUGAGACC | 1771 |
| Pr-6 | CAUAAAU | | GCAUCAA | |
| MDM2.NM_002392- | GUUGUACCCAAA | 1772 | UAACUCAUUUGG | 1773 |
| Pr-12 | UGAGUUA | | GUACAAC | |
| MDM2.NM_002392- | AAAGUUUGGGUU | 1774 | AUUAACAAACCC | 1775 |
| Pr-22 | UGUUAAU | | AAACUUU | |
| MDM2.NM_002392- | GGAGACAAGUCA | 1776 | UAAGUCCUGACU | 1777 |
| Pr-23 | GGACUUA | | UGUCUCC | |
| MDM2.NM_002392- | GGUGGAAACUGC | 1778 | AUUUGUCGCAGU | 1779 |
| Pr-24 | GACAAAU | | UUCCACC | |
| MDM2.NM_002392- | GUUUGGGUUUGU | 1780 | UAGAUUAACAAA | 1781 |
| Pr-26 | UAAUCUA | | CCCAAAC | |
| MDM2.NM_002392- | GCUCCAUCUUUC | 1782 | UGUGUCGGAAAG | 1783 |
| Pr-33 | CGACACA | | AUGGAGC | |
| MDM2.NM_002392- | AAGGUUUGGCGC | 1784 | UGUCACAGCGCC | 1785 |
| Pr-34 | UGUGACA | | AAACCUU | |

TABLE 45
Sequences of MDM2-saRNAs (with 3′ overhang)

| | | Sense | | Antisense |
|---|---|---|---|---|
| saRNA ID | Sense (passenger) | SEQ ID | Antisense (guide) | SEQ ID |
| MDM2.NM_001145337- | AAUUGAGGUAUACG | 1786 | AAUUUCGUAUACC | 1787 |
| Pr-10′ | AAAUUUU | | UCAAUUUU | |
| MDM2.NM_001145337- | GUUUCAUCUUGUUA | 1788 | UGUACUAACAAGA | 1789 |
| Pr-14′ | GUACAUU | | UGAAACUU | |
| MDM2.NM_001145337- | AGCGGCACUAUCUG | 1790 | UAUUACAGAUAGU | 1791 |
| Pr-18′ | UAAUAUU | | GCCGCUUU | |
| MDM2.NM_001145337- | CGAAAUUUAGUUUA | 1792 | AGUGAUAAACUAA | 1793 |
| Pr-20′ | UCACUUU | | AUUUCGUU | |
| MDM2.NM_001145337- | CGGCACUAUCUGUA | 1794 | AGUAUUACAGAUA | 1795 |
| Pr-22′ | AUACUUU | | GUGCCGUU | |
| MDM2.NM_001145337- | GGAGACAAGUCAGG | 1796 | UAAGUCCUGACUU | 1797 |
| Pr-33′ | ACUUAUU | | GUCUCCUU | |
| MDM2.NM_001145337- | GUAUACUCAUGCUG | 1798 | UUCCACAGCAUGAG | 1799 |
| Pr-35′ | UGGAAUU | | UAUACUU | |
| MDM2.NM_001145337- | AACUGGAAAUCAUG | 1800 | UGUUUCAUGAUUU | 1801 |
| Pr-40′ | AAACAUU | | CCAGUUUU | |
| MDM2.NM_002392-Pr-1′ | UGGAGUUGUACCCA | 1802 | UCAUUUGGGUACA | 1803 |
| | AAUGAUU | | ACUCCAUU | |
| MDM2.NM_002392-Pr-6′ | UUGAUGCGGUCUCA | 1804 | AUUUAUGAGACCG | 1805 |
| | UAAAUUU | | CAUCAAUU | |
| MDM2.NM_002392-Pr- | GUUGUACCCAAAUG | 1806 | UAACUCAUUUGGG | 1807 |
| 12′ | AGUUAUU | | UACAACUU | |
| MDM2.NM_002392-Pr- | AAAGUUUGGGUUUG | 1808 | AUUAACAAACCCAA | 1809 |
| 22′ | UUAAUUU | | ACUUUUU | |
| MDM2.NM_002392-Pr- | GGAGACAAGUCAGG | 1810 | UAAGUCCUGACUU | 1811 |
| 23′ | ACUUAUU | | GUCUCCUU | |
| MDM2.NM_002392-Pr- | GGUGGAAACUGCGA | 1812 | AUUUGUCGCAGUU | 1813 |
| 24′ | CAAAUUU | | UCCACCUU | |
| MDM2.NM_002392-Pr- | GUUUGGGUUUGUUA | 1814 | UAGAUUAACAAAC | 1815 |
| 26′ | AUCUAUU | | CCAAACUU | |
| MDM2.NM_002392-Pr- | GCUCCAUCUUUCCG | 1816 | UGUGUCGGAAAGA | 1817 |
| 33′ | ACACAUU | | UGGAGCUU | |
| MDM2.NM_002392-Pr- | AAGGUUUGGCGCUG | 1818 | UGUCACAGCGCCAA | 1819 |
| 34′ | UGACAUU | | ACCUUUU | |

MDM2 gene encodes Mouse double minute 2 homolog (MDM2) protein, also known as E3 ubiquitin-protein ligase. Recent studies have revealed the MDM2-p53 interaction to be complex involving multiple levels of regulation by numerous cellular proteins and epigenetic mechanisms (Nag et al., J Biomed Res., vol 27(4):254 (2013), the contents of which are incorporated herein by reference in their entirety).

In one embodiment, provided is a method of modulating MDM2 protein levels comprising administering MDM2-saRNA of the present invention, wherein the MDM2-saRNA targets an antisense RNA transcript of MDM2 gene. In one embodiment, MDM2 protein level is increased by at least 20, 30, 40%, or at least 45, 50, 55, 60, 65, 70, 75%, or at least 80% in the presence of the MDM2-saRNA of the present invention compared to MDM2 protein level in the absence of the MDM2-saRNA of the present invention. In a further embodiment, MDM2 protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the MDM2-saRNA of the present invention compared to MDM2 protein level in the absence of the MDM2-saRNA of the present invention. In another embodiment, provided is a method of modulating p53 protein levels, comprising administering MDM2-saRNA of the present invention, wherein the MDM2-saRNA targets an antisense RNA transcript of MDM2 gene.

In another embodiment, provided is a method of modulating p53 protein levels comprising administering MDM2-saRNA of the present invention, wherein the MDM2-saRNA targets an antisense RNA transcript of MDM2 gene. In another embodiment, provided is a method of modulating apoptosis of a tumor cell comprising administering MDM2-saRNA of the present invention, wherein the MDM2-saRNA targets an antisense RNA transcript of MDM2 gene.

In one embodiment, the MDM2-saRNA of the present invention is an antisense sequence such as, but not limited to, SEQ ID NO: 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 4910, 4912, 4914, 4916, 4918, 4920, 4922, 4924, 4926, 4928, 4930, 4932, 4934, 4936, 4938, 4940, 4942, 4944, 4946, 4948, 4950, 4952, 4954, 4956, 4958, 4960, 4962, 4964, 4966, 4968, 4970, 4972, 4974, 4976, 4978, 4980, 4982, 4984, 4986, 4988, 4990, 4992, 4994, 4996, 4998, 5000, 5002, 5004, 5006, 5008, 5010, 5012, 5014, 5016, 5018, 5020, 5022, 5024, 5026, 5028, 5030, 5032, 5034, 5036, 5038, 5040, 5042, 5044, 5046, 5048, 5050, 5052, 5054, 5056, 5058, 5060, 5062, 5064, 5066, 5068, 5070, 5072 and 5074. As a non-limiting example, these MDM2-saRNA sequences may be used to modulate MDM2 protein levels and/or modulating p53 protein levels.

In one embodiment, the MDM2-saRNA of the present invention is an saRNA duplex. The saRNA duplex may be a pair of sense and antisense sequences such as, but not limited to, SEQ ID NOs: 1752 and 1753; SEQ ID NOs: 1754 and 1755; SEQ ID NOs: 1756 and 1757; SEQ ID NOs: 1758 and 1759; SEQ ID NOs: 1760 and 1761; SEQ ID NOs: 1762 and 1763; SEQ ID NOs: 1764 and 1765; SEQ ID NOs: 1766 and 1767; SEQ ID NOs: 1768 and 1769; SEQ ID NOs: 1770 and 1771; SEQ ID NOs: 1772 and 1773; SEQ ID NOs: 1774 and 1775; SEQ ID NOs: 1776 and 1777; SEQ ID NOs: 1778 and 1779; SEQ ID NOs: 1780 and 1781; SEQ ID NOs: 1782 and 1783; SEQ ID NOs: 1784 and 1785; SEQ ID NOs: 1786 and 1787; SEQ ID NOs: 1788 and 1789; SEQ ID NOs: 1790 and 1791; SEQ ID NOs: 1792 and 1793; SEQ ID NOs: 1794 and 1795; SEQ ID NOs: 1796 and 1797; SEQ ID NOs: 1798 and 1799; SEQ ID NOs: 1800 and 1801; SEQ ID NOs: 1802 and 1803; SEQ ID NOs: 1804 and 1805; SEQ ID NOs: 1806 and 1807; SEQ ID NOs: 1808 and 1809; SEQ ID NOs: 1810 and 1811; SEQ ID NOs: 1812 and 1813; SEQ ID NOs: 1814 and 1815; SEQ ID NOs: 1816 and 1817; SEQ ID NOs: 1818 and 1819; SEQ ID NOs: 4909 and 4910; SEQ ID NOs: 4911 and 4912; SEQ ID NOs: 4913 and 4914; SEQ ID NOs: 4915 and 4916; SEQ ID NOs: 4917 and 4918; SEQ ID NOs: 4919 and 4920; SEQ ID NOs: 4921 and 4922; SEQ ID NOs: 4923 and 4924; SEQ ID NOs: 4925 and 4926; SEQ ID NOs: 4927 and 4928; SEQ ID NOs: 4929 and 4930; SEQ ID NOs: 4931 and 4932; SEQ ID NOs: 4933 and 4934; SEQ ID NOs: 4935 and 4936; SEQ ID NOs: 4937 and 4938; SEQ ID NOs: 4939 and 4940; SEQ ID NOs: 4941 and 4942; SEQ ID NOs: 4943 and 4944; SEQ ID NOs: 4945 and 4946; SEQ ID NOs: 4947 and 4948; SEQ ID NOs: 4949 and 4950; SEQ ID NOs: 4951 and 4952; SEQ ID NOs: 4953 and 4954; SEQ ID NOs: 4955 and 4956; SEQ ID NOs: 4957 and 4958; SEQ ID NOs: 4959 and 4960; SEQ ID NOs: 4961 and 4962; SEQ ID NOs: 4963 and 4964; SEQ ID NOs: 4965 and 4966; SEQ ID NOs: 4967 and 4968; SEQ ID NOs: 4969 and 4970; SEQ ID NOs: 4971 and 4972; SEQ ID NOs: 4973 and 4974; SEQ ID NOs: 4975 and 4976; SEQ ID NOs: 4977 and 4978; SEQ ID NOs: 4979 and 4980; SEQ ID NOs: 4981 and 4982; SEQ ID NOs: 4983 and 4984; SEQ ID NOs: 4985 and 4986; SEQ ID NOs: 4987 and 4988; SEQ ID NOs: 4989 and 4990; SEQ ID NOs: 4991 and 4992; SEQ ID NOs: 4993 and 4994; SEQ ID NOs: 4995 and 4996; SEQ ID NOs: 4997 and 4998; SEQ ID NOs: 4999 and 5000; SEQ ID NOs: 5001 and 5002; SEQ ID NOs: 5003 and 5004; SEQ ID NOs: 5005 and 5006; SEQ ID NOs: 5007 and 5008; SEQ ID NOs: 5009 and 5010; SEQ ID NOs: 5011 and 5012; SEQ ID NOs: 5013 and 5014; SEQ ID NOs: 5015 and 5016; SEQ ID NOs: 5017 and 5018; SEQ ID NOs: 5019 and 5020; SEQ ID NOs: 5021 and 5022; SEQ ID NOs: 5023 and 5024; SEQ ID NOs: 5025 and 5026; SEQ ID NOs: 5027 and 5028; SEQ ID NOs: 5029 and 5030; SEQ ID NOs: 5031 and 5032; SEQ ID NOs: 5033 and 5034; SEQ ID NOs: 5035 and 5036; SEQ ID NOs: 5037 and 5038; SEQ ID NOs: 5039 and 5040; SEQ ID NOs: 5041 and 5042; SEQ ID NOs: 5043 and 5044; SEQ ID NOs: 5045 and 5046; SEQ ID NOs: 5047 and 5048; SEQ ID NOs: 5049 and 5050; SEQ ID NOs: 5051 and 5052; SEQ ID NOs: 5053 and 5054; SEQ ID NOs: 5055 and 5056; SEQ ID NOs: 5057 and 5058; SEQ ID NOs: 5059 and 5060; SEQ ID NOs: 5061 and 5062; SEQ ID NOs: 5063 and 5064; SEQ ID NOs: 5065 and 5066; SEQ ID NOs: 5067 and 5068; SEQ ID NOs: 5069 and 5070; SEQ ID NOs: 5071 and 5072; SEQ ID NOs: 5073 and 5074. As a non-limiting example, these MDM2-saRNA sequences which are saRNA duplexes may be used to modulate MDM2 protein levels and/or modulating p53 protein levels.

### C19orf80 Gene

As another non-limiting example, provided is a method of modulating the expression of c19orf80 gene comprising administering saRNA of the present invention, wherein the saRNA targets an antisense RNA transcript of c19orf80 gene. These saRNAs are called c19orf80-saRNA. In one embodiment, the expression of c19orf80 gene is increased by at least 20, 30, 40%, or at least 45, 50, 55, 60, 65, 70, 75%, or at least 80% in the presence of the c19orf80-saRNA of the present invention compared to the expression of c19orf80 gene in the absence of the c19orf80-saRNA of the present invention. In a further embodiment, the expression of c19orf80 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the c19orf80-saRNA of the present invention compared to the expression of c19orf80 gene in the absence of the c19orf80-saRNA of the present invention. The modulation of the expression of c19orf80 gene may be reflected or determined by the change of c19orf80 mRNA levels.

C19ORF80-saRNAs may be single-stranded and comprise 14-30 nucleotides. The sequence of a single-stranded C19ORF80-saRNA may have at least 60%, 70%, 80% or 90% identity with a sequence selected from the sequences of the antisense strands in Table 46-2. In one embodiment, the single-stranded saRNA comprises a sequence selected from the sequences of the antisense strands in Table 46-2. In one embodiment, the single-stranded C19ORF80-saRNA may have a 3′ tail. The sequence of a single-stranded C19ORF80-saRNA with a 3′ tail may have at least 60%, 70%, 80% or 90% identity with a sequence selected from the sequences of the antisense strands in Table 47. In one embodiment, the single-stranded saRNA comprises a sequence selected from the sequences of the antisense strands in Table 47.

C19ORF80-saRNAs may be double-stranded. The two strands form a duplex and each strand comprises 14-30 nucleotides. The first strand of a double-stranded C19ORF80-saRNA may have at least 60%, 70%, 80% or 90% identity with a sequence selected from the sequences of the antisense strands in Table 46-2. In one embodiment, the first strand of the double-stranded saRNA comprises a sequence selected from the sequences of the antisense strands in Table 46-2. The second strand of a double-stranded C19ORF80-saRNA may have at least 60%, 70%, 80% or 90% identity with a sequence selected from the sequences of the sense strands in Table 46-2. In one embodiment, the second strand of the double-stranded saRNA comprises a sequence selected from the sequences of the sense strands in Table 46-2. In one embodiment, the double-stranded C19ORF80-saRNA may have a 3′ overhang on each strand. The first strand of a double-stranded C19ORF80-saRNA may have at least 60%, 70%, 80% or 90% identity with a sequence selected from the sequences of the antisense strands in Table 47. In one embodiment, the first strand of the double-stranded saRNA comprises a sequence selected from the sequences of the antisense strands in Table 47. The second strand of a double-stranded C19ORF80-saRNA may have at least 60%, 70%, 80% or 90% identity with a sequence selected from the sequences of the sense strands in Table 47. In one embodiment, the second strand of the double-stranded saRNA comprises a sequence selected from the sequences of the sense strands in Table 47.

C19ORF80-saRNAs may be modified or unmodified.

Table 46-1 describes the target gene, the targeted sequence, the genomic location of the targeted sequence and the relative location of C19orf80-saRNAs with no 3′ overhang. In Table 46-1, the targeted sequence is defined as a region on the template strand of the target gene which is identical to a region on the target antisense RNA transcript to which the antisense strand of an saRNA hybridizes. In Table 46-1, the relative location is the distance from the 5′ end of the targeted sequence to the TSS. A negative number represents a location upstream of the TSS and a positive number represents a location downstream of the TSS.

TABLE 46-1
Sequences of C19ORF80-saRNAs (with no 3′ overhang)

| | | | Targeted | | |
|---|---|---|---|---|---|
| | | | sequence | Genomic Location of the | Relative |
| saRNA ID | Target Gene | Targeted sequence | ID | Targeted sequence | Location |
| C19orf80.NM_018687- | C19orf80 | gcgagttctggccggaa | 1820 | chr19: 11351010 minus | 735 |
| Pr-1 | human | at | | strand | |
| C19orf80.NM_018687- | C19orf80 | gagttctggccggaaat | 1821 | chr19: 11351008 minus | 733 |
| Pr-6 | human | ca | | strand | |
| C19orf80.NM_018687- | C19orf80 | agcgagttctggccgga | 1822 | chr19: 11351011 minus | 736 |
| Pr-7 | human | aa | | strand | |
| C19orf80.NM_018687- | C19orf80 | gcctgagtttatcgctgaa | 1823 | chr19: 11350728 minus | 453 |
| Pr-11 | human | | | strand | |
| C19orf80.NM_018687- | C19orf80 | gcctgaagtgcccattata | 1824 | chr19: 11349513 minus | −762 |
| Pr-13 | human | | | strand | |
| C19orf80.NM_018687- | C19orf80 | cgagttctggccggaaa | 1825 | chr19: 11351009 minus | 734 |
| Pr-18 | human | tc | | strand | |
| C19orf80.NM_018687- | C19orf80 | aggacttgcctgagtttat | 1826 | chr19: 11350735 minus | 460 |
| Pr-19 | human | | | strand | |
| C19orf80.NM_018687- | C19orf80 | cgattatttcattgttgta | 1827 | chr19: 11349609 minus | −666 |
| Pr-21 | human | | | strand | |
| C19orf80.NM_018687- | C19orf80 | agctacttgtgagaagtaa | 1828 | chr19: 11349680 minus | −595 |
| Pr-23 | human | | | strand | |
| C19orf80.NM_018687- | C19orf80 | tgggagttcctgagact | 1829 | chr19: 11350050 minus | −225 |
| Pr-25 | human | ca | | strand | |
| C19orf80.NM_018687- | C19orf80 | tcatgagctacttgtgaga | 1830 | chr19: 11349685 minus | −590 |
| Pr-26 | human | | | strand | |
| C19orf80.NM_018687- | C19orf80 | gttgagtgcttagaaca | 1831 | chr19: 11349647 minus | −628 |
| Pr-28 | human | ga | | strand | |
| C19orf80.NM_018687- | C19orf80 | aatagtgtttggtgcaatt | 1832 | chr19: 11349588 minus | −687 |
| Pr-33 | human | | | strand | |
| C19orf80.NM_018687- | C19orf80 | gtaatagtgtttggtgcaa | 1833 | chr19: 11349590 minus | −685 |
| Pr-39 | human | | | strand | |

TABLE 46-2
Sequences of C19ORF80-saRNAs (with no 3′ overhang)

| | | Sense | | Anti-sense SEQ |
|---|---|---|---|---|
| saRNA ID | Sense (passenger) | SEQ ID | Antisense (guide) | ID |
| C19orf80.NM_018687- | GCGAGUUCUGGCC | 1834 | AUUUCCGGCCAGAAC | 1835 |
| Pr-1 | GGAAAU | | UCGC | |
| C19orf80.NM_018687- | GAGUUCUGGCCGG | 1836 | UGAUUUCCGGCCAGA | 1837 |
| Pr-6 | AAAUCA | | ACUC | |
| C19orf80.NM_018687- | AGCGAGUUCUGGC | 1838 | UUUCCGGCCAGAACU | 1839 |
| Pr-7 | CGGAAA | | CGCU | |
| C19orf80.NM_018687- | GCCUGAGUUUAUC | 1840 | UUCAGCGAUAAACUC | 1841 |
| Pr-11 | GCUGAA | | AGGC | |
| C19orf80.NM_018687- | GCCUGAAGUGCCC | 1842 | UAUAAUGGGCACUUC | 1843 |
| Pr-13 | AUUAUA | | AGGC | |
| C19orf80.NM_018687- | CGAGUUCUGGCCG | 1844 | GAUUUCCGGCCAGAA | 1845 |
| Pr-18 | GAAAUC | | CUCG | |
| C19orf80.NM_018687- | AGGACUUGCCUGA | 1846 | AUAAACUCAGGCAAG | 1847 |
| Pr-19 | GUUUAU | | UCCU | |
| C19orf80.NM_018687- | CGAUUAUUUCAUU | 1848 | UACAACAAUGAAAUA | 1849 |
| Pr-21 | GUUGUA | | AUCG | |
| C19orf80.NM_018687- | AGCUACUUGUGAG | 1850 | UUACUUCUCACAAGU | 1851 |
| Pr-23 | AAGUAA | | AGCU | |
| C19orf80.NM_018687- | UGGGAGUUCCUGA | 1852 | UGAGUCUCAGGAACU | 1853 |
| Pr-25 | GACUCA | | CCCA | |
| C19orf80.NM_018687- | UCAUGAGCUACUU | 1854 | UCUCACAAGUAGCUC | 1855 |
| Pr-26 | GUGAGA | | AUGA | |
| C19orf80.NM_018687- | GUUGAGUGCUUAG | 1856 | UCUGUUCUAAGCACU | 1857 |
| Pr-28 | AACAGA | | CAAC | |
| C19orf80.NM_018687- | AAUAGUGUUUGG | 1858 | AAUUGCACCAAACAC | 1859 |
| Pr-33 | UGCAAUU | | UAUU | |
| C19orf80.NM_018687- | GUAAUAGUGUUU | 1860 | UUGCACCAAACACUA | 1861 |
| Pr-39 | GGUGCAA | | UUAC | |

TABLE 47
Sequences of c19orf80-saRNAs (with 3′ overhang)

| | | Sense SEQ | | Antisense |
|---|---|---|---|---|
| saRNA ID | Sense (passenger) | ID | Antisense (guide) | SEQ ID |
| C19orf80.NM_018687- | GCGAGUUCUGGCCGG | 1862 | AUUUCCGGCCAGAA | 1863 |
| Pr-1′ | AAAUUU | | CUCGCUU | |
| C19orf80.NM_018687- | GAGUUCUGGCCGGA | 1864 | UGAUUUCCGGCCAG | 1865 |
| Pr-6′ | AAUCAUU | | AACUCUU | |
| C19orf80.NM_018687- | AGCGAGUUCUGGCCG | 1866 | UUUCCGGCCAGAAC | 1867 |
| Pr-7′ | GAAAUU | | UCGCUUU | |
| C19orf80.NM_018687- | GCCUGAGUUUAUCGC | 1868 | UUCAGCGAUAAACU | 1869 |
| Pr-11′ | UGAAUU | | CAGGCUU | |
| C19orf80.NM_018687- | GCCUGAAGUGCCCAU | 1870 | UAUAAUGGGCACUU | 1871 |
| Pr-13′ | UAUAUU | | CAGGCUU | |
| C19orf80.NM_018687- | CGAGUUCUGGCCGGA | 1872 | GAUUUCCGGCCAGA | 1873 |
| Pr-18′ | AAUCUU | | ACUCGUU | |
| C19orf80.NM_018687- | AGGACUUGCCUGAG | 1874 | AUAAACUCAGGCAA | 1875 |
| Pr-19′ | UUUAUUU | | GUCCUUU | |
| C19orf80.NM_018687- | CGAUUAUUUCAUUG | 1876 | UACAACAAUGAAAU | 1877 |
| Pr-21′ | UUGUAUU | | AAUCGUU | |
| C19orf80.NM_018687- | AGCUACUUGUGAGA | 1878 | UUACUUCUCACAAG | 1879 |
| Pr-23′ | AGUAAUU | | UAGCUUU | |
| C19orf80.NM_018687- | UGGGAGUUCCUGAG | 1880 | UGAGUCUCAGGAAC | 1881 |
| Pr-25′ | ACUCAUU | | UCCCAUU | |
| C19orf80.NM_018687- | UCAUGAGCUACUUG | 1882 | UCUCACAAGUAGCU | 1883 |
| Pr-26′ | UGAGAUU | | CAUGAUU | |
| C19orf80.NM_018687- | GUUGAGUGCUUAGA | 1884 | UCUGUUCUAAGCAC | 1885 |
| Pr-28′ | ACAGAUU | | UCAACUU | |
| C19orf80.NM_018687- | AAUAGUGUUUGGUG | 1886 | AAUUGCACCAAACA | 1887 |
| Pr-33′ | CAAUUUU | | CUAUUUU | |
| C19orf80.NM_018687- | GUAAUAGUGUUUGG | 1888 | UUGCACCAAACACU | 1889 |
| Pr-39′ | UGCAAUU | | AUUACUU | |

C19orf80 gene encodes betatrophin, a protein that modulates autophagy, lipid metabolism and fat metabolism (Ren et al., Am J Physiol Endocrinol Metab., vol. 303 (3):E334 (2012); Tseng et al., Autophagy, vol. 10 (1):20 (2014), the contents of each of which are incorporated herein by reference in their entirety).

In one embodiment, provided is a method of modulating betatrophin protein levels comprising administering c19orf80-saRNA of the present invention, wherein the c19orf80-saRNA targets an antisense RNA transcript of c19orf80 gene. In one embodiment, betatrophin protein level is increased by at least 20, 30, 40%, or at least 45, 50, 55, 60, 65, 70, 75%, or at least 80% in the presence of the c19orf80-saRNA of the present invention compared to betatrophin protein level in the absence of the c19orf80-saRNA of the present invention. In a further embodiment, betatrophin protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the c19orf80-saRNA of the present invention compared to betatrophin protein level in the absence of the c19orf80-saRNA of the present invention.

In another embodiment, provided is a method of regulate autophagy and lipid metabolism comprising administering c19orf80-saRNA of the present invention, wherein the c19orf80-saRNA targets an antisense RNA transcript of c19orf80 gene. In another embodiment, provided is a method of regulating insulin or triglyceride levels comprising administering c19orf80-saRNA of the present invention, wherein the c19orf80-saRNA targets an antisense RNA transcript of c19orf80 gene. In another embodiment, provided is a method of treating obesity comprising administering c19orf80-saRNA of the present invention, wherein the c19orf80-saRNA targets an antisense RNA transcript of c19orf80 gene. In another embodiment, provided is a method of treating type II or type I diabetes comprising administering saRNA of the present invention, wherein the saRNA targets an antisense RNA transcript of c19orf80 gene.

In one embodiment, the C19orf80-saRNA of the present invention is an antisense sequence such as, but not limited to, SEQ ID NO: 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258 and 2260. As a non-limiting example, these C19orf80-saRNA sequences may be used to modulate betatrophin protein levels, regulating autophagy and lipid metabolism, treating obesity and/or treating type II or type I diabetes.

In one embodiment, the C19orf80-saRNA of the present invention is an saRNA duplex. The saRNA duplex may be a pair of sense and antisense sequences such as, but not limited to, SEQ ID NOs: 1834 and 1835; SEQ ID NOs: 1836 and 1837; SEQ ID NOs: 1838 and 1839; SEQ ID NOs: 1840 and 1841; SEQ ID NOs: 1842 and 1843; SEQ ID NOs: 1844 and 1845; SEQ ID NOs: 1846 and 1847; SEQ ID NOs: 1848 and 1849; SEQ ID NOs: 1850 and 1851; SEQ ID NOs: 1852 and 1853; SEQ ID NOs: 1854 and 1855; SEQ ID NOs: 1856 and 1857; SEQ ID NOs: 1858 and 1859; SEQ ID NOs: 1860 and 1861; SEQ ID NOs: 1862 and 1863; SEQ ID NOs: 1864 and 1865; SEQ ID NOs: 1866 and 1867; SEQ ID NOs: 1868 and 1869; SEQ ID NOs: 1870 and 1871; SEQ ID NOs: 1872 and 1873; SEQ ID NOs: 1874 and 1875; SEQ ID NOs: 1876 and 1877; SEQ ID NOs: 1878 and 1879; SEQ ID NOs: 1880 and 1881; SEQ ID NOs: 1882 and 1883; SEQ ID NOs: 1884 and 1885; SEQ ID NOs: 1886 and 1887; SEQ ID NOs: 1888 and 1889; SEQ ID NOs: 2161 and 2162; SEQ ID NOs: 2163 and 2164; SEQ ID NOs: 2165 and 2166; SEQ ID NOs: 2167 and 2168; SEQ ID NOs: 2169 and 2170; SEQ ID NOs: 2171 and 2172; SEQ ID NOs: 2173 and 2174; SEQ ID NOs: 2175 and 2176; SEQ ID NOs: 2177 and 2178; SEQ ID NOs: 2179 and 2180; SEQ ID NOs: 2181 and 2182; SEQ ID NOs: 2183 and 2184; SEQ ID NOs: 2185 and 2186; SEQ ID NOs: 2187 and 2188; SEQ ID NOs: 2189 and 2190; SEQ ID NOs: 2191 and 2192; SEQ ID NOs: 2193 and 2194; SEQ ID NOs: 2195 and 2196; SEQ ID NOs: 2197 and 2198; SEQ ID NOs: 2199 and 2200; SEQ ID NOs: 2201 and 2202; SEQ ID NOs: 2203 and 2204; SEQ ID NOs: 2205 and 2206; SEQ ID NOs: 2207 and 2208; SEQ ID NOs: 2209 and 2210; SEQ ID NOs: 2211 and 2212; SEQ ID NOs: 2213 and 2214; SEQ ID NOs: 2215 and 2216; SEQ ID NOs: 2217 and 2218; SEQ ID NOs: 2219 and 2220; SEQ ID NOs: 2221 and 2222; SEQ ID NOs: 2223 and 2224; SEQ ID NOs: 2225 and 2226; SEQ ID NOs: 2227 and 2228; SEQ ID NOs: 2229 and 2230; SEQ ID NOs: 2231 and 2232; SEQ ID NOs: 2233 and 2234; SEQ ID NOs: 2235 and 2236; SEQ ID NOs: 2237 and 2238; SEQ ID NOs: 2239 and 2240; SEQ ID NOs: 2241 and 2242; SEQ ID NOs: 2243 and 2244; SEQ ID NOs: 2245 and 2246; SEQ ID NOs: 2247 and 2248; SEQ ID NOs: 2249 and 2250; SEQ ID NOs: 2251 and 2252; SEQ ID NOs: 2253 and 2254; SEQ ID NOs: 2255 and 2256; SEQ ID NOs: 2257 and 2258; SEQ ID NOs: 2259 and 2260. As a non-limiting example, these C19orf80-saRNA sequences which are saRNA duplexes may be used to modulate betatrophin protein levels, regulating autophagy and lipid metabolism, treating obesity and/or treating type II or type I diabetes.

### F7 Gene

As another non-limiting example, provided is a method of modulating the expression of F7 gene comprising administering saRNA of the present invention, wherein the saRNA targets an antisense RNA transcript of F7 gene. These saRNAs are called F7-saRNA. In one embodiment, the expression of F7 gene is increased by at least 20, 30, 40%, or at least 45, 50, 55, 60, 65, 70, 75%, or at least 80% in the presence of the F7-saRNA of the present invention compared to the expression of F7 gene in the absence of the F7-saRNA of the present invention. In a further embodiment, the expression of F7 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the F7-saRNA of the present invention compared to the expression of F7 gene in the absence of the F7-saRNA of the present invention. The modulation of the expression of F7 gene may be reflected or determined by the change of F7 mRNA levels.

F7-saRNAs may be single-stranded and comprise 14-30 nucleotides. The sequence of a single-stranded F7-saRNA may have at least 60%, 70%, 80% or 90% identity with a sequence selected from the sequences of the antisense strands in Table 48. In one embodiment, the single-stranded saRNA comprises a sequence selected from the sequences of the antisense strands in Table 48. In one embodiment, the single-stranded F7-saRNA may have a 3′ tail. The sequence of a single-stranded F7-saRNA with a 3′ tail may have at least 60%, 70%, 80% or 90% identity with a sequence selected from the sequences of the antisense strands in Table 49. In one embodiment, the single-stranded saRNA comprises a sequence selected from the sequences of the antisense strands in Table 49.

F7-saRNAs may be double-stranded. The two strands form a duplex and each strand comprises 14-30 nucleotides. The first strand of a double-stranded F7-saRNA may have at least 60%, 70%, 80% or 90% identity with a sequence selected from the sequences of the antisense strands in Table 48. In one embodiment, the first strand of the double-stranded saRNA comprises a sequence selected from the sequences of the antisense strands in Table 48. The second strand of a double-stranded F7-saRNA may have at least 60%, 70%, 80% or 90% identity with a sequence selected from the sequences of the sense strands in Table 48. In one embodiment, the second strand of the double-stranded saRNA comprises a sequence selected from the sequences of the sense strands in Table 48. In one embodiment, the double-stranded F7-saRNA may have a 3′ overhang on each strand. The first strand of a double-stranded F7-saRNA may have at least 60%, 70%, 80% or 90% identity with a sequence selected from the sequences of the antisense strands in Table 49. In one embodiment, the first strand of the double-stranded saRNA comprises a sequence selected from the sequences of the antisense strands in Table 49. The second strand of a double-stranded F7-saRNA may have at least 60%, 70%, 80% or 90% identity with a sequence selected from the sequences of the sense strands in Table 49. In one embodiment, the second strand of the double-stranded saRNA comprises a sequence selected from the sequences of the sense strands in Table 49.

F7-saRNAs may be modified or unmodified.

Table 48-1 describes the target gene, the targeted sequence, the genomic location of the targeted sequence and the relative location of F7-saRNAs with no 3′ overhang. In Table 48-1, the targeted sequence is defined as a region on the template strand of the target gene which is identical to a region on the target antisense RNA transcript to which the antisense strand of an saRNA hybridizes. In Table 48-1, the relative location is the distance from the 5′ end of the targeted sequence to the TSS. A negative number represents a location upstream of the TSS and a positive number represents a location downstream of the TSS.

TABLE 48-1
Sequences of F7-saRNAs (with no 3′ overhang)

| saRNA | Target | | Targeted | Genomic Location of | Relative |
|---|---|---|---|---|---|
| ID | Gene | Targeted sequence | sequence ID | the Targeted sequence | Location |
| F7.NM_019616- | F7 | agcaatcatgcgagtcaaa | 1890 | chr13: 113759763 | −319 |
| Pr-3 | human | | | minus strand | |
| F7.NM_019616- | F7 | agtcaaatctcaagactgt | 1891 | chr13: 113759751 | −331 |
| Pr- | human | | | minus strand | |
| 20 | | | | | |
| F7.NM_019616- | F7 | ctgagaagtgccgggaata | 1892 | chr13: 113759628 | −454 |
| Pr- | human | | | minus strand | |
| 21 | | | | | |
| F7.NM_019616- | F7 | gggtgtggatgtaaatatt | 1893 | chr13: 113759727 | −355 |
| Pr- | human | | | minus strand | |
| 23 | | | | | |
| F7.NM_019616- | F7 | caaatacgatctagaattc | 1894 | chr13: 113759840 | −242 |
| Pr- | human | | | minus strand | |
| 27 | | | | | |
| F7.NM_019616- | F7 | tagcaatcatgcgagtcaa | 1895 | chr13: 113759764 | −318 |
| Pr- | human | | | minus strand | |
| 28 | | | | | |
| F7.NM_019616- | F7 | gggttctcagaatgtggaa | 1896 | chr13: 113759116 | −966 |
| Pr- | human | | | minus strand | |
| 30 | | | | | |
| F7.NM_019616- | F7 | atgctggtttctagaagga | 1897 | chr13: 113760365 | 283 |
| Pr- | human | | | minus strand | |
| 38 | | | | | |
| F7.NM_019616- | F7 | agatttaaactgcagatga | 1898 | chr13: 113759795 | −287 |
| Pr- | human | | | minus strand | |
| 39 | | | | | |

TABLE 48-2
Sequences of F7-saRNAs (with no 3′ overhang)

| | | Sense SEQ | | Anti-sense |
|---|---|---|---|---|
| saRNA ID | Sense (passenger) | ID | Antisense (guide) | SEQ ID |
| F7.NM_019616- | AGCAAUCAUGCGAGUC | 1899 | UUUGACUCGCAUG | 1900 |
| Pr-3 | AAA | | AUUGCU | |
| F7.NM_019616- | AGUCAAAUCUCAAGAC | 1901 | ACAGUCUUGAGAU | 1902 |
| Pr-20 | UGU | | UUGACU | |
| F7.NM_019616- | CUGAGAAGUGCCGGG | 1903 | UAUUCCCGGCACUU | 1904 |
| Pr-21 | AAUA | | CUCAG | |
| F7.NM_019616- | GGGUGUGGAUGUAAA | 1905 | AAUAUUUACAUCC | 1906 |
| Pr-23 | UAUU | | ACACCC | |
| F7.NM_019616- | CAAAUACGAUCUAGA | 1907 | GAAUUCUAGAUCG | 1908 |
| Pr-27 | AUUC | | UAUUUG | |
| F7.NM_019616- | UAGCAAUCAUGCGAG | 1909 | UUGACUCGCAUGA | 1910 |
| Pr-28 | UCAA | | UUGCUA | |
| F7.NM_019616- | GGGUUCUCAGAAUGU | 1911 | UUCCACAUUCUGAG | 1912 |
| Pr-30 | GGAA | | AACCC | |
| F7.NM_019616- | AUGCUGGUUUCUAGA | 1913 | UCCUUCUAGAAACC | 1914 |
| Pr-38 | AGGA | | AGCAU | |
| F7.NM_019616- | AGAUUUAAACUGCAG | 1915 | UCAUCUGCAGUUU | 1916 |
| Pr-39 | AUGA | | AAAUCU | |

TABLE 49
Sequences of F7-saRNAs (with 3′ overhang)

| | | Sense | | Antisense |
|---|---|---|---|---|
| saRNA ID | Sense (passenger) | SEQ ID | Antisense (guide) | SEQ ID |
| F7.NM_019616- | AGCAAUCAUGCGAGUCA | 1917 | UUUGACUCGCAUGAU | 1918 |
| Pr-3′ | AAUU | | UGCUUU | |
| F7.NM_019616- | AGUCAAAUCUCAAGACU | 1919 | ACAGUCUUGAGAUUU | 1920 |
| Pr-20′ | GUUU | | GACUUU | |
| F7.NM_019616- | CUGAGAAGUGCCGGGAA | 1921 | UAUUCCCGGCACUUCU | 1922 |
| Pr-21′ | UAUU | | CAGUU | |
| F7.NM_019616- | GGGUGUGGAUGUAAAUA | 1923 | AAUAUUUACAUCCAC | 1924 |
| Pr-23′ | UUUU | | ACCCUU | |
| F7.NM_019616- | CAAAUACGAUCUAGAAU | 1925 | GAAUUCUAGAUCGUA | 1926 |
| Pr-27′ | UCUU | | UUUGUU | |
| F7.NM_019616- | UAGCAAUCAUGCGAGUC | 1927 | UUGACUCGCAUGAUU | 1928 |
| Pr-28′ | AAUU | | GCUAUU | |
| F7.NM_019616- | GGGUUCUCAGAAUGUGG | 1929 | UUCCACAUUCUGAGA | 1930 |
| Pr-30′ | AAUU | | ACCCUU | |
| F7.NM_019616- | AUGCUGGUUUCUAGAAG | 1931 | UCCUUCUAGAAACCA | 1932 |
| Pr-38′ | GAUU | | GCAUUU | |
| F7.NM_019616- | AGAUUUAAACUGCAGAU | 1933 | UCAUCUGCAGUUUAA | 1934 |
| Pr-39′ | GAUU | | AUCUUU | |

F7 gene encodes coagulation factor VII which is a vitamin K-dependent factor essential for hemostasis. Coagulation factor VII protein is converted to an active form (factor VIIa) by either factor IXa, factor Xa, factor XIIa, or thrombin by minor proteolysis. The activated factor VIIa then further activates the coagulation cascade that forms a blood blot by converting factor IX to factor IXa and/or factor X to factor Xa. Mutations in F7 gene can cause coagulopathy and factor VII deficiency. Factor VII deficiency is characterized by a bleeding phenotype varying from mild to severe (Elmahmoudi et al., Diagn Pathol., vol. 7:92 (2012), the contents of which are incorporated herein by reference in their entirety). It may lead to early occurrence of intracerebral hemorrhage, repeated hemarthroses, cutaneous-mucosal hemorrhages (epistaxis, menorrhagia), or hemorrhages provoked by a surgical intervention.

In one embodiment, provided is a method of modulating coagulation factor VII protein levels comprising administering F7-saRNA of the present invention, wherein the F7-saRNA targets an antisense RNA transcript of F7 gene. In another embodiment, provided is a method of treating factor VII deficiency comprising administering F7-saRNA of the present invention, wherein the F7-saRNA targets an antisense RNA transcript of F7 gene. In one embodiment, coagulation factor VII protein level is increased by at least 20, 30, 40%, or at least 45, 50, 55, 60, 65, 70, 75%, or at least 80% in the presence of the F7-saRNA of the present invention compared to coagulation factor VII protein level in the absence of the F7-saRNA of the present invention. In a further embodiment, coagulation factor VII protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the F7-saRNA of the present invention compared to coagulation factor VII protein level in the absence of the F7-saRNA of the present invention.

In one embodiment, the F7-saRNA of the present invention is an antisense sequence such as, but not limited to, SEQ ID NO: 1900, 1902, 1904, 1906, 1908, 1910, 1912, 1914, 1916, 1918, 1920, 1922, 1924, 1926, 1928, 1930, 1932, 1934, 3218, 3220, 3222, 3224, 3226, 3228, 3230, 3232, 3234, 3236, 3238, 3240, 3242, 3244, 3246, 3248, 3250, 3252, 3254, 3256, 3258, 3260, 3262, 3264, 3266, 3268, 3270, 3272, 3274, 3276, 3278, 3280, 3282, 3284 and 3286. As a non-limiting example, these F7-saRNA sequences may be used to modulate coagulation factor VII protein levels.

In one embodiment, the F7-saRNA of the present invention is an saRNA duplex. The saRNA duplex may be a pair of sense and antisense sequences such as, but not limited to, SEQ ID NOs: 1899 and 1900: SEQ ID NOs: 1901 and 1902: SEQ ID NOs: 1903 and 1904: SEQ ID NOs: 1905 and 1906: SEQ ID NOs: 1907 and 1908: SEQ ID NOs: 1909 and 1910: SEQ ID NOs: 1911 and 1912: SEQ ID NOs: 1913 and 1914: SEQ ID NOs: 1915 and 1916: SEQ ID NOs: 1917 and 1918: SEQ ID NOs: 1919 and 1920: SEQ ID NOs: 1921 and 1922: SEQ ID NOs: 1923 and 1924: SEQ ID NOs: 1925 and 1926: SEQ ID NOs: 1927 and 1928: SEQ ID NOs: 1929 and 1930: SEQ ID NOs: 1931 and 1932: SEQ ID NOs: 1933 and 1934: SEQ ID NOs: 3217 and 3218: SEQ ID NOs: 3219 and 3220: SEQ ID NOs: 3221 and 3222: SEQ ID NOs: 3223 and 3224: SEQ ID NOs: 3225 and 3226: SEQ ID NOs: 3227 and 3228: SEQ ID NOs: 3229 and 3230: SEQ ID NOs: 3231 and 3232: SEQ ID NOs: 3233 and 3234: SEQ ID NOs: 3235 and 3236: SEQ ID NOs: 3237 and 3238: SEQ ID NOs: 3239 and 3240: SEQ ID NOs: 3241 and 3242: SEQ ID NOs: 3243 and 3244: SEQ ID NOs: 3245 and 3246: SEQ ID NOs: 3247 and 3248: SEQ ID NOs: 3249 and 3250: SEQ ID NOs: 3251 and 3252: SEQ ID NOs: 3253 and 3254: SEQ ID NOs: 3255 and 3256: SEQ ID NOs: 3257 and 3258: SEQ ID NOs: 3259 and 3260: SEQ ID NOs: 3261 and 3262: SEQ ID NOs: 3263 and 3264: SEQ ID NOs: 3265 and 3266: SEQ ID NOs: 3267 and 3268: SEQ ID NOs: 3269 and 3270: SEQ ID NOs: 3271 and 3272: SEQ ID NOs: 3273 and 3274: SEQ ID NOs: 3275 and 3276: SEQ ID NOs: 3277 and 3278: SEQ ID NOs: 3279 and 3280: SEQ ID NOs: 3281 and 3282: SEQ ID NOs: 3283 and 3284: SEQ ID NOs: 3285 and 3286. As a non-limiting example, these F7-saRNA sequences which are saRNA duplexes may be used to modulate coagulation factor VII protein levels.

### F8 Gene

As another non-limiting example, provided is a method of modulating the expression of F8 gene comprising administering saRNA of the present invention, wherein the saRNA targets an antisense RNA transcript of F8 gene. These saRNAs are called F8-saRNA. In one embodiment, the expression of F8 gene is increased by at least 20, 30, 40%, or at least 45, 50, 55, 60, 65, 70, 75%, or at least 80% in the presence of the F8-saRNA of the present invention compared to the expression of F8 gene in the absence of the F8-saRNA of the present invention. In a further embodiment, the expression of F8 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the F8-saRNA of the present invention compared to the expression of F8 gene in the absence of the F8-saRNA of the present invention. The modulation of the expression of F8 gene may be reflected or determined by the change of F8 mRNA levels.

F8-saRNAs may be single-stranded and comprise 14-30 nucleotides. The sequence of a single-stranded F8-saRNA may have at least 60%, 70%, 80% or 90% identity with a sequence selected from the sequences of the antisense strands in Table 50-2. In one embodiment, the single-stranded saRNA comprises a sequence selected from the sequences of the antisense strands in Table 50-2. In one embodiment, the single-stranded F8-saRNA may have a 3′ tail. The sequence of a single-stranded F8-saRNA with a 3′ tail may have at least 60%, 70%, 80% or 90% identity with a sequence selected from the sequences of the antisense strands in Table 51. In one embodiment, the single-stranded saRNA comprises a sequence selected from the sequences of the antisense strands in Table 51.

F8-saRNAs may be double-stranded. The two strands form a duplex and each strand comprises 14-30 nucleotides. The first strand of a double-stranded F8-saRNA may have at least 60%, 70%, 80% or 90% identity with a sequence selected from the sequences of the antisense strands in Table 50-2. In one embodiment, the first strand of the double-stranded saRNA comprises a sequence selected from the sequences of the antisense strands in Table 50-2. The second strand of a double-stranded F8-saRNA may have at least 60%, 70%, 80% or 90% identity with a sequence selected from the sequences of the sense strands in Table 50-2. In one embodiment, the second strand of the double-stranded saRNA comprises a sequence selected from the sequences of the sense strands in Table 50-2. In one embodiment, the double-stranded F8-saRNA may have a 3′ overhang on each strand. The first strand of a double-stranded F8-saRNA may have at least 60%, 70%, 80% or 90% identity with a sequence selected from the sequences of the antisense strands in Table 51. In one embodiment, the first strand of the double-stranded saRNA comprises a sequence selected from the sequences of the antisense strands in Table 51. The second strand of a double-stranded F8-saRNA may have at least 60%, 70%, 80% or 90% identity with a sequence selected from the sequences of the sense strands in Table 51. In one embodiment, the second strand of the double-stranded saRNA comprises a sequence selected from the sequences of the sense strands in Table 51.

F8-saRNAs may be modified or unmodified.

Table 50-1 describes the target gene, the targeted sequence, the genomic location of the targeted sequence and the relative location of F8-saRNAs with no 3′ overhang. In Table 50-1, the targeted sequence is defined as a region on the template strand of the target gene which is identical to a region on the target antisense RNA transcript to which the antisense strand of an saRNA hybridizes. In Table 50-1, the relative location is the distance from the 5′ end of the targeted sequence to the TSS. A negative number represents a location upstream of the TSS and a positive number represents a location downstream of the TSS.

TABLE 50-1
Sequences of F8-saRNAs (with no 3′ overhang)

| | | | | Genomic Location | |
|---|---|---|---|---|---|
| | | | Targeted | of the Targeted | Relative |
| saRNA ID | Target Gene | Targeted sequence | sequence ID | sequence | Location |
| F8.NM_000132- | F8 human | ggcataagtctgctttaat | 1935 | chrX: 154251110 | −112 |
| Pr-1 | | | | plus strand | |
| F8.NM_000132- | F8 human | acaggagactcttacataa | 1936 | chrX: 154250003 | 995 |
| Pr-2 | | | | plus strand | |
| F8.NM_000132- | F8 human | ccgagatcactttgcatat | 1937 | chrX: 154250705 | 293 |
| Pr-4 | | | | plus strand | |
| F8.NM_000132- | F8 human | tagattgctttctatgaaa | 1938 | chrX: 154250056 | 942 |
| Pr-5 | | | | plus strand | |
| F8.NM_000132- | F8 human | gggcataagtctgctttaa | 1939 | chrX: 154251109 | −111 |
| Pr-7 | | | | plus strand | |
| F8.NM_000132- | F8 human | cgagatcactttgcatata | 1940 | chrX: 154250706 | 292 |
| Pr-12 | | | | plus strand | |
| F8.NM_000132- | F8 human | tgtcgaagattctagaatt | 1941 | chrX: 154251258 | −260 |
| Pr-13 | | | | plus strand | |
| F8.NM_000132- | F8 human | agcatttctgctaataaca | 1942 | chrX: 154250517 | 481 |
| Pr-31 | | | | plus strand | |
| F8.NM_000132- | F8 human | tggagagctctatttgcat | 1943 | chrX: 154250808 | 190 |
| Pr-32 | | | | plus strand | |
| F8.NM_000132- | F8 human | ggatatatctctttacatc | 1944 | chrX: 154251961 | −963 |
| Pr-35 | | | | plus strand | |
| F8.NM_000132- | F8 human | gctctatttgcatgactta | 1945 | chrX: 154250814 | 184 |
| Pr-40 | | | | plus strand | |
| F8.NM_019863- | F8 human | Gggacaacagtctagaaat | 1946 | chrX: 154114250 | 327 |
| Pr-2 | | | | plus strand | |
| F8.NM_019863- | F8 human | acgagtttcgtttgggtca | 1947 | chrX: 154114291 | 286 |
| Pr-14 | | | | plus strand | |
| F8.NM_019863- | F8 human | tctagaaatgcgcagacat | 1948 | chrX: 154114260 | 317 |
| Pr-23 | | | | plus strand | |
| F8.NM_019863- | F8 human | tcacgagtttcgtttgggt | 1949 | chrX: 154114289 | 288 |
| Pr-24 | | | | plus strand | |
| F8.NM_019863- | F8 human | cgagtttcgtttgggtcat | 1950 | chrX: 154114292 | 285 |
| Pr-29 | | | | plus strand | |
| F8.NM_019863- | F8 human | ctagaaatgcgcagacatg | 1951 | chrX: 154114261 | 316 |
| Pr-33 | | | | plus strand | |

TABLE 50-2
Sequences of F8-saRNAs (with no 3′ overhang)

| | | | | Anti-sense |
|---|---|---|---|---|
| saRNA ID | Sense (passenger) | Sense SEQ ID | Antisense (guide) | SEQ ID |
| F8.NM_000132-Pr-1 | GGCAUAAGUCUG | 1952 | AUUAAAGCAGAC | 1953 |
| | CUUUAAU | | UUAUGCC | |
| F8.NM_000132-Pr-2 | ACAGGAGACUCU | 1954 | UUAUGUAAGAG | 1955 |
| | UACAUAA | | UCUCCUGU | |
| F8.NM_000132-Pr-4 | CCGAGAUCACUU | 1956 | AUAUGCAAAGU | 1957 |
| | UGCAUAU | | GAUCUCGG | |
| F8.NM_000132-Pr-5 | UAGAUUGCUUUC | 1958 | UUUCAUAGAAA | 1959 |
| | UAUGAAA | | GCAAUCUA | |
| F8.NM_000132-Pr-7 | GGGCAUAAGUCU | 1960 | UUAAAGCAGACU | 1961 |
| | GCUUUAA | | UAUGCCC | |
| F8.NM_000132-Pr-12 | CGAGAUCACUUU | 1962 | UAUAUGCAAAG | 1963 |
| | GCAUAUA | | UGAUCUCG | |
| F8.NM_000132-Pr-13 | UGUCGAAGAUUC | 1964 | AAUUCUAGAAUC | 1965 |
| | UAGAAUU | | UUCGACA | |
| F8.NM_000132-Pr-31 | AGCAUUUCUGCU | 1966 | UGUUAUUAGCA | 1967 |
| | AAUAACA | | GAAAUGCU | |
| F8.NM_000132-Pr-32 | UGGAGAGCUCUA | 1968 | AUGCAAAUAGA | 1969 |
| | UUUGCAU | | GCUCUCCA | |
| F8.NM_000132-Pr-35 | GGAUAUAUCUCU | 1970 | GAUGUAAAGAG | 1971 |
| | UUACAUC | | AUAUAUCC | |
| F8.NM_000132-Pr-40 | GCUCUAUUUGCA | 1972 | UAAGUCAUGCAA | 1973 |
| | UGACUUA | | AUAGAGC | |
| F8.NM_019863-Pr-2 | GGGACAACAGUC | 1974 | AUUUCUAGACUG | 1975 |
| | UAGAAAU | | UUGUCCC | |
| F8.NM_019863-Pr-14 | ACGAGUUUCGUU | 1976 | UGACCCAAACGA | 1977 |
| | UGGGUCA | | AACUCGU | |
| F8.NM_019863-Pr-23 | UCUAGAAAUGCG | 1978 | AUGUCUGCGCAU | 1979 |
| | CAGACAU | | UUCUAGA | |
| F8.NM_019863-Pr-24 | UCACGAGUUUCG | 1980 | ACCCAAACGAAA | 1981 |
| | UUUGGGU | | CUCGUGA | |
| F8.NM_019863-Pr-29 | CGAGUUUCGUUU | 1982 | AUGACCCAAACG | 1983 |
| | GGGUCAU | | AAACUCG | |
| F8.NM_019863-Pr-33 | CUAGAAAUGCGC | 1984 | CAUGUCUGCGCA | 1985 |
| | AGACAUG | | UUUCUAG | |

TABLE 51
Sequences of F8-saRNAs (with 3′ overhang)

| | | | | Antisense |
|---|---|---|---|---|
| saRNA ID | Sense (passenger) | Sense SEQ ID | Antisense (guide) | SEQ ID |
| F8.NM_000132-Pr-1′ | GGCAUAAGUCUG | 1986 | AUUAAAGCAGAC | 1987 |
| | CUUUAAUUU | | UUAUGCCUU | |
| F8.NM_000132-Pr-2′ | ACAGGAGACUCU | 1988 | UUAUGUAAGAG | 1989 |
| | UACAUAAUU | | UCUCCUGUUU | |
| F8.NM_000132-Pr-4′ | CCGAGAUCACUU | 1990 | AUAUGCAAAGU | 1991 |
| | UGCAUAUUU | | GAUCUCGGUU | |
| F8.NM_000132-Pr-5′ | UAGAUUGCUUUC | 1992 | UUUCAUAGAAA | 1993 |
| | UAUGAAAUU | | GCAAUCUAUU | |
| F8.NM_000132-Pr-7′ | GGGCAUAAGUCU | 1994 | UUAAAGCAGACU | 1995 |
| | GCUUUAAUU | | UAUGCCCUU | |
| F8.NM_000132-Pr-12′ | CGAGAUCACUUU | 1996 | UAUAUGCAAAG | 1997 |
| | GCAUAUAUU | | UGAUCUCGUU | |
| F8.NM_000132-Pr-13′ | UGUCGAAGAUUC | 1998 | AAUUCUAGAAUC | 1999 |
| | UAGAAUUUU | | UUCGACAUU | |
| F8.NM_000132-Pr-31′ | AGCAUUUCUGCU | 2000 | UGUUAUUAGCA | 2001 |
| | AAUAACAUU | | GAAAUGCUUU | |
| F8.NM_000132-Pr-32′ | UGGAGAGCUCUA | 2002 | AUGCAAAUAGA | 2003 |
| | UUUGCAUUU | | GCUCUCCAUU | |
| F8.NM_000132-Pr-35′ | GGAUAUAUCUCU | 2004 | GAUGUAAAGAG | 2005 |
| | UUACAUCUU | | AUAUAUCCUU | |
| F8.NM_000132-Pr-40′ | GCUCUAUUUGCA | 2006 | UAAGUCAUGCAA | 2007 |
| | UGACUUAUU | | AUAGAGCUU | |
| F8.NM_019863-Pr-2′ | GGGACAACAGUC | 2008 | AUUUCUAGACUG | 2009 |
| | UAGAAAUUU | | UUGUCCCUU | |
| F8.NM_019863-Pr-14′ | ACGAGUUUCGUU | 2010 | UGACCCAAACGA | 2011 |
| | UGGGUCAUU | | AACUCGUUU | |
| F8.NM_019863-Pr-23′ | UCUAGAAAUGCG | 2012 | AUGUCUGCGCAU | 2013 |
| | CAGACAUUU | | UUCUAGAUU | |
| F8.NM_019863-Pr-24′ | UCACGAGUUUCG | 2014 | ACCCAAACGAAA | 2015 |
| | UUUGGGUUU | | CUCGUGAUU | |
| F8.NM_019863-Pr-29′ | CGAGUUUCGUUU | 2016 | AUGACCCAAACG | 2017 |
| | GGGUCAUUU | | AAACUCGUU | |
| F8.NM_019863-Pr-33′ | CUAGAAAUGCGC | 2018 | CAUGUCUGCGCA | 2019 |
| | AGACAUGUU | | UUUCUAGUU | |

F8 gene encodes coagulation factor VIII, which circulates in the bloodstream in an inactive form, bound to another molecule called von Willebrand factor. When an injury of blood vessels occurs, factor VIII is activated to factor VIIIa and separates from von Willebrand factor to participate in the coagulation cascade that forms a blood clot. Mutations of F8 gene cause hemophilia A, a bleeding disorder, because factor VIII protein level is reduced or the function of factor VIII protein is lost (Seo et al., Blood Res., vol. 48(3):206 (2013), the contents of which are incorporated herein by reference in their entirety).

In one embodiment, provided is a method of modulating coagulation factor VIII protein levels comprising administering F8-saRNA of the present invention, wherein the F8-saRNA targets an antisense RNA transcript of F8 gene. In another embodiment, provided is a method of treating hemophilia A comprising administering F8-saRNA of the present invention, wherein the F8-saRNA targets an antisense RNA transcript of F8 gene. In one embodiment, coagulation factor VIII protein level is increased by at least 20, 30, 40%, or at least 45, 50, 55, 60, 65, 70, 75%, or at least 80% in the presence of the F8-saRNA of the present invention compared to coagulation factor VIII protein level in the absence of the F8-saRNA of the present invention. In a further embodiment, coagulation factor VIII protein level is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the F8-saRNA of the present invention compared to coagulation factor VIII protein level in the absence of the F8-saRNA of the present invention.

In one embodiment, the F8-saRNA of the present invention is an antisense sequence such as, but not limited to, SEQ ID NO: 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 3288, 3290, 3292, 3294, 3296, 3298, 3300, 3302, 3304, 3306, 3308, 3310, 3312, 3314, 3316, 3318, 3320, 3322, 3324, 3326, 3328, 3330, 3332, 3334, 3336, 3338, 3340, 3342, 3344, 3346, 3348, 3350, 3352, 3354, 3356, 3358, 3360, 3362, 3364, 3366, 3368, 3370, 3372, 3374, 3376, 3378, 3380, 3382, 3384, 3386, 3388, 3390, 3392, 3394 and 3396. As a non-limiting example, these F8-saRNA sequences may be used to modulate coagulation factor VIII protein levels and/or treat hemophilia A.

In one embodiment, the F8-saRNA of the present invention is an saRNA duplex. The saRNA duplex may be a pair of sense and antisense sequences such as, but not limited to, SEQ ID NOs: 1952 and 1953; SEQ ID NOs: 1954 and 1955; SEQ ID NOs: 1956 and 1957; SEQ ID NOs: 1958 and 1959; SEQ ID NOs: 1960 and 1961; SEQ ID NOs: 1962 and 1963; SEQ ID NOs: 1964 and 1965; SEQ ID NOs: 1966 and 1967; SEQ ID NOs: 1968 and 1969; SEQ ID NOs: 1970 and 1971; SEQ ID NOs: 1972 and 1973; SEQ ID NOs: 1974 and 1975; SEQ ID NOs: 1976 and 1977; SEQ ID NOs: 1978 and 1979; SEQ ID NOs: 1980 and 1981; SEQ ID NOs: 1982 and 1983; SEQ ID NOs: 1984 and 1985; SEQ ID NOs: 1986 and 1987; SEQ ID NOs: 1988 and 1989; SEQ ID NOs: 1990 and 1991; SEQ ID NOs: 1992 and 1993; SEQ ID NOs: 1994 and 1995; SEQ ID NOs: 1996 and 1997; SEQ ID NOs: 1998 and 1999; SEQ ID NOs: 2000 and 2001; SEQ ID NOs: 2002 and 2003; SEQ ID NOs: 2004 and 2005; SEQ ID NOs: 2006 and 2007; SEQ ID NOs: 2008 and 2009; SEQ ID NOs: 2010 and 2011; SEQ ID NOs: 2012 and 2013; SEQ ID NOs: 2014 and 2015; SEQ ID NOs: 2016 and 2017; SEQ ID NOs: 2018 and 2019; SEQ ID NOs: 3287 and 3288; SEQ ID NOs: 3289 and 3290; SEQ ID NOs: 3291 and 3292; SEQ ID NOs: 3293 and 3294; SEQ ID NOs: 3295 and 3296; SEQ ID NOs: 3297 and 3298; SEQ ID NOs: 3299 and 3300; SEQ ID NOs: 3301 and 3302; SEQ ID NOs: 3303 and 3304; SEQ ID NOs: 3305 and 3306; SEQ ID NOs: 3307 and 3308; SEQ ID NOs: 3309 and 3310; SEQ ID NOs: 3311 and 3312; SEQ ID NOs: 3313 and 3314; SEQ ID NOs: 3315 and 3316; SEQ ID NOs: 3317 and 3318; SEQ ID NOs: 3319 and 3320; SEQ ID NOs: 3321 and 3322; SEQ ID NOs: 3323 and 3324; SEQ ID NOs: 3325 and 3326; SEQ ID NOs: 3327 and 3328; SEQ ID NOs: 3329 and 3330; SEQ ID NOs: 3331 and 3332; SEQ ID NOs: 3333 and 3334; SEQ ID NOs: 3335 and 3336; SEQ ID NOs: 3337 and 3338; SEQ ID NOs: 3339 and 3340; SEQ ID NOs: 3341 and 3342; SEQ ID NOs: 3343 and 3344; SEQ ID NOs: 3345 and 3346; SEQ ID NOs: 3347 and 3348; SEQ ID NOs: 3349 and 3350; SEQ ID NOs: 3351 and 3352; SEQ ID NOs: 3353 and 3354; SEQ ID NOs: 3355 and 3356; SEQ ID NOs: 3357 and 3358; SEQ ID NOs: 3359 and 3360; SEQ ID NOs: 3361 and 3362; SEQ ID NOs: 3363 and 3364; SEQ ID NOs: 3365 and 3366; SEQ ID NOs: 3367 and 3368; SEQ ID NOs: 3369 and 3370; SEQ ID NOs: 3371 and 3372; SEQ ID NOs: 3373 and 3374; SEQ ID NOs: 3375 and 3376; SEQ ID NOs: 3377 and 3378; SEQ ID NOs: 3379 and 3380; SEQ ID NOs: 3381 and 3382; SEQ ID NOs: 3383 and 3384; SEQ ID NOs: 3385 and 3386; SEQ ID NOs: 3387 and 3388; SEQ ID NOs: 3389 and 3390; SEQ ID NOs: 3391 and 3392; SEQ ID NOs: 3393 and 3394; SEQ ID NOs: 3395 and 3396. As a non-limiting example, these F8-saRNA sequences which are saRNA duplexes may be used to modulate coagulation factor VIII protein levels and/or treat hemophilia A.

Additional saRNA Sequences

Additional saRNAs are designed from the method disclosed in the present application. In some embodiments, the saRNAs are single-stranded. They are identified in the Sequence Listing as antisense. The reverse complement of the antisense saRNA strand (identified in the Sequence Listing as sense) has sequence identity with a target site or targeted sequence on the template strand of a target gene. The location of the target site is identified by its relative location to the TSS on the template strand (a subportion of which comprises the TSS core sequence). In some embodiments, the single-stranded saRNA may have a 3′ tail. The single-stranded saRNA molecule binds to the target antisense RNA transcript in the nucleus.

In some embodiments, the saRNAs are double-stranded. Each saRNA molecule comprises an antisense strand and a sense strand. Each strand is identified in the Sequence Listing as antisense or sense. In some embodiments, each strand may have a 3′ tail.

The sequences of the saRNAs (SEQ ID Nos 2061-9946) and the sequences of the TSS cores (SEQ ID Nos 2020-2060) are provided in the Sequence Listing. The nature of the sequence, antisense or sense, is identified in the description field of the Sequence Listing for each saRNA sequence. The antisense saRNA may be administered as a single-stranded saRNA. The antisense saRNA may also form a double-stranded saRNA duplex with its sense saRNA and the double-stranded saRNA is administered. The SEQ ID No. of the sense, i.e., reverse complement, of the antisense saRNA is identified in the description field. The sense saRNA has sequence identity with a target site within the TSS core sequence. The SEQ ID No. of the TSS core and the start position of the target site within the TSS core is identified in the description field of the sense saRNA. Each TSS core is mapped to, or derived from, a target transcript. Therefore, the target transcript of the sense saRNA and the antisense saRNA can each be identified by the TSS core SEQ ID No. in the description field of the sense saRNA.

The target genes, target transcript RefSeq ID numbers, the protein encoded by the target transcript, and the TSS locations of the target genes are disclosed in Table 52.

TABLE 52
Target genes of additional saRNAs

| | | Protein | | | |
|---|---|---|---|---|---|
| | Target | encoded | Nature | Location on the coding strand | SEQ ID |
| Target | transcript | by target | of target | that corresponds to the target | of TSS |
| Gene | RefSeq No. | transcript | transcript | transcript's TSS | core |
| APOA1 | NM_000039 | NP_000030 | Coding | chr11: 116837622 minus strand | 2020 |
| C19orf80 | NM_018687 | NP_061157 | Coding | chr19: 11239618 plus strand | 2021 |
| CDKN1B | NM_004064 | NP_004055 | Coding | chr12: 12717269 plus strand | 2022 |
| DMD | NM_000109 | NP_000100 | Coding | chrX: 33339609 minus strand | 2023 |
| DMD | NM_004006 | NP_003997 | Coding | chrX: 33211556 minus strand | 2024 |
| DMD | NM_004009 | NP_004000 | Coding | chrX: 33128428 minus strand | 2025 |
| DMD | NM_004010 | NP_004001 | Coding | chrX: 33128427 minus strand | 2026 |
| DMD | NM_004011 | NP_004002 | Coding | chrX: 32412254 minus strand | 2027 |
| DMD | NM_004012 | NP_004003 | Coding | chrX: 32412254 minus strand | 2027 |
| DMD | NM_004013 | NP_004004 | Coding | chrX: 32155469 minus strand | 2028 |
| DMD | NM_004014 | NP_004005 | Coding | chrX: 31508298 minus strand | 2029 |
| DMD | NM_004015 | NP_004006 | Coding | chrX: 31266907 minus strand | 2030 |
| DMD | NM_004016 | NP_004007 | Coding | chrX: 31266907 minus strand | 2030 |
| DMD | NM_004017 | NP_004008 | Coding | chrX: 31266907 minus strand | 2030 |
| DMD | NM_004018 | NP_004009 | Coding | chrX: 31266907 minus strand | 2030 |
| DMD | NM_004019 | NP_004010 | Coding | chrX: 31508298 minus strand | 2030 |
| DMD | NM_004020 | NP_004011 | Coding | chrX: 32412254 minus strand | 2028 |
| DMD | NM_004021 | NP_004012 | Coding | chrX: 32412254 minus strand | 2028 |
| DMD | NM_004022 | NP_004013 | Coding | chrX: 32155469 minus strand | 2028 |
| DMD | NM_004023 | NP_004014 | Coding | chrX: 31508298 minus strand | 2028 |
| EPO | NM_000799 | NP_000790 | Coding | chr7: 100720799 plus strand | 2031 |
| F7 | NM_000131 | NP_000122 | Coding | chr13: 113105787 plus strand | 2032 |
| F7 | NM_001267554 | NP_001254483 | Coding | chr13: 113105787 plus strand | 2032 |
| F7 | NM_019616 | NP_062562 | Coding | chr13: 113105787 plus strand | 2032 |
| F8 | NM_000132 | NP_000123 | Coding | chrX: 155022723 minus strand | 2033 |
| F8 | NM_019863 | NP_063916 | Coding | chrX: 154886302 minus strand | 2034 |
| FNDC5 | NM_001171940 | NP_001165411 | Coding | chr1: 32870813 minus strand | 2035 |
| FNDC5 | NM_001171941 | NP_001165412 | Coding | chr1: 32872492 minus strand | 2036 |
| FNDC5 | NM_153756 | NP_715637 | Coding | chr1: 32870813 minus strand | 2035 |
| FOXA2 | NM_021784 | NP_068556 | Coding | chr20: 22585463 minus strand | 2037 |
| FOXA2 | NM_153675 | NP_710141 | Coding | chr20: 22584463 minus strand | 2038 |
| FOXP3 | NM_001114377 | NP_001107849 | Coding | chrX: 49264826 minus strand | 2039 |
| FOXP3 | NM_014009 | NP_054728 | Coding | chrX: 49264826 minus strand | 2039 |
| HNF4A | NM_000457 | NP_000448 | Coding | chr20: 44401255 plus strand | 2040 |
| HNF4A | NM_001030003 | NP_001025174 | Coding | chr20: 44355800 plus strand | 2041 |
| HNF4A | NM_001030004 | NP_001025175 | Coding | chr20: 44355800 plus strand | 2041 |
| HNF4A | NM_001258355 | NP_001245284 | Coding | chr20: 44401255 plus strand | 2040 |
| HNF4A | NM_001287182 | NP_001274111 | Coding | chr20: 44355800 plus strand | 2041 |
| HNF4A | NM_001287183 | NP_001274112 | Coding | chr20: 44355800 plus strand | 2041 |
| HNF4A | NM_001287184 | NP_001274113 | Coding | chr20: 22585463 minus strand | 2041 |
| HNF4A | NM_175914 | NP_787110 | Coding | chr20: 44355800 plus strand | 2041 |
| HNF4A | NM_178849 | NP_849180 | Coding | chr20: 44401255 plus strand | 2040 |
| HNF4A | NM_178850 | NP_849181 | Coding | chr20: 44401255 plus strand | 2040 |
| IDUA | NM_000203 | NP_000194 | Coding | chr4: 986996 plus strand | 2042 |
| IFNG | NM_000619 | NP_000610 | Coding | chr12: 68159741 minus strand | 2043 |
| IL10 | NM_000572 | NP_000563 | Coding | chr1: 206772494 minus strand | 2044 |
| IL19 | NM_013371 | NP_037503 | Coding | chr1: 206828876 plus strand | 2045 |
| IL19 | NM_153758 | NP_715639 | Coding | chr1: 206798869 plus strand | 2046 |
| IL2 | NM_000586 | NP_000577 | Coding | chr4: 122456495 minus strand | 2047 |
| LDLR | NM_000527 | NP_000518 | Coding | chr19: 11089361 plus strand | 2048 |
| LDLR | NM_001195798 | NP_001182727 | Coding | chr19: 11089361 plus strand | 2048 |
| LDLR | NM_001195799 | NP_001182728 | Coding | chr19: 11089361 plus strand | 2048 |
| LDLR | NM_001195800 | NP_001182729 | Coding | chr19: 11089361 plus strand | 2048 |
| LDLR | NM_001195803 | NP_001182732 | Coding | chr19: 11089361 plus strand | 2048 |
| LMX1A | NM_001174069 | NP_001167540 | Coding | chr1: 165356241 minus strand | 2049 |
| LMX1A | NM_177398 | NP_796372 | Coding | chr1: 165356241 minus strand | 2050 |
| MDM2 | NM_001145337 | NP_001138809 | Coding | chr12: 68809016 plus strand | 2051 |
| MDM2 | NM_001145339 | NP_001138811 | Coding | chr12: 68808171 plus strand | 2052 |
| MDM2 | NM_001145340 | NP_001138812 | Coding | chr12: 68809016 plus strand | 2051 |
| MDM2 | NM_001278462 | NP_001265391 | Coding | chr12: 68809016 plus strand | 2051 |
| MDM2 | NM_002392 | NP_002383 | Coding | chr12: 68808171 plus strand | 2052 |
| METRNL | NM_001004431 | NP_001004431 | Coding | chr17: 83079690 plus strand | 2053 |
| NR4A2 | NM_006186 | NP_006177 | Coding | chr2: 156332775 minus strand | 2054 |
| PAX5 | NM_001280547 | NP_001267476 | Coding | chr9: 37034479 minus strand | 2055 |
| PAX5 | NM_001280548 | NP_001267477 | Coding | chr9: 37034479 minus strand | 2055 |
| PAX5 | NM_001280549 | NP_001267478 | Coding | chr9: 37034479 minus strand | 2055 |
| PAX5 | NM_001280550 | NP_001267479 | Coding | chr9: 37034479 minus strand | 2055 |
| PAX5 | NM_001280551 | NP_001267480 | Coding | chr9: 37034479 minus strand | 2055 |
| PAX5 | NM_001280552 | NP_001267481 | Coding | chr9: 37034479 minus strand | 2055 |
| PAX5 | NM_001280553 | NP_001267482 | Coding | chr9: 37034479 minus strand | 2055 |
| PAX5 | NM_001280554 | NP_001267483 | Coding | chr9: 37034479 minus strand | 2055 |
| PAX5 | NM_001280555 | NP_001267484 | Coding | chr9: 37034479 minus strand | 2055 |
| PAX5 | NM_001280556 | NP_001267485 | Coding | chr9: 37034479 minus strand | 2055 |
| PAX5 | NM_016734 | NP_057953 | Coding | chr9: 37034479 minus strand | 2055 |
| SCN1A | NM_001165963 | NP_001159435 | Coding | chr2: 166073639 minus strand | 2056 |
| SCN1A | NM_001165964 | NP_001159436 | Coding | chr2: 166073639 minus strand | 2056 |
| SCN1A | NM_001202435 | NP_001189364 | Coding | chr2: 166149132 minus strand | 2057 |
| SCN1A | NM_006920 | NP_008851 | Coding | chr2: 166073639 minus strand | 2056 |
| SIRT1 | NM_001142498 | NP_001135970 | Coding | chr10: 67885180 plus strand | 2058 |
| SIRT1 | NM_012238 | NP_036370 | Coding | chr10: 67884668 plus strand | 2059 |
| TH | NM_000360 | NP_000351 | Coding | chr11: 2171805 minus strand | 2060 |
| TH | NM_199292 | NP_954986 | Coding | chr11: 2171805 minus strand | 2060 |
| TH | NM_199293 | NP_954987 | Coding | chr11: 2171805 minus strand | 2060 |

### IV. Kits and Devices

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for regulate the expression of genes in vitro or in vivo, comprising saRNA of the present invention or a combination of saRNA of the present invention, saRNA modulating other genes, siRNAs, miRNAs or other oligonucleotide molecules.

The kit may further comprise packaging and instructions and/or a delivery agent to form a formulation composition. The delivery agent may comprise a saline, a buffered solution, a lipidoid, a dendrimer or any delivery agent disclosed herein.

Non-limiting examples of genes include apolipoprotein A1 (APOA1), low density lipoprotein (LDLR), dystrophin (DMD), paired box 5 (PAX5), sodium channel voltage-gated type I alpha subunit (SCN1A), iduronidase alpha-L (IDUA), fibronectin type III domain containing 5 (FNDC5), forkhead box A2 (FOXA2), forkhead box P3 (FOXP3), hepatocyte nuclear factor 4, alpha (HNF4A), interferon, gamma (IFNG), interleukin 10 (IL10), interleukin 2 (IL2), LIM homeobox transcription factor I, alpha (LMXIA), meteorin, glial cell differentiation regulator-like (METRNL), nuclear receptor subfamily 4, group A, member 2 (NR4A2), sirtuin I (SIRTI), tyrosine hydroxylase (TH), erythropoietin (EPO), cyclin-dependent kinase inhibitor I B (p27, Kip I) (CDKNIB), MDM2 oncogene, E3 ubiquitin protein ligase (MDM2), interleukin 19 (IL19), chromosome 19 open reading frame 80 (C19orf90), coagulation factor VII (F7), coagulation factor VIII (F8), C/EBPα, other members of C/EBP family, albumin gene, alphafectoprotein gene, liver specific factor genes, growth factors, nuclear factor genes, tumor suppressing genes, and pluripotency factor genes.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated or it may be lyophilized. The amount of each component may be varied to enable consistent, reproducible higher concentration saline or simple buffer formulations. The components may also be varied in order to increase the stability of saRNA in the buffer solution over a period of time and/or under a variety of conditions.

### Devices

The present invention provides for devices which may incorporate saRNA of the present invention. These devices contain in a stable formulation available to be immediately delivered to a subject in need thereof, such as a human patient.

Non-limiting examples of such a subject include a subject with high LDL cholesterol, type I or type II diabetes, obesity, metabolic disorders, hyperproliferative disorders, or neurological disorders.

Non-limiting examples of the devices include a pump, a catheter, a needle, a transdermal patch, a pressurized olfactory delivery device, iontophoresis devices, multi-layered microfluidic devices. The devices may be employed to deliver saRNA of the present invention according to single, multi- or split-dosing regiments. The devices may be employed to deliver saRNA of the present invention across biological tissue, intradermal, subcutaneously, or intramuscularly. More examples of devices suitable for delivering oligonucleotides are disclosed in International Publication WO 2013/090648 filed Dec. 14, 2012, the contents of which are incorporated herein by reference in their entirety.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunction or Bifunctional: As used herein, the terms “bifunction” and “bifunctional” refers to any substance, molecule or moiety which is capable of or maintains at least two functions. The functions may affect the same outcome or a different outcome. The structure that produces the function may be the same or different. For example, bifunctional saRNA of the present invention may comprise a cytotoxic peptide (a first function) while those nucleosides which comprise the saRNA are, in and of themselves, cytotoxic (second function).

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present invention may be considered biologically active if even a portion of the saRNA is biologically active or mimics an activity considered biologically relevant.

Cancer. As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a saRNA of the present invention to targeted cells.

Destabilized. As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the oligonucleotides disclosed herein. They may be within the nucleotides or located at the 5′ or 3′ terminus.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the invention. The equivalent subject is “untreated” in that he does not receive treatment with an saRNA according to the invention. However, he may receive a conventional anti-cancer treatment, provided that the subject who is treated with the saRNA of the invention receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one saRNA of the present invention and a delivery agent.

Fragment: A “fragment,” as used herein, refers to a portion. For example, fragments of proteins may comprise polypeptides obtained by digesting full-length protein isolated from cultured cells. Fragments of oligonucleotides may comprise nucleotides, or regions of nucleotides.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g. from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MRI or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein.

Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the invention. Molecules may be modified in many ways including chemically, structurally, and functionally. In one embodiment, the saRNAs of the present invention are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent.

The saRNA of the present invention, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in one embodiment, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, in one embodiment, a mammal, or a human and most In one embodiment, a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)²×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

The invention is further illustrated by the following non-limiting examples.

### EXAMPLES

Materials and Procedures:

Transfection of saRNA

Sense and antisense strands of saRNA are synthesized. They are first annealed in water following a denaturing step at 90° C., followed by a gradual anneal step to room temperature.

Western Blotting

For the Western blot analysis, saRNA is transfected into 1×10⁵ cells using the Lipofectamine 2000 Life Technologies, US) following the manufacturer's recommendation. Total protein lysates are collected at 48 hours and 72 hours post-transfection in a lysis buffer (1% NP-40 and 1% Triton-X100 in PBS). The 72 hours harvested RNA receives two sequential transfection of saRNA. The protein concentration is measured using the protein DC assay (Bio-rad). Approximately 100 ug of protein is loaded and resolved using standard SDS-PAGE on to Novex 4-20% Tris-Glycine Gels (Invitrogen). Proteins are separated by gel electrophoresis and transferred onto nitrocellulose membrane using a semi-dry blotting apparatus (Bio-Rad). The membranes are blocked in TBS containing 5% non-fat milk for 1 hour before incubating with primary antibodies for 1 hour at room temperature. After several washes, the blots are detected using BCIP/NTB reagent (Calbiochem). The blots are imaged using Geldoc system (UVP).

RT-PCR

saRNA is transfected into 1×10⁵ cells. Total RNA is harvested post-transfection at 48 hours and 72 hours. The RNA isolated at 72 hours receives two sequential transfection of oligonucleotide. Total RNA is recovered using the RNAqueous-Micro kit (Ambion) following the manufacturer's recommendation. The RNA is quantified using a Nanodrop 2000 micro-sample quantitator. Approximately 200 ng of total RNA from each sample is reverse transcribed using the One Step RT-PCR kit (Qiagen) following the manufacturer's recommendation.

### Example 1. Upregulation of APOA1 Gene Expression Through Transfection with APOA1-saRNA In Vitro

20 nM, 50 nM or 100 nM of annealed APOA1-saRNA was transfected onto a monolayer of HepG2 cells using lipofectamine 2000 (Life Technologies, US) following the manufacturer's instructions. This process was repeated three times before cells were harvested for isolation of total RNA for mRNA analysis. APOA1-saRNAs used for this study were selected from APOA1-Pr-1′, APOA1-Pr-2′, APOA1-Pr-3′, APOA1-Pr-4′, APOA1-mm10-Pr-1′, APOA1-mm10-Pr-2′, APOA1-mm10-Pr-3′, APOA1-mm10-Pr-4′, APOA1-rn5-Pr-1′, APOA1-rn5-Pr-2′, APOA1-rn5-Pr-3′, APOA1-rn5-Pr-4′, APOA1.NM_000039-Pr-1′, APOA1.NM_000039-Pr-3′, APOA1.NM_000039-Pr-6′, APOA1.NM_000039-Pr-16′, APOA1.NM_000039-Pr-21′, APOA1.NM_000039-Pr-23′, APOA1.NM_000039-Pr-25′, and APOA1.NM_000039-Pr-37′ in Table 3.

After transfection, the isolation of total RNA was performed using the RNAqueous-Micro kit (Ambion, UK) following the manufacturer's instructions. Briefly, the cells were gently centrifuged followed by 3 pulses of sonication at Output 3 in lysis buffer (AMBION, UK). The cell lysates were then processed through an RNA binding column, followed by multiple washes and elution. The total RNA isolated was quantified by a NANODROP 2000 spectrophotometer. 500 ng of total extracted RNA was processed for elimination of genomic DNA followed by reverse transcription using the QUANTITECT® Reverse Transcription kit from QIAGEN.

The isolated RNA extracts were analyzed using quantitative reverse transcriptase (qRT-PCR). Briefly, the extracts were reverse transcribed using First Strand cDNA synthesis kit (Qiagen). The cDNA was then amplified for quantitative analysis using QuantiFast® SYBR® Green PCT Kit from Qiagen. Amplification was performed using Applied Biosystems 7900HT FAST-Real-Time System with 40 cycle conditions at 95° C. for 15 seconds and 60° C. for 45 seconds with a total volume of 25 uL per sample. Amplified products were then analyzed using Applied Biosystems RQ Manager 1.2.1. 5 independent experiments were amplified in triplicates for quantitative analysis. Student T-Test scoring was performed at 99% confidence intervals.

APOA1 production was determined within the cells through the use of an APOA1 ELISA. Briefly, the cells were grown in phenol-red free RPMI media in the presence of charcoal stripped FCS. Following three sets of saRNA transfections at 8 hrs, 16 hrs and 24 hrs, the culture media was collected for total APOA1 ELISA.

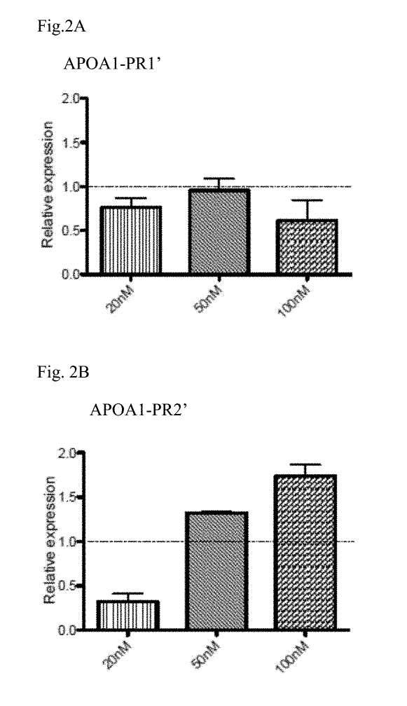

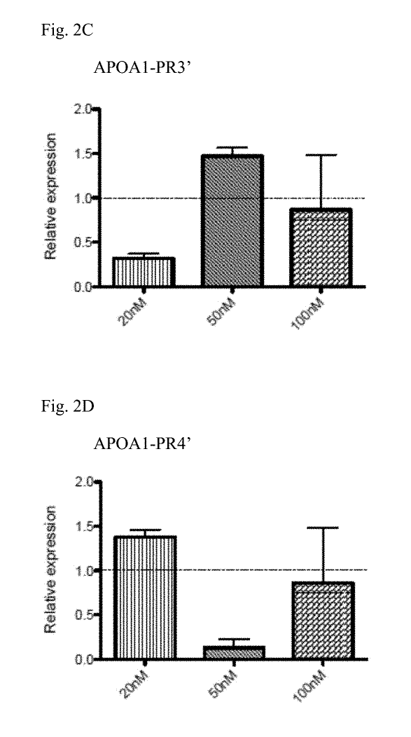

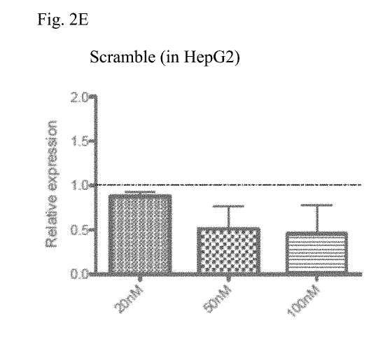

As shown in FIG. 2A-2E, an increase in APOA1 mRNA levels only occurred in cells transfected with APOA1-saRNAs, not in control cells transfected with scramble saRNA.

### Example 2. Upregulation of APOA1 Expression Through the Transfection with APOA1-saRNA in Mouse Liver In Vivo

Ten Male C57BI6/J, 8 week old mice are used for the experiment (control group N=5). APOA1-saRNA used for this study is the same as Example 1.

APOA1-saRNA reconstituted with 100 μL of RNase/Dnase free H20. 50 μL of complex A and 50 μL of complex B (InvivoFectamine, Invitrogen, CA, USA) are mixed, incubated at 50° C. for 30 minutes and are used for tail vein injections. Control animals are injected with equal volume of PBS while a positive control animal received siRNA against APOA1; a total of 5 control and 5 experimental animals are injected.

After administration, the total RNA is isolated. Frozen tissue sections are placed into scintillation vials containing Trizol and homogenised for 30 seconds. The homogenate is then transferred in Falcon tubes for a further 2 minutes of homogenisation. Chloroform is then added to this and mixed by vortexing followed by a centrifugation step at 12,000 rpm for 15 minutes at 4° C. The aqueous upper phase is then transferred into a fresh microfuge tube where RNA is precipitated using 5 mg/ml of linear acrylamide (Ambion) and isopropanol overnight at −20° C. The RNA is pelleted by centrifugation at 12,000 rpm for 15 minutes at 4° C. and washed with ice cold 70% ethanol. The RNA is pelleted again at 7,500 rpm for 5 minutes at 4° C. The supernatant is removed immediately and the RNA pellet allowed to air dry. The RNA is dissolved in nuclease free water for immediate analysis for RNA integrity using a Bionanalyser.

The isolated RNA is analyzed using qRT-PCR as described in Example 1. APOA1 production is determined using an APOA1 ELISA as described in Example 1. The administration of APOA1-saRNA using a dendrimer delivery vehicle to a mouse leads to a significant increase in APOA1 within the blood circulation.

The effects of administration of APOA1-saRNA on overall liver function are determined according to the liver function markers gamma glutamyl transpeptidase, alanine aminotransferase, and aspartate aminotransferase or bilirubin.

APOA1-saRNA also affects other the expression of other genes according to the mRNA levels of these genes. Tissue samples from the treated mice are used to measure the expression levels of various genes. The mRNA transcript levels of these genes are measured by RT-PCT mRNA expression level.

### Example 3. APOA1-saRNA Regulates Cholesterol, HDL and LDL Levels In Vitro

APOA1-saRNA is administered to mice in 0.1 nmol/uL standard dose, 2× doses, and 3× doses. Cholesterol, HDL and LDL levels in blood are measured. Dose increase of APOA1-saRNA altered circulating levels of cholesterol, HDL and LDL.

### Example 4. Upregulation of LDLR Gene Expression Through Transfection with LDLR-saRNA In Vitro

The upregulation effects of LDLR-saRNA in vitro were measured with the method described in Example 1. 10 nM, 20 nM, 50 nM or 100 nM of annealed LDLR-saRNA was transfected onto a monolayer of AML12 cells using lipofectamine 2000 (Life Technologies, US) following the manufacturer's instructions. This process was repeated three times before cells were harvested for isolation of total RNA for mRNA analysis. LDLR-saRNAs used for this study were selected from LDLR-Pr-1′, LDLR-Pr-2′, LDLR-Pr-3′, LDLR-Pr-4′, LDLR-mm10-Pr-1′, LDLR-mm10-Pr-2′, LDLR-mm10-Pr-3′, LDLR-mm10-Pr-4′, LDLR-rn5-Pr-1′, LDLR-rn5-Pr-2′, LDLR-rn5-Pr-3′, LDLR-rn5-Pr-4′, LDLR.NM_000527-Pr-4′, LDLR.NM_000527-Pr-6′, LDLR.NM_000527-Pr-7′, LDLR.NM_000527-Pr-13′, LDLR.NM_000527-Pr-22′, LDLR.NM_000527-Pr-23′, and LDLR.NM_000527-Pr-28′ in Table 5.

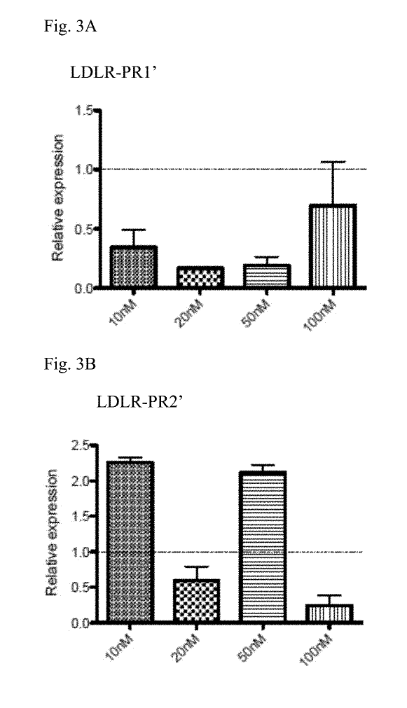

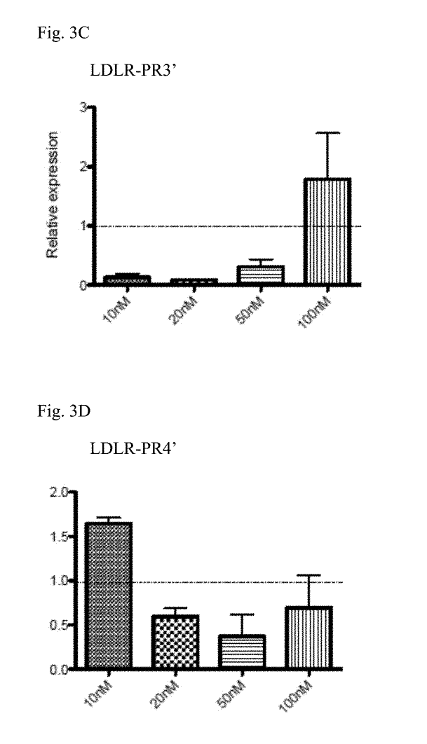

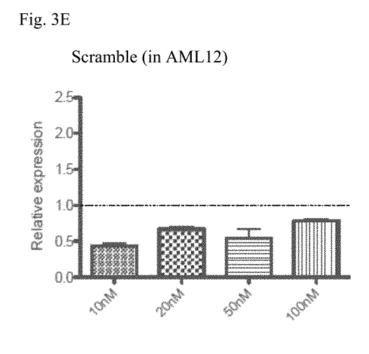

As shown in FIG. 3A-3E, LDLR mRNA levels showed an increase only in cells transfected with LDLR-saRNAs, not in control cells transfected with scramble saRNA.

### Example 5. Upregulation of LDLR Expression Through the Transfection with LDLR-saRNA in Mouse Liver In Vivo

The upregulation effects of LDLR-saRNA in vivo are measured with the method described in Example 2 Ten Male C57BI6/J, 8 week old mice are used for the experiment (control group N=5). LDLR-saRNA used for this study is the same as Example 4.

LDLR-saRNA reconstituted with 100 μL of RNase/Dnase free H20; 50 μL of complex A and 50 μL of complex B (InvivoFectamine, Invitrogen, CA, USA) are mixed, incubated at 50° C. for 30 minutes and are used for tail vein injections. Control animals are injected with equal volume of PBS while a positive control animal received siRNA against LDLR; a total of 5 control and 5 experimental animals are injected.

Tissue sample are analyzed for LDLR mRNA levels and mRNA levels of other genes. The administration of LDLR-saRNA using a dendrimer delivery vehicle to a mouse leads to a significant increase in LDLR within the blood circulation. LDLR-saRNA also modulates the expression of other genes.

### Example 6. LDLR-saRNA Regulates Cholesterol, HDL and LDL Levels In Vitro

LDLR-saRNA is administered to mice in 0.1 nmol/uL standard dose, 2× doses, and 3× doses. Cholesterol, HDL and LDL levels in blood are measured. Dose increase of LDLR-saRNA altered circulating levels of cholesterol, HDL and LDL.

### Example 7. Upregulation of HNF4A Expression Through Transfection with its saRNA In Vitro

20 nM, 50 nM or 100 nM of saRNA of HNF4A-saRNA is transfected onto a monolayer of cells using lipofectamine 2000 (Life Technologies, US) following the manufacturer's instructions. The saRNA may be single stranded or double-stranded. The cells may be HepG2 cells. This process is repeated three times before cells are harvested for isolation of total RNA for mRNA analysis.

After transfection, the isolation of total RNA is performed using the RNAqueous-Micro kit (Ambion, UK) following the manufacturer's instructions. Briefly, the cells are gently centrifuged followed by 3 pulses of sonication at Output 3 in lysis buffer (AMBION, UK). The cell lysates are then processed through an RNA binding column, followed by multiple washes and elution. The total RNA isolated is quantified by a NANODROP 2000 spectrophotometer. 500 ng of total extracted RNA is processed for elimination of genomic DNA followed by reverse transcription using the QUANTITECT® Reverse Transcription kit from QIAGEN.

The isolated RNA extracts are analyzed using quantitative reverse transcriptase (qRT-PCR). Briefly, the extracts are reverse transcribed using First Strand cDNA synthesis kit (Qiagen). The cDNA is then amplified for quantitative analysis using QuantiFast® SYBR® Green PCT Kit from Qiagen. Amplification is performed using Applied Biosystems 7900HT FAST-Real-Time System. Amplified products are then analyzed using Applied Biosystems RQ Manager 1.2.1. 5 independent experiments are amplified in triplicates for quantitative analysis. Student T-Test scoring is performed at 99% confidence intervals.

mRNA production of the target gene is determined within the cells through the use of ELISA. Briefly, the cells are grown in phenol-red free RPMI media in the presence of charcoal stripped FCS. Following three sets of saRNA transfections at 8 hrs, 16 hrs and 24 hrs, the culture media is collected for total ELISA.

An increase in mRNA levels only occurred in cells transfected with saRNAs, not in control cells transfected with scramble saRNA.

### Example 8. Upregulation of HNF4A Expression Through the Transfection with saRNA in Mouse In Vivo

Ten male C57BI6/J, 8 week old mice are used for the experiment (control group N=5).

saRNA of the HNF4A reconstituted with 100 μL of RNase/Dnase free H20; 50 μL of complex A and 50 μL of complex B (InvivoFectamine, Invitrogen, CA, USA) are mixed, incubated at 50° C. for 30 minutes and are used for tail vein injections. Control animals are injected with equal volume of PBS while a positive control animal received siRNA against the target gene; a total of 5 control and 5 experimental animals are injected.

After administration, the total RNA is isolated. Frozen tissue sections from the mice are placed into scintillation vials containing Trizol and homogenised for 30 seconds. The tissue may be from the liver of the mice. The homogenate is then transferred in Falcon tubes for a further 2 minutes of homogenisation. Chloroform is then added to this and mixed by vortexing followed by a centrifugation step at 12,000 rpm for 15 minutes at 4° C. The aqueous upper phase is then transferred into a fresh microfuge tube where RNA is precipitated using 5 mg/ml of linear acrylamide (Ambion) and isopropanol overnight at −20° C. The RNA is pelleted by centrifugation at 12,000 rpm for 15 minutes at 4° C. and washed with ice cold 70% ethanol. The RNA is pelleted again at 7,500 rpm for 5 minutes at 4° C. The supernatant is removed immediately and the RNA pellet allowed to air dry. The RNA is dissolved in nuclease free water for immediate analysis for RNA integrity using a Bionanalyser.

The isolated RNA is analyzed using qRT-PCR as described in Example 7. mRNA production of the target gene is determined using an ELISA as described in Example 7. The administration of saRNA using a dendrimer delivery vehicle to a mouse leads to a significant increase in the mRNA encoded by the target gene within the blood circulation.

The effects of administration of saRNA on overall mice organ function may be studied. As a non-limiting example, the effects of administration of saRNA on overall liver function are determined according to the liver function markers gamma glutamyl transpeptidase, alanine aminotransferase, and aspartate aminotransferase or bilirubin.

saRNA also affects other the expression of other genes according to the mRNA levels of these genes. Tissue samples from the treated mice are used to measure the expression levels of various genes. The mRNA transcript levels of these genes are measured by RT-PCT mRNA expression level.

### Example 9. Upregulation of a Target Gene Expression Through Transfection with its saRNA In Vitro

20 nM, 50 nM or 100 nM of saRNA of an saRNA for a target gene is transfected onto a monolayer of cells using lipofectamine 2000 (Life Technologies, US) following the manufacturer's instructions. The target genes may be DMD, PAX5, SCN1A, IDUA, FNDC5, FOXA2, FOXP3, HNF4A, IFNG, IL10, IL2, LMXIA, METRNL, NR4A2, SIRTI, TH, EPO, CDKNIB, MDM2, IL19, C19orf90, F7, or F8. The saRNA may be single stranded or double-stranded. The cells may be HepG2 cells. This process is repeated three times before cells are harvested for isolation of total RNA for mRNA analysis.

After transfection, the isolation of total RNA is performed using the RNAqueous-Micro kit (Ambion, UK) following the manufacturer's instructions. Briefly, the cells are gently centrifuged followed by 3 pulses of sonication at Output 3 in lysis buffer (AMBION, UK). The cell lysates are then processed through an RNA binding column, followed by multiple washes and elution. The total RNA isolated is quantified by a NANODROP 2000 spectrophotometer. 500 ng of total extracted RNA is processed for elimination of genomic DNA followed by reverse transcription using the QUANTITECT® Reverse Transcription kit from QIAGEN.

The isolated RNA extracts are analyzed using quantitative reverse transcriptase (qRT-PCR). Briefly, the extracts are reverse transcribed using First Strand cDNA synthesis kit (Qiagen). The cDNA is then amplified for quantitative analysis using QuantiFast® SYBR® Green PCT Kit from Qiagen. Amplification is performed using Applied Biosystems 7900HT FAST-Real-Time System. Amplified products are then analyzed using Applied Biosystems RQ Manager 1.2.1. 5 independent experiments are amplified in triplicates for quantitative analysis. Student T-Test scoring is performed at 99% confidence intervals.

mRNA production of the target gene is determined within the cells through the use of ELISA. Briefly, the cells are grown in phenol-red free RPMI media in the presence of charcoal stripped FCS. Following three sets of saRNA transfections at 8 hrs, 16 hrs and 24 hrs, the culture media is collected for total ELISA.

An increase in mRNA levels only occurred in cells transfected with saRNAs, not in control cells transfected with scramble saRNA.

### Example 10. Upregulation of Target Gene Expression Through the Transfection with saRNA in Mouse In Vivo

Ten male C57BI6/J, 8 week old mice are used for the experiment (control group N=5). The target genes may be DMD, PAX5, SCN1A, IDUA, FNDC5, FOXA2, FOXP3, HNF4A, IFNG, IL10, IL2, LMXIA, METRNL, NR4A2, SIRTI, TH, EPO, CDKNIB, MDM2, IL19, C19orf90, F7, or F8.

saRNA of the target gene reconstituted with 100 μL of RNase/Dnase free H20; 50 μL of complex A and 50 μL of complex B (InvivoFectamine, Invitrogen, CA, USA) are mixed, incubated at 50° C. for 30 minutes and are used for tail vein injections. Control animals are injected with equal volume of PBS while a positive control animal received siRNA against the target gene; a total of 5 control and 5 experimental animals are injected.

After administration, the total RNA is isolated. Frozen tissue sections from the mice are placed into scintillation vials containing Trizol and homogenised for 30 seconds. The tissue may be from the liver of the mice. The homogenate is then transferred in Falcon tubes for a further 2 minutes of homogenisation. Chloroform is then added to this and mixed by vortexing followed by a centrifugation step at 12,000 rpm for 15 minutes at 4° C. The aqueous upper phase is then transferred into a fresh microfuge tube where RNA is precipitated using 5 mg/ml of linear acrylamide (Ambion) and isopropanol overnight at −20° C. The RNA is pelleted by centrifugation at 12,000 rpm for 15 minutes at 4° C. and washed with ice cold 70% ethanol. The RNA is pelleted again at 7,500 rpm for 5 minutes at 4° C. The supernatant is removed immediately and the RNA pellet allowed to air dry. The RNA is dissolved in nuclease free water for immediate analysis for RNA integrity using a Bionanalyser.

The isolated RNA is analyzed using qRT-PCR as described in Example 9. mRNA production of the target gene is determined using an ELISA as described in Example 9. The administration of saRNA using a dendrimer delivery vehicle to a mouse leads to a significant increase in the mRNA encoded by the target gene within the blood circulation.

The effects of administration of saRNA on overall mice organ function may be studied. As a non-limiting example, the effects of administration of saRNA on overall liver function are determined according to the liver function markers gamma glutamyl transpeptidase, alanine aminotransferase, and aspartate aminotransferase or bilirubin.

saRNA also affects other the expression of other genes according to the mRNA levels of these genes. Tissue samples from the treated mice are used to measure the expression levels of various genes. The mRNA transcript levels of these genes are measured by RT-PCT mRNA expression level.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

1. A synthetic isolated saRNA which comprises a strand that targets a region between 2000 nucleotides upstream and 2000 nucleotides downstream of the transcription start site (TSS core) of a target gene,
wherein the strand is 14-30 nucleotides in length, and
wherein the target gene has a sequence selected from the group consisting of SEQ ID NO: 1 (APOA1), SEQ ID NO: 2 (APOA1), SEQ ID NO: 3 (APOA1), SEQ ID NO: 4 (LDLR), SEQ ID NO: 5 (LDLR), SEQ ID NO: 6 (LDLR), SEQ ID NO: 13 (DMD), SEQ ID NO: 14 (DMD), SEQ ID NO: 15 (DMD), SEQ ID NO: 16 (DMD), SEQ ID NO: 17 (DMD), SEQ ID NO: 18 (DMD), SEQ ID NO: 19 (DMD), SEQ ID NO: 20 (PAX5), SEQ ID NO: 21 (SCN1A), SEQ ID NO: 22 (SCN1A), SEQ ID NO: 23 (IDUA), SEQ ID NO: 24 (FNDC5), SEQ ID NO: 25 (FNDC5), SEQ ID NO: 26 (FOXA2), SEQ ID NO: 27 (FOXA2), SEQ ID NO: 28 (FOXP3), SEQ ID NO: 29 (HNF4A), SEQ ID NO: 30 (HNF4A), SEQ ID NO: 31 (IFNG), SEQ ID NO: 32 (IL10), SEQ ID NO: 33 (IL2), SEQ ID NO: 34 (IL19), SEQ ID NO: 35 (LMX1A), SEQ ID NO: 36 (LMX1A), SEQ ID NO: 37 (METRNL), SEQ ID NO: 38 (NR4A2), SEQ ID NO: 39 (SIRT1), SEQ ID NO: 40 (TH), SEQ ID NO: 41 (EPO), SEQ ID NO: 42 (CDKN1B), SEQ ID NO: 43 (MDM2), SEQ ID NO: 44 (MDM2), SEQ ID NO: 45 (C19orf80), SEQ ID NO: 46 (F7), SEQ ID NO: 47 (F8) and SEQ ID NO: 48 (F8).

2. The saRNA of claim 1, wherein the region between 2000 nucleotides upstream and 2000 nucleotides downstream of the transcription start site has a sequence selected from the group consisting of SEQ ID NO: 2020 (APOA1), 2021 (C19orf80), 2022 (CDKN1B), 2023 (DMD), 2024 (DMD), 2025 (DMD), 2026 (DMD), 2027 (DMD), 2028 (DMD), 2029 (DMD), 2030 (DMD), 2031 (EPO), 2032 (F7), 2033 (F8), 2034 (F8), 2035 (FNDC5), 2036 (FNDC5), 2037 (FOXA2), 2038 (FOXA2), 2039 (FOXP3), 2040 (HNF4A), 2041 (HNF4A), 2042 (IDUA), 2043 (IFNG), 2044 (IL10), 2045 (IL19), 2046 (IL19), 2047 (IL2), 2048 (LDLR), 2049 (LMX1A), 2050 (LMX1A), 2051 (MDM2), 2052 (MDM2), 2053 (METRNL), 2054 (NR4A2), 2055 (PAX5), 2056 (SCN1A), 2057 (SCN1A), 2058 (SIRT1), 2059 (SIRT1), and 2060 (TH).

3. The saRNA of claim 1, wherein the saRNA is single-stranded.

4. The saRNA of claim 3, wherein the saRNA is at least 80% identical to the reverse complement of a nucleotide sequence selected from the group consisting of SEQ ID NOs: 72-90, 167-184, 257-308, 517-525, 562-576, 637-642, 667-688, 777-802, 907-913, 942-964, 1057-1061, 1090-1100, 1145-1149, 1170-1185, 1250-1268, 1345-1359, 1425-1435, 1480-1500, 1605-1611, 1640-1647, 1680-1690, 1735-1751, 1820-1833, 1890-1898, 1935-1951, and 2020-2060.

5. The saRNA of claim 3, wherein the saRNA comprises a sequence selected from the group consisting of SEQ ID NO: 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 4610, 4612, 4614, 4616, 4618, 4620, 4622, 4624, 4626, 4628, 4630, 4632, 4634, 4636, 4638, 4640, 4642, 4644, 4646, 4648, 4650, 4652, 4654, 4656, 4658, 4660, 4662, 4664, 4666, 4668, 4670, 4672, 4674, 4676, 4678, 4680, 4682, 4684, 4686, 4688, 4690, 4692, 4694, 4696, 4698, 4700, 4702, 4704, 4706, 4708, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 2362, 2364, 2366, 2368, 2370, 2372, 2374, 2376, 2378, 2380, 2382, 2384, 2386, 2388, 2390, 2392, 2394, 2396, 2398, 2400, 2402, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 2808, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, 3058, 3060, 3062, 3064, 3066, 3068, 3070, 3072, 3074, 3076, 3078, 3080, 3082, 3084, 3086, 3088, 3090, 3092, 3094, 3096, 3098, 3100, 3102, 3104, 3106, 3108, 3110, 3112, 3114, 3116, 3118, 3120, 3122, 3124, 3126, 3128, 3130, 3132, 3134, 3136, 3138, 527, 529, 531, 533, 535, 537, 539, 541, 543, 545, 547, 549, 551, 553, 555, 557, 559, 561, 5216, 5218, 5220, 5222, 5224, 5226, 5228, 5230, 5232, 5234, 5236, 5238, 5240, 5242, 5244, 5246, 5248, 5250, 5252, 5254, 5256, 5258, 5260, 5262, 5264, 5266, 5268, 5270, 5272, 5274, 5276, 5278, 5280, 5282, 5284, 5286, 5288, 5290, 5292, 5294, 5296, 5298, 5300, 5302, 5304, 5306, 5308, 5310, 5312, 5314, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 5316, 5318, 5320, 5322, 5324, 5326, 5328, 5330, 5332, 5334, 5336, 5338, 5340, 5342, 5344, 5346, 5348, 5350, 5352, 5354, 5356, 5358, 5360, 5362, 5364, 5366, 5368, 5370, 5372, 5374, 5376, 5378, 5380, 5382, 5384, 5386, 5388, 5390, 5392, 5394, 5396, 5398, 5400, 5402, 5404, 5406, 5408, 5410, 5412, 5414, 5416, 5418, 5420, 5422, 5424, 5426, 5428, 5430, 5432, 5434, 5436, 5438, 5440, 5442, 5444, 5446, 5448, 5450, 5452, 5454, 5456, 5458, 5460, 5462, 5464, 5466, 5468, 5470, 5472, 5474, 5476, 5478, 5480, 5482, 5484, 5486, 5488, 5490, 5492, 5494, 5496, 5498, 5500, 5502, 5504, 5506, 5508, 5510, 5512, 5514, 5516, 5518, 644, 646, 648, 650, 652, 654, 656, 658, 660, 662, 664, 666, 4042, 4044, 4046, 4048, 4050, 4052, 4054, 4056, 4058, 4060, 4062, 4064, 4066, 4068, 4070, 4072, 4074, 4076, 4078, 4080, 4082, 4084, 4086, 4088, 4090, 4092, 4094, 4096, 4098, 4100, 4102, 4104, 4106, 4108, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 3398, 3400, 3402, 3404, 3406, 3408, 3410, 3412, 3414, 3416, 3418, 3420, 3422, 3424, 3426, 3428, 3430, 3432, 3434, 3436, 3438, 3440, 3442, 3444, 3446, 3448, 3450, 3452, 3454, 3456, 3458, 3460, 3462, 3464, 3466, 3468, 3470, 3472, 3474, 3476, 3478, 3480, 3482, 3484, 3486, 3488, 3490, 3492, 3494, 3496, 3498, 3500, 3502, 3504, 3506, 3508, 3510, 3512, 3514, 3516, 3518, 3520, 3522, 3524, 3526, 3528, 3530, 3532, 3534, 3536, 3538, 3540, 3542, 3544, 3546, 3548, 3550, 3552, 3554, 3556, 3558, 3560, 3562, 3564, 3566, 3568, 3570, 3572, 3574, 3576, 3578, 3580, 3582, 3584, 3586, 3588, 3590, 3592, 3594, 3596, 3598, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 3600, 3602, 3604, 3606, 3608, 3610, 3612, 3614, 3616, 3618, 3620, 3622, 3624, 3626, 3628, 3630, 3632, 3634, 3636, 3638, 3640, 3642, 3644, 3646, 3648, 3650, 3652, 3654, 3656, 3658, 3660, 3662, 3664, 3666, 3668, 3670, 3672, 3674, 3676, 3678, 3680, 3682, 3684, 3686, 3688, 3690, 3692, 3694, 3696, 3698, 3700, 3702, 3704, 3706, 3708, 3710, 3712, 3714, 3716, 3718, 3720, 3722, 3724, 3726, 3728, 3730, 3732, 3734, 3736, 3738, 3740, 3742, 3744, 3746, 3748, 3750, 3752, 3754, 3756, 3758, 3760, 915, 917, 919, 921, 923, 925, 927, 929, 931, 933, 935, 937, 939, 941, 3762, 3764, 3766, 3768, 3770, 3772, 3774, 3776, 3778, 3780, 3782, 3784, 3786, 3788, 3790, 3792, 3794, 3796, 3798, 3800, 3802, 3804, 3806, 3808, 3810, 3812, 3814, 3816, 3818, 3820, 3822, 3824, 3826, 3828, 3830, 3832, 3834, 3836, 3838, 3840, 3842, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 3844, 3846, 3848, 3850, 3852, 3854, 3856, 3858, 3860, 3862, 3864, 3866, 3868, 3870, 3872, 3874, 3876, 3878, 3880, 3882, 3884, 3886, 3888, 3890, 3892, 3894, 3896, 3898, 3900, 3902, 3904, 3906, 3908, 3910, 3912, 3914, 3916, 3918, 3920, 3922, 3924, 3926, 3928, 3930, 3932, 3934, 3936, 3938, 3940, 3942, 3944, 3946, 3948, 3950, 3952, 3954, 3956, 3958, 3960, 3962, 3964, 3966, 3968, 3970, 3972, 3974, 3976, 3978, 3980, 3982, 3984, 3986, 3988, 3990, 3992, 3994, 3996, 3998, 4000, 4002, 4004, 4006, 4008, 4010, 4012, 4014, 4016, 4018, 4020, 4022, 4024, 4026, 4028, 4030, 4032, 4034, 4036, 4038, 4040, 1071, 1073, 1075, 1077, 1079, 1081, 1083, 1085, 1087, 1089, 4110, 4112, 4114, 4116, 4118, 4120, 4122, 4124, 4126, 4128, 4130, 4132, 4134, 4136, 4138, 4140, 4142, 4144, 4146, 4148, 4150, 4152, 4154, 4156, 4158, 4160, 4162, 4164, 4166, 4168, 4170, 4172, 4174, 4176, 4178, 4180, 4182, 4184, 4186, 4188, 4190, 4192, 4194, 4196, 4198, 4200, 4202, 4204, 4206, 4208, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 4210, 4212, 4214, 4216, 4218, 4220, 4222, 4224, 4226, 4228, 4230, 4232, 4234, 4236, 4238, 4240, 4242, 4244, 4246, 4248, 4250, 4252, 4254, 4256, 4258, 4260, 4262, 4264, 4266, 4268, 4270, 4272, 4274, 4276, 4278, 4280, 4282, 4284, 4286, 4288, 4290, 4292, 4294, 4296, 4298, 4300, 4302, 4304, 4306, 4308, 1151, 1153, 1155, 1157, 1159, 1161, 1163, 1165, 1167, 1169, 4510, 4512, 4514, 4516, 4518, 4520, 4522, 4524, 4526, 4528, 4530, 4532, 4534, 4536, 4538, 4540, 4542, 4544, 4546, 4548, 4550, 4552, 4554, 4556, 4558, 4560, 4562, 4564, 4566, 4568, 4570, 4572, 4574, 4576, 4578, 4580, 4582, 4584, 4586, 4588, 4590, 4592, 4594, 4596, 4598, 4600, 4602, 4604, 4606, 4608, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 4310, 4312, 4314, 4316, 4318, 4320, 4322, 4324, 4326, 4328, 4330, 4332, 4334, 4336, 4338, 4340, 4342, 4344, 4346, 4348, 4350, 4352, 4354, 4356, 4358, 4360, 4362, 4364, 4366, 4368, 4370, 4372, 4374, 4376, 4378, 4380, 4382, 4384, 4386, 4388, 4390, 4392, 4394, 4396, 4398, 4400, 4402, 4404, 4406, 4408, 4410, 4412, 4414, 4416, 4418, 4420, 4422, 4424, 4426, 4428, 4430, 4432, 4434, 4436, 4438, 4440, 4442, 4444, 4446, 4448, 4450, 4452, 4454, 4456, 4458, 4460, 4462, 4464, 4466, 4468, 4470, 4472, 4474, 4476, 4478, 4480, 4482, 4484, 4486, 4488, 4490, 4492, 4494, 4496, 4498, 4500, 4502, 4504, 4506, 4508, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 4710, 4712, 4714, 4716, 4718, 4720, 4722, 4724, 4726, 4728, 4730, 4732, 4734, 4736, 4738, 4740, 4742, 4744, 4746, 4748, 4750, 4752, 4754, 4756, 4758, 4760, 4762, 4764, 4766, 4768, 4770, 4772, 4774, 4776, 4778, 4780, 4782, 4784, 4786, 4788, 4790, 4792, 4794, 4796, 4798, 4800, 4802, 4804, 4806, 4808, 4810, 4812, 4814, 4816, 4818, 4820, 4822, 4824, 4826, 4828, 4830, 4832, 4834, 4836, 4838, 4840, 4842, 4844, 4846, 4848, 4850, 4852, 4854, 4856, 4858, 4860, 4862, 4864, 4866, 4868, 4870, 4872, 4874, 4876, 4878, 4880, 4882, 4884, 4886, 4888, 4890, 4892, 4894, 4896, 4898, 4900, 4902, 4904, 4906, 4908, 1362, 1364, 1366, 1368, 1370, 1372, 1374, 1376, 1378, 1380, 1382, 1384, 1386, 1388, 1390, 1394, 1396, 1398, 1400, 1402, 1404, 1406, 1408, 1410, 1412, 1414, 1416, 1418, 1420, 1422, 5076, 5078, 5080, 5082, 5084, 5086, 5088, 5090, 5092, 5094, 5096, 5098, 5100, 5102, 5104, 5106, 5108, 5110, 5112, 5114, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 5116, 5118, 5120, 5122, 5124, 5126, 5128, 5130, 5132, 5134, 5136, 5138, 5140, 5142, 5144, 5146, 5148, 5150, 5152, 5154, 5156, 5158, 5160, 5162, 5164, 5166, 5168, 5170, 5172, 5174, 5176, 5178, 5180, 5182, 5184, 5186, 5188, 5190, 5192, 5194, 5196, 5198, 5200, 5202, 5204, 5206, 5208, 5210, 5212, 5214, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 5520, 5522, 5524, 5526, 5528, 5530, 5532, 5534, 5536, 5538, 5540, 5542, 5544, 5546, 5548, 5550, 5552, 5554, 5556, 5558, 5560, 5562, 5564, 5566, 5568, 5570, 5572, 5574, 5576, 5578, 5580, 5582, 5584, 5586, 5588, 5590, 5592, 5594, 5596, 5598, 5600, 5602, 5604, 5606, 5608, 5610, 5612, 5614, 5616, 5618, 5620, 5622, 5624, 5626, 5628, 5630, 5632, 5634, 5636, 5638, 5640, 5642, 5644, 5646, 5648, 5650, 5652, 5654, 5656, 5658, 5660, 5662, 5664, 5666, 5668, 5670, 5672, 5674, 5676, 5678, 5680, 5682, 5684, 5686, 5688, 5690, 5692, 5694, 5696, 5698, 5700, 5702, 5704, 5706, 5708, 5710, 5712, 5714, 5716, 5718, 1613, 1615, 1617, 1619, 1621, 1623, 1625, 1627, 1629, 1631, 1633, 1635, 1637, 1639, 5720, 5722, 5724, 5726, 5728, 5730, 5732, 5734, 5736, 5738, 5740, 5742, 5744, 5746, 5748, 5750, 5752, 5754, 5756, 5758, 5760, 5762, 5764, 5766, 5768, 5770, 5772, 5774, 5776, 5778, 5780, 5782, 5784, 5786, 5788, 5790, 1649, 1651, 1653, 1655, 1657, 1659, 1661, 1663, 1665, 1667, 1669, 1671, 1673, 1675, 1677, 1679, 3140, 3142, 3144, 3146, 3148, 3150, 3152, 3154, 3156, 3158, 3160, 3162, 3164, 3166, 3168, 3170, 3172, 3174, 3176, 3178, 3180, 3182, 3184, 3186, 3188, 3190, 3192, 3194, 3196, 3198, 3200, 3202, 3204, 3206, 3208, 3210, 3212, 3214, 3216, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 4910, 4912, 4914, 4916, 4918, 4920, 4922, 4924, 4926, 4928, 4930, 4932, 4934, 4936, 4938, 4940, 4942, 4944, 4946, 4948, 4950, 4952, 4954, 4956, 4958, 4960, 4962, 4964, 4966, 4968, 4970, 4972, 4974, 4976, 4978, 4980, 4982, 4984, 4986, 4988, 4990, 4992, 4994, 4996, 4998, 5000, 5002, 5004, 5006, 5008, 5010, 5012, 5014, 5016, 5018, 5020, 5022, 5024, 5026, 5028, 5030, 5032, 5034, 5036, 5038, 5040, 5042, 5044, 5046, 5048, 5050, 5052, 5054, 5056, 5058, 5060, 5062, 5064, 5066, 5068, 5070, 5072, 5074, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 1900, 1902, 1904, 1906, 1908, 1910, 1912, 1914, 1916, 1918, 1920, 1922, 1924, 1926, 1928, 1930, 1932, 1934, 3218, 3220, 3222, 3224, 3226, 3228, 3230, 3232, 3234, 3236, 3238, 3240, 3242, 3244, 3246, 3248, 3250, 3252, 3254, 3256, 3258, 3260, 3262, 3264, 3266, 3268, 3270, 3272, 3274, 3276, 3278, 3280, 3282, 3284, 3286, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 3288, 3290, 3292, 3294, 3296, 3298, 3300, 3302, 3304, 3306, 3308, 3310, 3312, 3314, 3316, 3318, 3320, 3322, 3324, 3326, 3328, 3330, 3332, 3334, 3336, 3338, 3340, 3342, 3344, 3346, 3348, 3350, 3352, 3354, 3356, 3358, 3360, 3362, 3364, 3366, 3368, 3370, 3372, 3374, 3376, 3378, 3380, 3382, 3384, 3386, 3388, 3390, 3392, 3394 and 3396.

6. The saRNA of claim 3, wherein the saRNA comprises a 3′ tail.

7. The saRNA of claim 3, wherein the saRNA comprises at least one modification.

8. The saRNA of claim 1, wherein the saRNA is an saRNA duplex comprising an antisense strand and a sense strand wherein each strand is independently 14-30 nucleotides in length.

9. The saRNA of claim 8, wherein the antisense strand of the saRNA duplex is at least 80% identical to the reverse complement of a nucleotide sequence selected from the group consisting of SEQ ID NOs: 72-90, 167-184, 257-308, 517-525, 562-576, 637-642, 667-688, 777-802, 907-913, 942-964, 1057-1061, 1090-1100, 1145-1149, 1170-1185, 1250-1268, 1345-1359, 1425-1435, 1480-1500, 1605-1611, 1640-1647, 1680-1690, 1735-1751, 1820-1833, 1890-1898, 1935-1951, and 2020-2060.

10. The saRNA of claim 8, wherein the saRNA comprises a sense sequence and an antisense sequence selected from the group of sequence pairs consisting of SEQ ID NOs: 91 and 92; 93 and 94; 95 and 96; 97 and 98; 99 and 100; 101 and 102; 103 and 104; 105 and 106; 107 and 108; 109 and 110; 111 and 112; 113 and 114; 115 and 116; 117 and 118; 119 and 120; 121 and 122; 123 and 124; 125 and 126; 127 and 128; 129 and 130; 131 and 132; 133 and 134; 135 and 136; 137 and 138; 139 and 140; 141 and 142; 143 and 144; 145 and 146; 147 and 148; 149 and 150; 151 and 152; 153 and 154; 155 and 156; 157 and 158; 159 and 160; 161 and 162; 163 and 164; 165 and 166; 2061 and 2062; 2063 and 2064; 2065 and 2066; 2067 and 2068; 2069 and 2070; 2071 and 2072; 2073 and 2074; 2075 and 2076; 2077 and 2078; 2079 and 2080; 2081 and 2082; 2083 and 2084; 2085 and 2086; 2087 and 2088; 2089 and 2090; 2091 and 2092; 2093 and 2094; 2095 and 2096; 2097 and 2098; 2099 and 2100; 2101 and 2102; 2103 and 2104; 2105 and 2106; 2107 and 2108; 2109 and 2110; 2111 and 2112; 2113 and 2114; 2115 and 2116; 2117 and 2118; 2119 and 2120; 2121 and 2122; 2123 and 2124; 2125 and 2126; 2127 and 2128; 2129 and 2130; 2131 and 2132; 2133 and 2134; 2135 and 2136; 2137 and 2138; 2139 and 2140; 2141 and 2142; 2143 and 2144; 2145 and 2146; 2147 and 2148; 2149 and 2150; 2151 and 2152; 2153 and 2154; 2155 and 2156; 2157 and 2158; 2159 and 2160; 185 and 186; 187 and 188; 189 and 190; 191 and 192; 193 and 194; 195 and 196; 197 and 198; 199 and 200; 201 and 202; 203 and 204; 205 and 206; 207 and 208; 209 and 210; 211 and 212; 213 and 214; 215 and 216; 217 and 218; 219 and 220; 221 and 222; 223 and 224; 225 and 226; 227 and 228; 229 and 230; 231 and 232; 233 and 234; 235 and 236; 237 and 238; 239 and 240; 241 and 242; 243 and 244; 245 and 246; 247 and 248; 249 and 250; 251 and 252; 253 and 254; 255 and 256; 4609 and 4610; 4611 and 4612; 4613 and 4614; 4615 and 4616; 4617 and 4618; 4619 and 4620; 4621 and 4622; 4623 and 4624; 4625 and 4626; 4627 and 4628; 4629 and 4630; 4631 and 4632; 4633 and 4634; 4635 and 4636; 4637 and 4638; 4639 and 4640; 4641 and 4642; 4643 and 4644; 4645 and 4646; 4647 and 4648; 4649 and 4650; 4651 and 4652; 4653 and 4654; 4655 and 4656; 4657 and 4658; 4659 and 4660; 4661 and 4662; 4663 and 4664; 4665 and 4666; 4667 and 4668; 4669 and 4670; 4671 and 4672; 4673 and 4674; 4675 and 4676; 4677 and 4678; 4679 and 4680; 4681 and 4682; 4683 and 4684; 4685 and 4686; 4687 and 4688; 4689 and 4690; 4691 and 4692; 4693 and 4694; 4695 and 4696; 4697 and 4698; 4699 and 4700; 4701 and 4702; 4703 and 4704; 4705 and 4706; 4707 and 4708; 309 and 310; 311 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; 341 and 342; 343 and 344; 345 and 346; 347 and 348; 349 and 350; 351 and 352; 353 and 354; 355 and 356; 357 and 358; 359 and 360; 361 and 362; 363 and 364; 365 and 366; 367 and 368; 369 and 370; 371 and 372; 373 and 374; 375 and 376; 377 and 378; 379 and 380; 381 and 382; 383 and 384; 385 and 386; 387 and 388; 389 and 390; 391 and 392; 393 and 394; 395 and 396; 397 and 398; 399 and 400; 401 and 402; 403 and 404; 405 and 406; 407 and 408; 409 and 410; 411 and 412; 413 and 414; 415 and 416; 417 and 418; 419 and 420; 421 and 422; 423 and 424; 425 and 426; 427 and 428; 429 and 430; 431 and 432; 433 and 434; 435 and 436; 437 and 438; 439 and 440; 441 and 442; 443 and 444; 445 and 446; 447 and 448; 449 and 450; 451 and 452; 453 and 454; 455 and 456; 457 and 458; 459 and 460; 461 and 462; 463 and 464; 465 and 466; 467 and 468; 469 and 470; 471 and 472; 473 and 474; 475 and 476; 477 and 478; 479 and 480; 481 and 482; 483 and 484; 485 and 486; 487 and 488; 489 and 490; 491 and 492; 493 and 494; 495 and 496; 497 and 498; 499 and 500; 501 and 502; 503 and 504; 505 and 506; 507 and 508; 509 and 510; 511 and 512; 513 and 514; 515 and 516; 2361 and 2362; 2363 and 2364; 2365 and 2366; 2367 and 2368; 2369 and 2370; 2371 and 2372; 2373 and 2374; 2375 and 2376; 2377 and 2378; 2379 and 2380; 2381 and 2382; 2383 and 2384; 2385 and 2386; 2387 and 2388; 2389 and 2390; 2391 and 2392; 2393 and 2394; 2395 and 2396; 2397 and 2398; 2399 and 2400; 2401 and 2402; 2403 and 2404; 2405 and 2406; 2407 and 2408; 2409 and 2410; 2411 and 2412; 2413 and 2414; 2415 and 2416; 2417 and 2418; 2419 and 2420; 2421 and 2422; 2423 and 2424; 2425 and 2426; 2427 and 2428; 2429 and 2430; 2431 and 2432; 2433 and 2434; 2435 and 2436; 2437 and 2438; 2439 and 2440; 2441 and 2442; 2443 and 2444; 2445 and 2446; 2447 and 2448; 2449 and 2450; 2451 and 2452; 2453 and 2454; 2455 and 2456; 2457 and 2458; 2459 and 2460; 2461 and 2462; 2463 and 2464; 2465 and 2466; 2467 and 2468; 2469 and 2470; 2471 and 2472; 2473 and 2474; 2475 and 2476; 2477 and 2478; 2479 and 2480; 2481 and 2482; 2483 and 2484; 2485 and 2486; 2487 and 2488; 2489 and 2490; 2491 and 2492; 2493 and 2494; 2495 and 2496; 2497 and 2498; 2499 and 2500; 2501 and 2502; 2503 and 2504; 2505 and 2506; 2507 and 2508; 2509 and 2510; 2511 and 2512; 2513 and 2514; 2515 and 2516; 2517 and 2518; 2519 and 2520; 2521 and 2522; 2523 and 2524; 2525 and 2526; 2527 and 2528; 2529 and 2530; 2531 and 2532; 2533 and 2534; 2535 and 2536; 2537 and 2538; 2539 and 2540; 2541 and 2542; 2543 and 2544; 2545 and 2546; 2547 and 2548; 2549 and 2550; 2551 and 2552; 2553 and 2554; 2555 and 2556; 2557 and 2558; 2559 and 2560; 2561 and 2562; 2563 and 2564; 2565 and 2566; 2567 and 2568; 2569 and 2570; 2571 and 2572; 2573 and 2574; 2575 and 2576; 2577 and 2578; 2579 and 2580; 2581 and 2582; 2583 and 2584; 2585 and 2586; 2587 and 2588; 2589 and 2590; 2591 and 2592; 2593 and 2594; 2595 and 2596; 2597 and 2598; 2599 and 2600; 2601 and 2602; 2603 and 2604; 2605 and 2606; 2607 and 2608; 2609 and 2610; 2611 and 2612; 2613 and 2614; 2615 and 2616; 2617 and 2618; 2619 and 2620; 2621 and 2622; 2623 and 2624; 2625 and 2626; 2627 and 2628; 2629 and 2630; 2631 and 2632; 2633 and 2634; 2635 and 2636; 2637 and 2638; 2639 and 2640; 2641 and 2642; 2643 and 2644; 2645 and 2646; 2647 and 2648; 2649 and 2650; 2651 and 2652; 2653 and 2654; 2655 and 2656; 2657 and 2658; 2659 and 2660; 2661 and 2662; 2663 and 2664; 2665 and 2666; 2667 and 2668; 2669 and 2670; 2671 and 2672; 2673 and 2674; 2675 and 2676; 2677 and 2678; 2679 and 2680; 2681 and 2682; 2683 and 2684; 2685 and 2686; 2687 and 2688; 2689 and 2690; 2691 and 2692; 2693 and 2694; 2695 and 2696; 2697 and 2698; 2699 and 2700; 2701 and 2702; 2703 and 2704; 2705 and 2706; 2707 and 2708; 2709 and 2710; 2711 and 2712; 2713 and 2714; 2715 and 2716; 2717 and 2718; 2719 and 2720; 2721 and 2722; 2723 and 2724; 2725 and 2726; 2727 and 2728; 2729 and 2730; 2731 and 2732; 2733 and 2734; 2735 and 2736; 2737 and 2738; 2739 and 2740; 2741 and 2742; 2743 and 2744; 2745 and 2746; 2747 and 2748; 2749 and 2750; 2751 and 2752; 2753 and 2754; 2755 and 2756; 2757 and 2758; 2759 and 2760; 2761 and 2762; 2763 and 2764; 2765 and 2766; 2767 and 2768; 2769 and 2770; 2771 and 2772; 2773 and 2774; 2775 and 2776; 2777 and 2778; 2779 and 2780; 2781 and 2782; 2783 and 2784; 2785 and 2786; 2787 and 2788; 2789 and 2790; 2791 and 2792; 2793 and 2794; 2795 and 2796; 2797 and 2798; 2799 and 2800; 2801 and 2802; 2803 and 2804; 2805 and 2806; 2807 and 2808; 2809 and 2810; 2811 and 2812; 2813 and 2814; 2815 and 2816; 2817 and 2818; 2819 and 2820; 2821 and 2822; 2823 and 2824; 2825 and 2826; 2827 and 2828; 2829 and 2830; 2831 and 2832; 2833 and 2834; 2835 and 2836; 2837 and 2838; 2839 and 2840; 2841 and 2842; 2843 and 2844; 2845 and 2846; 2847 and 2848; 2849 and 2850; 2851 and 2852; 2853 and 2854; 2855 and 2856; 2857 and 2858; 2859 and 2860; 2861 and 2862; 2863 and 2864; 2865 and 2866; 2867 and 2868; 2869 and 2870; 2871 and 2872; 2873 and 2874; 2875 and 2876; 2877 and 2878; 2879 and 2880; 2881 and 2882; 2883 and 2884; 2885 and 2886; 2887 and 2888; 2889 and 2890; 2891 and 2892; 2893 and 2894; 2895 and 2896; 2897 and 2898; 2899 and 2900; 2901 and 2902; 2903 and 2904; 2905 and 2906; 2907 and 2908; 2909 and 2910; 2911 and 2912; 2913 and 2914; 2915 and 2916; 2917 and 2918; 2919 and 2920; 2921 and 2922; 2923 and 2924; 2925 and 2926; 2927 and 2928; 2929 and 2930; 2931 and 2932; 2933 and 2934; 2935 and 2936; 2937 and 2938; 2939 and 2940; 2941 and 2942; 2943 and 2944; 2945 and 2946; 2947 and 2948; 2949 and 2950; 2951 and 2952; 2953 and 2954; 2955 and 2956; 2957 and 2958; 2959 and 2960; 2961 and 2962; 2963 and 2964; 2965 and 2966; 2967 and 2968; 2969 and 2970; 2971 and 2972; 2973 and 2974; 2975 and 2976; 2977 and 2978; 2979 and 2980; 2981 and 2982; 2983 and 2984; 2985 and 2986; 2987 and 2988; 2989 and 2990; 2991 and 2992; 2993 and 2994; 2995 and 2996; 2997 and 2998; 2999 and 3000; 3001 and 3002; 3003 and 3004; 3005 and 3006; 3007 and 3008; 3009 and 3010; 3011 and 3012; 3013 and 3014; 3015 and 3016; 3017 and 3018; 3019 and 3020; 3021 and 3022; 3023 and 3024; 3025 and 3026; 3027 and 3028; 3029 and 3030; 3031 and 3032; 3033 and 3034; 3035 and 3036; 3037 and 3038; 3039 and 3040; 3041 and 3042; 3043 and 3044; 3045 and 3046; 3047 and 3048; 3049 and 3050; 3051 and 3052; 3053 and 3054; 3055 and 3056; 3057 and 3058; 3059 and 3060; 3061 and 3062; 3063 and 3064; 3065 and 3066; 3067 and 3068; 3069 and 3070; 3071 and 3072; 3073 and 3074; 3075 and 3076; 3077 and 3078; 3079 and 3080; 3081 and 3082; 3083 and 3084; 3085 and 3086; 3087 and 3088; 3089 and 3090; 3091 and 3092; 3093 and 3094; 3095 and 3096; 3097 and 3098; 3099 and 3100; 3101 and 3102; 3103 and 3104; 3105 and 3106; 3107 and 3108; 3109 and 3110; 3111 and 3112; 3113 and 3114; 3115 and 3116; 3117 and 3118; 3119 and 3120; 3121 and 3122; 3123 and 3124; 3125 and 3126; 3127 and 3128; 3129 and 3130; 3131 and 3132; 3133 and 3134; 3135 and 3136; 3137 and 3138; 526 and 527; 528 and 529; 530 and 531; 532 and 533; 534 and 535; 536 and 537; 538 and 539; 540 and 541; 542 and 543; 544 and 545; 546 and 547; 548 and 549; 550 and 551; 552 and 553; 554 and 555; 556 and 557; 558 and 559; 560 and 561; 5215 and 5216; 5217 and 5218; 5219 and 5220; 5221 and 5222; 5223 and 5224; 5225 and 5226; 5227 and 5228; 5229 and 5230; 5231 and 5232; 5233 and 5234; 5235 and 5236; 5237 and 5238; 5239 and 5240; 5241 and 5242; 5243 and 5244; 5245 and 5246; 5247 and 5248; 5249 and 5250; 5251 and 5252; 5253 and 5254; 5255 and 5256; 5257 and 5258; 5259 and 5260; 5261 and 5262; 5263 and 5264; 5265 and 5266; 5267 and 5268; 5269 and 5270; 5271 and 5272; 5273 and 5274; 5275 and 5276; 5277 and 5278; 5279 and 5280; 5281 and 5282; 5283 and 5284; 5285 and 5286; 5287 and 5288; 5289 and 5290; 5291 and 5292; 5293 and 5294; 5295 and 5296; 5297 and 5298; 5299 and 5300; 5301 and 5302; 5303 and 5304; 5305 and 5306; 5307 and 5308; 5309 and 5310; 5311 and 5312; 5313 and 5314; 577 and 578; 579 and 580; 581 and 582; 583 and 584; 585 and 586; 587 and 588; 589 and 590; 591 and 592; 593 and 594; 595 and 596; 597 and 598; 599 and 600; 601 and 602; 603 and 604; 605 and 606; 607 and 608; 609 and 610; 611 and 612; 613 and 614; 615 and 616; 617 and 618; 619 and 620; 621 and 622; 623 and 624; 625 and 626; 627 and 628; 629 and 630; 631 and 632; 633 and 634; 635 and 636; 5315 and 5316; 5317 and 5318; 5319 and 5320; 5321 and 5322; 5323 and 5324; 5325 and 5326; 5327 and 5328; 5329 and 5330; 5331 and 5332; 5333 and 5334; 5335 and 5336; 5337 and 5338; 5339 and 5340; 5341 and 5342; 5343 and 5344; 5345 and 5346; 5347 and 5348; 5349 and 5350; 5351 and 5352; 5353 and 5354; 5355 and 5356; 5357 and 5358; 5359 and 5360; 5361 and 5362; 5363 and 5364; 5365 and 5366; 5367 and 5368; 5369 and 5370; 5371 and 5372; 5373 and 5374; 5375 and 5376; 5377 and 5378; 5379 and 5380; 5381 and 5382; 5383 and 5384; 5385 and 5386; 5387 and 5388; 5389 and 5390; 5391 and 5392; 5393 and 5394; 5395 and 5396; 5397 and 5398; 5399 and 5400; 5401 and 5402; 5403 and 5404; 5405 and 5406; 5407 and 5408; 5409 and 5410; 5411 and 5412; 5413 and 5414; 5415 and 5416; 5417 and 5418; 5419 and 5420; 5421 and 5422; 5423 and 5424; 5425 and 5426; 5427 and 5428; 5429 and 5430; 5431 and 5432; 5433 and 5434; 5435 and 5436; 5437 and 5438; 5439 and 5440; 5441 and 5442; 5443 and 5444; 5445 and 5446; 5447 and 5448; 5449 and 5450; 5451 and 5452; 5453 and 5454; 5455 and 5456; 5457 and 5458; 5459 and 5460; 5461 and 5462; 5463 and 5464; 5465 and 5466; 5467 and 5468; 5469 and 5470; 5471 and 5472; 5473 and 5474; 5475 and 5476; 5477 and 5478; 5479 and 5480; 5481 and 5482; 5483 and 5484; 5485 and 5486; 5487 and 5488; 5489 and 5490; 5491 and 5492; 5493 and 5494; 5495 and 5496; 5497 and 5498; 5499 and 5500; 5501 and 5502; 5503 and 5504; 5505 and 5506; 5507 and 5508; 5509 and 5510; 5511 and 5512; 5513 and 5514; 5515 and 5516; 5517 and 5518; 643 and 644; 645 and 646; 647 and 648; 649 and 650; 651 and 652; 653 and 654; 655 and 656; 657 and 658; 659 and 660; 661 and 662; 663 and 664; 665 and 666; 4041 and 4042; 4043 and 4044; 4045 and 4046; 4047 and 4048; 4049 and 4050; 4051 and 4052; 4053 and 4054; 4055 and 4056; 4057 and 4058; 4059 and 4060; 4061 and 4062; 4063 and 4064; 4065 and 4066; 4067 and 4068; 4069 and 4070; 4071 and 4072; 4073 and 4074; 4075 and 4076; 4077 and 4078; 4079 and 4080; 4081 and 4082; 4083 and 4084; 4085 and 4086; 4087 and 4088; 4089 and 4090; 4091 and 4092; 4093 and 4094; 4095 and 4096; 4097 and 4098; 4099 and 4100; 4101 and 4102; 4103 and 4104; 4105 and 4106; 4107 and 4108; 689 and 690; 691 and 692; 693 and 694; 695 and 696; 697 and 698; 699 and 700; 701 and 702; 703 and 704; 705 and 706; 707 and 708; 709 and 710; 711 and 712; 713 and 714; 715 and 716; 717 and 718; 719 and 720; 721 and 722; 723 and 724; 725 and 726; 727 and 728; 729 and 730; 731 and 732; 733 and 734; 735 and 736; 737 and 738; 739 and 740; 741 and 742; 743 and 744; 745 and 746; 747 and 748; 749 and 750; 751 and 752; 753 and 754; 755 and 756; 757 and 758; 759 and 760; 761 and 762; 763 and 764; 765 and 766; 767 and 768; 769 and 770; 771 and 772; 773 and 774; 775 and 776; 3397 and 3398; 3399 and 3400; 3401 and 3402; 3403 and 3404; 3405 and 3406; 3407 and 3408; 3409 and 3410; 3411 and 3412; 3413 and 3414; 3415 and 3416; 3417 and 3418; 3419 and 3420; 3421 and 3422; 3423 and 3424; 3425 and 3426; 3427 and 3428; 3429 and 3430; 3431 and 3432; 3433 and 3434; 3435 and 3436; 3437 and 3438; 3439 and 3440; 3441 and 3442; 3443 and 3444; 3445 and 3446; 3447 and 3448; 3449 and 3450; 3451 and 3452; 3453 and 3454; 3455 and 3456; 3457 and 3458; 3459 and 3460; 3461 and 3462; 3463 and 3464; 3465 and 3466; 3467 and 3468; 3469 and 3470; 3471 and 3472; 3473 and 3474; 3475 and 3476; 3477 and 3478; 3479 and 3480; 3481 and 3482; 3483 and 3484; 3485 and 3486; 3487 and 3488; 3489 and 3490; 3491 and 3492; 3493 and 3494; 3495 and 3496; 3497 and 3498; 3499 and 3500; 3501 and 3502; 3503 and 3504; 3505 and 3506; 3507 and 3508; 3509 and 3510; 3511 and 3512; 3513 and 3514; 3515 and 3516; 3517 and 3518; 3519 and 3520; 3521 and 3522; 3523 and 3524; 3525 and 3526; 3527 and 3528; 3529 and 3530; 3531 and 3532; 3533 and 3534; 3535 and 3536; 3537 and 3538; 3539 and 3540; 3541 and 3542; 3543 and 3544; 3545 and 3546; 3547 and 3548; 3549 and 3550; 3551 and 3552; 3553 and 3554; 3555 and 3556; 3557 and 3558; 3559 and 3560; 3561 and 3562; 3563 and 3564; 3565 and 3566; 3567 and 3568; 3569 and 3570; 3571 and 3572; 3573 and 3574; 3575 and 3576; 3577 and 3578; 3579 and 3580; 3581 and 3582; 3583 and 3584; 3585 and 3586; 3587 and 3588; 3589 and 3590; 3591 and 3592; 3593 and 3594; 3595 and 3596; 3597 and 3598; 803 and 804; 805 and 806; 807 and 808; 809 and 810; 811 and 812; 813 and 814; 815 and 816; 817 and 818; 819 and 820; 821 and 822; 823 and 824; 825 and 826; 827 and 828; 829 and 830; 831 and 832; 833 and 834; 835 and 836; 837 and 838; 839 and 840; 841 and 842; 843 and 844; 845 and 846; 847 and 848; 849 and 850; 851 and 852; 853 and 854; 855 and 856; 857 and 858; 859 and 860; 861 and 862; 863 and 864; 865 and 866; 867 and 868; 869 and 870; 871 and 872; 873 and 874; 875 and 876; 877 and 878; 879 and 880; 881 and 882; 883 and 884; 885 and 886; 887 and 888; 889 and 890; 891 and 892; 893 and 894; 895 and 896; 897 and 898; 899 and 900; 901 and 902; 903 and 904; 3599 and 3600; 3601 and 3602; 3603 and 3604; 3605 and 3606; 3607 and 3608; 3609 and 3610; 3611 and 3612; 3613 and 3614; 3615 and 3616; 3617 and 3618; 3619 and 3620; 3621 and 3622; 3623 and 3624; 3625 and 3626; 3627 and 3628; 3629 and 3630; 3631 and 3632; 3633 and 3634; 3635 and 3636; 3637 and 3638; 3639 and 3640; 3641 and 3642; 3643 and 3644; 3645 and 3646; 3647 and 3648; 3649 and 3650; 3651 and 3652; 3653 and 3654; 3655 and 3656; 3657 and 3658; 3659 and 3660; 3661 and 3662; 3663 and 3664; 3665 and 3666; 3667 and 3668; 3669 and 3670; 3671 and 3672; 3673 and 3674; 3675 and 3676; 3677 and 3678; 3679 and 3680; 3681 and 3682; 3683 and 3684; 3685 and 3686; 3687 and 3688; 3689 and 3690; 3691 and 3692; 3693 and 3694; 3695 and 3696; 3697 and 3698; 3699 and 3700; 3701 and 3702; 3703 and 3704; 3705 and 3706; 3707 and 3708; 3709 and 3710; 3711 and 3712; 3713 and 3714; 3715 and 3716; 3717 and 3718; 3719 and 3720; 3721 and 3722; 3723 and 3724; 3725 and 3726; 3727 and 3728; 3729 and 3730; 3731 and 3732; 3733 and 3734; 3735 and 3736; 3737 and 3738; 3739 and 3740; 3741 and 3742; 3743 and 3744; 3745 and 3746; 3747 and 3748; 3749 and 3750; 3751 and 3752; 3753 and 3754; 3755 and 3756; 3757 and 3758; 3759 and 3760; 914 and 915; SEQ ID NO: 916 and 917; SEQ ID NO: 918 and 919; SEQ ID NO: 920 and 921; SEQ ID NO: 922 and 923; SEQ ID NO: 924 and 925; SEQ ID NO: 926 and 927; SEQ ID NO: 928 and 929; SEQ ID NO: 930 and 931; SEQ ID NO: 932 and 933; SEQ ID NO: 934 and 935; SEQ ID NO: 936 and 937; SEQ ID NO: 938 and 939; SEQ ID NO: 940 and 941; SEQ ID NO: 3761 and 3762; SEQ ID NO: 3763 and 3764; SEQ ID NO: 3765 and 3766; SEQ ID NO: 3767 and 3768; SEQ ID NO: 3769 and 3770; SEQ ID NO: 3771 and 3772; SEQ ID NO: 3773 and 3774; SEQ ID NO: 3775 and 3776; SEQ ID NO: 3777 and 3778; SEQ ID NO: 3779 and 3780; SEQ ID NO: 3781 and 3782; SEQ ID NO: 3783 and 3784; SEQ ID NO: 3785 and 3786; SEQ ID NO: 3787 and 3788; SEQ ID NO: 3789 and 3790; SEQ ID NO: 3791 and 3792; SEQ ID NO: 3793 and 3794; SEQ ID NO: 3795 and 3796; SEQ ID NO: 3797 and 3798; SEQ ID NO: 3799 and 3800; SEQ ID NO: 3801 and 3802; SEQ ID NO: 3803 and 3804; SEQ ID NO: 3805 and 3806; SEQ ID NO: 3807 and 3808; SEQ ID NO: 3809 and 3810; SEQ ID NO: 3811 and 3812; SEQ ID NO: 3813 and 3814; SEQ ID NO: 3815 and 3816; SEQ ID NO: 3817 and 3818; SEQ ID NO: 3819 and 3820; SEQ ID NO: 3821 and 3822; SEQ ID NO: 3823 and 3824; SEQ ID NO: 3825 and 3826; SEQ ID NO: 3827 and 3828; SEQ ID NO: 3829 and 3830; SEQ ID NO: 3831 and 3832; SEQ ID NO: 3833 and 3834; SEQ ID NO: 3835 and 3836; SEQ ID NO: 3837 and 3838; SEQ ID NO: 3839 and 3840; SEQ ID NO: 3841 and 3842; 965 and 966; 967 and 968; 969 and 970; 971 and 972; 973 and 974; 975 and 976; 977 and 978; 979 and 980; 981 and 982; 983 and 984; 985 and 986; 987 and 988; 989 and 990; 991 and 992; 993 and 994; 995 and 996; 997 and 998; 999 and 1000; 1001 and 1002; 1003 and 1004; 1005 and 1006; 1007 and 1008; 1009 and 1010; 1011 and 1012; 1013 and 1014; 1015 and 1016; 1017 and 1018; 1019 and 1020; 1021 and 1022; 1023 and 1024; 1025 and 1026; 1027 and 1028; 1029 and 1030; 1031 and 1032; 1033 and 1034; 1035 and 1036; 1037 and 1038; 1039 and 1040; 1041 and 1042; 1043 and 1044; 1045 and 1046; 1047 and 1048; 1049 and 1050; 1051 and 1052; 1053 and 1054; 1055 and 1056; 3843 and 3844; 3845 and 3846; 3847 and 3848; 3849 and 3850; 3851 and 3852; 3853 and 3854; 3855 and 3856; 3857 and 3858; 3859 and 3860; 3861 and 3862; 3863 and 3864; 3865 and 3866; 3867 and 3868; 3869 and 3870; 3871 and 3872; 3873 and 3874; 3875 and 3876; 3877 and 3878; 3879 and 3880; 3881 and 3882; 3883 and 3884; 3885 and 3886; 3887 and 3888; 3889 and 3890; 3891 and 3892; 3893 and 3894; 3895 and 3896; 3897 and 3898; 3899 and 3900; 3901 and 3902; 3903 and 3904; 3905 and 3906; 3907 and 3908; 3909 and 3910; 3911 and 3912; 3913 and 3914; 3915 and 3916; 3917 and 3918; 3919 and 3920; 3921 and 3922; 3923 and 3924; 3925 and 3926; 3927 and 3928; 3929 and 3930; 3931 and 3932; 3933 and 3934; 3935 and 3936; 3937 and 3938; 3939 and 3940; 3941 and 3942; 3943 and 3944; 3945 and 3946; 3947 and 3948; 3949 and 3950; 3951 and 3952; 3953 and 3954; 3955 and 3956; 3957 and 3958; 3959 and 3960; 3961 and 3962; 3963 and 3964; 3965 and 3966; 3967 and 3968; 3969 and 3970; 3971 and 3972; 3973 and 3974; 3975 and 3976; 3977 and 3978; 3979 and 3980; 3981 and 3982; 3983 and 3984; 3985 and 3986; 3987 and 3988; 3989 and 3990; 3991 and 3992; 3993 and 3994; 3995 and 3996; 3997 and 3998; 3999 and 4000; 4001 and 4002; 4003 and 4004; 4005 and 4006; 4007 and 4008; 4009 and 4010; 4011 and 4012; 4013 and 4014; 4015 and 4016; 4017 and 4018; 4019 and 4020; 4021 and 4022; 4023 and 4024; 4025 and 4026; 4027 and 4028; 4029 and 4030; 4031 and 4032; 4033 and 4034; 4035 and 4036; 4037 and 4038; 4039 and 4040; 1062 and 1071; 1064 and 1073; 1066 and 1075; 1068 and 1077; 1070 and 1079; 1080 and 1081; 1082 and 1083; 1084 and 1085; 1086 and 1087; 1088 and 1089; 4109 and 4110; 4111 and 4112; 4113 and 4114; 4115 and 4116; 4117 and 4118; 4119 and 4120; 4121 and 4122; 4123 and 4124; 4125 and 4126; 4127 and 4128; 4129 and 4130; 4131 and 4132; 4133 and 4134; 4135 and 4136; 4137 and 4138; 4139 and 4140; 4141 and 4142; 4143 and 4144; 4145 and 4146; 4147 and 4148; 4149 and 4150; 4151 and 4152; 4153 and 4154; 4155 and 4156; 4157 and 4158; 4159 and 4160; 4161 and 4162; 4163 and 4164; 4165 and 4166; 4167 and 4168; 4169 and 4170; 4171 and 4172; 4173 and 4174; 4175 and 4176; 4177 and 4178; 4179 and 4180; 4181 and 4182; 4183 and 4184; 4185 and 4186; 4187 and 4188; 4189 and 4190; 4191 and 4192; 4193 and 4194; 4195 and 4196; 4197 and 4198; 4199 and 4200; 4201 and 4202; 4203 and 4204; 4205 and 4206; 4207 and 4208; 1101 and 1102; 1103 and 1104; 1105 and 1106; 1107 and 1108; 1109 and 1110; 1111 and 1112; 1113 and 1114; 1115 and 1116; 1117 and 1118; 1119 and 1120; 1121 and 1122; 1123 and 1124; 1125 and 1126; 1127 and 1128; 1129 and 1130; 1131 and 1132; 1133 and 1134; 1135 and 1136; 1137 and 1138; 1139 and 1140; 1141 and 1142; 1143 and 1144; 4209 and 4210; 4211 and 4212; 4213 and 4214; 4215 and 4216; 4217 and 4218; 4219 and 4220; 4221 and 4222; 4223 and 4224; 4225 and 4226; 4227 and 4228; 4229 and 4230; 4231 and 4232; 4233 and 4234; 4235 and 4236; 4237 and 4238; 4239 and 4240; 4241 and 4242; 4243 and 4244; 4245 and 4246; 4247 and 4248; 4249 and 4250; 4251 and 4252; 4253 and 4254; 4255 and 4256; 4257 and 4258; 4259 and 4260; 4261 and 4262; 4263 and 4264; 4265 and 4266; 4267 and 4268; 4269 and 4270; 4271 and 4272; 4273 and 4274; 4275 and 4276; 4277 and 4278; 4279 and 4280; 4281 and 4282; 4283 and 4284; 4285 and 4286; 4287 and 4288; 4289 and 4290; 4291 and 4292; 4293 and 4294; 4295 and 4296; 4297 and 4298; 4299 and 4300; 4301 and 4302; 4303 and 4304; 4305 and 4306; 4307 and 4308; 1150 and 1151; 1152 and 1153; 1154 and 1155; 1156 and 1157; 1158 and 1159; 1160 and 1161; 1162 and 1163; 1164 and 1165; 1166 and 1167; 1168 and 1169; 4509 and 4510; 4511 and 4512; 4513 and 4514; 4515 and 4516; 4517 and 4518; 4519 and 4520; 4521 and 4522; 4523 and 4524; 4525 and 4526; 4527 and 4528; 4529 and 4530; 4531 and 4532; 4533 and 4534; 4535 and 4536; 4537 and 4538; 4539 and 4540; 4541 and 4542; 4543 and 4544; 4545 and 4546; 4547 and 4548; 4549 and 4550; 4551 and 4552; 4553 and 4554; 4555 and 4556; 4557 and 4558; 4559 and 4560; 4561 and 4562; 4563 and 4564; 4565 and 4566; 4567 and 4568; 4569 and 4570; 4571 and 4572; 4573 and 4574; 4575 and 4576; 4577 and 4578; 4579 and 4580; 4581 and 4582; 4583 and 4584; 4585 and 4586; 4587 and 4588; 4589 and 4590; 4591 and 4592; 4593 and 4594; 4595 and 4596; 4597 and 4598; 4599 and 4600; 4601 and 4602; 4603 and 4604; 4605 and 4606; 4607 and 4608; 1186 and 1187; 1188 and 1189; 1190 and 1191; 1192 and 1193; 1194 and 1195; 1196 and 1197; 1198 and 1199; 1200 and 1201; 1202 and 1203; 1204 and 1205; 1206 and 1207; 1208 and 1209; 1210 and 1211; 1212 and 1213; 1214 and 1215; 1216 and 1217; 1218 and 1219; 1220 and 1221; 1222 and 1223; 1224 and 1225; 1226 and 1227; 1228 and 1229; 1230 and 1231; 1232 and 1233; 1234 and 1235; 1236 and 1237; 1238 and 1239; 1240 and 1241; 1242 and 1243; 1244 and 1245; 1246 and 1247; 1248 and 1249; 4309 and 4310; 4311 and 4312; 4313 and 4314; 4315 and 4316; 4317 and 4318; 4319 and 4320; 4321 and 4322; 4323 and 4324; 4325 and 4326; 4327 and 4328; 4329 and 4330; 4331 and 4332; 4333 and 4334; 4335 and 4336; 4337 and 4338; 4339 and 4340; 4341 and 4342; 4343 and 4344; 4345 and 4346; 4347 and 4348; 4349 and 4350; 4351 and 4352; 4353 and 4354; 4355 and 4356; 4357 and 4358; 4359 and 4360; 4361 and 4362; 4363 and 4364; 4365 and 4366; 4367 and 4368; 4369 and 4370; 4371 and 4372; 4373 and 4374; 4375 and 4376; 4377 and 4378; 4379 and 4380; 4381 and 4382; 4383 and 4384; 4385 and 4386; 4387 and 4388; 4389 and 4390; 4391 and 4392; 4393 and 4394; 4395 and 4396; 4397 and 4398; 4399 and 4400; 4401 and 4402; 4403 and 4404; 4405 and 4406; 4407 and 4408; 4409 and 4410; 4411 and 4412; 4413 and 4414; 4415 and 4416; 4417 and 4418; 4419 and 4420; 4421 and 4422; 4423 and 4424; 4425 and 4426; 4427 and 4428; 4429 and 4430; 4431 and 4432; 4433 and 4434; 4435 and 4436; 4437 and 4438; 4439 and 4440; 4441 and 4442; 4443 and 4444; 4445 and 4446; 4447 and 4448; 4449 and 4450; 4451 and 4452; 4453 and 4454; 4455 and 4456; 4457 and 4458; 4459 and 4460; 4461 and 4462; 4463 and 4464; 4465 and 4466; 4467 and 4468; 4469 and 4470; 4471 and 4472; 4473 and 4474; 4475 and 4476; 4477 and 4478; 4479 and 4480; 4481 and 4482; 4483 and 4484; 4485 and 4486; 4487 and 4488; 4489 and 4490; 4491 and 4492; 4493 and 4494; 4495 and 4496; 4497 and 4498; 4499 and 4500; 4501 and 4502; 4503 and 4504; 4505 and 4506; 4507 and 4508; 1269 and 1270; 1271 and 1272; 1273 and 1274; 1275 and 1276; 1277 and 1278; 1279 and 1280; 1281 and 1282; 1283 and 1284; 1285 and 1286; 1287 and 1288; 1289 and 1290; 1291 and 1292; 1293 and 1294; 1295 and 1296; 1297 and 1298; 1299 and 1300; 1301 and 1302; 1303 and 1304; 1305 and 1306; 1307 and 1308; 1309 and 1310; 1311 and 1312; 1313 and 1314; 1315 and 1316; 1317 and 1318; 1319 and 1320; 1321 and 1322; 1323 and 1324; 1325 and 1326; 1327 and 1328; 1329 and 1330; 1331 and 1332; 1333 and 1334; 1335 and 1336; 1337 and 1338; 1339 and 1340; 1341 and 1342; 1343 and 1344; 4709 and 4710; 4711 and 4712; 4713 and 4714; 4715 and 4716; 4717 and 4718; 4719 and 4720; 4721 and 4722; 4723 and 4724; 4725 and 4726; 4727 and 4728; 4729 and 4730; 4731 and 4732; 4733 and 4734; 4735 and 4736; 4737 and 4738; 4739 and 4740; 4741 and 4742; 4743 and 4744; 4745 and 4746; 4747 and 4748; 4749 and 4750; 4751 and 4752; 4753 and 4754; 4755 and 4756; 4757 and 4758; 4759 and 4760; 4761 and 4762; 4763 and 4764; 4765 and 4766; 4767 and 4768; 4769 and 4770; 4771 and 4772; 4773 and 4774; 4775 and 4776; 4777 and 4778; 4779 and 4780; 4781 and 4782; 4783 and 4784; 4785 and 4786; 4787 and 4788; 4789 and 4790; 4791 and 4792; 4793 and 4794; 4795 and 4796; 4797 and 4798; 4799 and 4800; 4801 and 4802; 4803 and 4804; 4805 and 4806; 4807 and 4808; 4809 and 4810; 4811 and 4812; 4813 and 4814; 4815 and 4816; 4817 and 4818; 4819 and 4820; 4821 and 4822; 4823 and 4824; 4825 and 4826; 4827 and 4828; 4829 and 4830; 4831 and 4832; 4833 and 4834; 4835 and 4836; 4837 and 4838; 4839 and 4840; 4841 and 4842; 4843 and 4844; 4845 and 4846; 4847 and 4848; 4849 and 4850; 4851 and 4852; 4853 and 4854; 4855 and 4856; 4857 and 4858; 4859 and 4860; 4861 and 4862; 4863 and 4864; 4865 and 4866; 4867 and 4868; 4869 and 4870; 4871 and 4872; 4873 and 4874; 4875 and 4876; 4877 and 4878; 4879 and 4880; 4881 and 4882; 4883 and 4884; 4885 and 4886; 4887 and 4888; 4889 and 4890; 4891 and 4892; 4893 and 4894; 4895 and 4896; 4897 and 4898; 4899 and 4900; 4901 and 4902; 4903 and 4904; 4905 and 4906; 4907 and 4908; 1361 and 1362; 1363 and 1364; 1365 and 1366; 1367 and 1368; 1369 and 1370; 1371 and 1372; 1373 and 1374; 1375 and 1376; 1377 and 1378; 1379 and 1380; 1381 and 1382; 1383 and 1384; 1385 and 1386; 1387 and 1388; 1389 and 1390; 1393 and 1394; 1395 and 1396; 1397 and 1398; 1399 and 1400; 1401 and 1402; 1403 and 1404; 1405 and 1406; 1407 and 1408; 1409 and 1410; 1411 and 1412; 1413 and 1414; 1415 and 1416; 1417 and 1418; 1419 and 1420; 1421 and 1422; 5075 and 5076; 5077 and 5078; 5079 and 5080; 5081 and 5082; 5083 and 5084; 5085 and 5086; 5087 and 5088; 5089 and 5090; 5091 and 5092; 5093 and 5094; 5095 and 5096; 5097 and 5098; 5099 and 5100; 5101 and 5102; 5103 and 5104; 5105 and 5106; 5107 and 5108; 5109 and 5110; 5111 and 5112; 5113 and 5114; 1436 and 1437; 1438 and 1439; 1440 and 1441; 1442 and 1443; 1444 and 1445; 1446 and 1447; 1448 and 1449; 1450 and 1451; 1452 and 1453; 1454 and 1455; 1456 and 1457; 1458 and 1459; 1460 and 1461; 1462 and 1463; 1464 and 1465; 1466 and 1467; 1468 and 1469; 1470 and 1471; 1472 and 1473; 1474 and 1475; 1476 and 1477; 1478 and 1479; 5115 and 5116; 5117 and 5118; 5119 and 5120; 5121 and 5122; 5123 and 5124; 5125 and 5126; 5127 and 5128; 5129 and 5130; 5131 and 5132; 5133 and 5134; 5135 and 5136; 5137 and 5138; 5139 and 5140; 5141 and 5142; 5143 and 5144; 5145 and 5146; 5147 and 5148; 5149 and 5150; 5151 and 5152; 5153 and 5154; 5155 and 5156; 5157 and 5158; 5159 and 5160; 5161 and 5162; 5163 and 5164; 5165 and 5166; 5167 and 5168; 5169 and 5170; 5171 and 5172; 5173 and 5174; 5175 and 5176; 5177 and 5178; 5179 and 5180; 5181 and 5182; 5183 and 5184; 5185 and 5186; 5187 and 5188; 5189 and 5190; 5191 and 5192; 5193 and 5194; 5195 and 5196; 5197 and 5198; 5199 and 5200; 5201 and 5202; 5203 and 5204; 5205 and 5206; 5207 and 5208; 5209 and 5210; 5211 and 5212; 5213 and 5214; 1501 and 1502; 1503 and 1504; 1505 and 1506; 1507 and 1508; 1509 and 1510; 1511 and 1512; 1513 and 1514; 1515 and 1516; 1517 and 1518; 1519 and 1520; 1521 and 1522; 1523 and 1524; 1525 and 1526; 1527 and 1528; 1529 and 1530; 1531 and 1532; 1533 and 1534; 1535 and 1536; 1537 and 1538; 1539 and 1540; 1541 and 1542; 1543 and 1544; 1545 and 1546; 1547 and 1548; 1549 and 1550; 1551 and 1552; 1553 and 1554; 1555 and 1556; 1557 and 1558; 1559 and 1560; 1561 and 1562; 1563 and 1564; 1565 and 1566; 1567 and 1568; 1569 and 1570; 1571 and 1572; 1573 and 1574; 1575 and 1576; 1577 and 1578; 1579 and 1580; 1581 and 1582; 1583 and 1584; 5519 and 5520; 5521 and 5522; 5523 and 5524; 5525 and 5526; 5527 and 5528; 5529 and 5530; 5531 and 5532; 5533 and 5534; 5535 and 5536; 5537 and 5538; 5539 and 5540; 5541 and 5542; 5543 and 5544; 5545 and 5546; 5547 and 5548; 5549 and 5550; 5551 and 5552; 5553 and 5554; 5555 and 5556; 5557 and 5558; 5559 and 5560; 5561 and 5562; 5563 and 5564; 5565 and 5566; 5567 and 5568; 5569 and 5570; 5571 and 5572; 5573 and 5574; 5575 and 5576; 5577 and 5578; 5579 and 5580; 5581 and 5582; 5583 and 5584; 5585 and 5586; 5587 and 5588; 5589 and 5590; 5591 and 5592; 5593 and 5594; 5595 and 5596; 5597 and 5598; 5599 and 5600; 5601 and 5602; 5603 and 5604; 5605 and 5606; 5607 and 5608; 5609 and 5610; 5611 and 5612; 5613 and 5614; 5615 and 5616; 5617 and 5618; 5619 and 5620; 5621 and 5622; 5623 and 5624; 5625 and 5626; 5627 and 5628; 5629 and 5630; 5631 and 5632; 5633 and 5634; 5635 and 5636; 5637 and 5638; 5639 and 5640; 5641 and 5642; 5643 and 5644; 5645 and 5646; 5647 and 5648; 5649 and 5650; 5651 and 5652; 5653 and 5654; 5655 and 5656; 5657 and 5658; 5659 and 5660; 5661 and 5662; 5663 and 5664; 5665 and 5666; 5667 and 5668; 5669 and 5670; 5671 and 5672; 5673 and 5674; 5675 and 5676; 5677 and 5678; 5679 and 5680; 5681 and 5682; 5683 and 5684; 5685 and 5686; 5687 and 5688; 5689 and 5690; 5691 and 5692; 5693 and 5694; 5695 and 5696; 5697 and 5698; 5699 and 5700; 5701 and 5702; 5703 and 5704; 5705 and 5706; 5707 and 5708; 5709 and 5710; 5711 and 5712; 5713 and 5714; 5715 and 5716; 5717 and 5718; 1612 and 1613; 1614 and 1615; 1616 and 1617; 1618 and 1619; 1620 and 1621; 1622 and 1623; 1624 and 1625; 1626 and 1627; 1628 and 1629; 1630 and 1631; 1632 and 1633; 1634 and 1635; 1636 and 1637; 1638 and 1639; 5719 and 5720; 5721 and 5722; 5723 and 5724; 5725 and 5726; 5727 and 5728; 5729 and 5730; 5731 and 5732; 5733 and 5734; 5735 and 5736; 5737 and 5738; 5739 and 5740; 5741 and 5742; 5743 and 5744; 5745 and 5746; 5747 and 5748; 5749 and 5750; 5751 and 5752; 5753 and 5754; 5755 and 5756; 5757 and 5758; 5759 and 5760; 5761 and 5762; 5763 and 5764; 5765 and 5766; 5767 and 5768; 5769 and 5770; 5771 and 5772; 5773 and 5774; 5775 and 5776; 5777 and 5778; 5779 and 5780; 5781 and 5782; 5783 and 5784; 5785 and 5786; 5787 and 5788; 5789 and 5790; 1648 and 1649; 1650 and 1651; 1652 and 1653; 1654 and 1655; 1656 and 1657; 1658 and 1659; 1660 and 1661; 1662 and 1663; 1664 and 1665; 1666 and 1667; 1668 and 1669; 1670 and 1671; 1672 and 1673; 1674 and 1675; 1676 and 1677; 1678 and 1679; 3139 and 3140; 3141 and 3142; 3143 and 3144; 3145 and 3146; 3147 and 3148; 3149 and 3150; 3151 and 3152; 3153 and 3154; 3155 and 3156; 3157 and 3158; 3159 and 3160; 3161 and 3162; 3163 and 3164; 3165 and 3166; 3167 and 3168; 3169 and 3170; 3171 and 3172; 3173 and 3174; 3175 and 3176; 3177 and 3178; 3179 and 3180; 3181 and 3182; 3183 and 3184; 3185 and 3186; 3187 and 3188; 3189 and 3190; 3191 and 3192; 3193 and 3194; 3195 and 3196; 3197 and 3198; 3199 and 3200; 3201 and 3202; 3203 and 3204; 3205 and 3206; 3207 and 3208; 3209 and 3210; 3211 and 3212; 3213 and 3214; 3215 and 3216; 1691 and 1692; 1693 and 1694; 1695 and 1696; 1697 and 1698; 1699 and 1700; 1701 and 1702; 1703 and 1704; 1705 and 1706; 1707 and 1708; 1709 and 1710; 1711 and 1712; 1713 and 1714; 1715 and 1716; 1717 and 1718; 1719 and 1720; 1721 and 1722; 1723 and 1724; 1725 and 1726; 1727 and 1728; 1729 and 1730; 1731 and 1732; 1733 and 1734; 2261 and 2262; 2263 and 2264; 2265 and 2266; 2267 and 2268; 2269 and 2270; 2271 and 2272; 2273 and 2274; 2275 and 2276; 2277 and 2278; 2279 and 2280; 2281 and 2282; 2283 and 2284; 2285 and 2286; 2287 and 2288; 2289 and 2290; 2291 and 2292; 2293 and 2294; 2295 and 2296; 2297 and 2298; 2299 and 2300; 2301 and 2302; 2303 and 2304; 2305 and 2306; 2307 and 2308; 2309 and 2310; 2311 and 2312; 2313 and 2314; 2315 and 2316; 2317 and 2318; 2319 and 2320; 2321 and 2322; 2323 and 2324; 2325 and 2326; 2327 and 2328; 2329 and 2330; 2331 and 2332; 2333 and 2334; 2335 and 2336; 2337 and 2338; 2339 and 2340; 2341 and 2342; 2343 and 2344; 2345 and 2346; 2347 and 2348; 2349 and 2350; 2351 and 2352; 2353 and 2354; 2355 and 2356; 2357 and 2358; 2359 and 2360; 1752 and 1753; 1754 and 1755; 1756 and 1757; 1758 and 1759; 1760 and 1761; 1762 and 1763; 1764 and 1765; 1766 and 1767; 1768 and 1769; 1770 and 1771; 1772 and 1773; 1774 and 1775; 1776 and 1777; 1778 and 1779; 1780 and 1781; 1782 and 1783; 1784 and 1785; 1786 and 1787; 1788 and 1789; 1790 and 1791; 1792 and 1793; 1794 and 1795; 1796 and 1797; 1798 and 1799; 1800 and 1801; 1802 and 1803; 1804 and 1805; 1806 and 1807; 1808 and 1809; 1810 and 1811; 1812 and 1813; 1814 and 1815; 1816 and 1817; 1818 and 1819; 4909 and 4910; 4911 and 4912; 4913 and 4914; 4915 and 4916; 4917 and 4918; 4919 and 4920; 4921 and 4922; 4923 and 4924; 4925 and 4926; 4927 and 4928; 4929 and 4930; 4931 and 4932; 4933 and 4934; 4935 and 4936; 4937 and 4938; 4939 and 4940; 4941 and 4942; 4943 and 4944; 4945 and 4946; 4947 and 4948; 4949 and 4950; 4951 and 4952; 4953 and 4954; 4955 and 4956; 4957 and 4958; 4959 and 4960; 4961 and 4962; 4963 and 4964; 4965 and 4966; 4967 and 4968; 4969 and 4970; 4971 and 4972; 4973 and 4974; 4975 and 4976; 4977 and 4978; 4979 and 4980; 4981 and 4982; 4983 and 4984; 4985 and 4986; 4987 and 4988; 4989 and 4990; 4991 and 4992; 4993 and 4994; 4995 and 4996; 4997 and 4998; 4999 and 5000; 5001 and 5002; 5003 and 5004; 5005 and 5006; 5007 and 5008; 5009 and 5010; 5011 and 5012; 5013 and 5014; 5015 and 5016; 5017 and 5018; 5019 and 5020; 5021 and 5022; 5023 and 5024; 5025 and 5026; 5027 and 5028; 5029 and 5030; 5031 and 5032; 5033 and 5034; 5035 and 5036; 5037 and 5038; 5039 and 5040; 5041 and 5042; 5043 and 5044; 5045 and 5046; 5047 and 5048; 5049 and 5050; 5051 and 5052; 5053 and 5054; 5055 and 5056; 5057 and 5058; 5059 and 5060; 5061 and 5062; 5063 and 5064; 5065 and 5066; 5067 and 5068; 5069 and 5070; 5071 and 5072; 5073 and 5074; 1834 and 1835; 1836 and 1837; 1838 and 1839; 1840 and 1841; 1842 and 1843; 1844 and 1845; 1846 and 1847; 1848 and 1849; 1850 and 1851; 1852 and 1853; 1854 and 1855; 1856 and 1857; 1858 and 1859; 1860 and 1861; 1862 and 1863; 1864 and 1865; 1866 and 1867; 1868 and 1869; 1870 and 1871; 1872 and 1873; 1874 and 1875; 1876 and 1877; 1878 and 1879; 1880 and 1881; 1882 and 1883; 1884 and 1885; 1886 and 1887; 1888 and 1889; 2161 and 2162; 2163 and 2164; 2165 and 2166; 2167 and 2168; 2169 and 2170; 2171 and 2172; 2173 and 2174; 2175 and 2176; 2177 and 2178; 2179 and 2180; 2181 and 2182; 2183 and 2184; 2185 and 2186; 2187 and 2188; 2189 and 2190; 2191 and 2192; 2193 and 2194; 2195 and 2196; 2197 and 2198; 2199 and 2200; 2201 and 2202; 2203 and 2204; 2205 and 2206; 2207 and 2208; 2209 and 2210; 2211 and 2212; 2213 and 2214; 2215 and 2216; 2217 and 2218; 2219 and 2220; 2221 and 2222; 2223 and 2224; 2225 and 2226; 2227 and 2228; 2229 and 2230; 2231 and 2232; 2233 and 2234; 2235 and 2236; 2237 and 2238; 2239 and 2240; 2241 and 2242; 2243 and 2244; 2245 and 2246; 2247 and 2248; 2249 and 2250; 2251 and 2252; 2253 and 2254; 2255 and 2256; 2257 and 2258; 2259 and 2260; 1899 and 1900: 1901 and 1902: 1903 and 1904: 1905 and 1906: 1907 and 1908: 1909 and 1910: 1911 and 1912: 1913 and 1914: 1915 and 1916: 1917 and 1918: 1919 and 1920: 1921 and 1922: 1923 and 1924: 1925 and 1926: 1927 and 1928: 1929 and 1930: 1931 and 1932: 1933 and 1934: 3217 and 3218: 3219 and 3220: 3221 and 3222: 3223 and 3224: 3225 and 3226: 3227 and 3228: 3229 and 3230: 3231 and 3232: 3233 and 3234: 3235 and 3236: 3237 and 3238: 3239 and 3240: 3241 and 3242: 3243 and 3244: 3245 and 3246: 3247 and 3248: 3249 and 3250: 3251 and 3252: 3253 and 3254: 3255 and 3256: 3257 and 3258: 3259 and 3260: 3261 and 3262: 3263 and 3264: 3265 and 3266: 3267 and 3268: 3269 and 3270: 3271 and 3272: 3273 and 3274: 3275 and 3276: 3277 and 3278: 3279 and 3280: 3281 and 3282: 3283 and 3284: 3285 and 3286; 1952 and 1953; 1954 and 1955; 1956 and 1957; 1958 and 1959; 1960 and 1961; 1962 and 1963; 1964 and 1965; 1966 and 1967; 1968 and 1969; 1970 and 1971; 1972 and 1973; 1974 and 1975; 1976 and 1977; 1978 and 1979; 1980 and 1981; 1982 and 1983; 1984 and 1985; 1986 and 1987; 1988 and 1989; 1990 and 1991; 1992 and 1993; 1994 and 1995; 1996 and 1997; 1998 and 1999; 2000 and 2001; 2002 and 2003; 2004 and 2005; 2006 and 2007; 2008 and 2009; 2010 and 2011; 2012 and 2013; 2014 and 2015; 2016 and 2017; 2018 and 2019; 3287 and 3288; 3289 and 3290; 3291 and 3292; 3293 and 3294; 3295 and 3296; 3297 and 3298; 3299 and 3300; 3301 and 3302; 3303 and 3304; 3305 and 3306; 3307 and 3308; 3309 and 3310; 3311 and 3312; 3313 and 3314; 3315 and 3316; 3317 and 3318; 3319 and 3320; 3321 and 3322; 3323 and 3324; 3325 and 3326; 3327 and 3328; 3329 and 3330; 3331 and 3332; 3333 and 3334; 3335 and 3336; 3337 and 3338; 3339 and 3340; 3341 and 3342; 3343 and 3344; 3345 and 3346; 3347 and 3348; 3349 and 3350; 3351 and 3352; 3353 and 3354; 3355 and 3356; 3357 and 3358; 3359 and 3360; 3361 and 3362; 3363 and 3364; 3365 and 3366; 3367 and 3368; 3369 and 3370; 3371 and 3372; 3373 and 3374; 3375 and 3376; 3377 and 3378; 3379 and 3380; 3381 and 3382; 3383 and 3384; 3385 and 3386; 3387 and 3388; 3389 and 3390; 3391 and 3392; 3393 and 3394; 3395 and 3396.

11. The saRNA of claim 8, wherein at least one of the sense strand or the antisense strand of the saRNA comprises a 3′ tail.

12. The saRNA of claim 8, wherein the antisense strand and the sense strand independently comprise at least one modification.

13. A pharmaceutical composition comprising the saRNA of claim 1 and at least one pharmaceutically acceptable excipient.

14. A method of modulating the expression of a target gene, comprising administering the saRNA of claim 1, wherein the target gene is selected from the group consisting of APOA1, C19orf80, CDKN1B, DMD, EPO, F7, F8, FNDC5, FOXA2, FOXP3, HNF4A, IDUA, IFNG, IL10, IL19, IL2, LDLR, LMX1A, MDM2, METRNL, NR4A2, PAX5, SCN1A, SIRT1, and TH.

15. The method of claim 14, wherein the expression of the target protein is increased.

16. The method of claim 15, wherein the expression of the target protein is increased by at least 30% compared to the expression of the target protein without administering the saRNA of claim 1.

17. The method of claim 16, wherein the expression of the target protein is increased by at least 50% compared to the expression of the target protein without administering the saRNA of claim 1.
